(19) United States
(12) Patent Application Publication
(10) Pub. No.: US 2025/0314636 A1
(43) Pub. Date: Oct. 9, 2025

(54) METHOD OF PREPARING AND EXPANDING A POPULATION OF IMMUNE CELLS FOR CANCER THERAPY, POTENCY ASSAY FOR TUMOR RECOGNITION, BIOLOGICAL VACCINE PREPARATION AND EPITOPETARGET FOR ANTIBODIES

(71) Applicants: FUNDAÇÃO D. ANNA DE SOMMER CHAMPALIMAUD E DR. CARLOS MONTEZ CHAMPALIMAUD - CE, Lisboa (PT); Human Cell Co., Naperville, IL (US)

(72) Inventors: Carolina MENDONÇA GORGULHO, Lisboa (PT); Eric Serafim RAMOS DE SOUSA, Lisboa (PT); Jéssica OLIVEIRA KAMIKI, Estoril (PT); Joana RAMOS RAPAZ MENDES LÉRIAS, Odivelas (PT); Markus MAEURER, Lisboa (PT); Patrícia Alexandra FLORES ANTÓNIO, Tomar (PT); Soon Seog JEONG, Naperville, IL (US); Ridong CHEN, Naperville, IL (US)

(21) Appl. No.: 18/864,625

(22) PCT Filed: May 11, 2023
(86) PCT No.: PCT/IB2023/054882

§ 371 Date: Nov. 11, 2024

(30) Foreign Application Priority Data
May 11, 2022 (PT) 117973

(51) Int. Cl. G01N 33/50; C12N 5/09

(52) U.S. Cl. CPC G01N 33/5011; C12N 5/0693; C12N 2501/2307; C12N 2501/2315; C12N 2501/2321; C12N 2501/998; C12N 2502/30

(57) ABSTRACT

The present invention relates to a method of preparing and expanding a population of immune cells, a potency assay for tumor recognition, a biological vaccine preparation to provide anti-tumor response or an antiviral response for cancer therapy and epitopes targets for antibodies which are useful for the construction of chimeric antigen receptors.

The present invention is based on the fact that private or commonly shared tumor-associated antigens or private target antigens can be recognized by clinically relevant immune cells. Such target antigens could be used to prepare a biological vaccine preparation to provide anti-tumor response or an antiviral response by expanding a certain set of T-cells or B-cells and boosting the immune response in cancer therapy.

The present invention guides the selection of viable target antigens in designing an anti-tumor vaccine to remove potentially harmful autoimmune responses or pro-tumorigenic immune responses and aids to select the biologically and clinically most relevant set of immune cells specifically directed against cancer cells harvested from tumor infiltrating lymphocytes or from different anatomical sites for the active cellular therapy of patients with cancer.

### TECHNICAL FIELD

The present invention relates to cancer immunotherapy and discloses a method of preparing and expanding a population of immune cells for anti-cancer directed therapy. The invention further discloses a potency assay for tumor recognition, a biological vaccine preparation to provide anti-tumor responses or antiviral responses and an epitope target for antibodies.

### BACKGROUND OF THE INVENTION

### Cancer

Cancer is a major cause of death worldwide with an annual toll of almost 10 million. Last year, approximately 19.3 million people were diagnosed with cancer 1 and pancreatic cancer was the 7^{th} leading cancer-related cause of death while also being the 12^{th} most incident cancer worldwide².

Standard cancer treatments, such as surgery, chemotherapy, and radiotherapy, have demonstrated limited efficacy for the treatment of patients with pancreatic cancer. Immunotherapy emerged as a path to bypass various immune evasion mechanisms and potentiate immune cells to improve their anti-tumor functions or—not mutually exclusive—to expand and activate immune cells that have not yet been effectively recruited in a patient to contribute to clinically relevant immune responses. Several different types of immunotherapies, from the least specific to the more tumor-directed, have been designed to overcome different tumor escape mechanisms.

Pancreatic adenocarcinoma (PDAC) is one of the most lethal cancers, having a 5-year survival rate of around 5-10%³. To this date, traditional chemotherapeutics have failed to produce significant improvements in pancreatic cancer survival and research efforts have been focused on the role of the immune system in the development and progression of this malignancy⁴⁻⁶. The PDAC microenvironment tends to be pro-tumorigenic and immunosuppressive, moreover, it presents a desmoplastic phenotype that prevents T-cell infiltration, resulting in a more pronounced immune-suppressive effect, although more clinical research is needed to define immunological subtypes in PDACs, since some tumor lesions show during tumor evolution T-cell infiltration—that may be blunted by different immune-escape mechanisms 4-9.

### Immune System and Oncology Interplay

Tumors are complex ecosystems composed of neoplastic cells, extracellular matrix and accessory nonneoplastic cells which may include a broad array of inflammatory immune cells infiltrating into tumor tissue, sometimes more than 90% of a cancer lesion are non-cancer cells, consisting of connective tissue and non-transformed cells. Crosstalk between cancer cells and accessory cells contributes to tumor development. During tumor formation, the tissue architecture evolves into a specialized microenvironment that may either be acting in a pro-tumor or anti-tumor 4 directed fashion.

The landscape of the tumor microenvironment (TME) represents a dynamic ecosystem controlled by the tumor to support its growth and survival via altered levels of metabolites, cytokines, nutrients, oxygen, and expression of immune checkpoints to promote immune evasion. Cancer cells use mechanisms to avoid immune detection and attack such as contact dependent factors (expression of immune system checkpoint ligands such as PD-L1, CTLA-4, LAG-3, TIM-3, TIGIT), production of soluble immunosuppressive factors (such as IL-10 and TGF-β) and downregulation of MHC class I on tumor cells. Through these mechanisms, cancer cells can achieve general immunosuppression and tumor progression⁴.

Conventional αβ T-cells orchestrate cellular immunity by recognizing short foreign peptides bound to specialized molecular ‘presentation proteins’ called MHC molecules complexed with the peptide on the surface of antigen-presenting cells (APCs) during the initiation of a cellular immune response, or on tumor cells during the immune effector phase. Under situations of infection and malignant transformation, the recognition of an MHC-peptide complex triggers elimination of the target by T-cells by effector mechanisms such as production of perforin and granzyme granules. One of the key factors in anti-tumor directed immune responses is IFN-γ (IFN-gamma). A vast array of other cytokines plays a decisive role in anti-tumor immune responses, for instance, IL-17 production appears to be a double-edged sword; it may—for some tumor types—be helpful to fight off cancer cells early in the disease while facilitating disease progression in more advanced cancer disease, associated with the molecular phenotype signature. However, some IL-17 production is also needed for T-cells to enter the central nervous system (CNS) and may therefore be helpful if T-cell responses are needed in mediating anti-tumor directed immune responses (e.g., for metastatic disease) in the CNS.

The T-cell receptor (TCR) is comprised of an α-chain and a β-chain, which are composed of complementary determining loops (CDR1, CDR2 and CDR3) that are collectively unique to every individual TCR and amount to the enormous diversity of TCRs. The CDR3 region is the region where T-cell specificity resides and the CDR3 regions is (for αβ T-cells) between 1 and 11 amino acids long. It provides exquisite specificity for MHC class I (for CD8⁺ T-cells) or MHC class II (for CD4⁺) restricted T-cells. The TCR engages with the 3D shape of the nominal target epitope that is buried and anchored in the MHC binding cleft within designated ‘pockets’ provided by the MHC class I molecules, while MHC class II presented peptides assume a different accommodation since MHC class I molecules are closed on both ends, while MHC class II molecules are open on one end and afford to accommodate larger peptide species. Similarities in the 3D recognition of nominal target epitopes have been described and are in part due to the similar shape, charge, size, and composition of amino acids recognized by ‘cross-reactive’ T-cells reacting to even non-related target epitopes between different human, bacterial or viral peptide antigens or non-related epitopes or different origins.

Mucosal-associated invariant T-cells (MAIT) represent a different immune effector population and recognize different molecular targets, in part since they express a semi-invariant TCR; most of the MAIT family express the Vα7.2 chain, while some other MAIT representatives express a different and more diverse set of TCRs. MAIT cells are a part of the unconventional T-cell family, like γδ T-cells, that recognize metabolite antigens presented by MHC class I-like molecule (MR1) and CD1, respectively^{4,10} MAIT cells recognize not only vitamin—metabolites, yet also drugs, intermediates of cellular metabolism and possibly shared and private tumor-associated antigens, which have not yet been defined up to now.

Some subpopulations of γδ T-cells, similarly as natural-killer T (NKT) cells, respond to phosphoantigens from infected cells and cancer cells within the context of butyrophilin molecules, γδ T-cells may recognize their nominal target antigens in the context of CD1a-d, other classical or non-classical MHC molecules or—for some γδ T-cell subsets—without classical or non-classical MHC molecules. Like other unconventional T-cell subsets, the MR1-reactive T-cell family and the γδ T-cell population is far more complex than initially believed and the same has been found to be true for their nominal target epitopes. Also, TCR γδ T-cells have been found to recognize the ‘underside’ of the MR1 molecule underlying that MR1 recognition is very diverse; in part reflected by the fact that also TRAV1-2 and TRAV36 TCRs are also able to bind in a different fashion to MR1 with a different docking behavior.

### Tumor Infiltrating Lymphocytes (TIL) Therapy

Therapy of patients with tumor infiltrating lymphocytes (TIL) has been successful in patients with melanoma and achieved using expansion of TIL with interleukin-2 (IL-2) and pre-conditioning of the patient with chemotherapy, usually cyclophosphamide and fludarabine, with a response rate of patients with melanoma in the range of 50-70% and a long-term overall 5-year survival of 93% in patients who experienced a complete response. Other cancers, like cervical cancer, respond also to TIL therapy.

Patients with melanoma who previously had ipilimumab and IL-2 showed an overall response rate of 42%. Clinical response has been associated with neoepitope reactivity in TIL and certain T-cell phenotypes, for instance with a precursor or central memory T-cell subset (CD45RA⁺CCR7⁺, CD45RA⁻CCR7⁺), or other phenotypes, e.g., the CD3⁺CD8⁺CD39⁻CD69⁻ phenotype. Although patients with melanoma profit from TIL therapy, progress in TIL therapy or using checkpoint inhibitors has been less successful in patients with cancers other than melanoma. Yet the use of TIL has been extended to patients with malignancies with different origins, i.e., patients with HPV-positive cancer or patients with non-small cell lung cancer (NSCLC). There are individual patients with breast cancer, or patients with cholangiocarcinoma in whom TIL against an individual epitope are associated with clinical success, or a patient with colorectal cancer (CRC) whose TIL were directed against a mutant KRAS target epitope.

Immunological therapy of patients with PDAC has been proven in individual patients i.e., using Autologous Hematopoietic Stem Cell Transplantation (aHSCT). The TME in PDAC and immunological situation bears a unique profile as compared to other tumor types, i.e., the desmoplastic stroma and immuno-suppressing myeloid cells¹¹. Sparse T-cell infiltration has been quoted as one of the key factors for less responses for patients with PDAC, as well as a low frequency of mutations.

TIL in patients with PDAC have been compared with TIL in patients with melanoma and, the landscape of TIL infiltrates in PDAC is different, i.e., sparse TIL within the tumor, TIL found at the rim between the tumor and non-malignant tissue. These paradigms have been changed, i.e., certain patients present with TIL detected in the tumor-associated stroma, TIL from individual patients with PDAC have been reported to recognize neoepitopes and autologous tumor cells.

TIL from multiple tumor regions provide more likely a more accurate coverage of anti-tumor reactive T-cells directed against tissue from which TIL were harvested, yet also directed against tumor cells at distant sites in a different tumor-organ microenvironment (e.g., with the primary tumor in the pancreas and metastatic lesions in the liver, lung, or other organs/tissues). Neo-epitope specific T-cells have also been attempted to be isolated from peripheral blood with limited success.

TIL from patients with PDAC have been reported to recognize autologous tumor cells and/or neoepitopes from patients with PDAC after expansion. A major challenge of expanding TIL from PDAC lesions is to obtain sufficient numbers of TIL and that these TIL are indeed tumor-reactive, which was estimated to be low in TIL from epithelial cancers, with bystander T-cells (i.e., not tumor-directed T-cells) or viral-directed T-cells present in TIL preparations. Tumor antigens may be derived from frame-shift mutations, point-mutations, non-mutant immune cell target antigens that are expressed during embryonal development, antigens that are not exposed during thymic selection, antigens to which the cellular and humoral immune system has not been ‘tolerized’. Other factors, influencing immunogenicity have been less explored at this point, e.g. the different glycosylation status of proteins or other post-translational modifications.

The state of T-cell differentiation, as well as access of TIL into tissue are favorable factors. Not only the anatomical landscape of TIL, yet also the composition of TIL is differently associated with their tissue of origin, i.e., the presence of TCR γδ T-cells or MAIT in patients with pancreatic cancer may shape the orchestrated cellular immune response of ‘classical’ MHC class I or MHC class II restricted responses, as well as the interplay with immune cells that are anti-tumor directed and restricted by ‘non-classical’ MHC molecules, e.g. CD1d or MR1.

Not only αβ TIL, yet γδ TIL react to epithelial cells. Anti-CD3 stimulation and allogeneic feeder cells, or, in some protocols, co-stimulation of matrix-bound CD3-CD28 have also been used to expand anti-tumor directed immune cells. Additional stimuli have been tested to obtain ‘better’ TIL i.e., increased potassium and acetate, inducing increased ‘T-cell stemness’, or reducing T-cell senescence¹².

Furthermore, bacterial, and fungal species have been described to be associated with pancreatic cancer progression¹³, and—vice versa—with better prognosis: long-term survivors with pancreatic cancer exhibit a different microbiome¹⁴, which suggests the possible role of MAIT in pancreatic cancer outcome. Riquelme et al. (2019)¹⁵ demonstrated an intra-tumoral microbiome signature involving Pseudoxanthomas, Streptomyces, Saccharopolyspora and Bacillus that was predictive of survival in patients with pancreatic ductal adenocarcinoma. Some of these bacterial species or related families have been implicated in inflammatory diseases associated with IFN-γ production. This could imply there is cross-reactivity between T-cells that recognize tumor antigens and microbial antigens present in the tumor tissue, or tumor-associated tissue components.

Due to the above, the present application suggests that target antigens could be recognized by clinically relevant immune cells and, therefore, boost the immune response in cancer therapy when administered to a patient in need thereof as a biological vaccine preparation.

The present application provides a method of preparing and expanding a population of clinically relevant immune cells using interleukins, cardiolipin and feeder cells, wherein the expansion is facilitated by adding an antibody directed against CD3, a component of the T-cell receptor, in order to increase tumor-reactive T-cells in an immune cell product.

### PRIOR ART DOCUMENTS

Document WO 2020/172202 A1 discloses methods for manufacturing T-cells which express a novel group of cell surface receptors that recognize peptides on the surface of a target cell, as well as populations of T cells produced by the methods and pharmaceutical compositions thereof.

More specifically, the method comprises the steps of processing a biological sample containing a population of T lymphocyte cells obtained from a donor subject that has a tumor to produce a population of T lymphocyte cells and then stimulating such population with one or more T-cell stimulating agents under conditions for expansion.

In KR 102182555 B1, a method for screening a common cancer antigen or neoantigen commonly expressed in cancer tissues is disclosed. The screening method efficiently screens a peptide capable of specifically binding to a human leukocyte antigen expressed in cancer tissues. The peptide has high immunogenicity, so it can be usefully used in cancer treatment vaccines.

In document WO 2020/180648 A1, it is disclosed a method for separately isolating antigen-binding T-cells and antigen-activated T-cells derived from an initial population of peripheral blood mononuclear cells, and for identifying clonotypes of the blood receptor overlapping T cells. In this specific document, antigens include personal and shared neoantigens as well as testicular cancer antigens. T cell receptor clonotypes identified by screening anti-cancer—associated antigens, can further be used to develop cancer treatment therapies, e.g. by cloning the nominal tumor-target specific T-cell receptor which could be used with an appropriate vector system—to transfer antigen specificity restricted by ‘classical’ (e.g. MHC class I or MHC class II) or non-classical (e.g. MR1 or CD1) restricting molecules—into recipient immune cells. Summary of the Invention

The present invention is based on the fact that private or commonly shared tumor-associated antigens could be recognized by clinically relevant immune cells. Such target antigens could be used to prepare a biological vaccine preparation to provide anti-tumor response or antiviral response by expanding a certain set of T-cells or B-cells and boosting the immune response in anti-cancer directed therapy.

Therefore, the present invention relates, in a first aspect, to a method of preparing and expanding a population of immune cells, wherein a body sample is first obtained from a mammal and then cultured for three expansion steps to obtain anti-tumor directed immune cells selected from tumor-infiltrating lymphocytes, peripheral blood mononuclear cells, or immune cells harvested from non-cancer tissue from a distant anatomical site, for instance skin (devoid of cancer cells) yet provides an environment that enriches for tumor antigen specific T-cells.

In a second aspect, the invention relates to a potency assay for tumor recognition, wherein the clinically relevant immune cells previously obtained are challenged with at least one target antigen to detect a change in a cytokine or immune effector molecule production.

The present invention further relates, in a third aspect, to a biological vaccine preparation to provide anti-tumor response or antiviral response, comprising target antigens that lead to the expansion of a certain set of T-cells or B-cells. Also, the target antigen serves as a target for antibody molecules, or protein that binds specifically to it, whose responses are directed against cancer cells, which could be used for antibody therapy or construction of a chimeric antigen receptor construct.

In a fourth aspect, the present invention discloses an epitope target for antibodies that serves as a viable target that can be used to construct chimeric antigen receptors.

### BRIEF DESCRIPTION OF DRAWINGS

In order to promote an understanding of the principles according to the modalities of the present invention, reference will be made to the embodiments illustrated in the figures and the language used to describe them.

It should also be understood that there is no intention to limit the scope of the invention to the content of the figures and that modifications to the inventive features illustrated herein, as well as additional applications of the principles and embodiments illustrated, which would normally occur to a person skilled in the art having the possession of this description, are considered within the scope of the claimed invention.

FIG. 1 shows an example of a tumor resection, the first steps of TIL propagation and examples of selection of tumor areas for TIL expansion guided by anatomical structures, wherein (a) is a gross specimen after resection, (b) is the anterior view of the specimen, (c) is the posterior view of the specimen, (d) is the opened tumor specimen for TIL propagation, the entire middle section is used for TIL expansion. Parallel sections to the left and right are prepared for standard histology, immunohistology and gene expression analysis, including spatial transcriptomics and (e) is the tissue piece where TIL mobilized by the method described in the current application. Parallel sections are prepared for histopathology to guide the selection of tissues for expansion and to understand better the biology of anti-tumor responses, as well as to select the most promising T-cell infiltrating area of the tumor, while avoiding areas that may contain immune cells that may facilitate tumor progression or mediate immune-suppression.

FIG. 2 shows examples of selection of tumor areas for TIL expansion, wherein (a) is the mobilized tumor piece for TIL propagation, (b) is the dissected tumor pieces and minced into 1-3 mm³ pieces to be placed into culture vessels, ice-cold PBS aids to identify distinct anatomical areas and the selection is guided by the tumor piece anatomy and if immediately available histopathology, marker analysis, e.g. by immunohistochemistry, or by gene expression analysis, (c) is the selection of distinct tumor areas along anatomical structures, cutting of tumor areas into distinct 1 mm³ pieces that are individually placed into 24 well plates and (d) is the HE staining parallel section of the tumor piece used for TIL propagation.

FIG. 3 shows examples of the HE staining parallel section of the tumor piece used for TIL propagation, histology aids to define regions with tumor cells and TIL infiltrates as well as tertiary lymphoid structures (TLS) to link with TIL propagation, wherein (a) corresponds to histopathology in 5 mm scale, (b) corresponds to histopathology in 1 mm scale, (c) corresponds to histopathology in 100 μm scale, (d) corresponds to histopathology in 50 μm scale and (e) corresponds to histopathology in 200 μm scale.

FIG. 4 is an immunohisto-fluorescence analysis showing the association of strong IL-17 expression in distinct regions of the tumor lesion surrounded by CD3⁺ T-lymphocytes—imprinting on T-cell biology and subsequent TIL expansion.

FIG. 5 is an immunohisto-fluorescence analysis showing the association of IL-7 expression in distinct regions of the tumor lesion surrounded by CD3⁺ T-lymphocytes—imprinting on T-cell biology and subsequent TIL expansion, wherein IL-7 binding to its nominal receptor CD127 represents a strong T-cell survival factor as well as a strong anti-TGF-beta antagonist, enriching tumor-reactive TIL.

FIG. 6 shows the production of IFN-γ and IL-17A in peripheral blood mononuclear cells (PBMCs) from buffy coats, PBMCs from healthy donors and unprocessed blood from healthy donors incubated with different concentrations of cardiolipin and in the presence of the nominal target antigens and controls, wherein (a) corresponds to production of IFN-γ with titration at 0 nM at day 3 in PBMCs from buffy coats, (b) corresponds to production of IFN-γ with titration at 0 nM at day 7 in PBMCs from buffy coats, (c) corresponds to production of IFN-γ with titration at 50 nM at day 3 in PBMCs from buffy coats, (d) corresponds to production of IFN-γ with titration at 50 nM at day 7 in PBMCs from buffy coats, (e) corresponds to production of IFN-γ with titration at 150 nM at day 3 in PBMCs from buffy coats, (f) corresponds to production of IFN-γ with titration at 150 nM at day 7 in PBMCs from buffy coats, (g) corresponds to production of IFN-γ with titration at 250 nM at day 3 in PBMCs from buffy coats, (h) corresponds to production of IFN-γ with titration at 250 nM at day 7 in PBMCs from buffy coats, (i) corresponds to production of IFN-γ with titration at 350 nM at day 3 in PBMCs from buffy coats, (j) corresponds to production of IFN-γ with titration at 350 nM at day 7 in PBMCs from buffy coats, (k) corresponds to production of IFN-γ with titration at 450 nM at day 3 in PBMCs from buffy coats, (l) corresponds to production of IFN-γ with titration at 450 nM at day 7 in PBMCs from buffy coats, (m) corresponds to production of IL-17A with titration at 0 nM at day 3 in PBMCs from buffy coats, (n) corresponds to production of IL-17A with titration at 0 nM at day 7 in PBMCs from buffy coats in PBMCs from buffy coats, (o) corresponds to production of IL-17A with titration at 50 nM at day 3 in PBMCs from buffy coats, (p) corresponds to production of IL-17A with titration at 50 nM at day 7 in PBMCs from buffy coats, (q) corresponds to production of IL-17A with titration at 150 nM at day 3 in PBMCs from buffy coats, (r) corresponds to production of IL-17A with titration at 150 nM at day 7 in PBMCs from buffy coats, (s) corresponds to production of IL-17A with titration at 250 nM at day 3 in PBMCs from buffy coats, (t) corresponds to production of IL-17A with titration at 250 nM at day 7 in PBMCs from buffy coats, (u) corresponds to production of IL-17A with titration at 350 nM at day 3 in PBMCs from buffy coats, (v) corresponds to production of IL-17A with titration at 350 nM at day 3 in PBMCs from buffy coats, (w) corresponds to production of IL-17A with titration at 450 nM at day 3 in PBMCs from buffy coats, (x) corresponds to production of IL-17A with titration at 450 nM at day 7 in PBMCs from buffy coats, (y) corresponds to production of IFN-γ with titration at 0 nM at day 3 in PBMCs from healthy donors, (z) corresponds to production of IFN-γ with titration at 0 nM at day 7 in PBMCs from healthy donors, (aa) corresponds to production of IFN-γ with titration at 50 nM at day 3 in PBMCs from healthy donors, (bb) corresponds to production of IFN-γ with titration at 50 nM at day 7 in PBMCs from healthy donors, (cc) corresponds to production of IFN-γ with titration at 150 nM at day 3 in PBMCs from healthy donors, (dd) corresponds to production of IFN-γ with titration at 150 nM at day 7 in PBMCs from healthy donors, (ee) corresponds to production of IFN-γ with titration at 250 nM at day 3 in PBMCs from healthy donors, (ff) corresponds to production of IFN-γ with titration at 250 nM at day 7 in PBMCs from healthy donors, (gg) corresponds to production of IFN-γ with titration at 350 nM at day 3 in PBMCs from healthy donors, (hh) corresponds to production of IFN-γ with titration at 350 nM at day 7 in PBMCs from healthy donors, (ii) corresponds to production of IFN-γ with titration at 450 nM at day 3 in PBMCs from healthy donors, (jj) corresponds to production of IFN-γ with titration at 450 nM at day 7 in PBMCs from healthy donors, (kk) corresponds to production of IL-17A with titration at 0 nM at day 3 in PBMCs from healthy donors, (ll) corresponds to production of IL-17A with titration at 0 nM at day 7 in PBMCs from healthy donors, (mm) corresponds to production of IL-17A with titration at 50 nM at day 3 in PBMCs from healthy donors, (nn) corresponds to production of IL-17A with titration at 50 nM at day 7 in PBMCs from healthy donors, (oo) corresponds to production of IL-17A with titration at 150 nM at day 3 in PBMCs from healthy donors, (pp) corresponds to production of IL-17A with titration at 150 nM at day 7 in PBMCs from healthy donors, (qq) corresponds to production of IL-17A with titration at 250 nM at day 3 in PBMCs from healthy donors, (rr) corresponds to production of IL-17A with titration at 250 nM at day 7 in PBMCs from healthy donors, (ss) corresponds to production of IL-17A with titration at 350 nM at day 3 in PBMCs from healthy donors, (tt) corresponds to production of IL-17A with titration at 350 nM at day 7 in PBMCs from healthy donors, (uu) corresponds to production of IL-17A with titration at 450 nM at day 3 in PBMCs from healthy donors, (vv) corresponds to production of IL-17A with titration at 450 nM at day 7 in PBMCs from healthy donors, (ww) corresponds to production of IFN-γ with titration at 0 nM at day 3 in whole blood from healthy donors, (xx) corresponds to production of IFN-γ with titration at 0 nM at day 7 in whole blood from healthy donors, (yy) corresponds to production of IFN-γ with titration at 50 nM at day 3 in whole blood from healthy donors, (zz) corresponds to production of IFN-γ with titration at 50 nM at day 7 in whole blood from healthy donors, (aaa) corresponds to production of IFN-γ with production of IFN-γ with titration at 150 nM at day 3 in whole blood from healthy donors, (bbb) corresponds to production of IFN-γ with titration at 150 nM at day 7 in whole blood from healthy donors, (ccc) corresponds to production of IFN-γ with titration at 250 nM at day 3 in whole blood from healthy donors, (ddd) corresponds to production of IFN-γ with titration at 250 nM at day 7 in whole blood from healthy donors, (eee) corresponds to production of IFN-γ with titration at 350 nM at day 3 in whole blood from healthy donors, (fff) corresponds to production of IFN-γ with titration at 350 nM at day 7 in whole blood from healthy donors, (ggg) corresponds to production of IFN-γ with titration at 450 nM at day 3 in whole blood from healthy donors, (hhh) corresponds to production of IFN-γ with titration at 450 nM at day 7 in whole blood from healthy donors, (iii) corresponds to production of IL-17A with titration at 0 nM at day 3 in whole blood from healthy donors, (jjj) corresponds to production of IL-17A with titration at 0 nM at day 7 in whole blood from healthy donors, (kkk) corresponds to production of IL-17A with titration at 50 nM at day 3 in whole blood from healthy donors, (lll) corresponds to production of IL-17A with titration at 50 nM at day 7 in whole blood from healthy donors, (mmm) corresponds to production of IL-17A with titration at 150 nM at day 3 in whole blood from healthy donors, (nnn) corresponds to production of IL-17A with titration at 150 nM at day 7 in whole blood from healthy donors, (ooo) corresponds to production of IL-17A with titration at 250 nM at day 3 in whole blood from healthy donors, (ppp) corresponds to production of IL-17A with titration at 250 nM at day 7 in whole blood from healthy donors, (qqq) corresponds to titration at 350 nM at day 3 in whole blood from healthy donors, (rrr) corresponds to production of IL-17A with titration at 350 nM at day 7 in whole blood from healthy donors, (sss) corresponds to production of IL-17A with titration at 450 nM at day 3 in whole blood from healthy donors, (ttt) corresponds to production of IL-17A with titration at 450 nM at day 7 in whole blood from healthy donors. The medium (as well as all positive and antigen responses) contained IL-7, IL-15, and IL-21, which represents the initial step of the TIL expansion process. Negative control: medium alone. Positive control: OKT3—T-cell receptor cross linking via the anti-CD3 directed antibody; PHA—phytohemagglutinin. Antigens: CMV—overlapping peptides covering CMV pp65; EBNA—overlapping peptides covering EBNA3; M1—peptides covering the M1 protein from the Flu virus, a target that is conserved among different strains; ESAT-6—peptides covering the immunodominant M. tuberculosis ESAT-6 target (LEEKKGNYVVTDH); HG—immunodominant hemagglutinin target (VEPGDKITFEATGNL).

FIG. 7 is a Western Blot assay to determine mitochondrial isolation from PBMC, PBMCs pre-treated with Doxirubicin or and the PANC-1 (pancreatic cancer) tumor cell line.

FIG. 8 are histopathology photographs showing the presence of γδ⁺ T-cells that can be expanded for TIL propagation.

FIG. 9 graphically shows that cardiolipin leads to increased frequency of γδ⁺ TIL, wherein (a) is the percentage of γδ⁺ TIL expanded and (b) is the percentage of Vδ1⁺ TIL with or without cardiolipin, (c) shows that the presence of feeder cells does not affect cardiolipin expanded γδ⁺ TIL and (d) Vδ1⁺ TIL.

FIG. 10 graphically shows no difference between cells expanded with and without cardiolipin wherein (a) is CD107a induction assay, (b) is the percentage of Tregs, (c) is the production of IFN-γ by 10⁵ cells in 24 hours and (d) is the production of IL-17A by 10⁵ cells in 24 hours.

FIG. 11 graphically shows similar frequency of activation and exhaustion markers, measured by flow cytometry, in different TIL subpopulations independent of cardiolipin, wherein (a) are CD4⁺ T-cells and (b) are CD8⁺ T-cells.

FIG. 12 graphically shows the differentiation and maturation status of peripheral blood mononuclear cells expanded with the triple TIL expansion protocol, i.e., first with IL-7/1 IL-15/IL-21, followed by IL-2/IL-7/IL-15 in the presence of Cardiolipin and cross-linking of the T-cell receptor using anti-CD3-IgG1. Note that no feeder cells were used. The data show that the frequency of precursor (CD45RA⁺CCR7⁺), central memory (CD45RA⁻CCR7⁺), effector memory (CD45RA⁻CCR7⁻) and terminally differentiated effector cells (CD45RA⁺CCR7⁻) in CD8⁺ PBMCs. Each dot represents PBMCs from an individual patient with cancer. The protocol does not drive peripheral T-cells into terminal differentiation, yet rather into a memory T-cell subset which is advantageous as recipient cells for transgenic immune recognition receptors, i.e., antibodies or T-cells for CAR-T-cell development. Note a decrease in frequency of terminally differentiated effector cells and enrichment in cellular memory cells which has been associated with increased clinical responsiveness in active cellular therapy.

FIG. 13 graphically shows the increased recognition of tumor associated antigens in the presence of cardiolipin in expanded TIL, wherein (a) is control, MART-1, and infectious diseases antigens, (b) is Mucin 4 peptides, (c) Mesothelin-derived peptides and (d) KRAS peptides from wildtype or mutant KRAS molecules.

FIG. 14 graphically shows that the MCC950 inhibitor reduces the tumor associated antigen recognition in TIL underlining that Cardiolipin uses the NRLP3 pathway.

FIG. 15 shows the genes differentially expressed in TIL grown with and without cardiolipin.

FIG. 16 shows the RNA expression profile of TIL with or without cardiolipin, wherein (a) genes associated with Th1 profile, (b) genes associated with Th2 profile, (c) genes associated with Th17 profile, (d) genes associated with Tfh profile, (e) genes associated with Treg profile, (f) genes associated with T-cell activation, (g) genes associated with immune response, (h) genes associated with memory phenotype, (i) genes associated with effector differentiation phenotype, (j) genes associated with exhaustion phenotype.

FIG. 17 graphically shows seven αβ⁺ TIL lines which were tested against autologous tumor. Strong and specific recognition shown be either blocking with an anti-MHC class I or -class II (HLA-DR) specific mAb.

FIG. 18 graphically shows five γδ⁺ TIL lines which were tested against autologous cancer cells. Recognition of tumor cells is CD1d restricted using a blocking anti-CD1d antibody.

FIG. 19 is an example of T-cell repertoire in the tumor tissue, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain and (d) is the TCR δ chain.

FIG. 20 is an example of T-cell repertoire in expanded TIL, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain, (d) is the TCR δ chain and (e) is a Tumor Autologous Recognition assay showing IFN-γ production in a HLA-DR restricted fashion.

FIG. 21 is an example of the negatively sorted TCR γδ repertoire in expanded TIL, wherein each VG/D family is visualized in a different color and the size of the CDR3 region is associated with the relative frequency of RNA TCR reads, wherein (a) is the TCR γ chain, (b) is the TCR δ chain, (c) represents an tumor autologous recognition assay showing IFN-γ production in a CD1d restricted fashion and (d) is a histopathology photograph showing the presence of γδ⁺ T-cells from which this TCR γδ T-cell line was originally derived.

FIG. 22 graphically shows that four γδ⁺ TIL lines were tested for recognition of autologous IL-4/GM-CSF generated autologous APCs. That were fed with mitochondria isolated from PBMCs. Mitochondria from non-manipulated PBMCs did not lead to IFN-γ production. Doxirubicin pretreated of PBMCs, followed by the isolation of mitochondria resulted in CD1d restricted recognition in 2/4 GD⁺ TIL. The same was found to be true for autologous APCs pulsed with mitochondria from PANC-1, a pancreatic cancer tumor cell line.

FIG. 23 graphically shows seven γδ⁺ TIL lines tested for recognition of PANC-1 cells. 7/9 γδ⁺ TIL recognized PANC1 in a CD1d restricted fashion. This could not be changed by incubating PANC1 with doxirubicin, leflunamide or rapamycin. Values are pg IFN-γ/7 days.

FIG. 24 shows the results of potency assay in forty different TIL lines.

FIG. 25 shows the results of potency assay in 12 different PBMCs samples.

FIG. 26 shows the results of potency assay in forty different TIL lines wherein (a) are the controls and (b) are the MUC4 antigens.

FIG. 27 shows the results of potency assay in 12 different PBMCs samples wherein (a) are the controls and (b) are the MUC4 antigens.

FIG. 28 graphically shows the frequency of TCRVα7.2⁺ in CD3⁺ cells in PBMCs and TIL expanded from pancreatic malignancies.

FIG. 29 graphically shows the frequency of MR1-tetramer binding events, wherein (a) is the frequency of MR1-6FP tetramer-binding events in PBMCs and TIL of patients with pancreatic malignancies, (b) is the frequency of TCRVα7.2⁺ events in MR1-6FP tetramer positive cells, both in PBMCs and TIL, (c) is the frequency of MR1-5-OP-RU tetramer-binding events in PBMCs and TIL of patients with pancreatic malignancies and (d) is the frequency of TCRVα7.2⁺ and CD161⁺ events in MR1-5-OP-RU tetramer positive cells, both in PBMCs and TIL.

FIG. 30 graphically shows the immunophenotypic analysis of MAIT, wherein (A) is the frequency of CD161 in TCRVα7.2⁺ cells in PBMCs and TIL from patients with pancreatic malignancies, (B) is the frequency of CD26 in TCRVα7.2⁺ cells in PBMCs and TIL from patients with pancreatic malignancies, (C) is the frequency of CD95 in TCRVα7.2⁺ cells in PBMCs and TIL from patients with pancreatic malignancies, (D) is the frequency of CD103⁺ in TCRVα7.2⁺ cells in PBMCs and TIL from patients with pancreatic malignancies and (E) is the frequency of CD69 in TCRVα7.2⁺ cells in PBMCs and TIL from patients with pancreatic malignancies.

FIG. 31 graphically shows the production of IFN-γ (pg/mL) produced from VA7.2 T-cells reacting against autologous tumor cells from five patients. Blocking T-cell reactivity with an anti-MR1 antibody shows that VA7-2 T-cells recognize the tumor target in an MR1-restricted fashion.

FIG. 32 graphically shows the production of IFN-γ (pg/mL) of TCRVα7.2⁺ cells challenged with PANC-1 after downregulation of MR1 with siRNA in VA7-2 sorted TIL from five different patients with cancer.

FIG. 33 graphically shows the frequencies of IFN-γ, IL-17A, CD107a and Perforin in the TCRVα7.2⁺ cells (MAIT), CD8⁺, CD4⁺, DP (double-positive, i.e. CD4⁻CD8⁻) and DN (double-negative) gated in CD3⁺ T-cells of patients with a pancreatic malignancy stimulated for 8 h with (a) bacteria and (b) bacterial supernatants (bacterial products).

FIG. 34 graphically shows the frequencies of IFN-γ, IL-17A, CD107a and Perforin in the TCRVα7.2⁺ cells (MAIT), CD8⁺, CD4⁺, DP and DN gated in CD3⁺ T-cells of patients with pancreatic malignancies stimulated for 8 h with (a) bacteria and (b) bacterial supernatants (bacterial products).

FIG. 35 is an example of T-cell repertoire in the tumor tissue, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain and (d) is the TCR δ chain.

FIG. 36 is an example of T-cell repertoire in expanded TIL, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain and (d) is the TCR δ chain.

FIG. 37 is an example of sorted TCRVα7.2 repertoire in expanded TIL, where TCR VA/B or VG/D family is visualized in a different color and the size of the CDR3 region is associated with the relative frequency of RNA TCR reads, wherein (a) is the TCR α chain and (b) is the TCR β chain.

FIG. 38 is an example of T-cell repertoire in the tumor tissue, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain and (d) is the TCR δ chain.

FIG. 39 is an example of T-cell repertoire in expanded TIL, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain and (d) is the TCR δ chain.

FIG. 40 is an example of sorted TCRVα7.2 repertoire in expanded TIL, where TCR VA/B or VG/D family is visualized in a different color and the size of the CDR3 region is associated with the relative frequency of RNA TCR reads, wherein (a) is the TCR α chain and (b) is the TCR β chain.

FIG. 41 is an example of sorted MR1-6FP tetramer-binding repertoire in expanded TIL, where TCR VA/B or VG/D family is visualized in a different color and the size of the CDR3 region is associated with the relative frequency of RNA TCR reads, wherein (a) is the TCR α chain and (b) is the TCR β chain and (c) graphically shows the production of IFN-γ (pg/mL) in TIL derived from a patient with cancer in α-MR1 restricted fashion.

FIG. 42 graphically shows the percentage of γδ⁺ and TCRVα7.2⁺ cells in MR1-6FP tetramer-binding cells.

FIG. 43 is an example of the is an example of T-cell repertoire in expanded TIL, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain and (d) is the TCR δ chain.

FIG. 44 is an example of sorted MR1-6FP tetramer-binding repertoire in expanded TIL, where TCR VA/B or VG/D family is visualized in a different color and the size of the CDR3 region is associated with the relative frequency of RNA TCR reads, wherein (a) is the TCR α chain, (b) is the TCR β chain, (c) is the TCR γ chain and (d) is the TCR δ chain.

### DETAILED DESCRIPTION OF THE INVENTION

Hereinafter, the best mode for carrying out the present invention is described in detail.

The present invention discloses, in a first aspect, a method of preparing and expanding a population of immune cells directed against tumor cells, tumor-precursor cells, non-tumor cells facilitating tumor transformation or cells facilitating tumor progression for cancer therapy, comprising the steps of:
(i) providing a body sample obtained from a mammal, in particular a tissue sample or a body liquid sample, comprising tumor cells, areas of non-tumor cells and immune cells or areas of tumor cells and immune cells, immune cells in close proximity to the tumor cells or distant to tumor cells or immune cells capable of killing tumor cells or controlling tumor cell proliferation, T-cells favoring or stopping cells that promote tumor transformation or tumor progression, tumor cell activity or tumor cell movement within tissues or in the organism over a longer period of time; and
(ii) culturing the body sample to expand the immune cell populations ex vivo.

In a preferred embodiment of the present method, when the body sample is a tissue sample, such tissue is selected from tissue containing tumor cells or tissue close to cancer lesions that does not contain tumor cells, as well as healthy tissue, for instance T-cell infiltrated skin. When the body sample is a body liquid sample, such body liquid sample is selected from cerebrospinal fluid, blood or synovial, pleural effusion, bone marrow or material from the peritoneum.

The tissue samples can be obtained from patients who did not undergo any prior therapy or patients who underwent radiotherapy, chemotherapy, small-molecule drugs, or therapy with checkpoint inhibitors, or any combination thereof.

Preferably, the tissue sample is a 1-3 mm³ piece collected from a 2-3 mm distance from the artery or vein or lymph vessel of the tumor based on surgically and clinically relevant locations where recurrences often take place or areas where cancer stem cells and immune cells are located. The tissue sample is further dissected based on its anatomical or microscopical architecture and/or based on an anti-tumorigenic or pro-tumorigenic protein or gene expression profile, including epigenetic differences and/or differences in microRNA.

The culturing of the body sample to expand the population of immune cells comprises three steps, namely:
a first expansion step comprising an incubation in culture medium supplemented with the interleukins selected from IL-7, IL-15, and IL-21 and cardiolipin and optionally human serum;
a second expansion step at day 2 following the day of tumor tissue harvest with an anti-CD3 antibody, crosslinking the T-cell receptor with or without cytokine-activated irradiated feeder cells cultured (prior to adding to the TIL culture) for 4 to 168 hours in the presence of interleukins; and
a third expansion step at day 4 or 5 comprising an incubation in culture medium changed to IL-2, IL-7 and IL-15 supplemented with cardiolipin over the entire culture period with or without repetitively adding an anti-CD3 antibody, crosslinking the T-cell receptor with or without cytokine-activated irradiated feeder cells cultured for 4 to 168 hours in the presence of interleukins.

The expansion steps are performed by adding solely culture medium, or by adding no culture medium or a limited amount of culture medium depending on the concentration of the starting solution plus
adding additional amino acids selected from essential amino acids plus acetate, wherein the amino acids are in the range of 0.001 mg/L to 1 mg/L and the acetate is in the range of 0.001 mg/L to 1 mg/L;
adjusting the pH with sodium bicarbonate to the range of 7.3 to 7.5 and the glucose concentration between 1.6 and 7 g/L by adding extra glucose to the medium; and
allowing increasing lactate in the range of 1 mmol/L to 60 mmol/L.

In a preferred embodiment of the present method, the culture medium in the first expansion step further comprises interleukins selected from IL-7 ranging from 10 IU/mL to 6000 IU/mL, IL-15 ranging from 5 IU/mL to 1000 IU/mL, IL-21 ranging from 0.001 IU/mL to 100 IU/mL, cardiolipin ranging from 10 to 10000 nM, and human serum from 0.1 up to 10%.

In a preferred embodiment of the present method, the culture medium in the second expansion step further comprises an anti-CD3 antibody selected from the anti-CD3 complex, which crosslinks the T-cell receptor with or without cytokine-activated irradiated feeder cells cultured for 4 to 168 hours in the presence of interleukins. The anti-CD3 antibody ranges from 10 to 3000 ng/mL and the cytokine-activated irradiated feeder cells, when present, are ranging from 0.1 to 5 million feeder cells/well, preferably 1 million cells/well.

In the third expansion step, the culture medium is changed to IL-2, IL-7 and IL-15 and the interleukins are selected from IL-2 ranging from 300 IU/mL to 6000 IU/mL, IL-7 ranging from 10 IU/mL to 6000 IU/mL and IL-15 ranging from 10 IU/mL to 1000 IU/mL and cardiolipin ranging from 10 to 10000 nM.

When present, the anti-CD3 antibody is selected from the anti-CD3 complex and ranges from 10 to 3000 ng/mL and the cytokine-activated irradiated feeder cells ranges from 0.1 to 5 million feeder cells/well, preferably 1 million cells/well.

The feeder cells/well are added every 7-14 days, preferably 7-10 days, in a ratio of feeder cells to the immune cells is in the range from 1:1 up to 400:1, preferably in a range of 10:1 (feeder cell: T-cell) along with a crosslinking anti-CD3 directed antibody in the range of 10 to 3000 ng/mL, preferably at 30 ng/mL.

The expanded population of immune cells directed against tumor cells are selected from the group consisting of tumor-infiltrating lymphocytes or peripheral blood mononuclear cells, preferably selected from the group consisting of double-positive (CD4⁺CD8⁺) T-cells, double-negative (CD4⁻ CD8⁻) T-cells, CD4⁺CD8⁻ or CD4⁻CD8⁺ T-cells, γδ T-cells, MAIT cells, MR1 reactive T-cells, T-cells producing Th1 cytokines, T-cells expressing cytotoxic molecules or producing IL-9, αβ T-cells, including mixed populations of tissue resident immune-cells comprising at least one of the listed immune cell subsets.

Preferably, the immune cells directed against tumor cells are tumor-infiltrating lymphocytes or T-cells isolated from PBMCs or skin, antibody-sorted or a recombinant classical or non-classical MHC molecule loaded with the appropriate target antigen guiding antigen-specific selection comprising, but not limited to, the CDR3 region as set for in any of SEQ ID NO 1 to SEQ ID NO 410.

As regards the sequence listing, TCRs sequences are shown as follows:
SEQ ID NOs 1-20, 41-80, 111-130, 253-272, 315-334, 355-394 list the most frequent TCRs in the tumor section from which TIL were expanded;
SEQ ID NOs 21-40, 131-152, 273-292, 335-354 exhibit the most frequent TCRs recognizing autologous pancreatic cancer cells in an MHC class I (or class II) restricted fashion;
SEQ ID NOs 81-110 show the most frequent TCRs CD1d restricted and tumor-reactive T-cells;
SEQ ID NOs 153-172 and 295-316 show the most frequent TCRs Va 7.2 enriched T-cells which recognize autologous tumor cells in an MR1 restricted fashion;
SEQ ID NOs 173-252 show the most frequent TCRs MR1-6FP binding T-cells;
SEQ ID NOs 395-400 show the most frequent antigen specific TCRs against LRP1B mutant (VSKRLKFSRDLSLDP);
SEQ ID NOs 401-404 show the most frequent antigen specific TCRs against Mesothelin (HRLSEPPEDLDALPL); and
SEQ ID NOs 405-410 most frequent antigen specific TCRs against UQCRFS1 mutant (FVSSMSASAVVLALAKIEIKLSD) restricted by DPB1*04:02.

The present invention further discloses, in a second aspect, a potency assay for tumor recognition comprising the steps of:
(i) challenging the clinically relevant immune cells as set for in SEQ ID NO 517 to SEQ ID NO 611 and for SEQ ID NO 796 to SEQ ID NO 832 with at least one target antigen of human, bacterial, viral, helminth, protozoan or bacteriophage origin or their related mimicry analogues, provided that the analogue presents 50-100% of similarity in amino acids or the analogue presents different amino acids, but with similarity based on size, structure, or charge and
(ii) detecting a change in a cytokine or immune effector molecule production selected from cytokines or chemokines, preferably in particular IFN-γ production, cell proliferation, cytotoxicity, immune signaling and/or intracellular phosphorylation of signaling molecules associated with an anti-tumor immune response.

Preferably, the target antigen is a wild-type, a mutated private or commonly shared tumor-associated antigen, an antigen that is preferentially expressed during embryonal or fetal development and/or specifically presented by tumor cells or non-tumor cells supporting tumor-cells or driving tumorigenesis. The mutation is selected from point mutations, frameshift mutations, or antigens preferentially expressed during fetal/embryonal development; the point mutation residing preferably in the middle of the antigen.

Preferably, the target antigen comprises a length of 7 to 25 amino acids. More preferably, the target antigen is a 15-mer peptide or binding to MHC class II, or between 8-11 amino acids, preferably 9 amino acids, preferably binding to MHC class I target antigens or antigens presented by CD1 molecules or MR1 molecules. The antigen may undergo pre- or post-translational modification concerning sugar or lipid moieties.

In a preferred embodiment of the present invention, the private tumor-associated antigen further comprises a sequence as set for in any of SEQ ID NO 556 to SEQ ID NO 611 and the commonly shared tumor-associated antigen further comprises a sequence as set for in any of SEQ ID NO 517 to SEQ ID NO 555 and SEQ ID NO 796 to SEQ ID NO 832.

In a particular embodiment of the present invention, the private tumor-associated targets are obtained from
providing a body sample from a patient, in particular a tumor sample;
performing whole tumor exome sequencing as compared to a non-tumor sample from the same patient;
performing RNA or DNA sequencing of the tumor sample;
preparing a construct with the mutation in the middle of the peptide sequence flanked by 7 amino acids to the left and to the right of the mutant amino acid residue;
constructing a synthetic peptide containing at least 12 to 17 amino acids of the identified frameshift mutation;
tailoring peptides to best fit according to the patient's individual MHC class I and MHC class II genetic background;
blocking classical MHC molecules, selected from MHC class I and MHC class II proteins, or non-classical MHC molecules, selected from MR1 or CD1a-d, preferably CD1d using appropriate blocking antibodies or using siRNA;
analyzing the tumor sample to identify a specific T-cell receptor reaction with a wild-type or mutant target from a tumor-associated antigen provided from a cellular protein or a mitochondrial target;
identifying a mutated target antigen or a series of mutated target antigens in the tumor sample, provided that the mutated target antigen(s) does not elicit pro-tumorigenic or immune-suppressive functions;
producing synthetic peptides with similar amino acid composition and/or a similar 3D structure with the mutated target antigen in lieu of the tumor cells or other target antigen presenting cells;
   wherein the specific T-cell receptor is T-cell receptor αβ, T-cell receptor γδ or T-cell receptors expressed by MAIT-cells or MR1 reactive T-cells.

Preferably, the specific T-cell receptor is a T-cell receptor as set for in any of SEQ ID NO 1 to SEQ ID NO 410 and the reaction between the specific T-cell receptor and the target from the tumor-associated antigen give rise to immune effector functions in responding T-cells that are anti-tumor directed or—more specifically, directed to tumor-stem cells, based on cytotoxicity, proliferation, apoptosis-inducing molecules, or the quality and quantity of cytokine-mediated tumor cell death, inhibition of tumor cell proliferation, induction of differentiation or aging of the tumor cells.

In a third aspect, the present invention further discloses a biological vaccine preparation to provide anti-tumor response or antiviral response, wherein it comprises target antigens that lead to the expansion of a certain set of T-cells or B-cells.

Additionally, the target antigens that show cross-reactivity to human self-proteins and antigenic structures that induce factors that are pro-tumorigenic and/or induce autoimmune responses are removed from the preparation, based on antigen-specific immune reactivity, e.g., cytokine production, using IFN-γ, IL-17 or Th2-based cytokines as the signature immune readouts or their related RNA signatures associated with Th1/Th2/Th17 or Th9 responses.

The target antigens are private or commonly shared tumor-associated targets specifically presented by tumor cells or non-tumor cells supporting tumor-cells or driving tumorigenesis by non-transformed cells that support malignant transformation, or support transformed cells.

Preferably, the private or commonly shared tumor-associated target is selected from wild-type or mutant, not excluding fusion proteins or frameshift mutations, 15-mer peptides or binding to or binding to MHC class II, or between 8-11 amino acids, preferably 9 amino acids, preferably binding to MHC class I target antigens or antigens presented by CD1 molecules or MR1 molecules. The private or commonly shared tumor-associated target can also be selected from wild-type or dysfunctional or damaged mitochondrial-associated molecules that act as tumor-associated targets in humans. The antigens may be glycosylated, phosphorylated or lactylated. The tumor-associated antigens may be differentially expressed during fetal or embryonal development or not readily accessible post-partum due to limited access of immune cells to the nominal target antigen.

Additionally, the private tumor-associated target is as set for in any of SEQ ID NO 556 to SEQ ID NO 611 and the commonly shared tumor-associated target is as set for in any of SEQ ID NO 517 to SEQ ID NO 555 and SEQ ID NO 796 to SEQ ID NO 832. For both cases, the tumor-associated target may also be an amino acid sequence presenting up to 70% or more in amino acid exchanges, provided that the individual amino acids comprise a similar chemically 3D structure or an amino acid sequence presenting different amino acids, but with similarity based on size, structure or charge, or an amino acid sequence which is part of a chimeric antigen receptor construct.

Preferably, the specific T-cell receptor is a T-cell receptor as set for in any of SEQ ID NO 411 to SEQ ID NO 516 to the target antigens as set for in any of SEQ ID NO 612 to SEQ ID NO 795, wherein:
SEQ ID NOs 411-442 show the most frequent antigen specific TCRs against LP(V)RDLPQGF from SARS-CoV-2 spike (positions 212-220 QHD43416.1) tailored for HLA-B3501
SEQ ID NOs 443-474 show the most frequent antigen specific TCRs against LP(V)RDLVTGF from Human U11/U12 snRNP 35 kDa protein (positions 81-90 Q16560.1) tailored for HLA-B3501;
SEQ ID NOs 475-478 show the most frequent antigen specific TCRs against LP(D)SKVGGNY from SARS-CoV-2 spike (positions 440-449 QHD43416.1) tailored for HLA-B3501;
SEQ ID NOs 479-484 show the most frequent antigen specific TCRs against LR(D)SKVGGNY from SARS-CoV-2 spike (positions 440-449 QHD43416.1) tailored for HLA-C0701;
SEQ ID NOs 485-496 show the most frequent antigen specific TCRs against LR(K)SKHGGNY from Human Tensin-1 (positions 47-55 Q9HBL0.2) tailored for HLA-C0701;
SEQ ID NOs 497-504 show the most frequent antigen specific TCRs against LR(V)RDLPQGF from SARS-CoV-2 spike (positions 212-220 QHD43416.1) tailored for HLA-C0701;
SEQ ID NOs 505-512 show the most frequent antigen specific TCRs against LVQDLAQGF from Tryptophan—tRNA ligase, mitochondrial (positions 188-196 Q9UGM6.1) tailored for HLA-C0701; and
SEQ ID NOs 513-516 show the most frequent antigen specific TCRs against LR(V)RDLVTGF from Human U11/U12 snRNP 35 kDa protein (positions 81-90 Q16560.1) tailored for HLA-C0701.

In a fourth aspect, the present invention discloses epitope target for antibodies that serves as a viable target that can be used to construct chimeric antigen receptors.

More specifically, the epitope targets for antibodies are directed against PVTSLSSVSTGDTTP from MUC4 or parts of said epitope, preferably 3-5 or 3-6, 3-7, or 3-8 amino acids or amino acids of a similar size and charge, resulting in a similar 3D structure binding to the epitope from MUC4, which can be used to construct chimeric antigen receptors targeting MUC4.

The MUC4 peptide is targeted by human IgG in serum from patients with cancer. This epitope is present in different areas of MUC4 and exhibits therefore multiple docking sites for an anti-MUC4 directed antibody response or as a target for chimeric antigen receptors, since it is targeted by naturally occurring antibody responses in patients with cancer.

### EXAMPLES

Hereinafter, reference is made to the examples of the present invention, which aim to describe it in more details as regards tests that have been conducted and experimental results that have been achieved.

Unless otherwise indicated, all technical and scientific terms used in this document have the same meaning as commonly understood by someone skilled in the art to which this invention belongs.

Methods and materials are described in this document for use in the present invention; other suitable methods and materials known in the art can also be used. The materials, methods and examples are illustrative only and are not intended to be limiting.

### Body Samples: Tissue

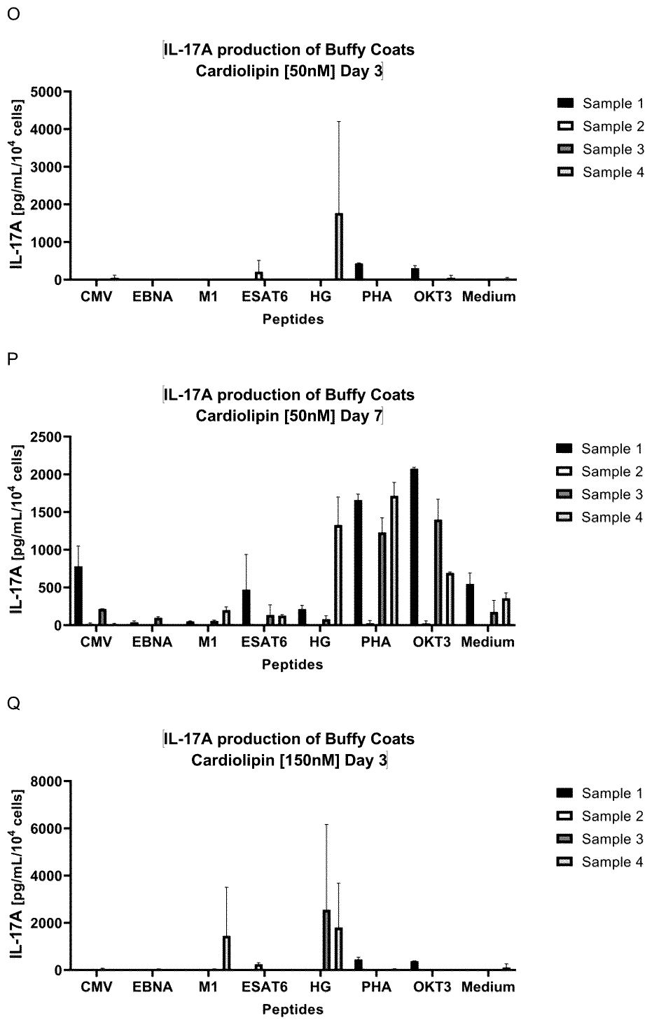

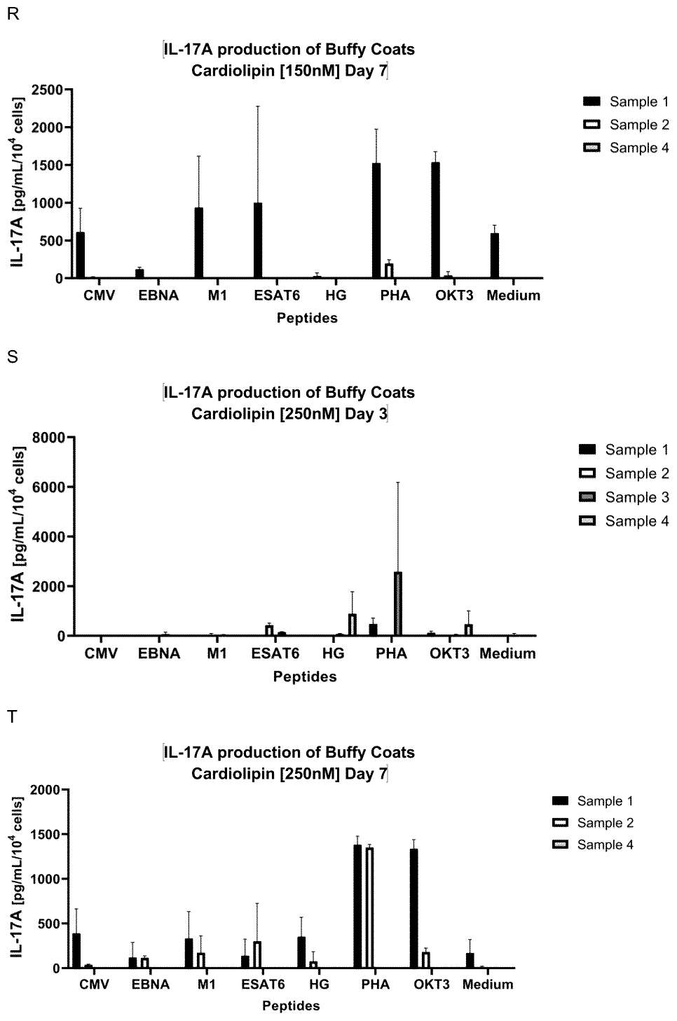

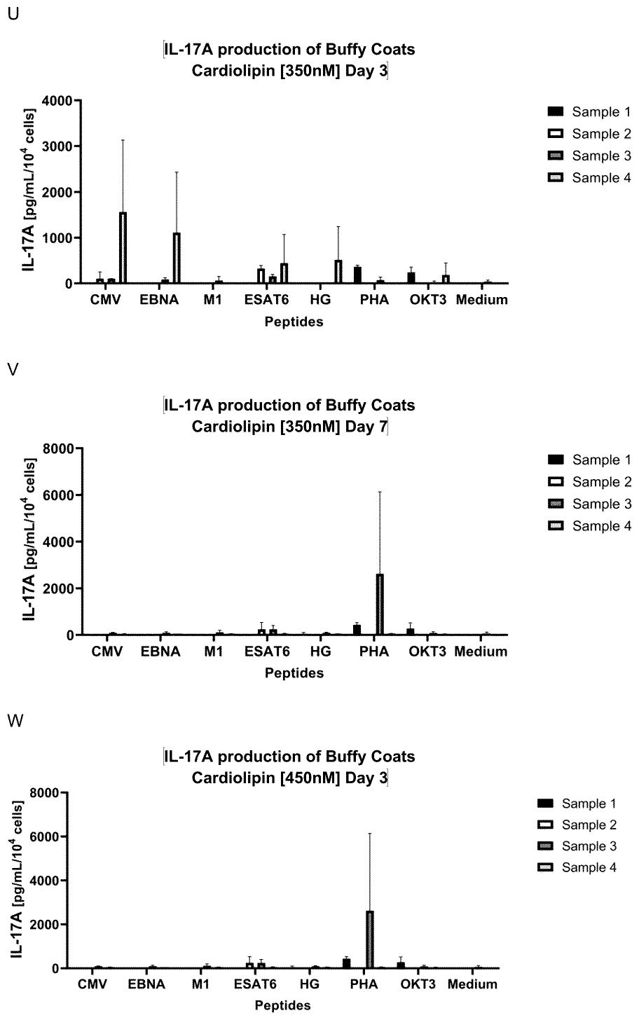

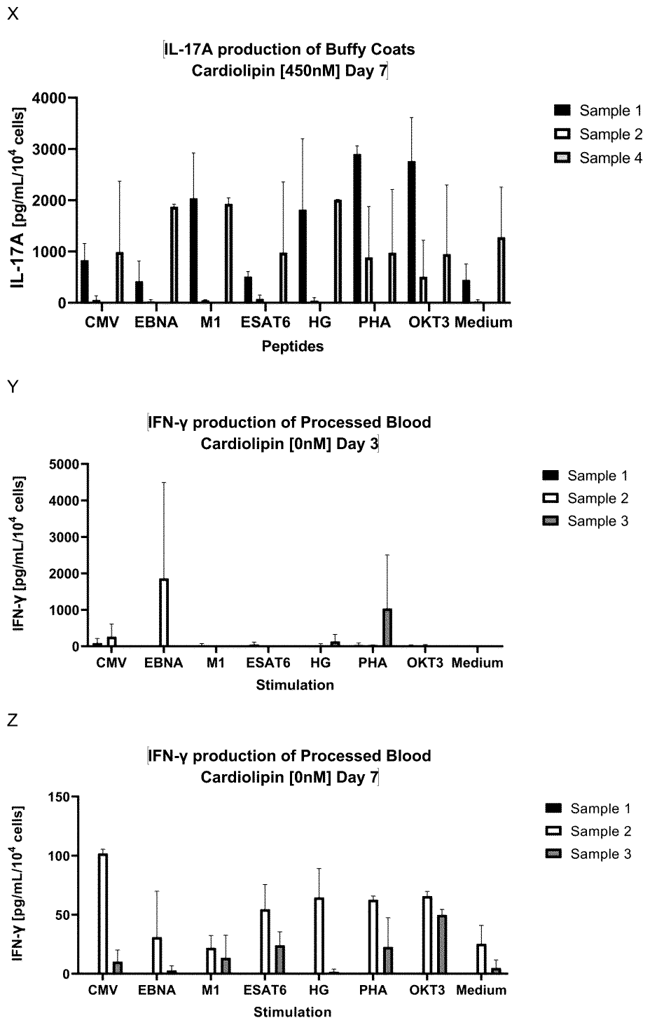

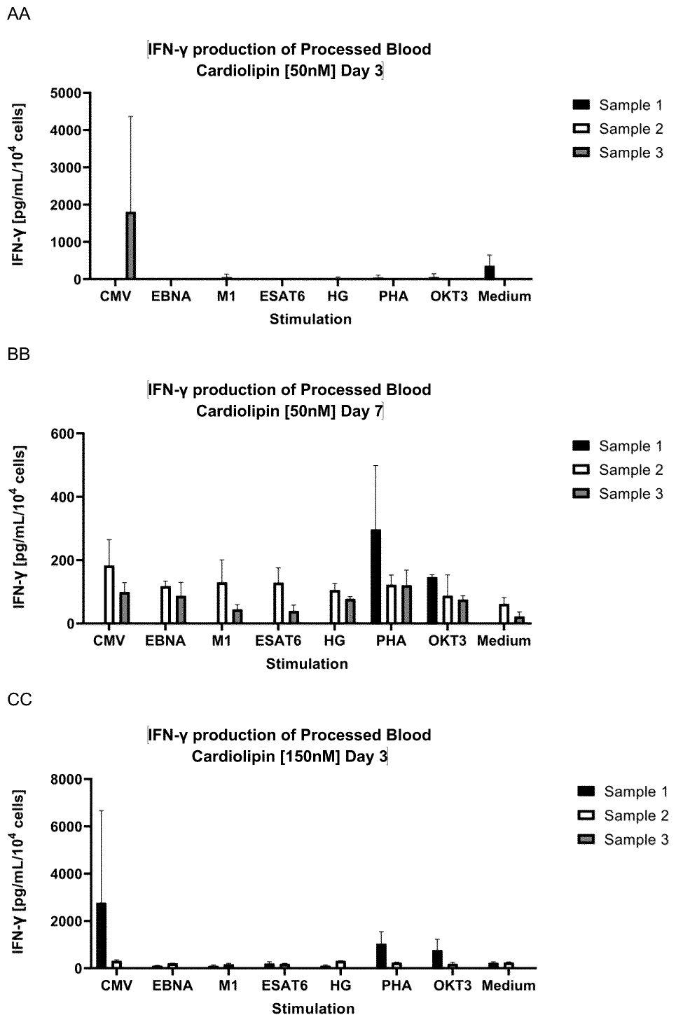

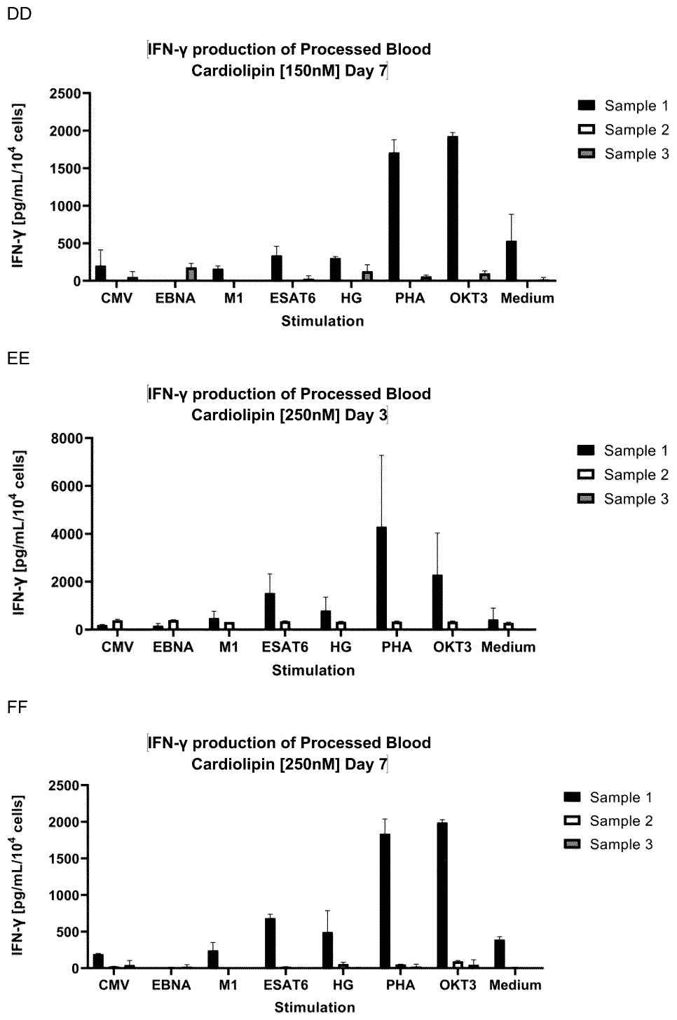

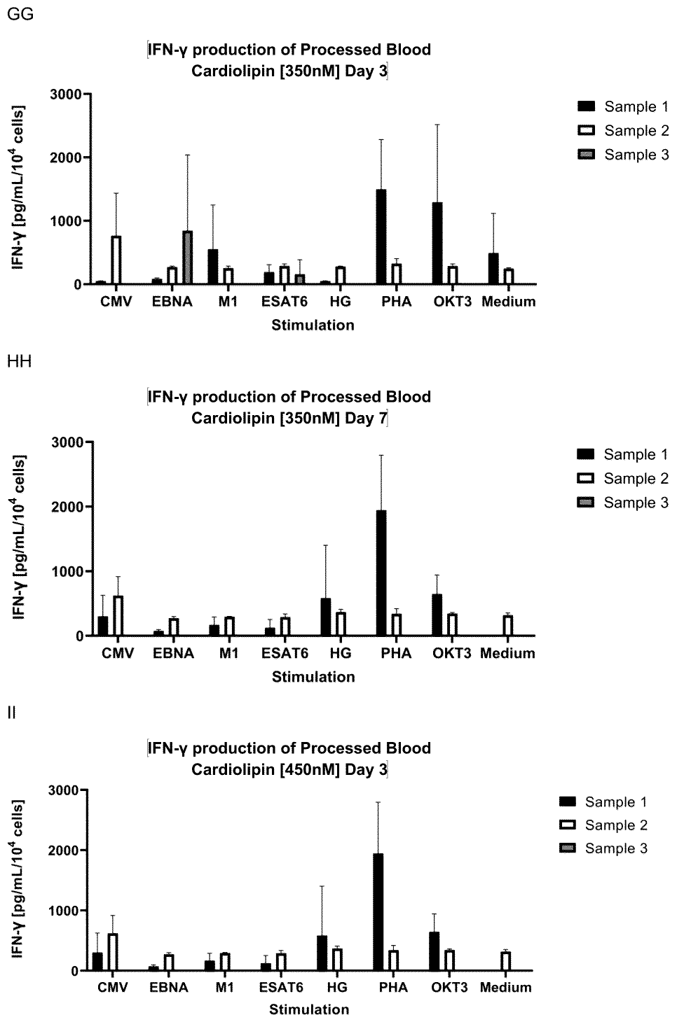

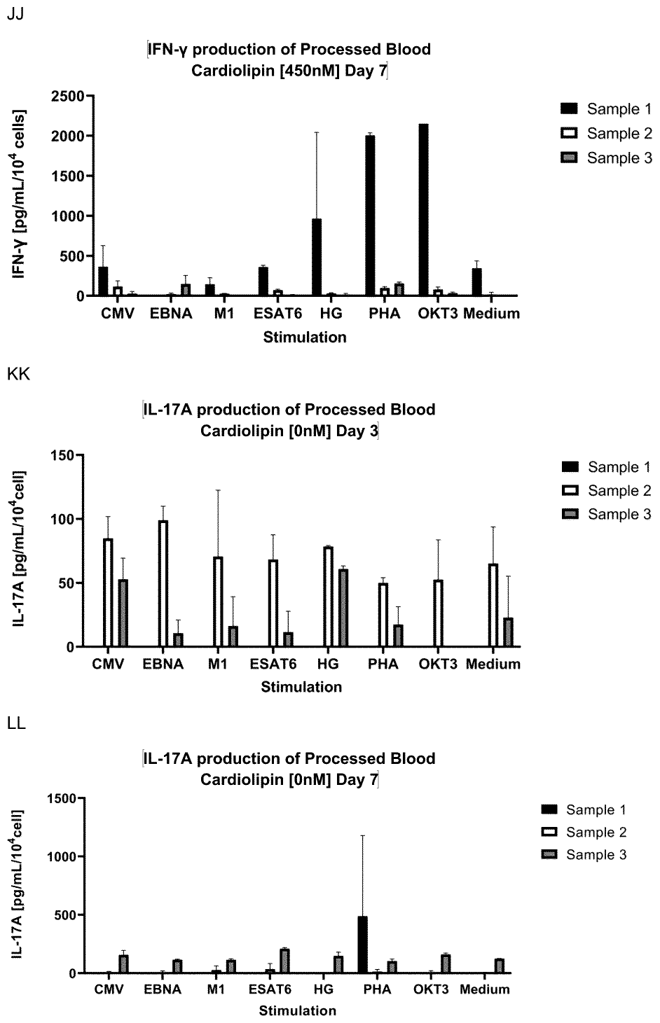

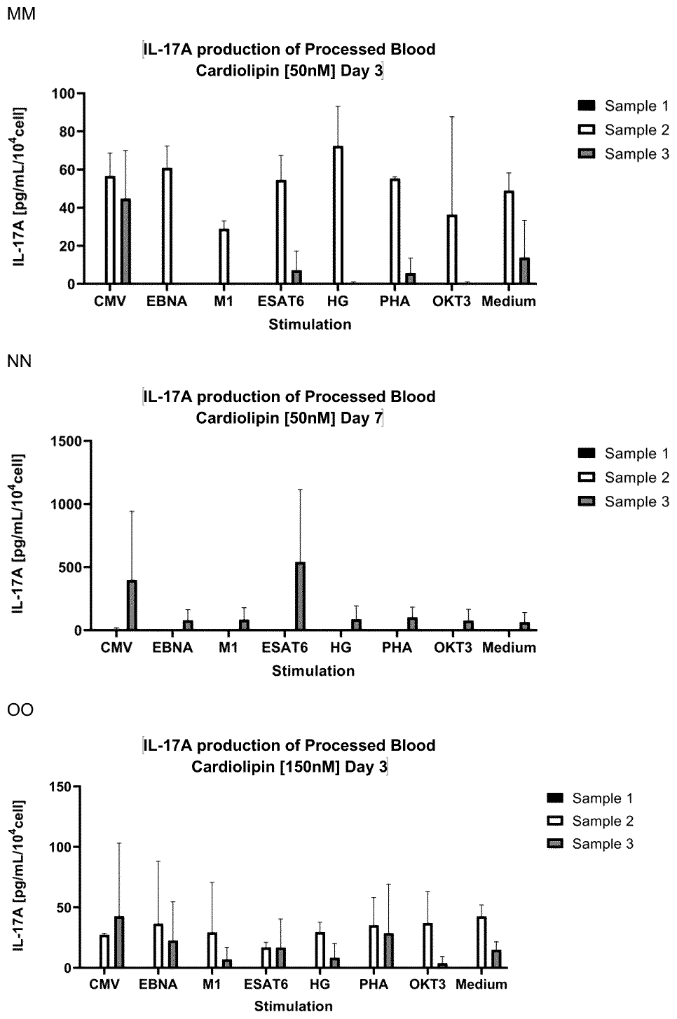

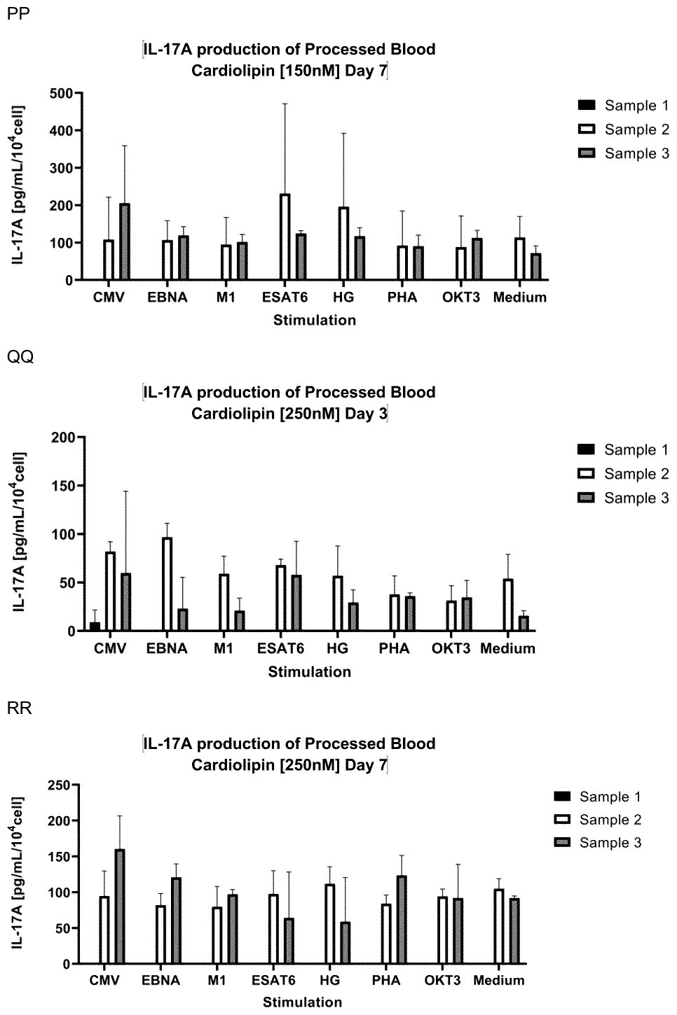

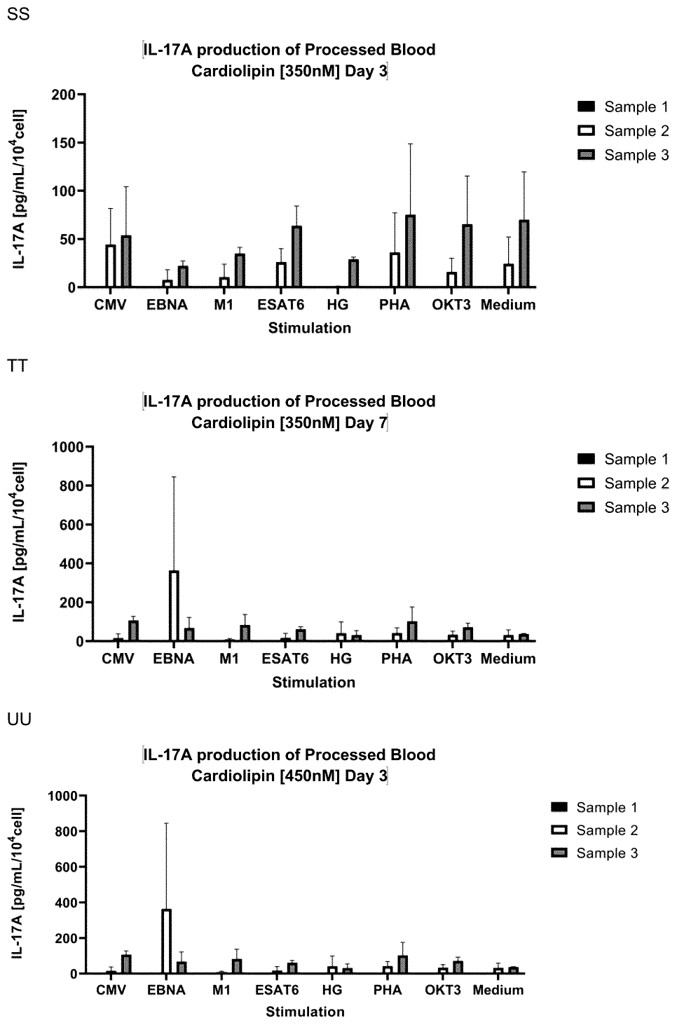

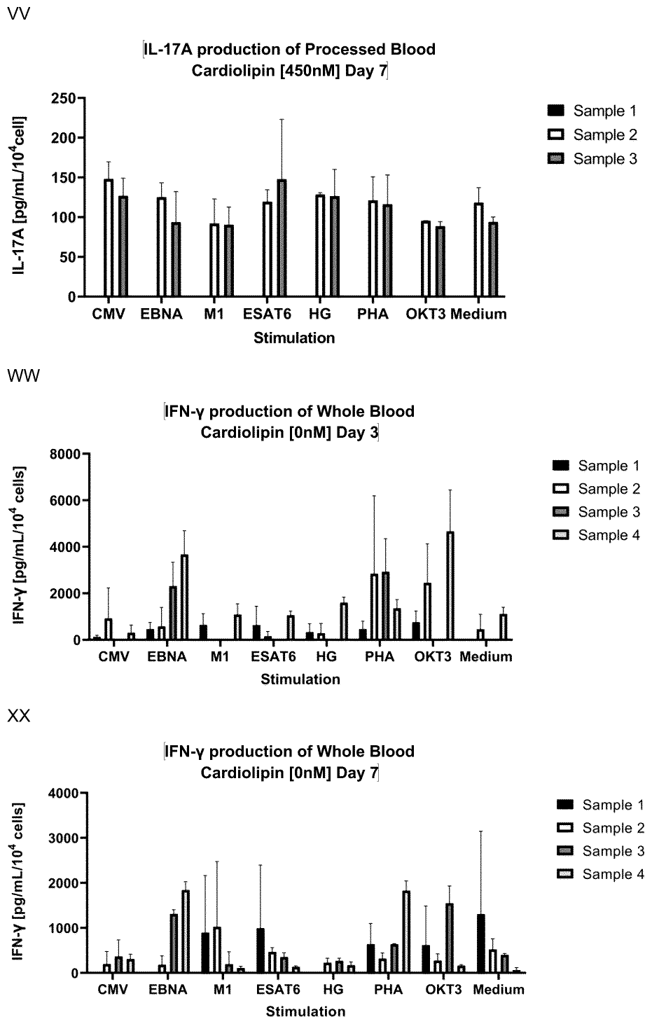

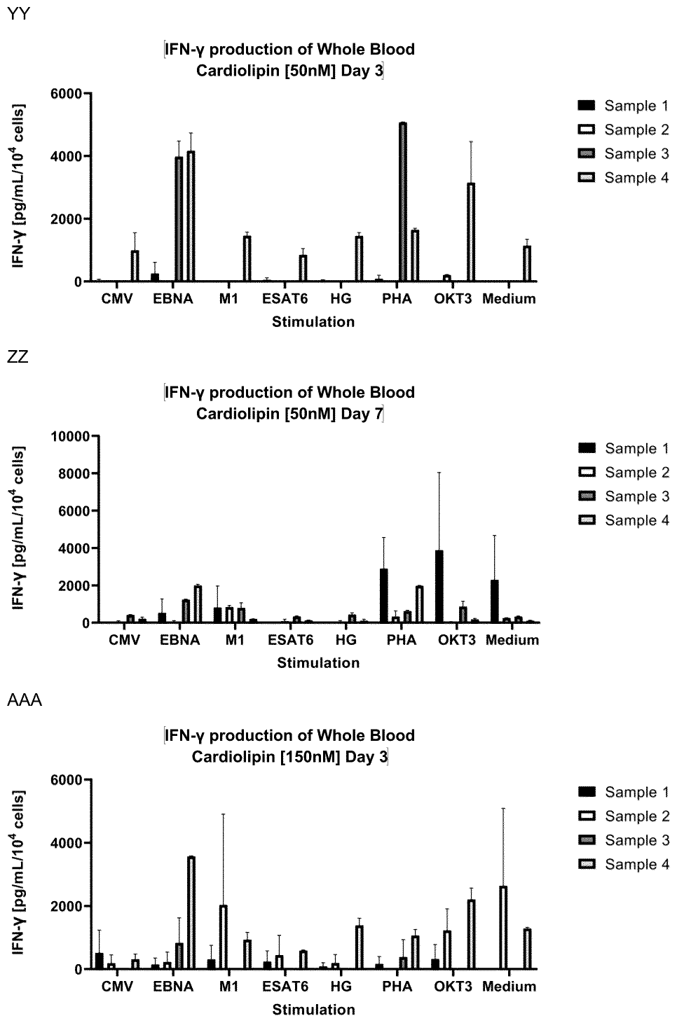

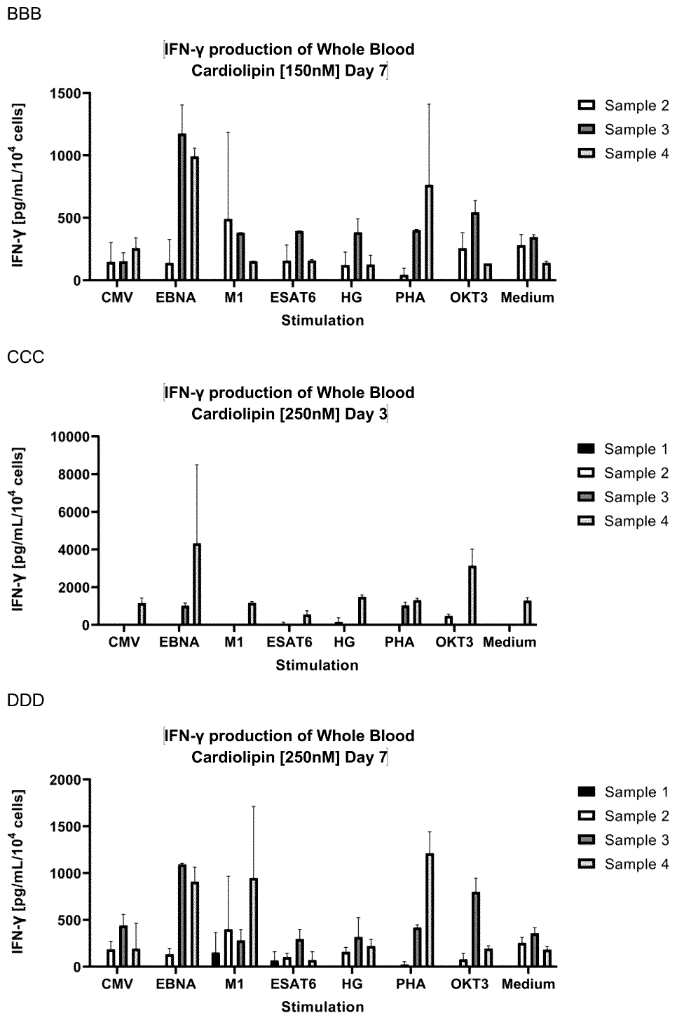

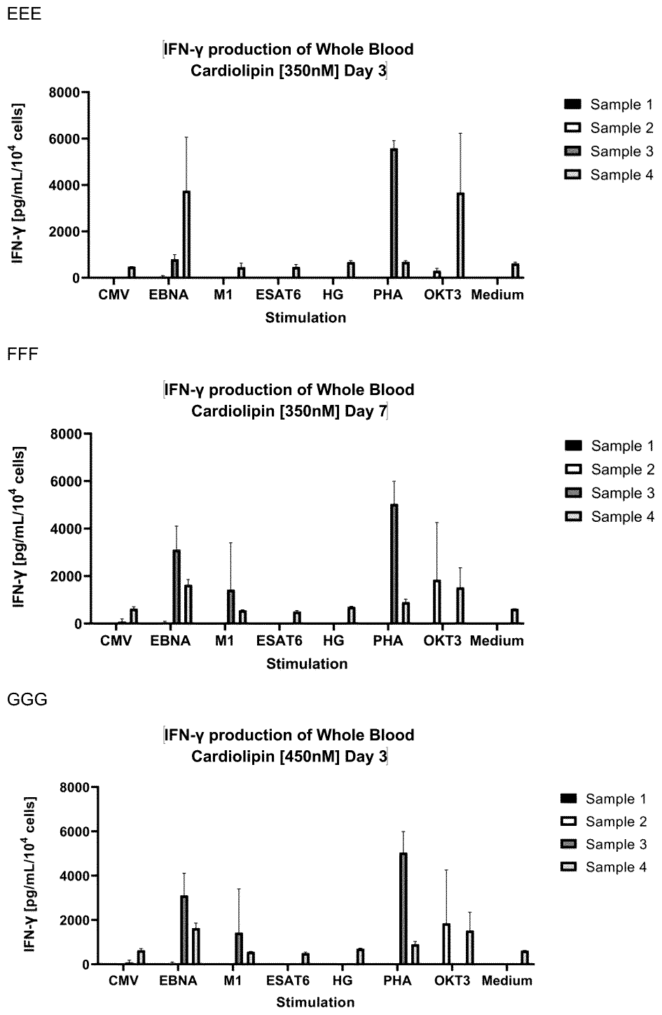

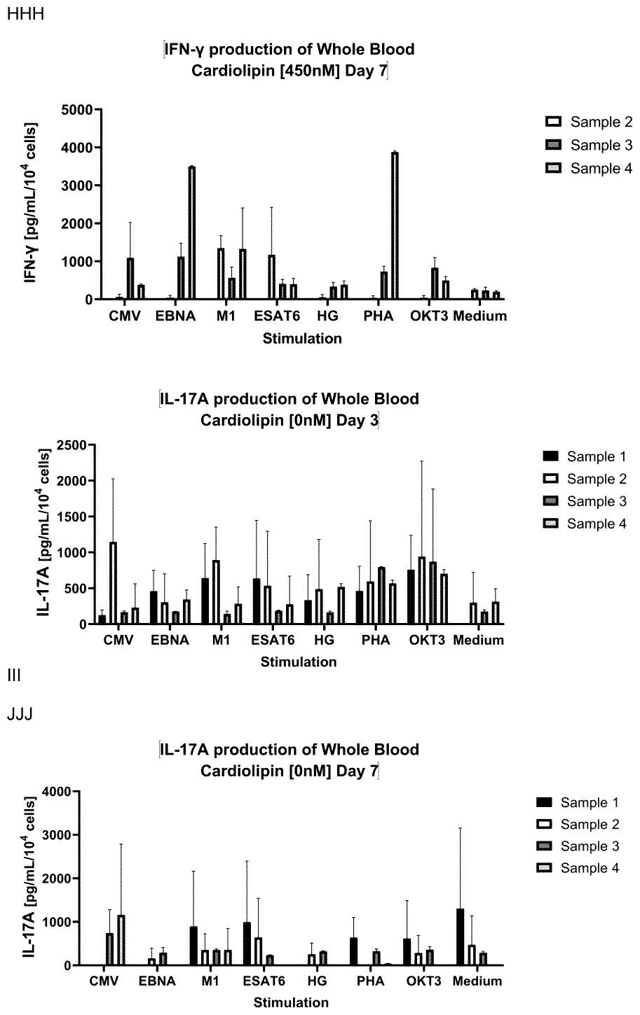

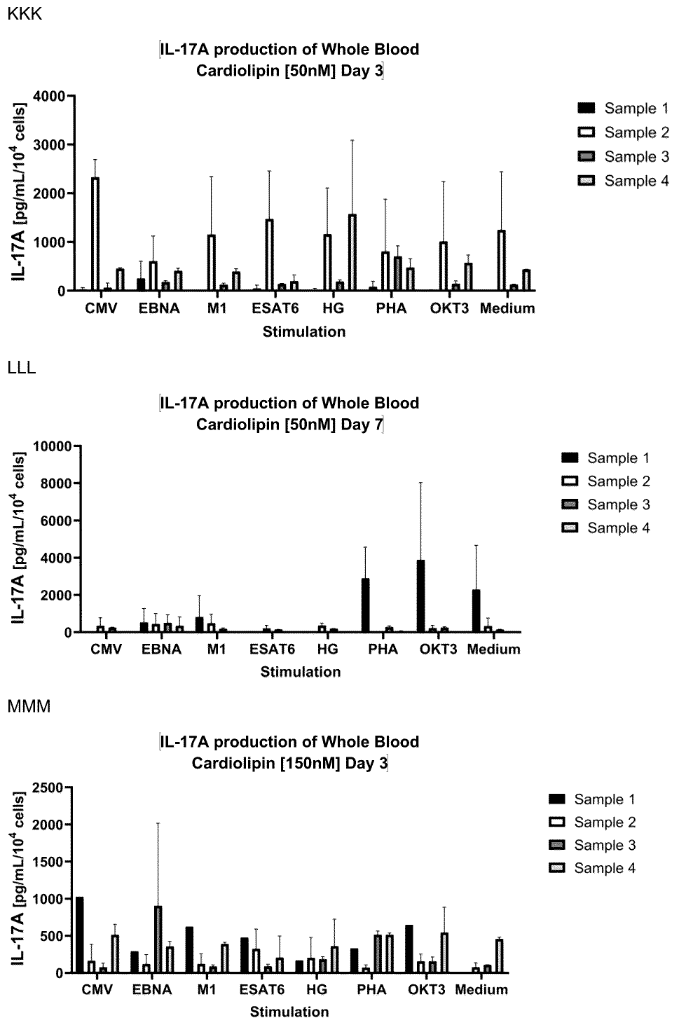

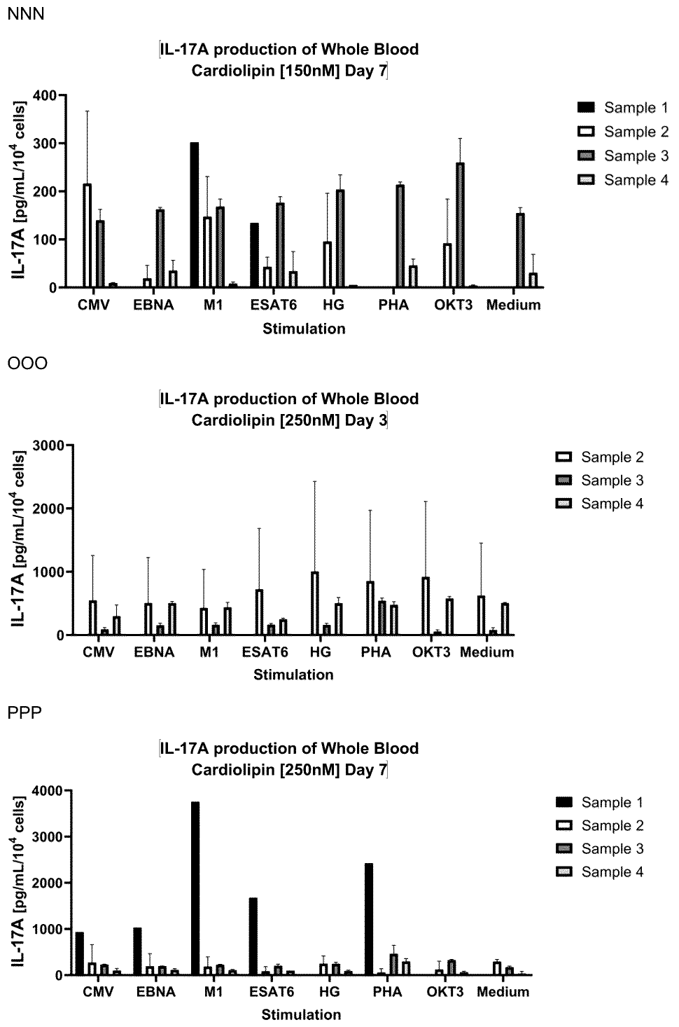

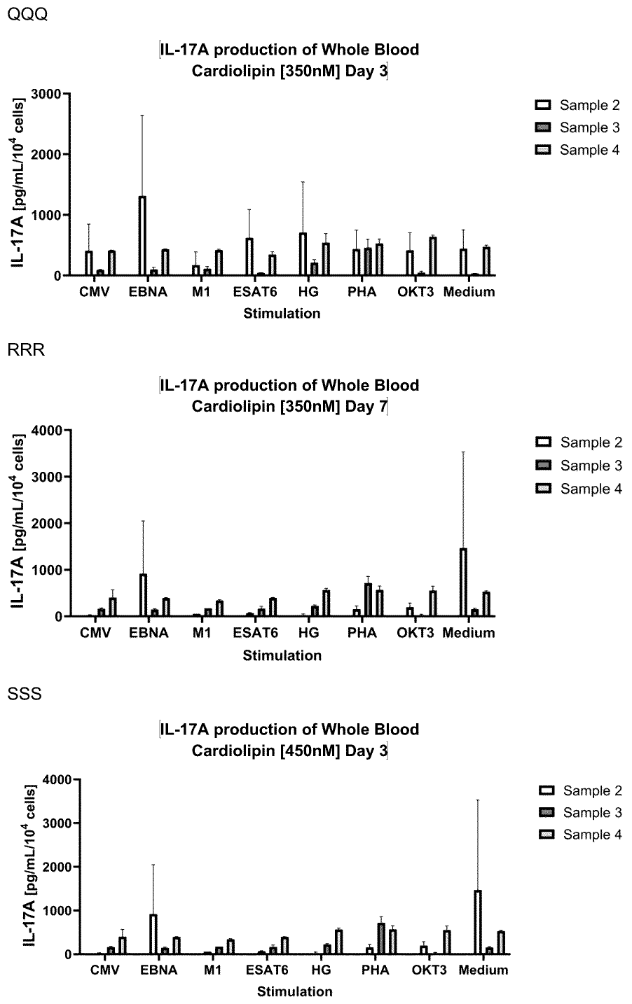

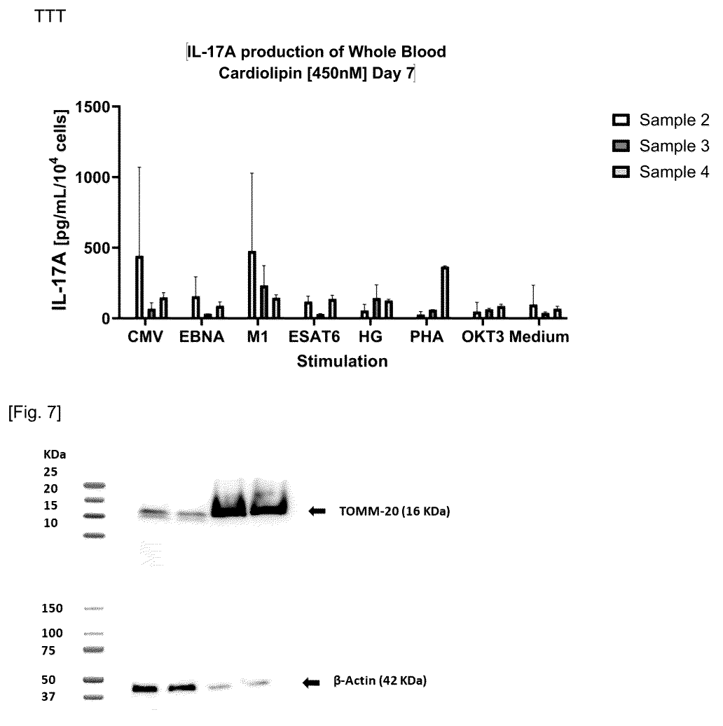

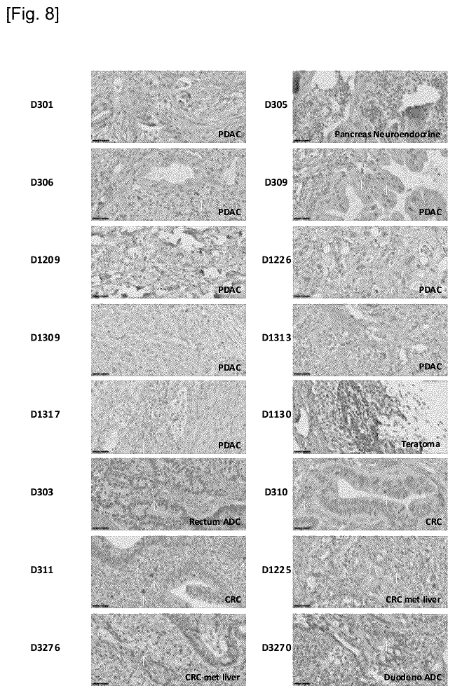

Tumor specimens were removed from primary or metastatic tumor lesions (see FIG. 8) listed in the Table 1 below. The tumor specimen was removed and a wedge from the tumor and parallel sections were performed by the pathologist.

TABLE 1

| | | Tumor | | Isolated/ | |
|---|---|---|---|---|---|
| Donor | Diagnostic | in | out | Clusters | Observations |
| D301 | PDAC | ± | + | Isolated | No γδ⁺ T-cells found in |
| S4657 | | | | (only 1 or 2) | the tumor. Only 1 or 2 |
| | | | | | found in the periphery |
| | | | | | (stroma desmoplastic). |
| D303 | Rectum ADC | − | ± | Isolated | Very few γδ⁺ T-cells, |
| S479 | | | | | only found in the stroma |
| | | | | | (intratumoral). |
| D305 | Pancreas | + | − | Isolated | γδ⁺ T-cells only found |
| S484 | neuroendocrine | | | | intraepithelial. |
| | tumor | | | | |
| D306 | PDAC | ± | + | Isolated | γδ⁺ T-cells found in the |
| S485 | | | | | periphery, very few in |
| | | | | | the intraepithelial region. |
| D309 | PDAC | ± | + | Isolated | γδ⁺ T-cells found in the |
| S489 | | | | | periphery. |
| D310 | CRC | + | + | Isolated | γδ⁺ T-cells found in the |
| S490 | | | | | stroma (intratumoral) |
| | | | | | and also in the |
| | | | | | intraepithelial region. |
| D311 | CRC | ± | + | Isolated | γδ⁺ T-cells found in the |
| S492 | | | | | stroma (intratumoral) |
| | | | | | and few in the |
| | | | | | intraepithelial region. |
| D1130 | Teratoma | − | ++ | Isolated and | γδ⁺ T-cells found in the |
| S4654 | | | | little clusters | periphery of the cyst |
| | | | | | surrounding the cyst of |
| | | | | | the teratoma. |
| D1209 | PDAC | + | ++ | Isolated | γδ⁺ T-cells found |
| | | | normal | | intraepithelial of a |
| | | | | | normal duct (stroma |
| | | | | | peritumoral). Normal |
| | | | | | pancreas has more γδ⁺ |
| | | | | | T-cells than the tumor. |
| D1225 | CRC met | ± | + | Isolated | Few γδ⁺ T-cells, more |
| | Liver | | | | concentrated in the |
| | | | | | tumor periphery near |
| | | | | | normal liver. |
| D1226 | PDAC | + | + | Isolated and | γδ⁺ T-cells found |
| S4656 | | | | little clusters | intraepithelial, |
| | | | | | peritumoral stroma and |
| | | | | | peripheric tumor. γδ⁺ |
| | | | | | T-cells found near |
| | | | | | tumor cells. |
| D1309 | PDAC | + | + | Isolated | Few tumor cells. γδ⁺ T- |
| | | | | | cells found outside of |
| | | | | | the tumor and in the |
| | | | | | tumor periphery. |
| D1313 | PDAC | + | + | Isolated | γδ⁺ T-cells found |
| | | | | | intratumoral in the |
| | | | | | stroma and also in the |
| | | | | | tumor periphery. |
| D1317 | PDAC | ++ | + | Isolated and | γδ⁺ T-cells found |
| | | | | clusters | intratumoral in the |
| | | | | | stroma and also in |
| | | | | | the periphery |
| D3270 | Duodeno ADC | ++ | + | Isolated | γδ⁺ T-cells found |
| | | | | | intratumoral in the |
| | | | | | stroma and also |
| | | | | | intraepithelial. |
| D3276 | CRC met Liver | +++ | +++ | Clusters | Normal liver with a lot |
| | | | | | of γδ⁺ T-cells and also |
| | | | | | in the tumor. |

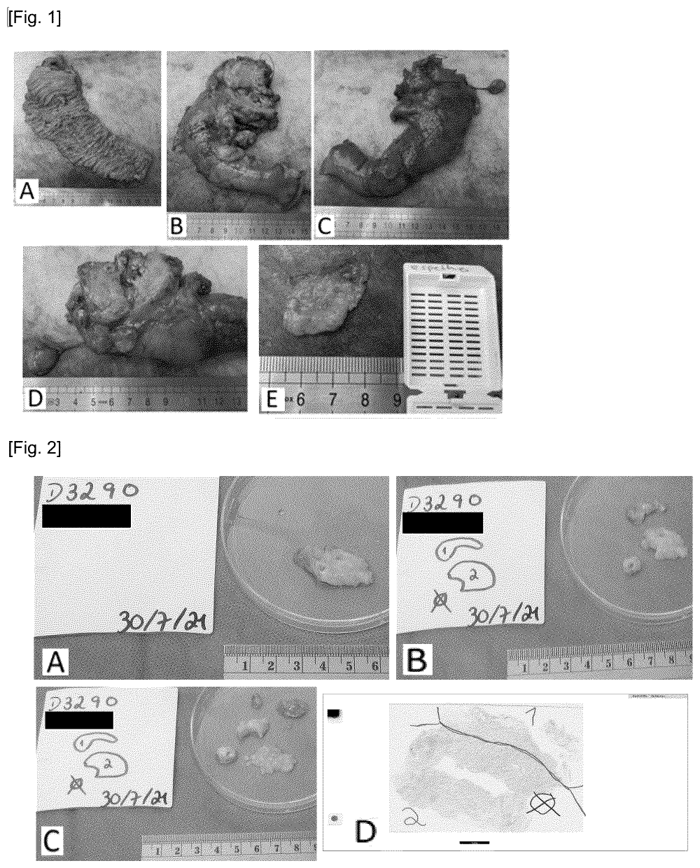

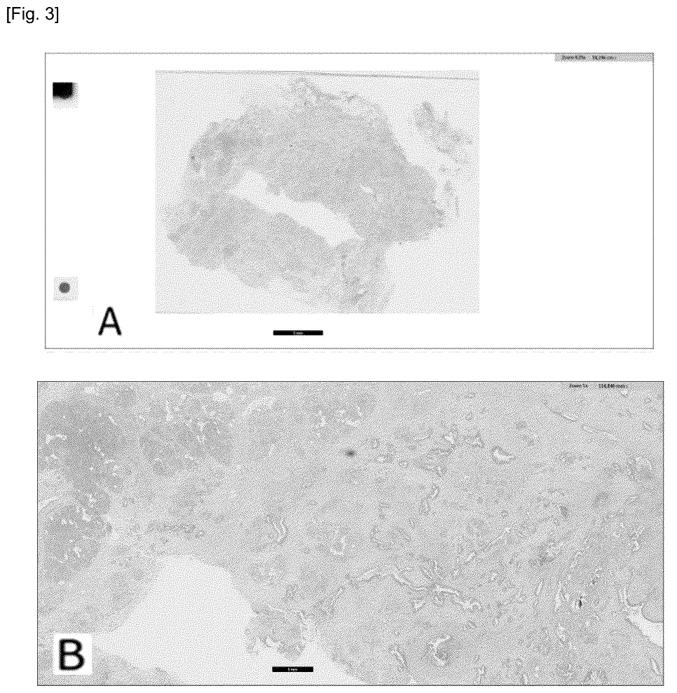

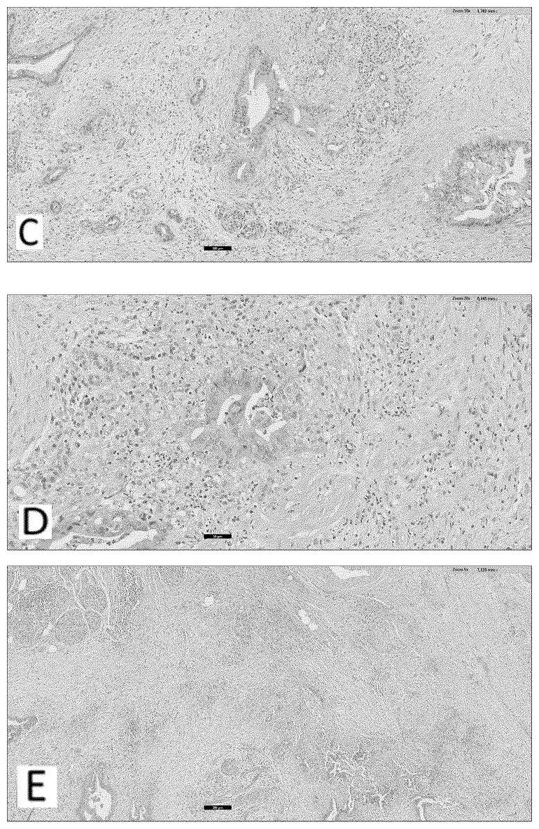

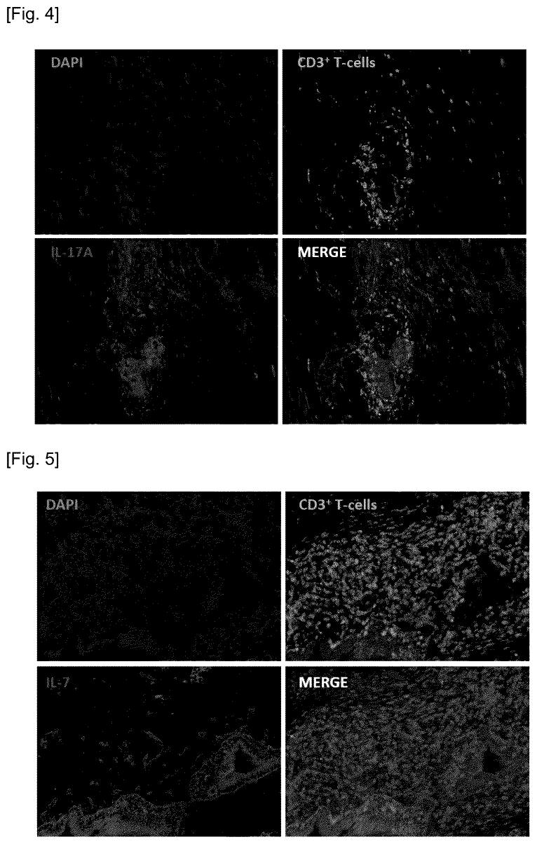

Different areas of the tumor tissue were dissected in individual pieces based on anatomical areas and tissue ‘stiffness’, defined by desmoplastic region, i.e., at least 2-3 mm from arterial vessels or from infiltration that is more visible upon bathing the piece in cold PBS (FIGS. 1 to 3), and/or based on differential gene expression and consequent cytokine expression in the tumor and adjacent tissue, for example IL-7 or IL-17. (FIGS. 4 and 5).

Each tumor area was dissected into small tumor pieces and placed in individual 24 well plates with 3-5 pieces/well of 1 mm³ in 1 mL of CellGenix GMP DC media, or alternatively Lonza X VIVO 15 media supplemented with 10% Human Serum and 1000 IU/mL IL-7, 150 IU/mL IL-15 and 1 IU/mL IL21. Serum free medium can also be used or a range of serum between 1-10%.

### Expansion of TIL

Outgrowth of TIL was differently associated with the different TIL infiltrates that had great impact on T-cell activation, exhaustion, composition, and maturation markers, as can be seen on Tables 2 and 3 below.

TABLE 2
Different TIL populations from different tumor regions (D3290).

| | | CD4⁺ | CD39⁺ | CD39⁺ | CD39⁻ | CD39⁻ | | | |
|---|---|---|---|---|---|---|---|---|---|
| | CD38⁺ | CD8⁺ | CD69⁺ | CD69⁻ | CD69⁺ | CD69⁻ | Precursor | TCM | Teff |
| Zone 1 | 70.7% | 13.5% | 52.6% | 21.9% | 14.8% | 10.7% | 5.88% | 36.3% | 1.13% |
| Zone 2 | 83.4% | 8.51% | 62.8% | 21.0% | 11.0% | 5.24% | 4.40% | 48.0% | 0.32% |
| | | CD4⁺ | | | | | | | |
| | | CD8⁻ | | | | | | | |
| Zone 1 | | 17.2% | 31.0% | 12.5% | 32.0% | 24.4% | 8.67% | 46.9% | 0.92% |
| Zone 2 | | 46.6% | 59.2% | 12.7% | 22.3% | 5.87% | 3.44% | 57.8% | 0.16% |
| | | CD4⁻ | | | | | | | |
| | | CD8⁺ | | | | | | | |
| Zone 1 | | 43.5% | 67.4% | 20.5% | 9.92% | 2.14% | 2.10% | 24.5% | 0.89% |
| Zone 2 | | 26.2% | 76.9% | 14.0% | 7.74% | 1.29% | 2.51% | 45.7% | 0.51% |
| | | CD4⁻ | | | | | | | |
| | | CD8⁻ | | | | | | | |
| Zone 1 | | 25.8% | 25.2% | 6.63% | 27.9% | 40.2% | 21.8% | 21.3% | 17.4% |
| Zone 2 | | 18.7% | 26.0% | 5.31% | 31.3% | 37.3% | 22.1% | 31.6% | 12.8% |

| | | CD4⁺ | | | | | | |
|---|---|---|---|---|---|---|---|---|
| | CD38⁺ | CD8⁺ | TEM | CD95 | CD103⁺ | GD⁺ | Lag3 | 4-1BB |
| Zone 1 | 70.7% | 13.5% | 56.7% | 68.3% | 10.2% | 5.67% | 28.2% | 2.16% |
| Zone 2 | 83.4% | 8.51% | 47.3% | 0.55% | 10.2% | 3.49% | 33.4% | 4.43% |
| | | CD4⁺ | | | | | | |
| | | CD8⁻ | | | | | | |
| Zone 1 | | 17.2% | 43.5% | 49.0% | 2.03% | 8.81% | 13.5% | 0.56% |
| Zone 2 | | 46.6% | 38.6% | 86.9% | 1.49% | 3.85% | 45.8% | 1.37% |
| | | CD4⁻ | | | | | | |
| | | CD8⁺ | | | | | | |
| Zone 1 | | 43.5% | 72.5% | 0.025% | 20.6% | 2.33% | 56.5% | 1.26% |
| Zone 2 | | 26.2% | 51.3% | 0.35% | 28.7% | 1.43% | 78.5% | 1.99% |
| | | CD4⁻ | | | | | | |
| | | CD8⁻ | | | | | | |
| Zone 1 | | 25.8% | 39.5% | 24.1% | 7.65% | 18.5% | 12.8% | 1.53% |
| Zone 2 | | 18.7% | 33.5% | 29.6% | 6.94% | 13.7% | 18.6% | 3.34% |

As can be seen in Table 2, TIL from tumor D3290 were expanded using identical conditions and flow cytometric analysis has been performed. Different phenotypes in TIL were observed, when using different tumor regions in the three-expansion step herein proposed.

CD4⁺ TIL and CD8⁺ TIL showed increased frequency in Zone 1. A CD39⁻CD69⁻ phenotype in the TIL product has been identified with increased responsiveness to therapy. Central memory T-cells (TCM) defined by the markers CD45RA⁻CCR7⁺ are also associated with increased responsiveness in the TIL product.

It is also of note that Zone 2 has—independent of the CD4⁺ or CD8⁺ components—an increased frequency of this T-cell subpopulation. In contrast, the frequency of TEM (effector memory T-cells) is increased in TIL from Zone 1. T-cell activation marker 4-1 BB has been associated with T-cell activation as well as with a T-cell population enriched in antigen-specific T-cells, which is higher in TIL from Zone 2 independent of the CD4⁺ or CD8⁺ T-cell phenotype.

It is also of note that CD4⁺ CD8⁺ (double-positive) CD3⁺ TIL represent highly activated CD4⁺ T-cells that express the CD8a chain; CD4⁻CD8⁻ (double-negative T-cells) represent here highly activated T-cells that have downregulated the CD4⁺ or CD8⁺ co-receptor. Rarely they represent thymic emigrants that are independent of CD4⁺ or CD8⁺ co-receptor help.

Phenotypic analysis of TIL from different regions represents biologically relevant differences in T-cell function, activation, tissue access, maturation, and differentiation—as well as markers that have been linked of the TIL phenotype to clinical responsiveness.

TABLE 3
Differential cytokine production in TIL associated with distinct tumor areas

| | IL-23 pg/10e5 cells | | | IL-6 pg/10e5 cells | | | IFN-y pg/10e5 cells | | | IL-17 pg/10e5 cells | | |
|---|---|---|---|---|---|---|---|---|---|---|---|---|
| | cells | | | cells | | | cells | | | cells | | |
| | in medium | OKT3 | Delta | in medium | OKT3 | Delta | in medium | OKT3 | Delta | in medium | OKT3 | Delta |
| | IL2/7/15 - | IgG1 - | (B - | IL2/7/15 - | IgG1 - | (B - | IL2/7/15 - | IgG1 - | (B - | IL2/7/15 - | IgG1 - | (B - |
| TIL | A | B | A) | A | B | A) | A | B | A) | A | B | A) |
| D16778 | 0.00 | 0.00 | 0.00 | 79.99 | 76.67 | 0.00 | 714.41 | 769.36 | 54.95 | 10.14 | 16.44 | 6.30 |
| Zone 2 | | | | | | | | | | | | |
| D1688 | 0.00 | 0.00 | 0.00 | 7.52 | 10.76 | 3.24 | 685.62 | 726.22 | 40.60 | 8.87 | 15.29 | 6.42 |
| Zone 3 | | | | | | | | | | | | |
| D1737 | 3.22 | 1.58 | 0.00 | 12.77 | 12.27 | 0.00 | 420.26 | 648.04 | 227.79 | 0.64 | 2.21 | 1.57 |
| Zone 1 | | | | | | | | | | | | |
| D1737 | 25.81 | 1279.40 | 1253.60 | 0.00 | 1.35 | 1.35 | 272.22 | 612.68 | 340.46 | 0.00 | 0.00 | 0.00 |
| Zone 2 | | | | | | | | | | | | |
| D1871 | 0.00 | 0.00 | 0.00 | 21.40 | 16.88 | 0.00 | 649.90 | 683.04 | 33.14 | 43.53 | 47.72 | 4.19 |
| D3290 S7370 | 0.67 | 433.95 | 433.28 | 6.70 | 18.09 | 11.38 | 648.36 | 696.27 | 47.91 | 29.49 | 31.49 | 2.00 |
| Zone 1 | | | | | | | | | | | | |
| D3290 S7370 | n.d. | n.d. | n.d. | 49.52 | 51.79 | 2.27 | 552.20 | 639.56 | 87.37 | 0.00 | 0.00 | 0.00 |
| Zone 2 | | | | | | | | | | | | |
| D3290 S7371 | n.d. | n.d. | n.d. | 15.28 | 9.46 | 0.00 | 126.65 | 267.27 | 140.62 | 0.00 | 0.17 | 0.17 |
| Zone 1 | | | | | | | | | | | | |
| D3290 S7371 | n.d. | n.d. | n.d. | 89.45 | 79.08 | 0.00 | 622.56 | 727.26 | 104.71 | 466.49 | 495.15 | 28.65 |
| Zone 2 | | | | | | | | | | | | |
| D1884 | n.d. | n.d. | n.d. | 0.00 | 0.00 | 0.00 | 506.81 | 564.87 | 58.07 | 0.00 | 0.00 | 0.00 |
| D273 S6045 | n.d. | n.d. | n.d. | n.d. | n.d. | n.d. | 72.90 | 497.00 | 424.09 | 0.00 | 0.00 | 0.00 |
| D273 S6046 | n.d. | n.d. | n.d. | n.d. | n.d. | n.d. | 267.58 | 707.61 | 440.03 | 5.64 | 44.95 | 39.31 |
| D1688 | n.d. | n.d. | n.d. | n.d. | n.d. | n.d. | 2.49 | 247.61 | 245.12 | 0.27 | 0.00 | 0.00 |
| Zone 1 | | | | | | | | | | | | |
| D1688 | n.d. | n.d. | n.d. | n.d. | n.d. | n.d. | 269.50 | 495.97 | 226.48 | 0.00 | 37.10 | 37.10 |
| Zone 2 | | | | | | | | | | | | |
| D1672 | n.d. | n.d. | n.d. | n.d. | n.d. | n.d. | 12.61 | 310.59 | 297.98 | 0.00 | 0.00 | 0.00 |
| D1682 | n.d. | n.d. | n.d. | n.d. | n.d. | n.d. | 13.70 | 373.76 | 360.06 | 12.03 | 50.34 | 38.31 |
| Zone 1 | | | | | | | | | | | | |
| D1682 | n.d. | n.d. | n.d. | n.d. | n.d. | n.d. | 64.54 | 466.28 | 401.74 | 0.00 | 1.92 | 1.92 |
| Zone 2 | | | | | | | | | | | | |
| D1682 | n.d. | n.d. | n.d. | n.d. | n.d. | n.d. | 31.69 | 333.52 | 301.82 | 117.24 | 10.03 | 0.00 |
| Zone 3 | | | | | | | | | | | | |
| D1682 | n.d. | n.d. | n.d. | n.d. | n.d. | n.d. | 23.23 | 357.35 | 334.12 | 27.36 | 48.57 | 21.21 |
| Zone 4 | | | | | | | | | | | | |

In table 3, TIL were seated in at 10e⁵ cells/well in triplicates in medium containing IL-2/IL-7/IL-15+cardiolipin (medium) or in IL-2/IL-7/IL-15+cardiolipin plus anti-CD3-IgG1 at 30 ng/mL to crosslink the T-cell receptor.

Supernatants were harvested after 24 hours in order to test for maximal T-cell activation defined in 24 hrs./10e⁵ cells for comparison. The delta between maximal stimulation (b) and constitutive cytokine production (A) is summarized in row “delta (B-A)”.

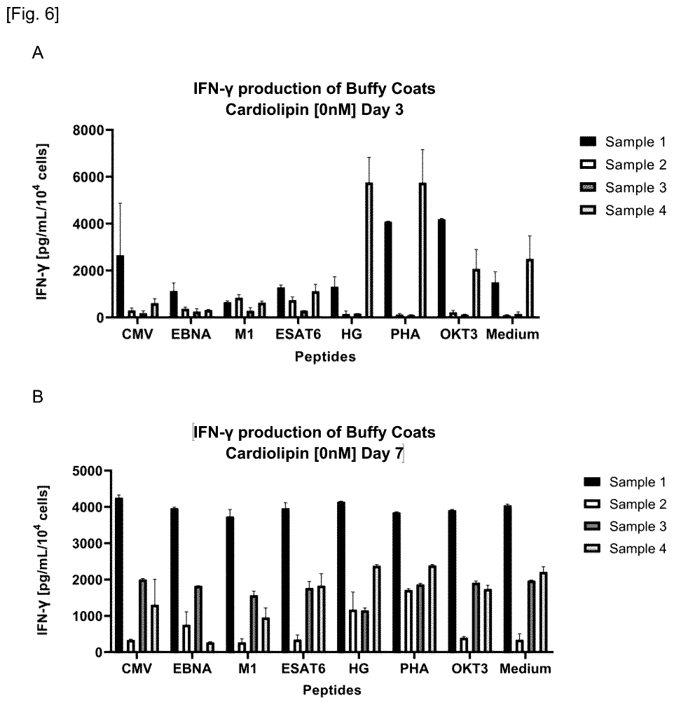

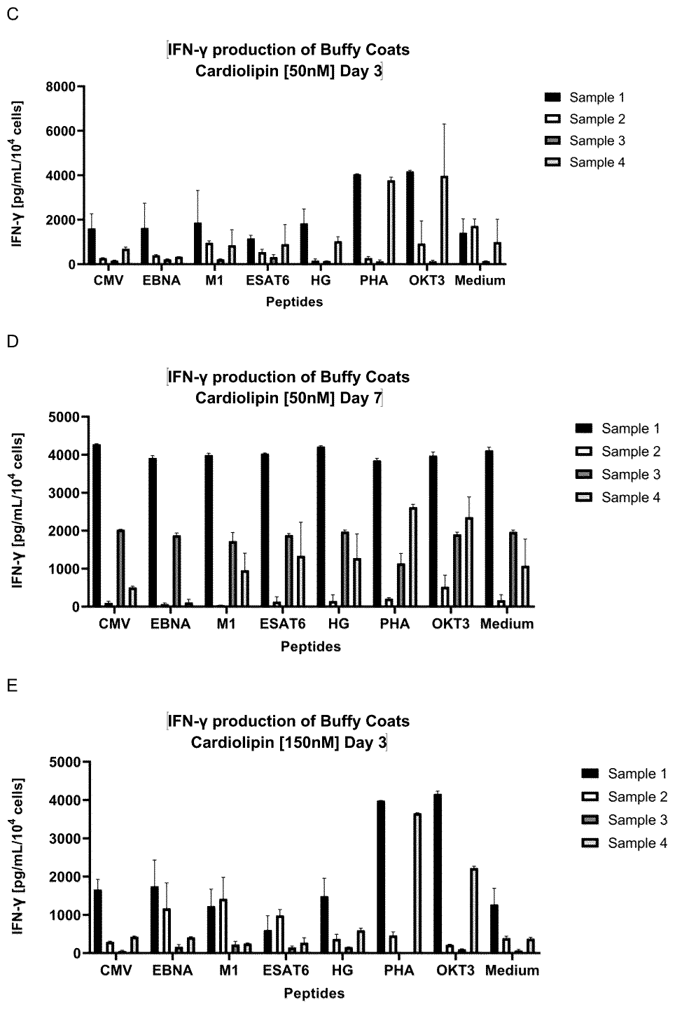

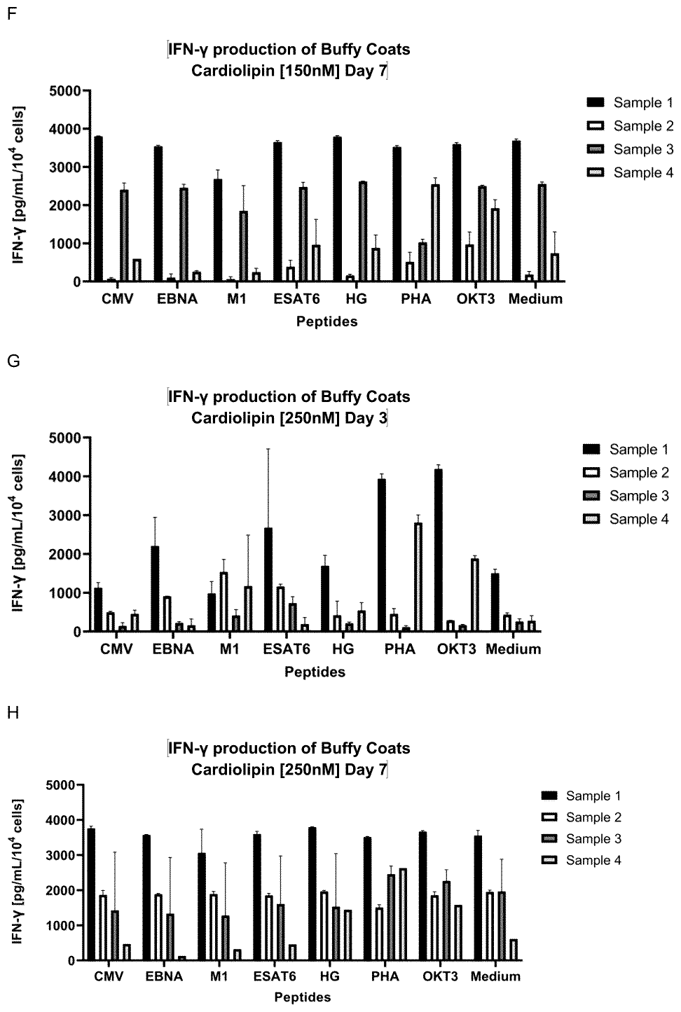

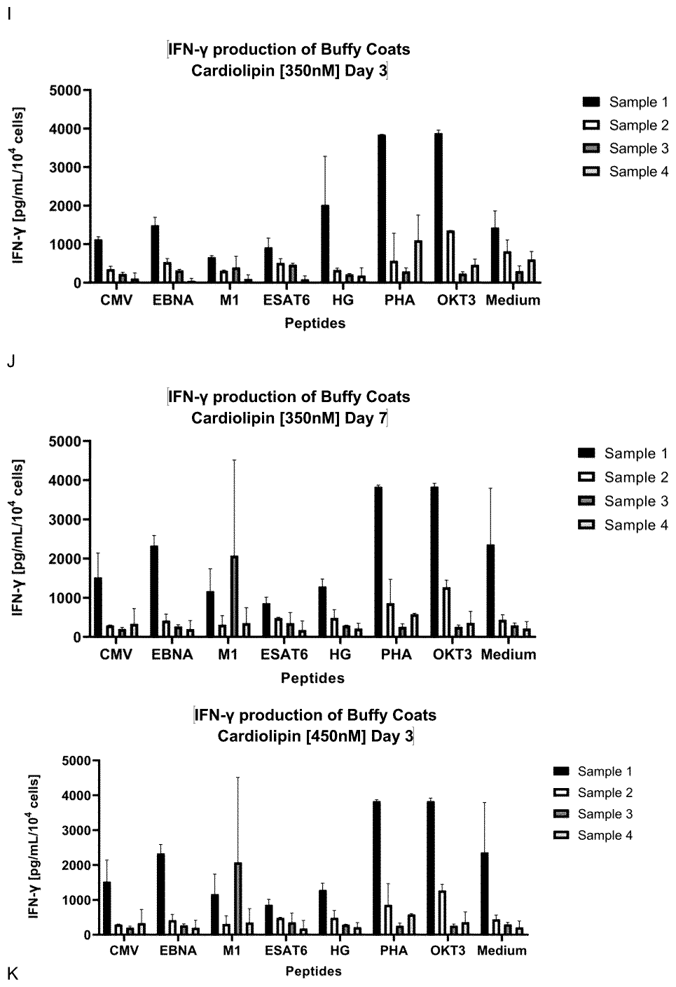

To compare the effect of cardiolipin in regard to cytokine production, the culture wells were supplemented with cardiolipin at different concentrations (0, 50, 150, 250, 350 and 450 nM) in culture wells with medium alone, positive controls, as well as commonly recognized target antigens, e.g., CMV, EBNA3, Flu-M1, ESAT-6, hemagglutinin (FIG. 6).

T-cell expansion medium was supplemented with 250 nM of cardiolipin, a concentration that showed favorable production of IFN-γ in PBMCs from healthy individuals stimulated with OKT3 (30 ng/mL) and cardiolipin.

PBMCs of healthy individuals expanded in the presence of cardiolipin without interleukins increased the frequency of γδ T-cells (Table 4).

TABLE 4
Percentage of γδ T-cells in healthy individuals
PBMCs in the presence or absence of cardiolipin.

| | w/o CL | w/CL |
|---|---|---|
| | 7.07% | 9.32% |
| | 14.8% | 26% |
| | 8.41% | 10.9% |
| | 18.8% | 22.9% |
| | 7.2% | 10.5% |
| Average | 11.26% | 15.92% |

TIL cultures were initiated by placing tumor pieces in individual 24 well culture vessels and fed as needed. The IL-7/IL-15/IL-21 cytokine mix was used to test whether cardiolipin would be able to (i) increase the frequency of TCR γδ T-cells, (ii) induce changes in T-cell phenotype or function and (iii) increase tumor antigen recognition defined by IFN-γ production.

Except for testing the effect of cardiolipin, TIL were expanded using the following protocol: tumor pieces were cultured for 4 days with IL-7 (1000 IU/mL), 15 (150 IU/mL) and IL-21 (1 IU/mL), supplemented with Cardiolipin (step 1), followed by adding anti-CD3 (30 ng/mL) and irradiated (with 40 Gy) allogeneic feeder cells at 10e6 cells/well (in a 24 well culture dish) from 3 donors at day 2 (step 2) and the medium was changed after 3-5 days to IL-2 (1000 IU/mL), IL-7 (1000 IU/mL) and IL-15 (150 IU/mL), also supplemented with Cardiolipin (step 3). Feeder cells were added for expansion every week starting tumor pieces were carefully removed around day 5-10 days after culture initiation. Cells were split as needed and expanded for individual patient TIL up to 1×10e⁹ TIL for testing. Cardiolipin was present during the entire culture period at 250 nM.

### Peptide Recognition Assays.

Peptides from tumor-associated antigens (TAAs), listed in Table 5 were placed at 1 ug/well in duplicates using anti-CD3 at 30 ng/mL as well as PHA at 5 μg/mL as positive controls.

TABLE 5

| Peptide | aa sequence |
|---|---|
| PHA | 5 μg/mL from Sigma Aldrich |
| OKT3 | 30 ng/ml from Biolegend or Human Cell |
| CMV | Peptide Pool of 138 15 mers with 11 aa overlap of |
| | pp65 (UniProt ID: P06725) |
| EBNA | Peptide Pool of 234 15 mers with 11 aa overlap of |
| | Epstein-Barr nuclear antigen 3 (UniProt ID: |
| | P12977) |
| M1 (mix) | Peptide Pool of 61 15 mers with 11 aa overlap of |
| | Matrix protein 1 (UniProt ID: B4UPA8) |
| ESAT6 | ELNNALQNL |
| EGFR | LEEKKGNYVVTDH |
| Hemagglutinin | VEPGDKITFEATGNL |
| Kras_P1 (G12WT) | YKLVVVGAGGVGKSALT |
| Kras_P2 (G12V) | YKLVVVGAVGVGKSALT |
| Kras_P3 (G12D) | YKLVVVGADGVGKSALT |
| Kras_P4 (G12R) | YKLVVVGARGVGKSALT |
| Kras_P5 (G12S) | YKLVVVGASGVGKSALT |
| Kras_P6 (G12C) | YKLVVVGACGVGKSALT |
| Kras_P7 (G12A) | YKLVVVGAAGVGKSALT |
| Kras_P8 (G13 WT) | KLVVVGAGGVGKSALTI |
| Kras_P9 (G13D) | KLVVVGAGDVGKSALTI |
| Kras_P10 (Q61 WT) | LDILDTAGQEEYSAMRD |
| Kras_P11 (Q61K) | LDILDTAGKEEYSAMRD |
| Kras_P12 (Q61H) | LDILDTAGHEEYSAMRD |
| Kras_P13 (Q61R) | LDILDTAGREEYSAMRD |
| Kras_P14 (A146 WT) | GIPFIETSAKTRQRVED |
| Kras_P15 (A146T) | GIPFIETSTKTRQRVED |
| KRAS_G12F | KLVVVGAVGVGKSAL |
| KRAS_G13C | LVVVGAGCVGKSALT |
| KRAS_G13S | LVVVGAGSVGKSALT |
| KRAS_G13R | LVVVGAGRVGKSALT |
| KRAS_G13A | LVVVGAGAVGKSALT |
| KRAS_G13V | LVVVGAGVVGKSALT |
| KRAS_Q61L | DILDTAGLEEYSAMR |
| KRAS_K117N | PMVLVGNNCDLPSRT |
| KRAS_A146V | IPFIETSVKTRQRVE |
| KRAS fitted for HLA- | LVVVGAVGVGKSALT |
| DQB1*06:02 | |
| SMAD4 [mutant private | LAFQPPISNHPAPEY |
| peptide] | |
| MART-1 (27-35) | AAGIGILTV |
| MART-1 (26-35) | ELAGIGILTV |
| Survivin (97-111) | TLGEFLKLDRERAKN |
| Survivin_1_HLA-A*02:01_1 | TLPPAWQPF |
| Survivin_1_HLA-A*02:01_2 | TLPPAWQPFL |
| Survivin_2_HLA-A*02:01_1 | QLYLKNYRI |
| NY-ESO (80-94) | ARGPERSLLEFYLAM |
| NY-ESO (89-103) | EFYLAMPFATPMEAE |
| NY-ESO (157-171) | SLLMWITQCFLPVFL |
| NY-ESO mix (pept. 1-43) | Peptide Mix of 43 15 mers with no overlap of NY- |
| | ESO (UniProt ID: P78358) |
| Mesothelin MPF-1 | MALPTARPLLGSCGT |
| Mesothelin MPF-8 | HRLSEPPEDLDALPL |
| Mesothelin MPF-11 | LLPRGAPERQRLLPA |
| Mesothelin MPF-12 | ALACWGVRGSLLSEA |
| Mesothelin MPF-14 | FVAESAEVLLPRLVS |
| Mesothelin MPF-17 | SVSTMDALRGLLPVL |
| Mesothelin GPI P40 (596- | LQGGIPNGYLVLDLS |
| 610) | |
| Mesothelin GPI P40 (601- | MQEALSGTPCLLGPG |
| 605) | |
| Mesothelin GPI P5 | YEQLDVLKHKLDELY |
| Mesothelin GPI P13 | WAVRPQDLDTCDPRQ |
| Mesothelin GPI P14 | LDVLYPKARLAFQNM |
| Mesothelin GPI mix (pept. | Peptide Mix of 23 15 mers with no overlap of NY- |
| 1-23) | ESO from position 286 to 630 (UniProt ID: |
| | Q13421) |
| MUC4-P1 [Mut][WT] | TPLPVTDASSASTGH |
| MUC4-P2 [WT] | TPLPVTDTSSASTGH |
| MUC4-P3 [Mut][WT] | PVTSLSSVSTGDTTP |
| MUC4-P4 [WT] | PVTSLSSASTGDTTP |
| MUC4-P5 [Mut][WT] | TTPLPVTIPSSASSG |
| MUC4-P6 [WT] | TTPLPVTSPSSASSG |
| MUC4-P7 [Mut][WT] | PVTSTSSVSTGHATP |
| MUC4-P8 [WT] | PVTSTSSASTGHATP |
| MUC4-P9 [Mut] | PVTSPSSESTGHAIP |
| MUC4-P10 [WT] | PVTSPSSASTGHAIP |
| MUC4-P11 [Mut] | TPLPVTDNSSASTGD |
| MUC4-P12 [WT] | TPLPVTDTSSASTGD |
| MUC4-P1 fitted for HLA- | VTDTSSASTGH |
| A*01:01 | |
| MUC4-P2_fitted for HLA- | VTDASSASTGH |
| A*01:01 | |
| MUC16-P1 [N1890K] | MGKSTHTSM |
| MUC16-P2 [Q5557K] | VMKHLLSPL |
| MUC16-P3 [T411N] | TTSPSNTLV |
| MUC16-P4 [T2630A] | EASSAVPTV |
| MUC16-P5 [WT] | MGNSTHTSM |
| MUC16-P6 [WT] | VMQHLLSPL |
| MUC16-P7 [WT] | TTSPSTTLV |
| MUC16-P8 [WT] | EASSTVPTV |
| MUC16 P9 | SKSLSMGKSTHTSMT |
| MUC16 P10 | NITDNVMKHLLSPLE |
| MUC16 P11 | LTTTSPSNTLVSEET |
| MUC16 P12 | HGAEASSAVPTVSPE |
| MUC16 P13 (WT) | SKSLSMGNSTHTSMT |
| MUC16 P14 (WT) | NITDNVMQHLLSPLE |
| MUC16 P15 (WT) | LTTTSPSTTLVSEET |
| MUC16 P16 (WT) | HGAEASSTVPTVSPE |

TIL were seeded at 10⁴/well in medium with 10% human serum added 24 h later and reduced cytokine concentrations (IL-2 at 100 IU/mL, IL-15 at 100 IU/mL). Supernatants were tested for IFN-γ production with the Human IFN-γ ELISA basic kit (HRP) (Mabtech, 3420-1H-6) according to the manufacturer's instructions. The assay can also be performed using IL-7 at 100 IU/mL and 100 IU IL-15/mL).

### Testing of the NRLP3 Pathway

Cardiolipin has been shown to mediate its effects via the NRLP3 pathway and in order to test this hypothesis, TIL were expanded in the presence of IL-7, IL-15, and IL-21 (1000 IU/mL, 150 IU/mL, 1 IU/mL) in CellGenix GMP DC media supplemented with 10% serum in the presence or absence of the NRLP3 pathway inhibitor MCC950 (10 uM/mL) throughout the culture.

TIL were expanded as outlined above and tested for recognition of a panel of 26 KRAS peptides (listed in Table 6 below). The number of peptide targets recognized as well as the amount of IFN-γ in pg/10e⁴ TIL/target/7 days was analyzed and compared in the cardiolipin-positive versus negative group testing with Human IFN-γ ELISA basic kit (HRP).

TABLE 6

| Name on vial | Peptide |
|---|---|
| KRAS_P1 | YKLVVVGAGGVGKSALT |
| KRAS_P2 | YKLVVVGAVGVGKSALT |
| KRAS_P3 | YKLVVVGADGVGKSALT |
| KRAS_P4 | YKLVVVGARGVGKSALT |
| KRAS_P5 | YKLVVVGASGVGKSALT |
| KRAS_P6 | YKLVVVGACGVGKSALT |
| KRAS_P7 | YKLVVVGAAGVGKSALT |
| KRAS_P8 | KLVVVGAGGVGKSALTI |
| KRAS_P9 | KLVVVGAGDVGKSALTI |
| KRAS_P10 | LDILDTAGQEEYSAMRD |
| KRAS_P11 | LDILDTAGKEEYSAMRD |
| KRAS_P12 | LDILDTAGHEEYSAMRD |
| KRAS_P13 | LDILDTAGREEYSAMRD |
| KRAS_P14 | GIPFIETSAKTRQRVED |
| KRAS_P15 | GIPFIETSTKTRORVED |
| KRAS_G12F | KLVVVGAVGVGKSAL |
| KRAS_G13C | LVVVGAGCVGKSALT |
| KRAS_G13S | LVVVGAGSVGKSALT |
| KRAS_G13R | LVVVGAGRVGKSALT |
| KRAS_G13A | LVVVGAGAVGKSALT |
| KRAS_G13V | LVVVGAGVVGKSALT |
| KRAS_Q61L | DILDTAGLEEYSAMR |
| KRAS_K117N | PMVLVGNNCDLPSRT |
| KRAS_A146V | IPFIETSVKTRORVE |
| DQ6KRAS | LVVVGAVGVGKSALT |
| restricted | |
| molecule | |
| M1 | 61 15 mers with 11 aa |
| | overlap of Matrix protein |
| | 1 UniProt ID: B4UPA8 |
| EBV | 234 15 mers with 11 aa |
| | overlap of Epstein-Barr |
| | nuclear antigen 3 UniProt |
| | ID: P12977 |
| CMV | Peptide Pool of 138 |
| | 15 mers with 11 aa |
| | overlap of pp65 (UniProt |
| | ID: P06725) |
| PHA | 5 ug/mL from Sigma |
| | Aldrich |
| OKT3 | 30 ng/ml from Biolegend |
| | or Human Cell |

### Blocking Antibodies

MHC class I responses were blocked using mAb clone w6/32 (mouse IgG2a) at 10 μg/mL, anti-HLA-DR clone L243 (mouse IgG2a) at 10 μg/mL, anti-HLA-DP clone B7/21 (mouse IgG3) at 10 μg/mL, anti-CD1d clone CD1d42 (mouse IgG1) at 10 μg/mL. Isotype control antibodies included mouse IgG2a and mouse IgG1 at 10 μg/mL. MR1 responses were blocked using the mAb clone 26.5 (mouse IgG2a) at 10 μg/mL. All antibodies are commercially available.

siRNA

siRNA against MR1 was used to silence MR1 expression using Silencer Select Pre-designed siRNA (Ambion, Thermo Fisher)). A scramble siRNA was used as negative control (Ambion, Thermo Fisher). Briefly, target cells were seeded 24 h before transfection, both siRNAs were used at 10 μM with lipofectamine RNA iMAX diluted in OptiMEM, incubated for 5 minutes, and added on top of the cells. Cells were incubated for 48 h before being used on the respective assays.

### γδ T-Cell Isolation

γδ T-cells were negatively sorted using a commercial kit from Stem Cell. Purity of negatively sorted cells was tested by flow cytometry using the DURAclone panel IM TCRs.

### Cellular Recognition Assay

Autologous tumor, confirmed by standard histology, was viably snap frozen and then thawed to be used as targets in recognition assays once TIL had been expanded. Small autologous tumor pieces (1 mm³) from tumor areas that contained tumor cells were tested in duplicates. TIL were incubated with tumor pieces or allogeneic tumor cells in CellGenix GMP DC media supplemented with 10% serum in the presence of IL-2 (50 IU/mL) and IL-15 (10 IU/mL) for testing MAIT—cells. TCR αβ T-cells or TCR γδ cells were tested for autologous or allogeneic tumor recognition using 100 IU IL-2/mL and 100 IU IL-15/mL; or using 100 IU IL-7/mL combined with 100 IU IL-15/mL. The allogeneic tumor cell line PANC-1 obtained from ATCC (CRL-1469) derived from pancreatic duct epithelioid carcinoma, was maintained in DMEM high glucose, GlutaMAX Supplement, and served as a target for sorted γδ T-cells or Vα7.2 sorted (MAIT) T-cells.

### Mitochondrial Isolation

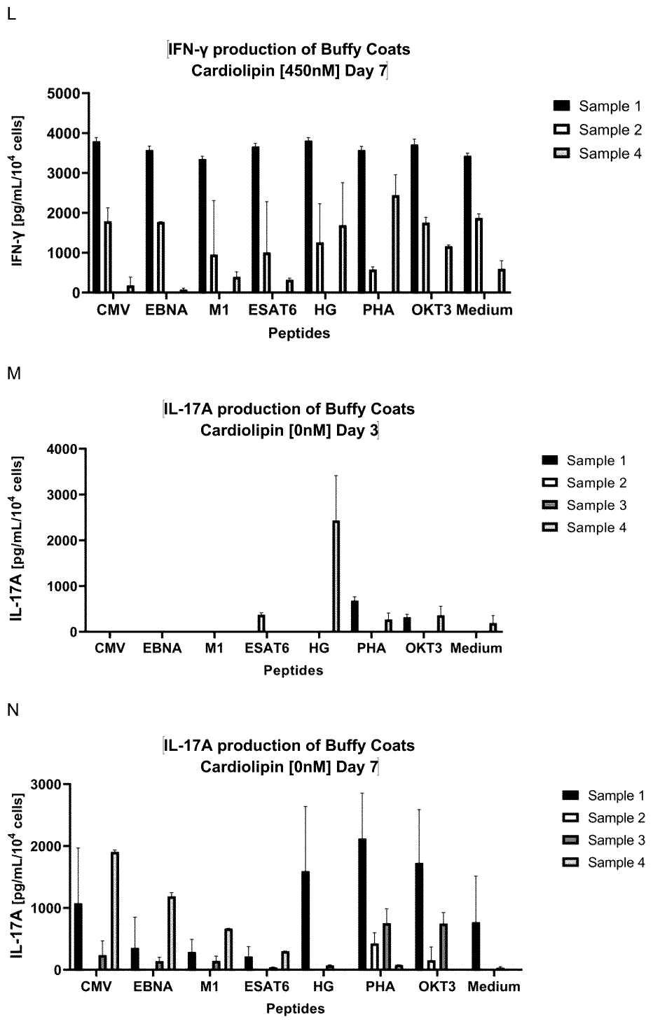

Mitochondria were isolated from PBMCs from healthy blood donors provided by the National Blood Bank Lisbon, Portugal, and were isolated using the commercial isolation kit Mitochondria Isolation Kit for culture cells (Thermo Scientific). Mitochondrial isolation was confirmed by Western Blot using the commercial antibody TOM20 (clone EPR15581-54) (FIG. 7) detecting an antigen-specific band at 16 kDa.

Mitochondria were isolated from PBMCs from healthy donors cultured for 48 h at 2×10⁶/mL without cytokines or cultured with doxorubicin 1 uM for 12 h, which induces mitochondrial abnormalities. PANC-1 cells were used as a source for mitochondria using the commercial isolation kit Mitochondria Isolation Kit for culture cells (Thermo Scientific). Mitochondria were also isolated from PANC1 cells treated with or without leflunomide (that induces mitochondrial fusion) at 50 μM for 48 h at ng/mL or rapamycin (10 nM for 12 h).

### Mitochondrial Presentation Assays.

For APC differentiation in vitro, PBMCs (from the patients whose isolated TCR γδ⁺ T-cells were tested) were obtained from heparinized blood and plated onto flat bottom 96-well culture plates in the presence of serum-free RPMI medium for 2 h at 37° C. and 5% CO₂. Then, non-adherent cells were washed with PBS and adherent cells were cultured in RPMI with 5% Human serum and cytokines GM-CSF (800 IU/mL) and IL-4 (800 IU/mL) for an additional 5 days.

APCs (antigen presenting cells) were exposed to different mitochondria solutions (100 μg/mL) overnight and the next day, γδ⁺ T-cells were seeded on APCs exposed to mitochondria at 10⁴ cells/well, blocking antibodies were added (e.g., anti-CD1d or isotype controls) and supernatants were tested for IFN-γ production.

### Whole Exome Sequencing Pipeline

Exome sequencing data was aligned using the Burrows-Wheeler Aligner to human genome build hg19. Duplicates were marked using Picard's MarkDuplicates tool and base recalibration was performed using GATK. Once the data was recalibrated, samtools was used to create the tumor tissue and, for comparison, the non-transformed tissue pileup files.

Four different tools were used to call mutations: Mutect2, Varscan2, Strelka2 and Lancet. A consensus VCF file containing mutations called from at least two tools is then annotated using the Ensembl Variant Effect Predictor. Candidate neoantigens were identified using pVACtools, wherein two types of peptides are predicted: peptides with 15 residues where the alteration is centered or the full downstream protein sequence in case of a frameshift, that are used for immunoassays, and peptides of different lengths tailored for the MHC typing of the patient that are candidates for PCV.

The entire set of tumor mutations for the TIL from patients D1309 and D1313 are listed in the Table 7 below. Peptides selected for TIL recognition were selected based on (i) driver mutations, (ii) frequency of reads/million reads, (iii) frameshift mutations, (iv) binding to MHC class I—or class II molecules and (v) the possibility to synthetically produce the peptides.

TABLE 7
D1309

| | | Location of |
|---|---|---|
| | | peptide in the |
| Peptide ID | Peptide sequence | protein |
| D1309_LRP1B | VSKRLKFSRDLSLDP | positions 3125- |
| | | 3139 UniProt ID: |
| | | Q9NZR2 with |
| | | P3132S mutation |
| D1309_ALMS1 | YSHTEKPDVFYQQVL | positions 1333- |
| | | 1347 Ensembl ID: |
| | | ENSP00000264448 |
| | | with G1340D |
| | | mutation |
| D1309_CDH12 | PVGTIIGVVTAQDLD | positions 398-412 |
| | | UniProt ID: |
| | | P55289-1 with |
| | | A405V mutation |
| D1309_GLIS3 | IGKGSCNSLVVTSSP | positions 141-155 |
| | | UniProt ID: |
| | | Q8NEA6-2 with |
| | | T148S mutation |
| D1309_HTR1E | LDRYWAISNAIEYAR | positions 118-132 |
| | | UniProt ID: |
| | | P28566 with |
| | | T125S mutation |
| D1309_MYT1L | PIAAAEKMAKAQEKH | positions 573-587 |
| | | UniProt ID: |
| | | Q9UL68-1 with |
| | | L580M mutation |
| D1309_ONECUT2 | TLTPLQPQPPISTVS | positions 150-164 |
| | | UniProt ID: |
| | | O95948 with |
| | | L157Q mutation |
| D1309_WFS1 | SMVKLILLWLTAIVL | positions 631-645 |
| | | UniProt ID: |
| | | O76024 with |
| | | V638L mutation |
| D1309_LRP1B_wt | VSKRLKFPRDLSLDP | positions 3125- |
| | | 3139 UniProt ID: |
| | | Q9NZR2 |
| D1309_ALMS1_wt | YSHTEKPGVFYQQVL | positions 1333- |
| | | 1347 Ensembl ID: |
| | | ENSP00000264448 |
| D1309_CDH12_wt | PVGTIIGAVTAQDLD | positions 398-412 |
| | | UniProt ID: |
| | | P55289-1 |
| D1309_GLIS3_wt | IGKGSCNNLVVTSSP | positions 141-155 |
| | | UniProt ID: |
| | | Q8NEA6-2 |
| D1309_HTR1E_wt | LDRYWAITNAIEYAR | positions 118-132 |
| | | UniProt ID: |
| | | P28566 |
| D1309_MYT1L_wt | PIAAAEKLAKAQEKH | positions 573-587 |
| | | UniProt ID: |
| | | Q9UL68-1 |
| D1309_ONECUT2_wt | TLTPLQPLPPISTVS | positions 150-164 |
| | | UniProt ID: |
| | | O95948 |
| D1309_WFS1_wt | SMVKLILVWLTAIVL | positions 631-645 |
| | | UniProt ID: |
| | | O76024 |

D1313

| | | Location of |
|---|---|---|
| | | peptide in the |
| Peptide ID | Peptide sequence | protein |
| D1313_TPR | TTNETATSDDGDEVF | positions 2253- |
| | | 2267 UniProt ID: |
| | | P12270-1 with |
| | | G2260S mutation |
| D1313_CCDC116 | EPCRSLYSNLPASRQ | positions 541-555 |
| | | UniProt ID: |
| | | Q8IYX3-2 with |
| | | T548S mutation |
| D1313_SPEF2 | QRCFDIEMQYLNRRR | positions 195-209 |
| | | UniProt ID: |
| | | Q9C093-1 with |
| | | K195M mutation |
| D1313_PTGIS | LHLEEMGMSEEMQAR | positions 261-275 |
| | | UniProt ID: |
| | | Q16647 with |
| | | V268M mutation |
| D1313_STAB1 | FSFVRGQLLFKGCDV | positions 20-34 |
| | | UniProt ID: |
| | | Q9NY15-1 with |
| | | V27L mutation |
| D1313_C6 | CQCAPCPKNGRPTLS | positions 521-535 |
| | | UniProt ID: |
| | | P13671 with |
| | | N528K mutation |
| D1313_B3GAT2 | VQKAELTGLANTFRQ | positions 93-107 |
| | | UniProt ID: |
| | | Q9NPZ5 with |
| | | R100G mutation |
| D1313_SFXN5 | LGAVISADSIAVGLN | positions 168-182 |
| | | UniProt ID: |
| | | Q8TD22 with |
| | | V175D mutation |
| D1313_GOLGA6L10 | GGAEEAAGHSFRAAE | positions 456-470 |
| | | Ensembl ID: |
| | | ENSP00000388606 |
| | | with C463G |
| | | mutation |
| D1313_VLDLR | KSRLYWLASKLHMLS | positions 611-625 |
| | | UniProt ID: |
| | | P98155-1 with |
| | | D618A mutation |
| D1313_C17orf53 | LLETCQNKLKPGSVL | positions 531-545 |
| | | UniProt ID: |
| | | Q8N3J3-1 with |
| | | E538K mutation |
| D1313_FAM161B | AELFRKICIQMRALD | positions 373-387 |
| | | UniProt ID: |
| | | Q96MY7-1 with |
| | | R380C mutation |
| D1313_KIAA0368 | LQSQSWKVKAQGAIA | positions 1708- |
| | | 1722 UniProt ID: |
| | | J3KN16 with |
| | | M1715V mutation |
| D1313_THSD7B | QSCDPHTIQRRTRHL | positions 1144- |
| | | 1158 UniProt ID: |
| | | Q9C014 with |
| | | M11511 mutation |
| D1313_PRKCE | PVLTLVDKAIVKQIN | positions 707-721 |
| | | UniProt ID: |
| | | Q02156 with |
| | | G714K mutation |
| D1313_PHEX | SYGAIGVTVGHEFTH | positions 570-584 |
| | | UniProt ID: |
| | | P78562 with |
| | | I577T mutation |
| D1313_POM121L2 | LMLKPILWPLHNSEI | positions 532-546 |
| | | UniProt ID: |
| | | Q96KW2 with |
| | | G539W mutation |
| D1313_ZNF518B | DQLIKCPCRNQPVIV | positions 931-945 |
| | | UniProt ID: |
| | | Q9C0D4 with |
| | | R938C mutation |
| D1313_HS3ST3A1 | LPQAIIIRVKKGGTR | positions 152-166 |
| | | UniProt ID: |
| | | Q9Y663 with |
| | | G159R mutation |
| D1313_LRRN3 | TNNPRLSFIHPNAFF | positions 317-331 |
| | | UniProt ID: |
| | | Q9H3W5 with |
| | | Y324F mutation |
| D1313_TPR_wt | TTNETATGDDGDEVF | positions 2253- |
| | | 2267 UniProt ID: |
| | | P12270-1 |
| D1313_CCDC116_wt | EPCRSLYTNLPASRQ | positions 541-555 |
| | | UniProt ID: |
| | | Q81YX3-2 |
| D1313_SPEF2_wt | QRCFDIEKQYLNRRR | positions 195-209 |
| | | UniProt ID: |
| | | Q9C093-1 |
| D1313_PTGIS_wt | LHLEEMGVSEEMQAR | positions 261-275 |
| | | UniProt ID: |
| | | Q16647 |
| D1313_STAB1_wt | FSFVRGQVLFKGCDV | positions 20-34 |
| | | UniProt ID: |
| | | Q9NY15-1 |
| D1313_C6_wt | CQCAPCPNNGRPTLS | positions 521-535 |
| | | UniProt ID: |
| | | P13671 |
| D1313_B3GAT2_wt | VQKAELTRLANTFRQ | positions 93-107 |
| | | UniProt ID: |
| | | Q9NPZ5 |
| D1313_SFXN5_wt | LGAVISAVSIAVGLN | positions 168-182 |
| | | UniProt ID: |
| | | Q8TD22 |
| vacD1313_PKN2_2 | KLVISPLEL | positions 209-217 |
| | | UniProt ID: |
| | | Q16513-1 with |
| | | P210L mutation |
| vacD1313_POM121L2 | LMLKPILWPL | positions 532-541 |
| | | UniProt ID: |
| | | Q96KW2 with |
| | | G539W mutation |
| vacD1313_KIAA0368 | ALQSQSWKV | positions 1707- |
| | | 1715 UniProt ID: |
| | | J3KN16 with |
| | | M1715V mutation |
| vacD1313_C17orf53 | RLLLETCONK | positions 529-538 |
| | | UniProt ID: |
| | | Q8N3J3-1 with |
| | | E538K mutation |
| vacD1313_C6 | CPKNGRPTL | positions 526-534 |
| | | UniProt ID: |
| | | P13671 with |
| | | N528K mutation |
| vacD1313_HS3ST3A1 | RVKKGGTRAL | positions 159-168 |
| | | UniProt ID: |
| | | Q9Y663 with |
| | | G159R mutation |
| vacD1313_SPEF2 | IEMQYLNRRRQNEI | positions 193-207 |
| | | UniProt ID: |
| | | Q9C093-1 with |
| | | K195M mutation |
| vacD1313_TPR | SDDGDEVFVEA | positions 2260- |
| | | 2270 UniProt ID: |
| | | P12270-1 with |
| | | G2260S mutation |
| vacD1313_PHEX | RSLSYGAIGVTVGHE | positions 567-581 |
| | | UniProt ID: |
| | | P78562 with |
| | | I577T mutation |
| vacD1313_PKN2 | KLVISPLELRMEEL | positions 209-222 |
| | | UniProt ID: |
| | | Q16513-1 with |
| | | P210L mutation |
| vacD1313_ZNF518B | LKDPSIFQVARQLRLIAAKPDQLIK | positions 911-938 |
| | CPC | UniProt ID: |
| | | Q9C0D4 with |
| | | R938C mutation |
| vacD1313_UQCRFS1 | FVSSMSASAVVLALAKIEIKLSD | positions 136-158 |
| | | UniProt ID: |
| | | P47985 with |
| | | D145V mutation |

### RNA Extraction and Sequencing

RNA from TIL was isolated using the commercially available RNeasy Mini Kit (Qiagen) and sequencing was done using the Illumina NovaSeq 6000 system with 100 bp paired end read lengths. Adapters were removed.

Transcriptome sequencing data alignment was performed using STAR two-pass method to human genome build hg19. Duplicates were marked using the Picard's MarkDuplicates tool and reads were split using the GATK SplitNCigarReads tool and variants were called using Varscan2. STAR-Fusion was used to identify candidate fusion transcripts and its output was annotated using AGFusion.

### TCR Library Preparation and Sequencing

The NGS libraries covering human TCRα, TCRβ, TCRγ and TCRδ CDR3 regions were prepared using the commercially available iR-RepSeq-plus 7-Chain Cassette. Briefly, 1 μg of total RNA was loaded into the disposable cassette which contains all necessary reagents for library amplification, purification and processed by iR-Processor (iRepertoire).

All four chains were amplified in a single assay using a strategy which allows the incorporation of unique molecular identifiers (UMIs) during the reverse transcription (RT) step. The final constructed library includes Illumina dual index sequencing adapters, a 10-nucleotide UMIs, and an 8-nucleotide internal barcode associated with the C-gene primer.

After quality control, amplified libraries were multiplexed and pooled for sequencing on the Illumina NextSeq platform with a 300-cycle kit (300 single-end reads). The samples were analyzed using iRepertoire's proprietary YPL5-UMI bioinformatics pipeline which condenses the sequences by UMI, removes sequencing artifacts, and identifies CDR3 regions by mapping the reads to reference sequences.

### Flow Cytometric Analysis

TIL and PBMCs were analyzed using several DuraClone (Beckman Coulter) Panels, always with addition of the LIVE/DEAD™ Fixable Yellow Dead Cell Stain Kit: DuraClone IM T-cell Subsets tube adding LAG-3 BV650 and CD95 BV785 and Duraclone IM TCRs tube. Besides, another panel with commercially available antibodies were used, which included CD4 Pacific Blue, CD8 KromeOrange, CXCR3 BV650, CD95 BV785, CD103 FITC, CCR7 PE, CCR4 PE/Dazzle, TCR pan-γ/δ PC5.5, CCR6 PC-7, CCR9 Alexa Fluor 647, CD45RA AF700, CD3 APC-A750 and LIVE/DEAD™ Fixable Yellow Dead Cell Stain Kit. After 15 minutes incubation, T-cells and PBMCs were washed in PBS-2% FBS and acquired using a CytoFlex LX flow cytometer from Beckman Coulter. Analysis was performed using FlowJo software.

### Treg Detection Assay

Briefly, cells were stained extracellularly with CD3 PE (clone UCHT-1, BD 555749), CD4 V450 (clone RPA-T4, BD 561838), CD8 APC-Cy7 (clone SK1, BD 557834), CD25 PE-Cy7 (clone 2A3, BD 335824), CD127 APC (clone R34.34, Beckman Coulter B42026) for 15 minutes in ice and then washed and permeabilized using Biolegend's True-Nuclear Transcription Factor Buffer Set according to manufacturer's instructions.

Cells were then stained intracellularly with FOXP3 Alexa488 antibody (clone 259D/C7, BD 560047) or an IgG1 isotype control Alexa 488 (BD 557702), and CD3 PE (clone UCHT-1, BD 555749). Tregs cells were identified by flow cytometry as CD3⁺ CD4⁺ CD25^{high} CD127⁻FoxP3⁺).

### CD107a Assay

Briefly, positive control cells were stimulated with 1 μg/mL of PMA; non-stimulated cells served as the control and data were performed in triplicates. Both stimulated and non-stimulated cells were incubated with a protein transport inhibitor containing monensin (BD, 51-2092kz) and CD107a PE antibody (clone H4A3, BD 555801) for 2 hours. Then an extracellular staining was performed using CD3 PE-Cy7 (clone UCHT1, BD 563423), CD4 V450 (clone RPA-T4, BD 561838) and CD8 APC-Cy7 (clone SK1, BD 557834). Control cells are PBMCs that had been cultured for 48-72 hours in the presence of 300 IU/mL IL-2 and 50 IU/mL IL-15. The assay is considered as positive if the positive control cells (PMA stimulated) have at least 2× the SD of the triplicates of the negative control.

### IFN-γ Induction Assay

This assay is used to measure the IFN-γ production after anti-CD3 induced activation during 24 h in 10⁵ cells. Briefly, TIL are seeded in triplicates (10⁵ cells/wells) in TIL medium supplemented with 10% Human Serum and 1000 IU/mL IL-2, IL-7 1000 IU/mL and 150 IU/mL IL-15 (i.e. the 3^{rd} step of the TIL expansion protocol) in the presence of an anti-CD3 crosslinking antibody 30 ng/mL (for the positive control wells. There are also 3 wells seeded with medium and no cells as an additional control for IFN-γ that may be present in the human serum as a part of the cell culture medium. After 24 h incubation at 37° C. and 5% CO2, supernatants are harvested and an IFN-γ ELISA (Human IFN-γ ELISA basic kit (HRP) (Mabtech, 3420-1H-6)) is run according to manufacturer's instructions.

### Western-Blot Assay

A Western-Blot assay was performed to assess the quality of the mitochondria extraction from PANC-1 and healthy PBMCs using a monoclonal antibody against the mitochondrial specific protein TOMM20. Briefly, mitochondrial protein extracts were first measured to assess their concentration by a Bradford assay and equal amounts of protein for each condition were lysed using RIPA buffer. The protein extracts were added to an SDS-PAGE gel and run at a constant voltage of 100V for 1 hour. Then the protein extracts were transferred from the gel to a membrane and blocked in PBS-5% non-fat milk. Then the membrane was incubated with primary antibody rabbit anti-human TOMM-20 (clone EPR15581-54, ab186735, abcam) diluted in PBS-5% non-fat milk overnight at 4° C., washed three times in TBS-Tween and incubated for 1 hour in secondary antibody anti-rabbit at room temperature. Finally, the membrane was washed three times and developed using the kit Pierce™ ECL Plus Western Blotting Substrate.

### Statistical Analysis

The statistical analysis was performed for antigen recognition only and used a t-student test with software Prism 7. Statistical significance was considered with p value minor than 0.05 to detect differences in tumor-associated target recognition by TIL.

### Vα7.2⁺ or MR1-Reactive T-Cell Analysis in TIL

TIL were washed with PBS (Corning Life Sciences, New York, USA) and incubated for 15 min with antibodies at room temperature (except the MR1-tetramers that required 40 minutes of incubation, then the cells were washed, and the second layer of antibodies was added for incubation). The MR1 tetramer technology was developed jointly by Dr. James McCluskey, Dr. Jamie Rossjohn, and Dr. David Fairlie, and the material was produced by the NIH Tetramer Core Facility as permitted to be distributed by the University of Melbourne.

After the staining, cells were washed with PBS (Corning Life Sciences, New York, USA), resuspended in 200 μL of PBS (Corning Life Sciences, New York, USA) and analyzed by flow cytometry (CytoFLEX, Beckman Coulter, Brea, CA, USA). Data analysis was performed using FlowJo software version 10.7.1. In detail, MR1-6FP and MR1-5-OP-RU tetramers were kindly provided by the NIH facility.

α-TCRVα7.2 (clone 3C10), α-CCR7 (clone G043H7), α-CD69 (clone FN50), α-CD161 (clone HP-3G10), α-CD3 (clone UCHT1), α-CD26 (clone BA5b), and α-CD95 (clone DX2) are from Biolegend, CA, USA; α-CD45RA (clone 2H4) is from BD Biosciences, CA, USA; α-CD161 (clone HP-3G10) is also from ExBIO, Vestec, Czech Republic; and α-CD4 (clone 13B8.2), α-CD8 (clone B9.11), α-CD103 (clone 2G5) are from Beckman Coulter, Brea, Ca, USA.MAIT were isolated with the immunomagnetic EasySep™ Release Human PE Positive Selection Kit (STEMCELL™ Technologies), using α-TCRVα7.2 PE-conjugated antibody (clone 3C10, Biolegend, CA, USA). The sorting comprises FcR blocking to prevent unwanted binding of antibodies and staining with the PE antibody. MAITs are labelled with α-TCRVα7.2 antibody followed by incubation with PE-binding magnetic beads. The desired cells are then magnetically separated and once removed from the magnet, are able to be collected from the tube. A release buffer is used to then remove the PE-binding beads.

After calculating cell concentration, MAIT underwent functional assays, i.e., recognition of autologous tumor cells and blocking with anti-MR1 specific monoclonal antibodies as well as appropriate control antibodies. The allogeneic tumor cell line PANC-1 was also tested a s target and MR1 restriction was shown using MR1-specific siRNA as well as scrambled RNA.

### Bacterial Recognition by MAIT

Five strains of bacteria were grown—Saccharopolyspora rectivirgula (strain 683 [DSM 43747 INMI 683, VKM-A-810] ATCC), Bacillus clausii (strain DSM 8716 [C360, NCIB 1039, PN-23] ATCC), Pseudoxanthomonas mexicana (strain AMX 26B ATCC), Streptomyces mycarofaciens (strain SF-837 ATCC) and Enterococcus faecalis (strain AGR 329 ATCC)—in 50 mL tubes Bacteria concentration was evaluated using a Bacteria Counting Kit (Invitrogen, Thermo Fisher Scientifics, MA, USA), which enumerates bacteria by flow cytometry using a calibrated suspension of microspheres beads and a nucleic acid that penetrates bacteria (both Gram-negative and positive).

The staining of beads plus bacteria are read in the green fluorescence channel and distinguished on a plot of forward scatter versus FITC. The density of the bacteria in the sample was calculated from the ratio of bacterial signals to microsphere signals. TIL were exposed to the different bacterial species which were reported to be associated with increased survival in patients with pancreatic cancer. Either the (washed) bacterial directly or the bacterial supernatants were used to stimulate TIL, supernatants were then harvested and tested for cytokine production by ELISA.

### Results

As previously disclosed, TIL from sixteen patients (listed in Table 1) with various amounts of γδ⁺ TIL infiltrations (see FIG. 8) were expanded in the presence of IL-7, IL-15, and IL-21 and in the presence or absence of cardiolipin.

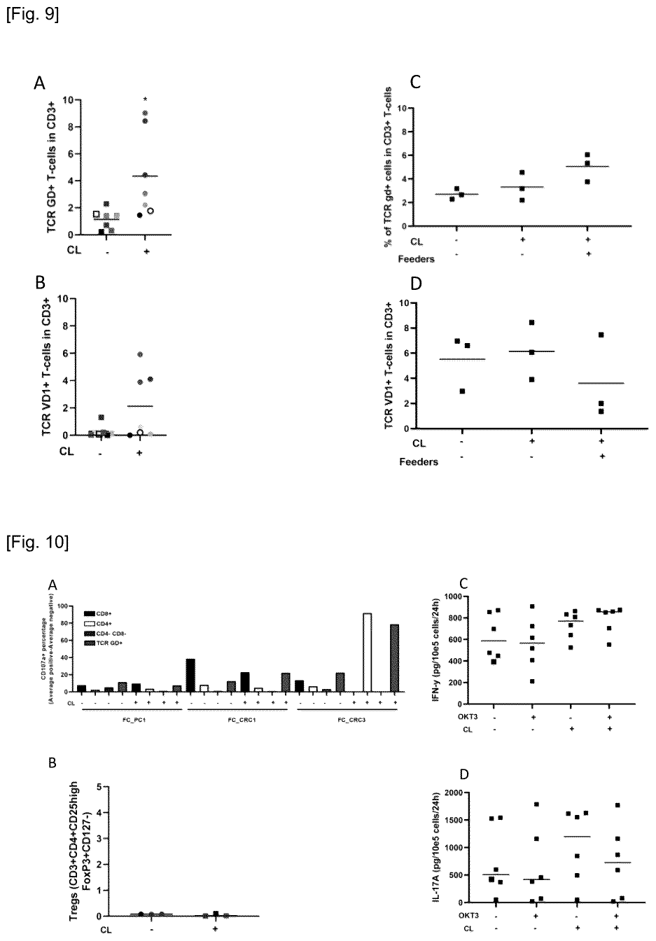

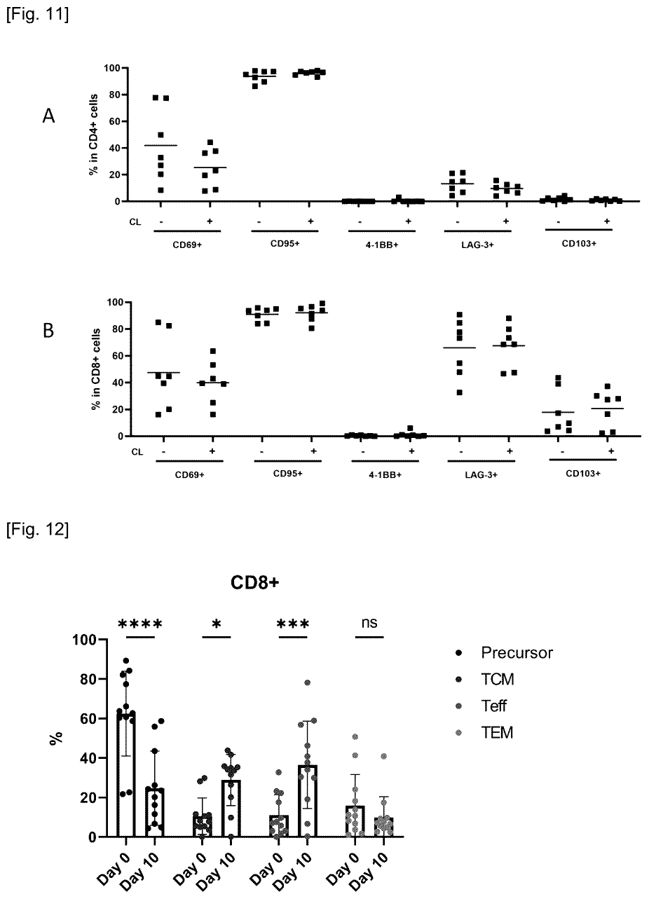

Differences were found in the frequency of γδ TIL (FIG. 9) which showed to be increased in TIL expanded in cardiolipin. However, no differences regarding IFN-γ production, IL-17 production, nor CD107 induction or regulatory T-cells (Treg) in TIL, without or without cardiolipin, were observed (FIG. 10). Differences regarding T-cell maturation, activation, or exhaustion markers in CD4⁺, CD8⁺, double-positive (CD4⁺CD8⁺), double-negative (CD4⁻CD8⁻) or γδ⁺ TIL (FIG. 11) were not observed.

Using the identical 3 step expansion process (using cardiolipin, IL-7/IL-15/IL-21 followed by IL-2/IL-7/IL-15 and anti-CD3 stimulation) in peripheral blood mononuclear cells (PBMCs) exhibited that the method does not drive T-cell into terminal differentiation.

It can be observed an decrease in precursor cells, yet also an increase in central memory and effector memory T-cells and a reduction in frequency of terminally differentiated T-cells (FIG. 12), which makes the expansion method suitable to activate and expand peripheral T-cells as recipients for transgenic immune recognition receptors, e.g. T-cell receptor or antibodies (chimeric antigen receptor technology) using different genetic transfer methods, for instance mRNA, CRISPR-platforms, retroviral or lentiviral vectors, not excluding alternative genetic delivery platforms.

Mutant tumor antigen-specific T-cells could also be expanded using the 3-step expansion method from (tumor cells) free healthy skin that exhibited T-cell infiltration microscopically and clinically as erythema/papule-like lesions. In contrast, PBMCs harvested at the same day did not yield tumor antigen specific T-cells demonstrating that i) the anatomical location as well as ii) the expansion method allowed for tumor—antigen specific T-cells that could give as a source for cell-therapy or antigen-specific-T-cell receptors, as can be seen in Table 8 below.

TABLE 8
Mutant tumor antigen specific T-cells expanded from
skin lesions from patients with solid cancer

| | PBMCs | PBMCs | |
|---|---|---|---|
| | IFN-y | IFN-y | Posterior skin |
| | pg/ml/ | pg/ml/ | lesion T-cells |
| Peptide | 7 days | 7 days | IFN-y pg/ml/7 days |
| PHA | 165.7 | 165.7 | 901.0 |
| OKT3 | 0.0 | 0.0 | 27.0 |
| CMV | 0.0 | 0.0 | 0.5 |
| M1 | 0.0 | 0.0 | 5.2 |
| EBNA | 0.0 | 0.0 | 10.2 |
| D143_AXIN2_1 | 0.0 | 0.0 | 18.1 |
| D143_AXIN2_2 | 0.0 | 0.0 | 7.7 |
| D143_AXIN2_3 | 0.0 | 0.0 | 15.5 |
| D143_AXIN2_4 | 0.0 | 0.0 | 53.1 |
| D143_BAX_1 | 0.0 | 0.0 | 37.1 |
| D143_BAX_2 | 0.0 | 0.0 | 43.9 |
| D143-FAT3_1 | 0.0 | 0.0 | 7.5 |
| D143-FAT3_2 | 0.0 | 0.0 | 16.9 |
| D143-FAT3_3 | 0.0 | 0.0 | 19.3 |
| D143-FAT3_4 | 0.0 | 0.0 | 4.5 |
| D143-FAT3_5 | 0.0 | 0.0 | 0.8 |
| D143_APC | 0.0 | 0.0 | 78.6 |
| D143_BCL9L | 0.0 | 0.0 | 28.4 |
| D143_CTNNB1 | 0.0 | 0.0 | 0.0 |
| D143_MDM2 | 0.0 | 0.0 | 0.0 |
| D143_PIK3CA | 0.0 | 0.0 | 14.0 |
| D143_PRPF40B | 0.0 | 0.0 | 18.0 |
| D143_PTPRK | 0.0 | 0.0 | 4.2 |
| D143_PTPRT | 0.0 | 0.0 | 8.7 |
| D143_ROBO2 | 0.0 | 0.0 | 24.8 |
| D143_STAG1 | 0.0 | 0.0 | 15.9 |
| D143_TGFBR2 | 0.0 | 0.0 | 0.0 |
| D143_SIRT3_1 | 0.0 | 0.0 | 13.8 |
| D143_SIRT3_2 | 0.0 | 0.0 | 21.4 |
| D143_MYC | 0.0 | 0.0 | 21.5 |
| D143_FAT4 | 0.0 | 0.0 | 12.8 |
| D143_ATM_1 | 0.0 | 0.0 | 9.9 |
| D143_ATM_2 | 0.0 | 0.0 | 4.8 |
| vacD143_BTNL3 | 0.0 | 0.0 | 15.6 |
| vacD143_RANBP17 | 0.0 | 0.0 | 9.7 |
| vacD143_ING2 | 0.0 | 0.0 | 16.9 |
| vacD143_IGF2R | 0.0 | 0.0 | 31.8 |
| vacD143_ENTPD3 | 0.0 | 0.0 | 13.9 |
| vacD143_TMEM184A | 0.0 | 0.0 | 0.0 |
| vacD143_S1PR2 | 0.0 | 0.0 | 1.8 |
| vacD143_SPG7 | 0.0 | 0.0 | 1.6 |
| vacD143_TAF15 | 0.0 | 0.0 | 5.8 |
| vacD143_SYNE2 | 0.0 | 0.0 | 9.4 |
| vacD143_SCAI | 0.0 | 0.0 | 9.3 |
| vacD143_NF1 | 0.0 | 0.0 | 13.7 |

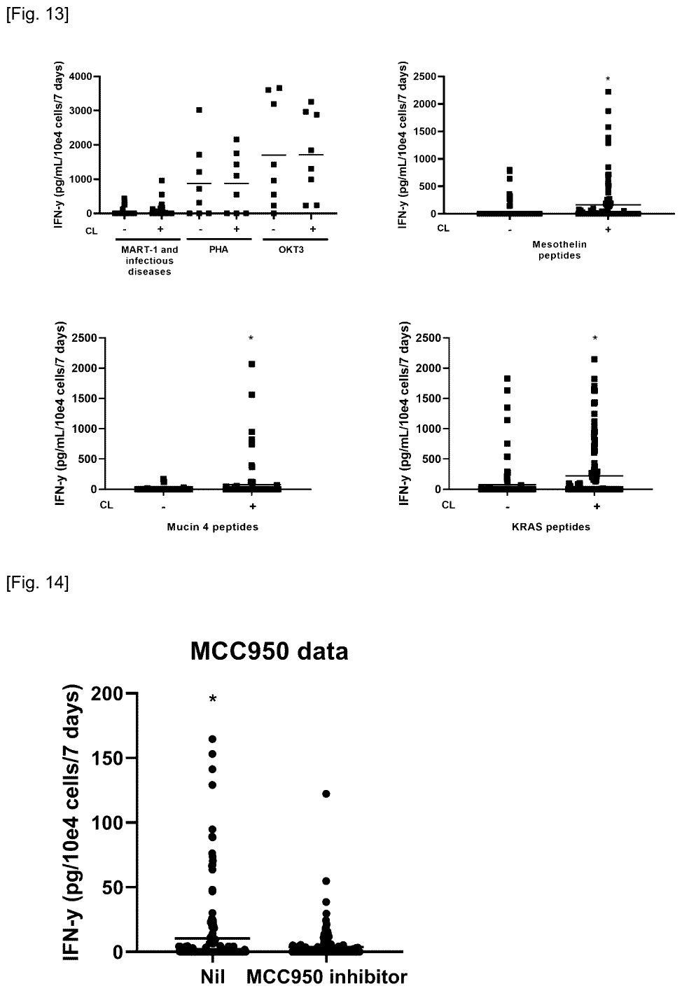

TIL expanded in the presence or absence of cardiolipin were tested for recognition of commonly shared tumor-associated antigens (e.g., mesothelin, KRAS listed in the Table 5), for the amount of IFN-γ production directed against each individual target. TIL expanded in cardiolipin recognized a high number of KRAS as well as mesothelin targets, which suggests that cardiolipin facilitates expansion of antigen-specific T-cells (FIG. 13).

Since cardiolipin has been reported to activate the NRLP3 inflammasome, aliquots of these same TIL preparation were expanded and the absence or presence of the NRLP3 inhibitor (MCC950) was investigated. The preparation was tested against a panel of wild-type and mutant KRAS epitopes (listed in the Table 6) to gauge different peptide-specific T-cell responses (FIG. 14) that showed an increased number of epitopes recognized and stronger IFN-γ production, suggesting the inhibition of the NRLP3 pathway statistically reduces the function of cardiolipin.

RNA sequencing was performed in seven of TIL expansions (i.e. from seven individual patients) with or without cardiolipin and tested for differential gene expression (FIG. 15). Genes with extremely low counts across samples were removed because they provide little evidence for differential expression and may interfere with some of the statistical approximations used.

DESeq2 was used with a False Discovery Rate <0.10 as a cut-off value to estimate differential expression between the TIL grown with and without cardiolipin, two genes were found to be over-expressed in TIL expanded with cardiolipin, CXCL9 and CXCL10, and the gene expression of KBTBD11 (a gene that influences the nuclear factor of activated T cell cytoplasmic-1 (NFATc1) pathway) was statistically reduced in TIL expanded with cardiolipin.

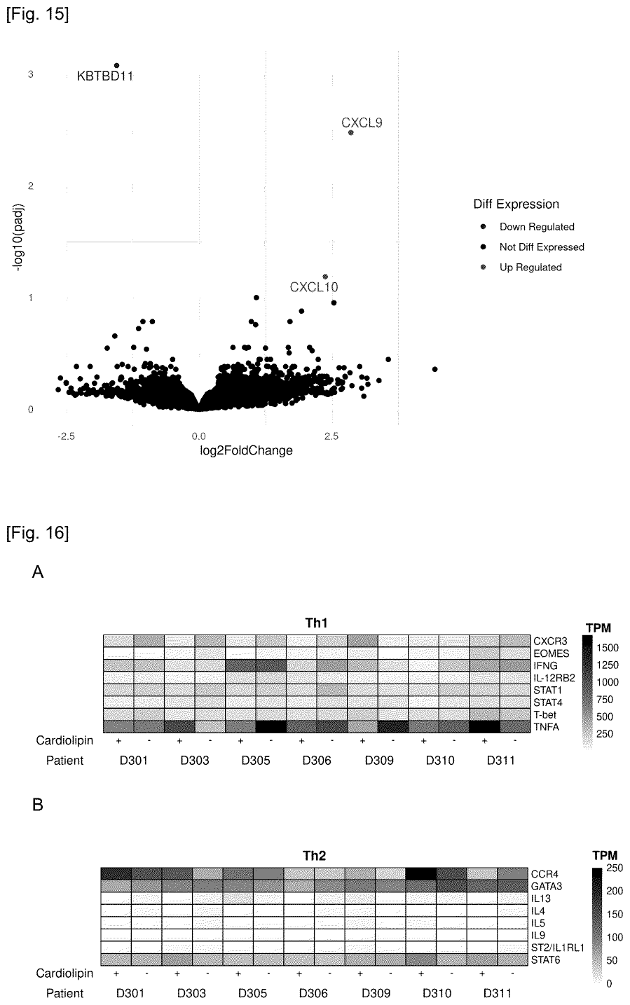

Both CXCL9 and CXCL10 are cytokines that belong to the intercrine alpha family and bind to CXCR3. They are a chemotactic factor for activated T-cells that affects the growth, movement, or activation state of cells that participate in immune and inflammatory response. CXCL9 and CSCL10 induce chemotaxis and facilitate extravasation of immune cells thereby enabling T-cells to enter tissues, a positive feature in the biology of tumor—infiltrating lymphocytes). The raw counts for individual RNA transcript were also normalized using transcripts per million (TPM) and heatmaps focusing on gene signatures and markers strongly associated with immune responses were built (FIG. 16).

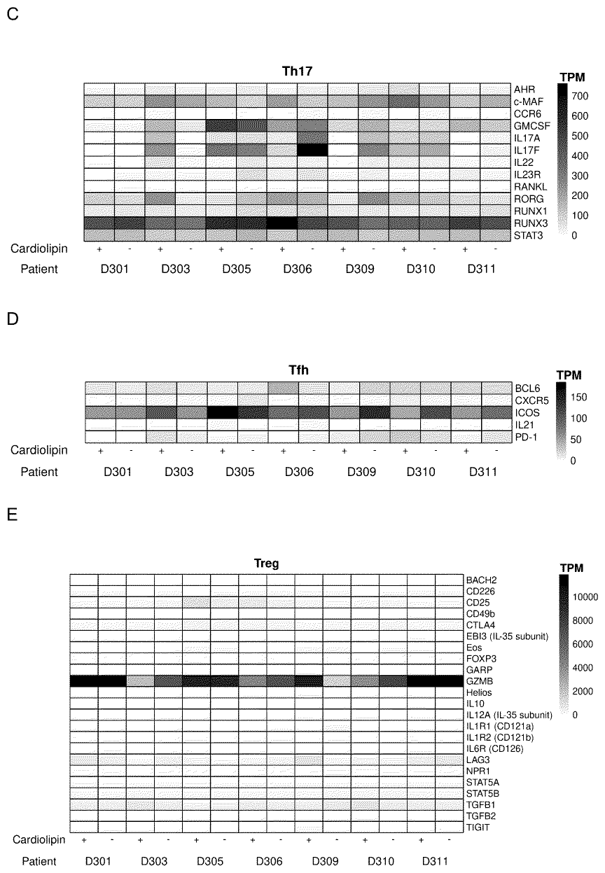

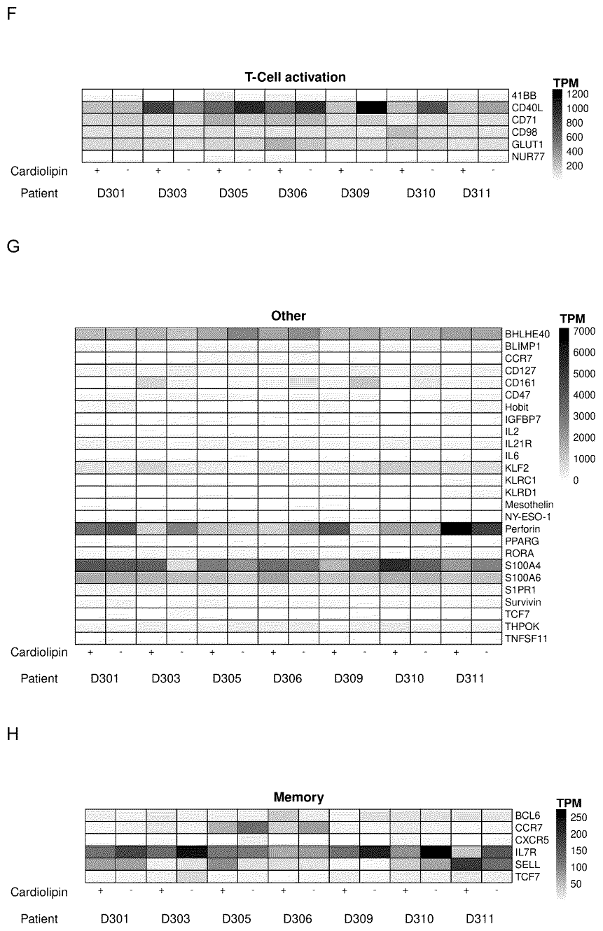

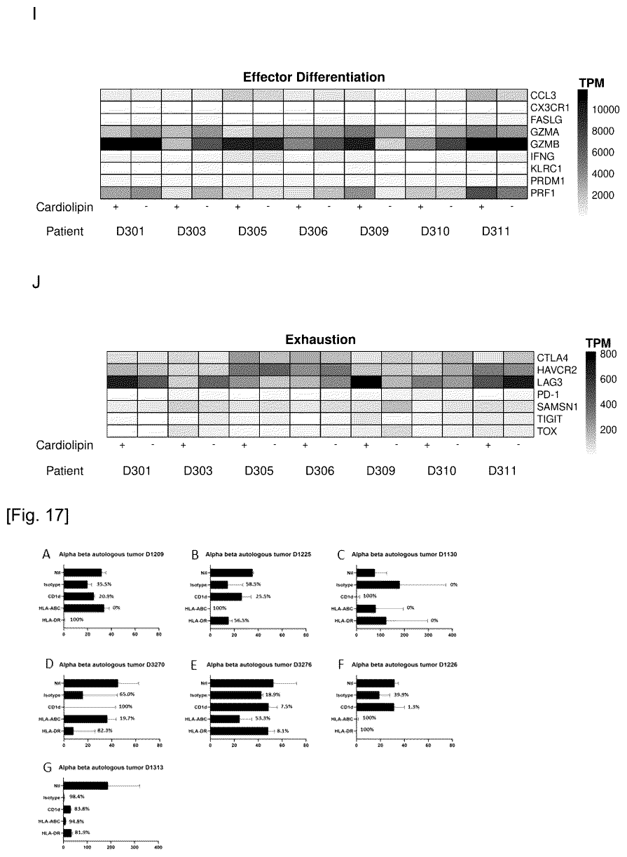

Next, TIL expansion from patients with various amounts and spatial presence of γδ T-cells were tested in the presence of cardiolipin for recognition of autologous tumor cells by TCR αβ and γδ TIL. TIL were expanded, for the first 3-5 days, with IL-7, IL-15, and IL-21, followed by a switch to IL2, IL-7 and IL-15. γδ TIL were then negatively sorted and γδ TIL purity was shown to be in the range of 98% (FIG. 16). Unsorted TIL or TCR αβ TIL, depleted of γδ TIL, recognized the autologous tumors (FIG. 17).

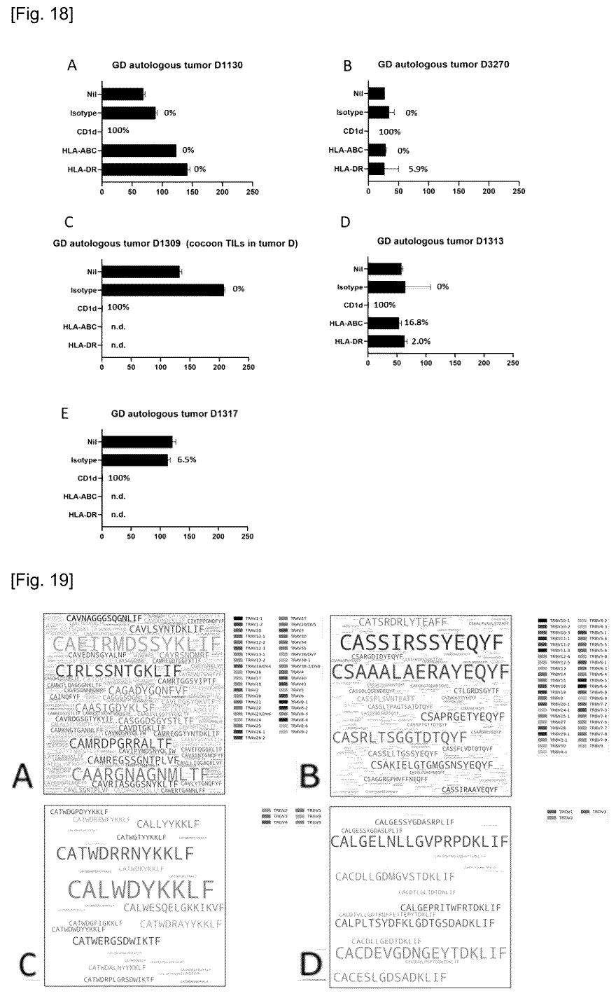

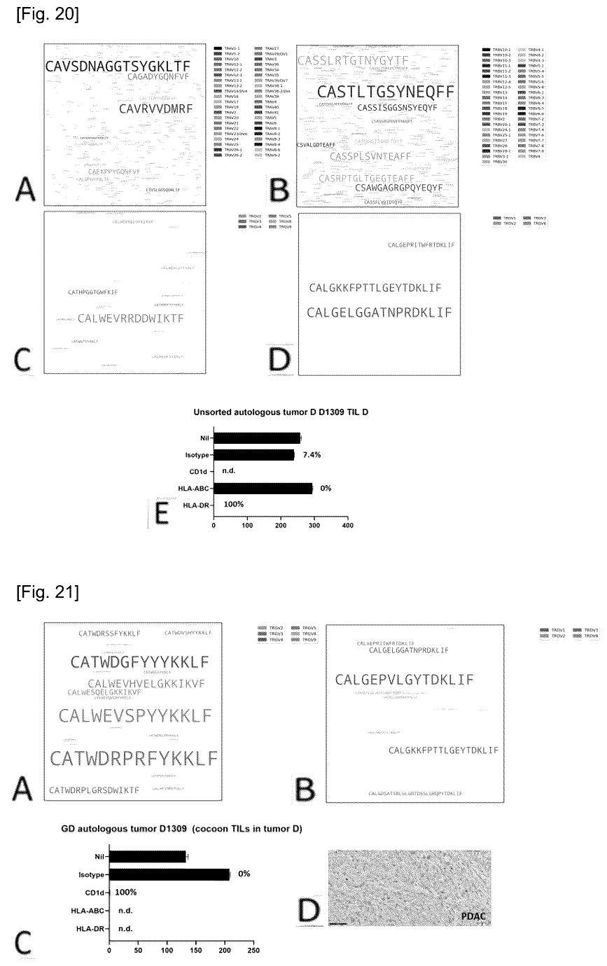

9 TIL were tested for recognition of autologous tumor cells, wherein 4/9 were HLA-DR restricted, 3/9 were HLA-A/B/C restricted, 1/9 was both HLA-DR and HLA-A/B/C restricted and 1/9 showed CD1d restriction (a teratoma) shown by blocking with specific antibodies. Pure sorted γδ TIL (5/5) recognized the autologous tumor and were shown to be blocked with anti-CD1d, yet not with control antibodies. TCR analysis was performed to gauge the presence of TCR αβ in MHC class I or MHC-class II, as well as in sorted TCR GD-reactive CD1d T-cells (FIGS. 18, 19, 20 and 21).

To identify the γδ and αβ TIL recognition, further experiments were conducted.

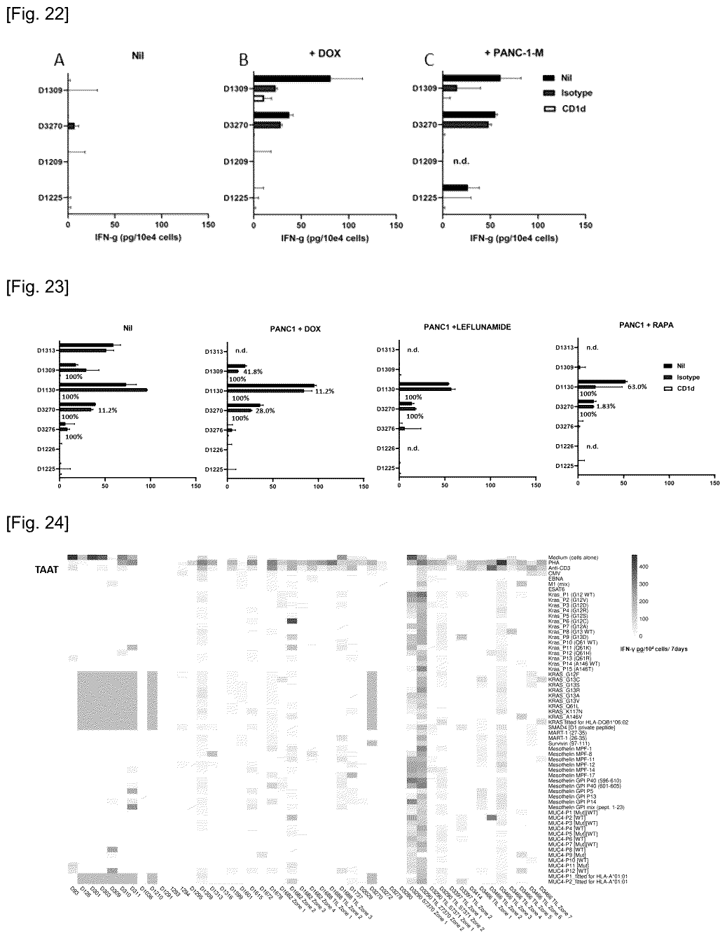

Autologous IL-4/GM-CSF antigen presenting cells were differentiated from monocytes prepared from PBMCs in 4/4 patients and were pulsed with different mitochondrial preparations (FIG. 22). Mitochondria isolated from healthy, non-manipulated PBMCs did not yield APC recognition defined by IFN-γ production. In contrast, 2/4 TIL recognized mitochondria prepared from PBMCs exposed to doxorubicin (which damages mitochondria) pretreated PBMCs, the same was also found to be true for mitochondria prepared from the pancreatic cancer cell line PANC-1, showing that cardiolipin expanded TCR GD⁺ TIL recognized both damaged mitochondria and mitochondria obtained from tumor cells but not mitochondria from healthy cells.

Next, 7 TCR γδ TIL lines were examined and 5/7 γδ⁺ TIL recognized PANC-1 cells in a CD1d restricted fashion shown by IFN-γ production and blocking with specific antibodies, yet not with the appropriate controls. PANC-1 recognition was not changed by pretreatment of PANC-1 cells with doxorubicin (which damages further mitochondria), nor with Leflunomide (that leads to fusion of mitochondria) or rapamycin (that interferes with the antigen processing and presentation pathway) (FIG. 23).

In order to examine more specificity in TIL from patient D1309 and D1313, synthetic peptides were tested for TIL recognition defined by IFN-γ production. Commonly shared tumor-associated antigens (listed in Table 5) and private tumor mutations (Table 7) were tested for TIL recognition from patient 1309 and D1313 that recognized several commonly shared epitopes and neoepitopes. TIL from patient D1313 recognized a mutant epitope from a mitochondrial protein which was found to be HLA-DP restricted. TIL from patient D1309 recognized a mutant driver gene involved in PDAC oncogenesis.

In summary, cardiolipin activated TIL resulted in increased frequencies of tumor-reactive T-cells in general, resulted in expansion of γδ TIL that were tumor-specific and CD1d restricted. In order to define in greater detail, the nature of the γδ TIL reactivity, mitochondria from healthy donor PBMCs, from doxorubicin-treated PBMCs or with mitochondria from the allogeneic tumor cell line PANC-1 were isolated. γδ TIL recognized the autologous tumor in a CD1d restricted fashion and the same γδ TIL recognized the pancreatic cancer cell line PANC1, also CD1d restricted.

One of the candidate targets are damaged mitochondria, either induced by doxorubicin from healthy cells or mitochondria isolated from cancer cells. Cardiolipin also facilitates the expansion of tumor-reactive αβ TIL, they recognized the autologous tumor cells in an MHC class I or class II—restricted fashion and analysis of 2 TIL in greater detail showed that commonly shared, as well as private mutant neoepitopes, are being recognized, e.g., from a mutant PDAC driver gene or a gene from a mitochondrion suggesting that mitochondrial targets are viable sources for TIL target recognition. Cardiolipin induced up-regulation of CXCL9 and CXCL10 expression in TIL, factors that facilitate the invasion of TIL into solid cancers. This makes this protocol unique, combining αβ and γδ T-cell expansion and taking into consideration the starting material in the process since PDAC lesions, or lesions from other solid tumors, are heterogenous in tumor cells, yet also in T-cell infiltrates. TIL can be expanded up to 1×10⁹ cells as shown in these experiments.

A tumor specific cellular immune response can be seen a very specific autoimmune response directed against mutant or non-mutant self-proteins that are preferentially or exclusively expressed in transformed tissue. The 3-step expansion method allowed to gauge for cytokine production in response to viral/cross-reactive epitopes recognizing human self-proteins, as can be seen on Table 9 below.

TABLE 9

| | | |
|---|---|---|
| | IFN-γ | |
| Sample | Production | IL-17 Production |

TIL that reacted against SARS-CoV-2 spike (positions
212-220 QHD43416.1) tailored for HLA-B3501

| | | |
|---|---|---|
| SARS-CoV-2 spike (positions 212-220 | 1457.59 | 462.99 |
| QHD43416.1) tailored for HLA-B3501 | | |
| SARS-CoV-2 spike (positions 212-220 | 138.58 | 0.00 |
| QHD43416.1) tailored for HLA- | | |
| B3501 + anti-ABC | | |
| SARS-CoV-2 spike (positions 212-220 | 1057.65 | 428.92 |
| QHD43416.1) tailored for HLA- | | |
| B3501 + anti-DR | | |
| Human U11/U12 snRNP 35 kDa | 1230.50 | 372.84 |
| protein (positions 81-90 Q16560.1) | | |
| tailored for HLA-B3501 | | |
| Human U11/U12 snRNP 35 kDa | 879.62 | 0.00 |
| protein (positions 81-90 Q16560.1) | | |
| tailored for HLA-B3501 + anti-ABC | | |
| Human U11/U12 snRNP 35 kDa | 1289.42 | 420.07 |
| protein (positions 81-90 Q16560.1) | | |
| tailored for HLA-B3501 + anti-DR | | |
| Tumor | 243.16 | 222.31 |
| Tumor + anti-ABC | 0.00 | 0.00 |
| Tumor + anti-DR | 775.13 | 495.99 |
| OKT3 | 2540.06 | 305.99 |
| CMV | 0.00 | 0.00 |
| Medium | 0.00 | 0.00 |

TIL that reacted against Human U11/U12 snRNP 35 kDa
protein (positions 81-90 Q16560.1) tailored for HLA-B3501

| | | |
|---|---|---|
| Human U11/U12 snRNP 35 kDa | 2444.61 | 330.14 |
| protein (positions 81-90 Q16560.1) | | |
| tailored for HLA-B3501 | | |
| Human U11/U12 snRNP 35 kDa | 1908.56 | 81.91 |
| protein (positions 81-90 Q16560.1) | | |
| tailored for HLA-B3501 + anti-ABC | | |
| Human U11/U12 snRNP 35 kDa | 1509.64 | 444.01 |
| protein (positions 81-90 Q16560.1) | | |
| tailored for HLA-B3501 + anti-DR | | |
| SARS-CoV-2 spike (positions 212-220 | 2611.08 | 238.70 |
| QHD43416.1) tailored for HLA-B3501 | | |
| SARS-CoV-2 spike (positions 212-220 | 2062.87 | 12.68 |
| QHD43416.1) tailored for HLA- | | |
| B3501 + anti-ABC | | |
| SARS-CoV-2 spike (positions 212-220 | 1974.19 | 257.46 |
| QHD43416.1) tailored for HLA- | | |
| B3501 + anti-DR | | |
| Tumor | 548.75 | 248.83 |
| Tumor + anti-ABC | 2586.93 | 19.80 |
| Tumor + anti-DR | 1818.86 | 371.12 |
| OKT3 | 3333.24 | 417.70 |
| CMV | 1680.45 | 7.50 |
| Medium | 0.00 | 0.00 |

Testing of commonly shared antigens or private antigens for immune effector functions in TIL versus PBMCs harvested at the same time point aid to show that the 3 step-expanded TIL contain cancer target specific T-cells that are not present in PBMCs, it will also aid to select for different TIL products. The simultaneous measurement of IFN-γ versus IL-17 aids also to select for T-cells that i) exclusively or preferentially produce IFN-γ, but not IL-17 in response to cancer target antigens, it also aids to define target antigens that elicit in the T-cell populations an antigen-specific IL-17 response but not an IFN-γ response. Such T-cell products may either not be considered for therapy since IL-17 may facilitate tumor—progression; alternatively, IL-17 antigens, including autoimmune, cancer or viral targets that elicit IL-17 responses may be removed from vaccine formulations.

The differential recognition cytokine production against different molecularly defined target antigens is shown in Table 10 below. Strong IFN-γ production in the positive controls (PHA or anti-CD3 cross-linking), only limited amount of IL-17 production in response to the positive control PHA. Yet distinct IL-17 production in response to defined peptide targets (e.g., KRAS or mesothelin) which may reflect antigen-driven specific T-cell proliferation leading to IL-17 production.

TABLE 10

| | | Pancreatic | Pancreatic |
|---|---|---|---|
| | | TIL D1599 | TILD1599 |
| | Tumor type: | IFN-y | IL 17 |
| Peptide/stimulus | aa sequence | pg/mL | pg/mL |
| PHA | — | 417 | 4 |
| OKT3 | — | 200 | 0 |
| CMV | Peptide Pool | 0 | 0 |
| EBNA | Peptide Pool | 0 | 0 |
| M1 (mix) | Peptide Pool | 0 | 0 |
| ESAT6 | ELNNALQNL | 0 | 0 |
| Kras_P1 (G12 | YKLVVVGAGGVGKSALT | 0 | 37 |
| WT) | | | |
| Kras_P2 (G12V) | YKLVVVGAVGVGKSALT | 0 | 2 |
| Kras_P3 (G12D) | YKLVVVGADGVGKSALT | 0 | 0 |
| Kras_P4 (G12R) | YKLVVVGARGVGKSALT | 0 | 0 |
| Kras_P5 (G12S) | YKLVVVGASGVGKSALT | 14 | 0 |
| Kras_P6 (G12C) | YKLVVVGACGVGKSALT | 0 | 0 |
| Kras_P7 (G12A) | YKLVVVGAAGVGKSALT | 0 | 65 |
| Kras_P8 (G13 | KLVVVGAGGVGKSALTI | 0 | 108 |
| WT) | | | |
| Kras_P9 (G13D) | KLVVVGAGDVGKSALTI | 0 | 3 |
| Kras_P10 (Q61 | LDILDTAGQEEYSAMRD | 0 | 0 |
| WT) | | | |
| Kras_P11 (Q61K) | LDILDTAGKEEYSAMRD | 0 | 0 |
| Kras_P12 (Q61H) | LDILDTAGHEEYSAMRD | 0 | 4 |
| Kras_P13 (Q61R) | LDILDTAGREEYSAMRD | 0 | 0 |
| Kras_P14 (A146 | GIPFIETSAKTRQRVED | 0 | 0 |
| WT) | | | |
| Kras_P15 | GIPFIETSTKTRQRVED | 0 | 0 |
| (A146T) | | | |
| KRAS_G12F | KLVVVGAVGVGKSAL | 36 | 0 |
| KRAS_G13C | LVVVGAGCVGKSALT | 26 | 0 |
| KRAS_G13S | LVVVGAGSVGKSALT | 24 | 0 |
| KRAS_G13R | LVVVGAGRVGKSALT | 29 | 0 |
| KRAS_G13A | LVVVGAGAVGKSALT | 19 | 0 |
| KRAS_G13V | LVVVGAGVVGKSALT | 10 | 0 |
| KRAS_Q61L | DILDTAGLEEYSAMR | 24 | 0 |
| KRAS_K117N | PMVLVGNNCDLPSRT | 32 | 0 |
| KRAS_A146V | IPFIETSVKTRORVE | 5 | 0 |
| KRAS fitted for | LVVVGAVGVGKSALT | 0 | 0 |
| HLA-DQB1*06:02 | | | |
| SMAD4 [D1 | LAFQPPISNHPAPEY | 0 | 0 |
| private peptide] | | | |
| Survivin (97-111) | TLGEFLKLDRERAKN | 0 | 0 |
| Survivin_1_HLA- | ALPQIWQL | 0 | 0 |
| A*02:01_1 | | | |
| Survivin_1_HLA- | ALPQIWQLY | 0 | 0 |
| A*02:01_2 | | | |
| Mesothelin MPF- | MALPTARPLLGSCGT | 0 | 0 |
| 1 | | | |
| Mesothelin MPF- | HRLSEPPEDLDALPL | 0 | 0 |
| 8 | | | |
| Mesothelin MPF- | LLPRGAPERQRLLPA | 0 | 0 |
| 11 | | | |
| Mesothelin MPF- | ALACWGVRGSLLSEA | 0 | 0 |
| 12 | | | |
| Mesothelin MPF- | FVAESAEVLLPRLVS | 0 | 0 |
| 14 | | | |
| Mesothelin MPF- | SVSTMDALRGLLPVL | 0 | 0 |
| 17 | | | |
| Mesothelin GPI | LQGGIPNGYLVLDLS | 0 | 0 |
| P40 (596-610) | | | |
| Mesothelin GPI | MQEALSGTPCLLGPG | 0 | 120 |
| P40 (601-605) | | | |
| Mesothelin GPI | YEQLDVLKHKLDELY | 0 | 0 |
| P5 | | | |
| Mesothelin GPI | WAVRPQDLDTCDPRQ | 0 | 0 |
| P13 | | | |
| Mesothelin GPI | LDVLYPKARLAFQNM | 0 | 11 |
| P14 | | | |
| Mesothelin GPI | Aminoacids 1-630 | 0 | 0 |
| mix (pept. 1-23) | | | |
| MUC4-P1 | TPLPVTDASSASTGH | 0 | 3 |
| [Mut][WT] | | | |
| MUC4-P2 [WT] | TPLPVTDTSSASTGH | 0 | 1 |
| MUC4-P3 | PVTSLSSVSTGDTTP | 0 | 2 |
| [Mut][WT] | | | |
| MUC4-P4 [WT] | PVTSLSSASTGDTTP | 17 | 0 |
| MUC4-P5 | TTPLPVTIPSSASSG | 12 | 0 |
| [Mut][WT] | | | |
| MUC4-P6 [WT] | TTPLPVTSPSSASSG | 0 | 0 |
| MUC4-P7 | PVTSTSSVSTGHATP | 0 | 0 |
| [Mut][WT] | | | |
| MUC4-P8 [WT] | PVTSTSSASTGHATP | 0 | 2 |
| MUC4-P9 [Mut] | PVTSPSSESTGHAIP | 0 | 0 |
| MUC4-P10 [WT] | PVTSPSSASTGHAIP | 5 | 0 |
| MUC4-P11 [Mut] | TPLPVTDNSSASTGD | 19 | 0 |
| MUC4-P12 [WT] | TPLPVTDTSSASTGD | 34 | 0 |
| MUC4-P1_fitted | VTDTSSASTGH | 0 | 24 |
| for HLA-A*01:01 | | | |
| MUC4-P2_fitted | VTDASSASTGH | 0 | 14 |
| for HLA-A*01:01 | | | |

The differences in TIL versus PBMCs can also be defined by peptide reactivity. 10e⁴ TIL or 10e⁴ PBMC/well were co-cultured with the peptides listed in Table 11 below for 7 days and the supernatants were tested for IFN-γ production. Differential recognition in TIL, but in PBMCs of KRAS peptide targets suggests that the expanded TIL product is enriched for mutant target specific T-cells and suitable for the active cellular therapy of patients with cancer.

TABLE 11

| aa sequence | TIL | PBMCs |
|---|---|---|
| PHA | 1336 | 71 |
| anti-CD3 | 0 | 63 |
| Peptide Pool CMV | 0 | 0 |
| Peptide Pool EBNA | 0 | 0 |
| Peptide Pool M1 | 0 | 0 |
| ELNNALQNL | 6 | 0 |
| YKLVVVGAGGVGKSALT | 0 | 0 |
| YKLVVVGAVGVGKSALT | 0 | 0 |
| YKLVVVGADGVGKSALT | 0 | 0 |
| YKLVVVGARGVGKSALT | 0 | 0 |
| YKLVVVGASGVGKSALT | 0 | 0 |
| YKLVVVGACGVGKSALT | 0 | 0 |
| YKLVVVGAAGVGKSALT | 0 | 0 |
| KLVVVGAGGVGKSALTI | 0 | 0 |
| KLVVVGAGDVGKSALTI | 0 | 0 |
| LDILDTAGQEEYSAMRD | 0 | 0 |
| LDILDTAGKEEYSAMRD | 0 | 0 |
| LDILDTAGHEEYSAMRD | 0 | 0 |
| LDILDTAGREEYSAMRD | 150 | 0 |
| GIPFIETSAKTRORVED | 97 | 0 |
| GIPFIETSTKTRQRVED | 85 | 0 |
| KLVVVGAVGVGKSAL | 0 | 0 |
| LVVVGAGCVGKSALT | 0 | 0 |
| LVVVGAGSVGKSALT | 0 | 0 |
| LVVVGAGRVGKSALT | 0 | 0 |
| LVVVGAGAVGKSALT | 0 | 0 |
| LVVVGAGVVGKSALT | 0 | 0 |
| DILDTAGLEEYSAMR | 0 | 0 |
| PMVLVGNNCDLPSRT | 0 | 0 |
| IPFIETSVKTRORVE | 33 | 0 |
| LVVVGAVGVGKSALT | 0 | 0 |
| LAFQPPISNHPAPEY | 0 | 0 |
| TLGEFLKLDRERAKN | 0 | 0 |
| ALPQIWQL | 0 | 0 |
| ALPQIWQLY | 0 | 0 |
| MALPTARPLLGSCGT | 0 | 0 |
| HRLSEPPEDLDALPL | 0 | 0 |
| LLPRGAPERQRLLPA | 0 | 0 |
| ALACWGVRGSLLSEA | 0 | 0 |
| FVAESAEVLLPRLVS | 0 | 0 |
| SVSTMDALRGLLPVL | 0 | 0 |
| LQGGIPNGYLVLDLS | 0 | 0 |
| MQEALSGTPCLLGPG | 0 | 0 |
| YEQLDVLKHKLDELY | 0 | 0 |
| WAVRPQDLDTCDPRQ | 0 | 0 |
| LDVLYPKARLAFQNM | 0 | 0 |
| Amino acids 1-630 | 0 | 0 |
| TPLPVTDASSASTGH | 0 | 0 |
| TPLPVTDTSSASTGH | 0 | 0 |
| PVTSLSSVSTGDTTP | 0 | 0 |
| PVTSLSSASTGDTTP | 0 | 0 |
| TTPLPVTIPSSASSG | 0 | 0 |
| TTPLPVTSPSSASSG | 0 | 0 |
| PVTSTSSVSTGHATP | 0 | 0 |
| PVTSTSSASTGHATP | 0 | 0 |
| PVTSPSSESTGHAIP | 0 | 0 |
| PVTSPSSASTGHAIP | 0 | 0 |
| TPLPVTDNSSASTGD | 0 | 0 |
| TPLPVTDTSSASTGD | 0 | 0 |
| VTDTSSASTGH | 0 | 0 |
| VTDASSASTGH | 0 | 0 |

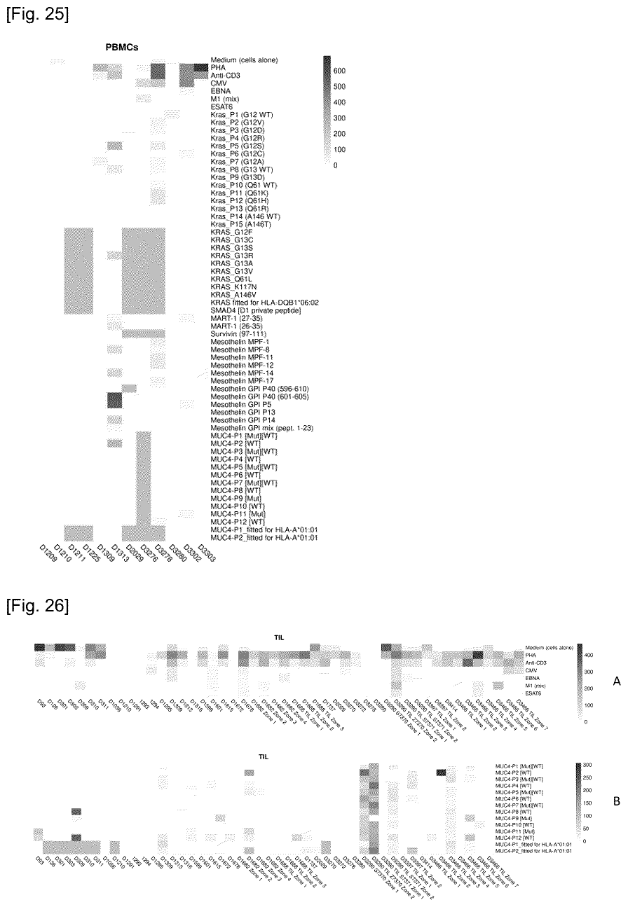

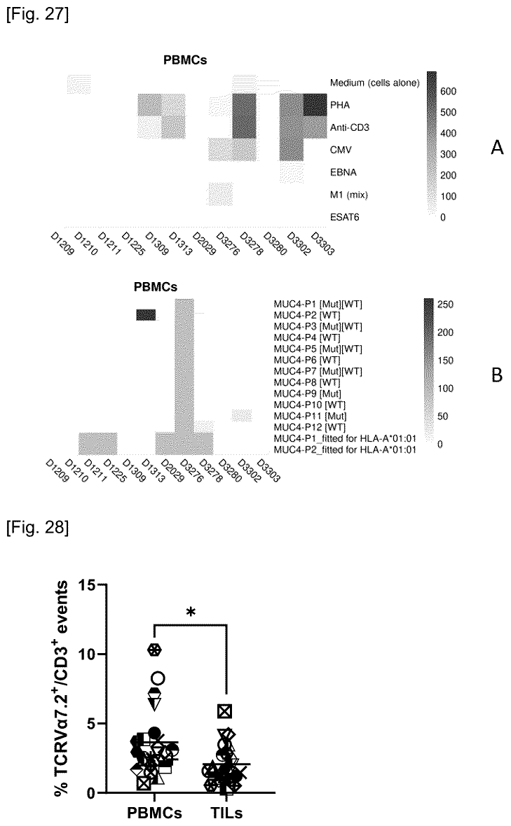

TIL lines (some from different areas) as well as PBMCs were evaluated for cytokine production to a broad panel commonly shared target antigens as described in this application that allowed to obtain a functional and molecular fingerprint of expanded TIL (FIGS. 24 and 25). This could also be done for a more specific questions, e.g., for MUC4 wild-type or mutant specific T-cell responses (FIGS. 26 and 27), or the detection of biologically relevant B-cell targets contained in MUC-4 repetitive epitopes.

In order to further characterize the immune cells expanded from a solid tissue or a liquid tissue sample, we analyzed the presence of MAIT cells in TIL and PBMCs (FIG. 28), since they may also contribute to anti-tumor directed immune responses either by direct recognition of MR-1 presented targets, or—not mutually exclusive—by MAIT cells that recognized bacterial species, or their related products. This is biologically relevant since the presence of distinct bacterial species has been reported to be associated with increased survival in patients with pancreatic cancer.

MAIT can discriminate between foreign and commensal bacteria by recognizing riboflavin precursors found in most bacteria and yeasts allowing the use of MR1 tetramers loaded with these molecules to selectively activate MAIT. 6-FP (6 Formyl-Pterin, a folic acid derivate) is recognized by MAIT but does not activate them, in contrast to 5-OP-RU which is a potent activator. Some TCR γδ⁺ T-cells have also been described to recognize the ‘underside’ of the MR1 molecule, most likely independent of the nominal ligand. Yet it could be possible that individual, as yet ill-defined MR1 ligands, shape the ‘underside’ of MR1 in such a way that TCR γδ⁺ T-cells preferentially bind to certain MR1 molecules loaded with targets that facilitate the interaction of the γδ⁺ T-cell receptor with the MR1 molecules.

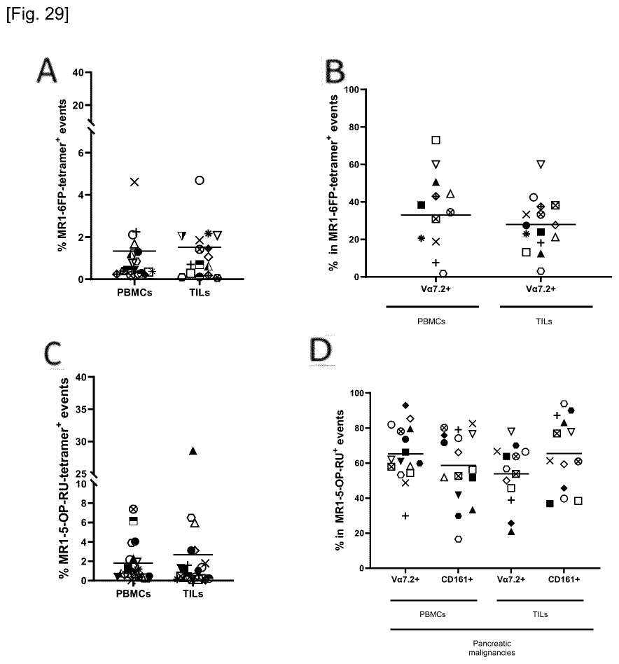

TIL expanded with the method described in the present invention also contain MAIT cells, either defined by the presence of the TCR VA7.2⁺ T-cells, or MAIT. 6-FP (6 Formyl-Pterin, a folic acid derivate) or MR1 presented 5-OP-RU antigens (FIGS. 29 and 30). VA7.2⁻ sorted MAIT cells recognize the autologous tumor cells in an MR1 restricted fashion, defined by IFN-γ production and they also recognize allogeneic tumor cell lines in an MR1 restricted fashion (FIG. 31).

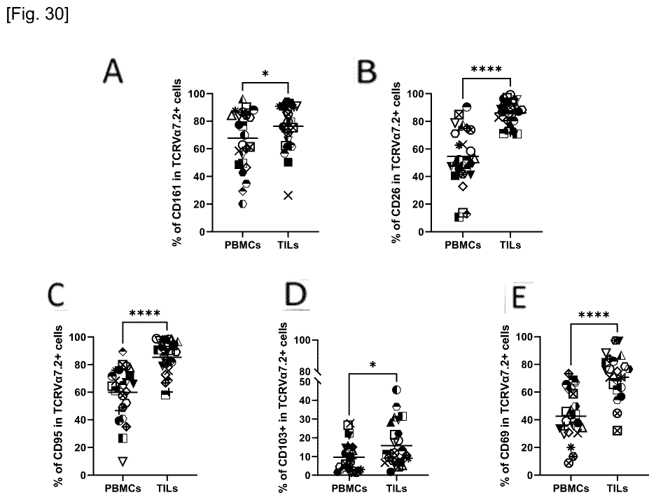

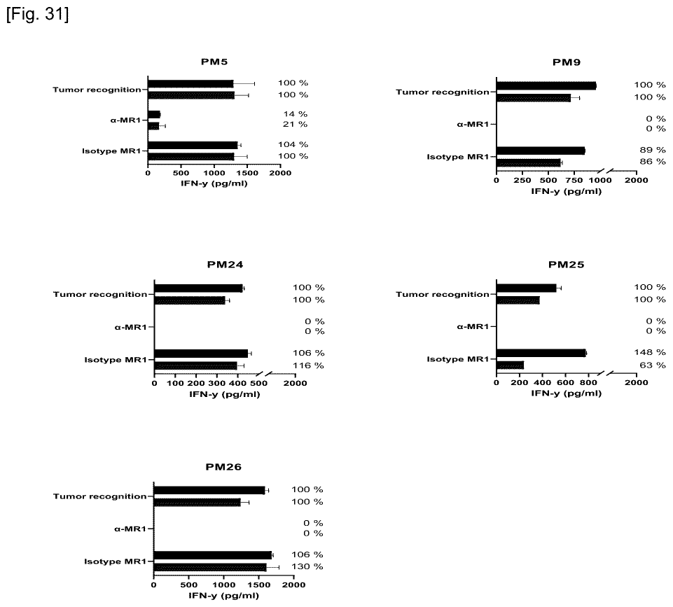

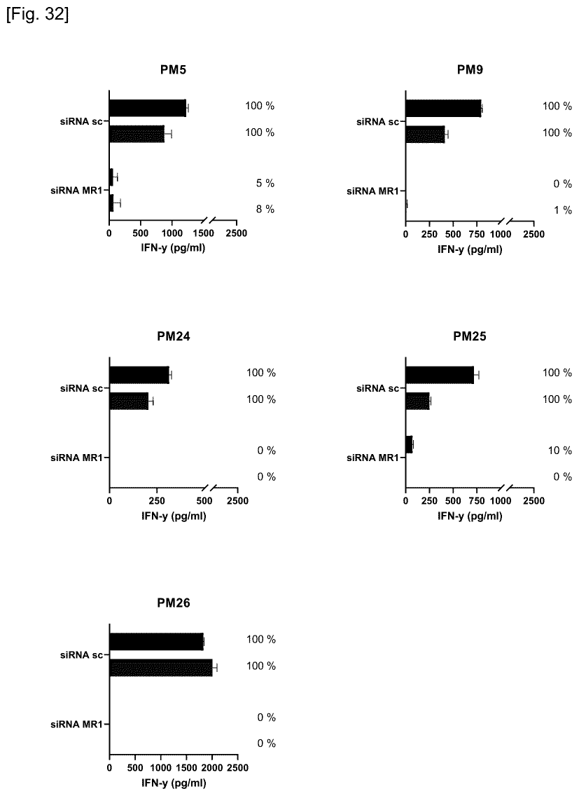

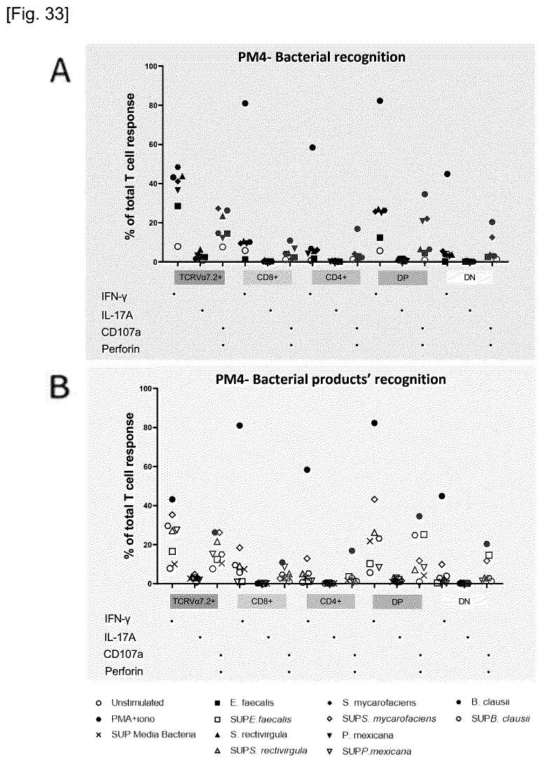

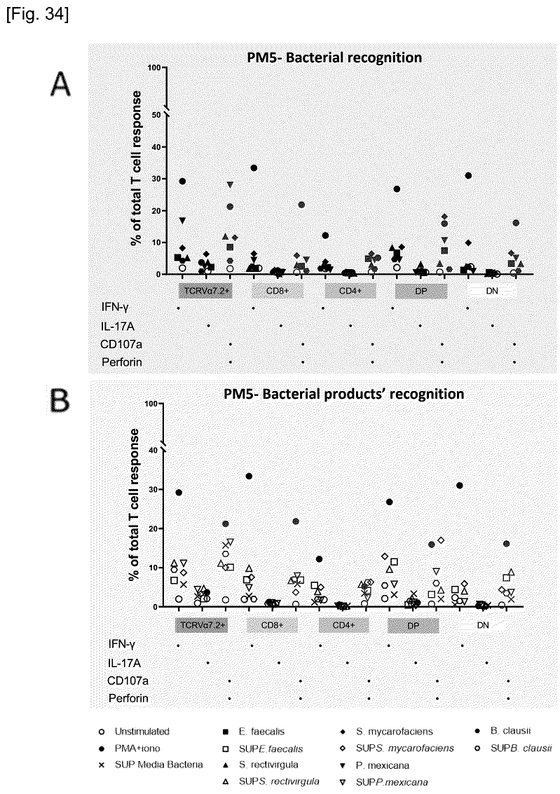

Specificity is shown by reducing MR1 expression using siRNA specifically targeting MR1, yet not scrambled control siRNA (FIG. 32). Bacterial recognition is detected in VA7.2 TIL against individual bacterial species associated with improved survival, as defined by IFN-γ⁺ frequency, or perforin/CD107a⁺ frequency, yet not IL17⁺ frequency, some of these bacterial responses are pronounced in the double-positive CD4⁺CD8⁺ T-cell TIL population (FIGS. 33 and 34).

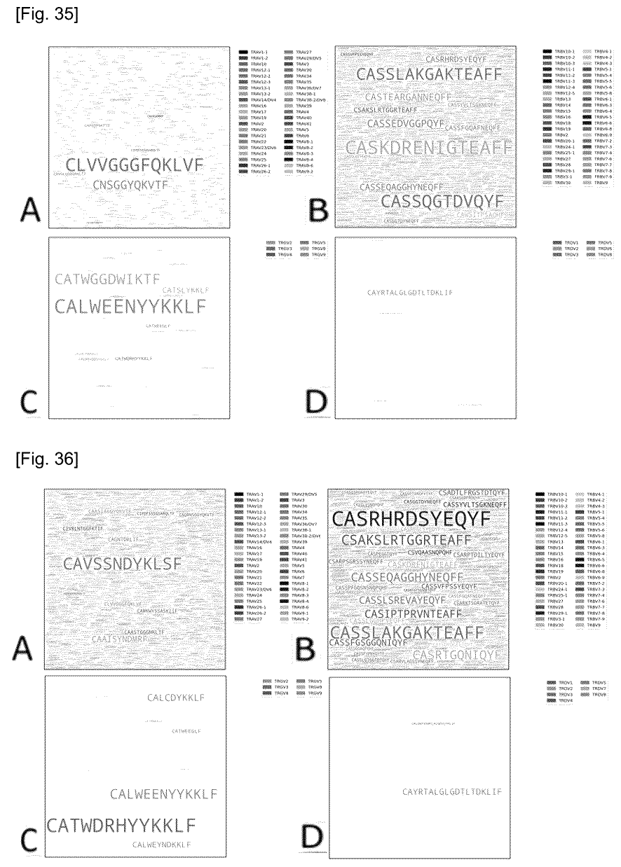

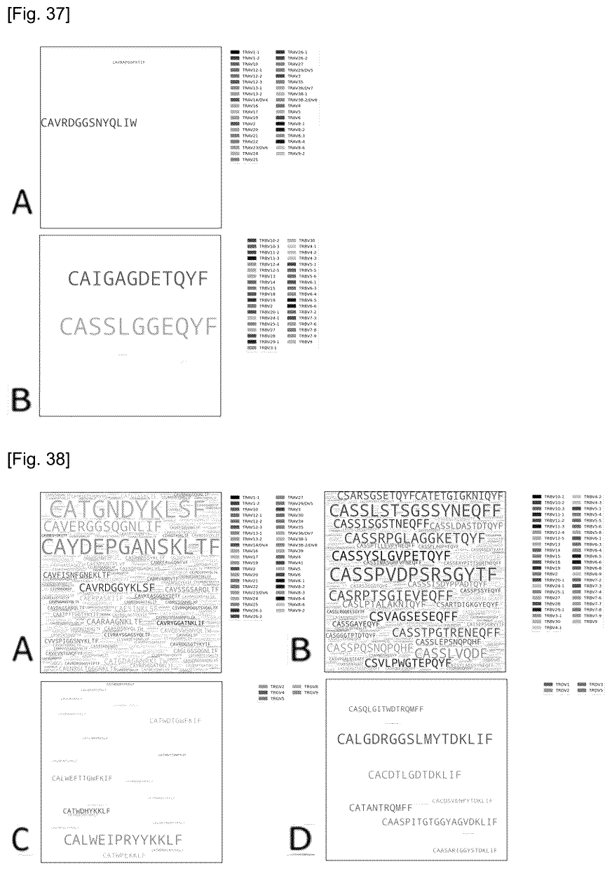

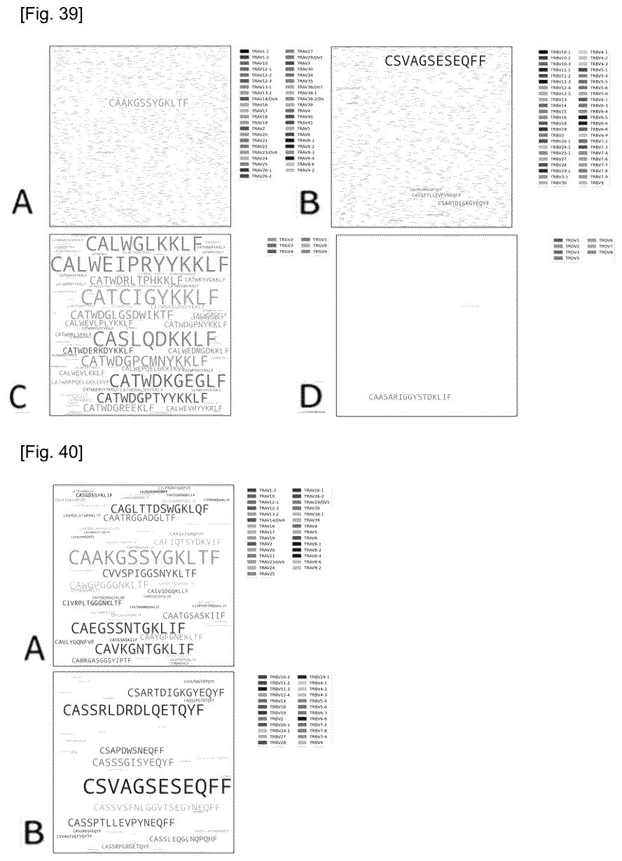

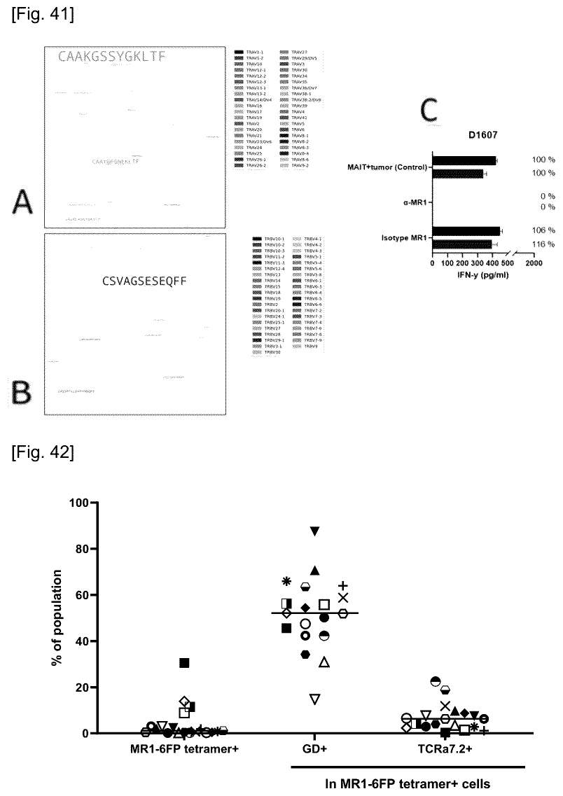

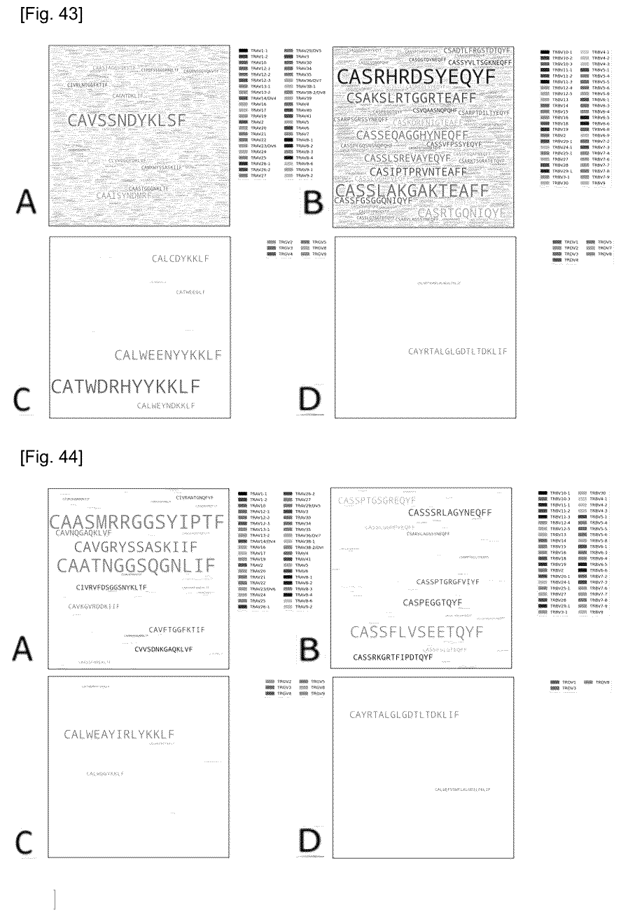

T-cell receptor analysis was performed in the tumor specimens (FIGS. 35 and 38), in the final expanded TIL product (FIG. 36, 39 43) as well as in the TCRVα7.2 sorted T-cells (FIGS. 37 and 40). MR1-6FP sorted cells show MR1 restricted tumor recognition (FIGS. 41 and 44). Flow cytometric analysis clearly showed that the MR-1 6-formylpterin reactive T-cells have a higher percentage of γδ⁺ T-cells than TCRVα7.2 cells (FIG. 42) suggesting that MR1-6FP+targets could be preferentially recognized by TCR γδ⁺ T-cells.

The MUC4 peptide is targeted by human IgG in serum from patients with cancer. This epitope is present in different areas of MUC4 and exhibits therefore multiple docking sites for an anti-MUC4 directed antibody response or as a target for chimeric antigen receptors, since it is targeted by naturally occurring antibody responses in patients with cancer, that implies immunogenicity. The epitope PVTSLSSVSTGDTTP was linked to an ELISA plate via a streptavidin-biotin scaffold and tested for IgG recognition in serum from different patients with pancreatic cancer. The optical density (OD) read at 405 nM wavelength was used to detect IgG binding in sera from patients showing that some individuals have a higher IgG concentration to this epitope as compared to other patients, some patients have no IgG reactivity at all showing that there is individual IgG reactivity to this distinct MUC4 epitopes. The anti-human IgG directed antibody A2064 clone GG-5 from Sigma-Aldrich coupled to Alkaline-Phosphatase was used to detect epitope reactivity. The assay layout and reactivity reading are shown in Tables 12 and 13 (layouts and ODs, respectively) below.

TABLE 12
Test assay layout

| | | | | | | | | | | | |
|---|---|---|---|---|---|---|---|---|---|---|---|
| IgG | IgG | 3 | 4 | 5 | 6 | 7 | 8 | 9 | 10 | 11 | 12 |

| | | | | | | | | | | |
|---|---|---|---|---|---|---|---|---|---|---|
| 1 | 15000 | 15000 | S1 1:2 | S1 1:2 | S5 1:2 | S5 1:2 | S9 1:2 | S9 1:2 | S13 1:2 | S13 1:2 |
| | ng/ml | ng/ml | | | | | | | | |
| 2 | 7500 | 7500 | S1 1:10 | S1 1:10 | S5 1:10 | S5 1:10 | S9 1:10 | S9 1:10 | S13 1:10 | S13 1:10 |
| | ng/ml | ng/ml | | | | | | | | |
| 3 | 3750 | 3750 | S2 1:2 | S2 1:2 | S6 1:2 | S6 1:2 | S10 1:2 | S10 1:2 | S14 1:2 | S14 1:2 |
| | ng/ml | ng/ml | | | | | | | | |
| 4 | 1875 | 1875 | S2 1:10 | S2 1:10 | S6 1:10 | S6 1:10 | S10 1:10 | S10 1:10 | S14 1:10 | S14 1:10 |
| | ng/ml | ng/ml | | | | | | | | |
| 5 | 937 | 937 | S3 1:2 | S3 1:2 | S7 1:2 | S7 1:2 | S11 1:2 | S11 1:2 | S15 1:2 | S15 1:2 |
| | ng/ml | ng/ml | | | | | | | | |
| 6 | 468 | 468 | S3 1:10 | S3 1:10 | S7 1:10 | S7 1:10 | S11 1:10 | S11 1:10 | S15 1:10 | S15 1:10 |
| | ng/ml | ng/ml | | | | | | | | |
| 7 | 234 | 234 | S4 1:2 | S4 1:2 | S8 1:2 | S8 1:2 | S12 1:2 | S12 1:2 | | |
| | ng/ml | ng/ml | | | | | | | | |
| 8 | 117 | 117 | S4 1:10 | S4 1:10 | S8 1:10 | S8 1:10 | S12 1:10 | S12 1:10 | | |
| | ng/ml | ng/ml | | | | | | | | |

TABLE 13
OD readout - corresponding to table 12

| 405 | | | | | | | | | | | | |
|---|---|---|---|---|---|---|---|---|---|---|---|---|
| nm | 1 | 2 | 3 | 4 | 5 | 6 | 7 | 8 | 9 | 10 | 11 | 12 |
| A | 0.2802 | 0.2542 | 0.5313 | 0.4483 | 0.1114 | 0.0793 | 0.1428 | 0.1489 | 0.6059 | 0.5168 | 0.2041 | 0.5025 |
| B | 0.1547 | 0.2177 | 0.5528 | 0.3164 | 0.1002 | 0.0862 | 0.5957 | 0.3125 | 0.1781 | 0.1986 | 0.2353 | 0.4286 |
| C | 0.0882 | 0.0827 | 0.9806 | 1.0047 | 0.0441 | 0.0315 | 0.1048 | 0.1689 | 0.0958 | 0.1275 | 0.0997 | 0.1831 |
| D | 0.0742 | 0.0548 | 0.2622 | 0.1714 | 0.1013 | 0.0889 | 0.4019 | 0.4211 | 0.1789 | 0.1426 | 0.1418 | 0.294 |
| E | 0.0763 | 0.049 | 0.1734 | 0.0634 | 0.1802 | 0.1287 | 0.1769 | 0.109 | 0.5957 | 0.2927 | 0.0564 | 0.1205 |
| F | 0.0711 | 0.0596 | 0.8503 | 0.4599 | 0.1557 | 0.1169 | 0.1745 | 0.1392 | 0.2938 | 0.1833 | 0.1538 | 0.2164 |
| G | 0.0477 | 0.0341 | 0.3929 | 0.1182 | 0.3837 | 0.1778 | 0.1564 | 0.0939 | 0.2049 | 0.2765 | 0.1866 | 0.116 |
| H | 0.0585 | 0.0412 | 0.4299 | 0.2463 | 0.1607 | 0.105 | 0.1004 | 0.0831 | 0.6712 | 0.6734 | 0.0996 | 0.0669 |

Sera from different patients with PDAC (labeled as S1-S15) were incubated with the target epitope at different dilutions in phosphate-buffered saline (PBS) (1:2 and 1:10) and reactivity was tested based on the OD reading. Differential responses with strong IgG binding reactivity to the nominal MUC4 epitope for instance in sera from patient S1 and S15 (see layout in Tables 12 and 13).

The subject matter described above is provided as an illustration of the present invention and, therefore, should not be construed to limit it. The terminology employed for the purpose of describing preferred embodiments of the present invention should not be restricted to them.

As used in the description, defined and indefinite articles, in their singular form, are intended for interpretation to also include plural forms, unless the context of the description explicitly indicates otherwise.

Undefined articles “one” should generally be interpreted as “one or more”, unless the meaning of a singular modality is clearly defined in a specific situation.

It will be understood that the terms “understand” and “include”, when used in this description, specify the presence of characteristics, elements, components, steps, and related operations, but do not exclude the possibility of other characteristics, elements, components, steps, and operations as well contemplated.

As used throughout this patent application, the term “or” is used in an inclusive sense rather than an exclusive sense, unless the exclusive meaning is clearly defined in a specific situation. In this context, a phrase of the type “X uses A or B” should be interpreted as including all relevant inclusive combinations, for example “X uses A”, “X uses B” and “X uses A and B”.

In addition, the articles “a” and “an” as used in this application and the appended claims should generally be construed to mean “one or more” unless specified otherwise or clear from the context to be directed to a singular form.

All changes, provided they do not modify the essential characteristics of the following claims, must be considered within the scope of the protection of the present invention.

### CITATION LIST

### Patent Literature

WO 2020/172202 A1
KR 102182555 B1
WO 2020/180648 A1

### Non-Patent Literature

1. GLOBOCAN. The Global Cancer Observatory—All cancers. International Agent for Research on Cancer—WHO 419, 199-200 (2020).
2. World Health Organization International Agency for Research on Cancer. GLOBOCAN 2020: Pancreatic Cancer Fact Sheet. 3-4 (2020).
3. Yao, W., Maitra, A. & Ying, H. Recent insights into the biology of pancreatic cancer. EBioMedicine 53, 102655 (2020).
4. Sideras, K. et al. Role of the immune system in pancreatic cancer progression and immune modulating treatment strategies. Cancer Treatment Reviews 40, 513-522 (2014).
5. Johnson, B. A., Yarchoan, M., Lee, V., Laheru, D. A. & Jaffee, E. M. Strategies for increasing pancreatic tumor immunogenicity. Clinical Cancer Research 23, 1656-1669 (2017).
6. Martinez-Bosch, N., Vinaixa, J. & Navarro, P. Immune evasion in pancreatic cancer: From mechanisms to therapy. Cancers 10, 1-16 (2018).
7. Das, S., Shapiro, B., Vucic, E. A., Vogt, S. & Bar-Sagi, D. Tumor Cell-Derived IL13 Promotes Desmoplasia and Immune Suppression in Pancreatic Cancer. Cancer research 80, 1088-1101 (2020).
8. Protti, M. P. & de Monte, L. Immune infiltrates as predictive markers of survival in pancreatic cancer patients. Frontiers in Physiology 4 August, 1-6 (2013).
9. Wörmann, S. M., Diakopoulos, K. N., Lesina, M. & Algül, H. The immune network in pancreatic cancer development and progression. Oncogene 33, 2956-2967 (2014).
10. Bachinsky, M. M., et al., Mapping and binding analysis of peptides derived from the tumor-associated antigen survivin for eight HLA alleles. Cancer Immun, 2005. 5: p. 6.
11. Krishna, S., et al., Stem-like CD8 T cells mediate response of adoptive cell immunotherapy against human cancer. Science, 370 (6522), 1328-1334 (2020).
12. Vodnala, S. K., et al., T cell stemness and dysfunction in tumors are triggered by a common mechanism. Science, 363(6434), eaau0135 (2019).
13. Aykut, B. et al. The fungal mycobiome promotes pancreatic oncogenesis via activation of MBL. Nature 574, 264-267 (2019).
14. Pushalkar, S. et al. The pancreatic cancer microbiome promotes oncogenesis by induction of innate and adaptive immune suppression. Cancer Discovery 8, 403-416 (2018).
15. Riquelme, E. et al. Tumor Microbiome Diversity and Composition Influence Pancreatic Cancer Outcomes. Cell 178, 795-806.e12 (2019).

1-37. (canceled)

38. A method of preparing and expanding a population of immune cells directed against tumor cells, tumor-precursor cells, non-tumor cells facilitating tumor transformation or cells facilitating tumor progression for cancer therapy, characterized by comprising the steps of:
(i) providing a body sample obtained from a mammal, in particular a tissue sample or a body liquid sample, comprising tumor cells, areas of non-tumor cells and immune cells or areas of tumor cells and immune cells, immune cells in close proximity to the tumor cells or distant to tumor cells or immune cells capable of killing tumor cells or controlling tumor cell proliferation, T-cells favoring or stopping cells that promote tumor transformation or tumor progression, tumor cell activity or tumor cell movement within tissues or in the organism over a longer period of time;
(ii) culturing the body sample to expand the immune cell populations ex vivo;
wherein the culturing comprises:
a first expansion step comprising an incubation in culture medium supplemented with the interleukins selected from IL-7, IL-15, and IL-21 and cardiolipin and optionally human serum;
a second expansion step at day 2 following the day of tumor tissue harvest with an anti-CD3 antibody, crosslinking the T-cell receptor with or without cytokine-activated irradiated feeder cells cultured for 4 to 168 hours in the presence of interleukins; and
a third expansion step at day 4 or 5 comprising an incubation in culture medium changed to IL-2, IL-7 and IL-15 supplemented with cardiolipin over the entire culture period with or without repetitively adding an anti-CD3 antibody, crosslinking the T-cell receptor with or without cytokine-activated irradiated feeder cells cultured for 4 to 168 hours in the presence of interleukins.

39. The method according to claim 38, wherein anti-tumor directed immune cells are selected from the group consisting of tumor-infiltrating lymphocytes or peripheral blood mononuclear cell, preferably selected from the group consisting of double-positive (CD4⁺CD8⁺) T-cells, double-negative (CD4⁻CD8⁻) T-cells, CD4⁺CD8⁻ or CD4⁻CD8⁺ T-cells, γδ T-cells, MAIT cells, MR1 reactive T-cells, T-cells producing Th1 cytokines, T-cells expressing cytotoxic molecules or producing IL-9, αβ T-cells, including mixed populations of tissue resident immune-cells comprising at least one of the listed immune cell subsets.

40. The method according to claim 39, wherein the anti-tumor directed immune cells are tumor-infiltrating lymphocytes or T-cells isolated from PBMCs or skin, antibody-sorted or a recombinant classical or non-classical MHC molecule loaded with the appropriate target antigen guiding antigen-specific selection comprising, but not limited to, the CDR3 region as set for in any of SEQ ID NO 1 to SEQ ID NO 410.

41. The method according to claim 38, wherein the tissue sample is tissue containing tumor cells or tissue close to cancer lesions that does not contain tumor cells obtained from patients who did not underwent any prior therapy, or underwent radiotherapy, chemotherapy, small-molecule drugs or therapy with checkpoint inhibitors, or any combination of the mentioned therapy categories.

42. The method according to claim 41, wherein the tissue sample is healthy skin tissue obtained from patients with cancer, infections, or autoimmune diseases, independent of their treatment status.

43. The method according to claim 41, wherein the tissue sample is a 1-3 mm piece collected from a 2-3 mm distance from the artery or vein or lymph vessel of the tumor based on surgically and clinically relevant locations where recurrences often take place or areas where cancer stem cells and immune cells are located.

44. The method according to claim 38, wherein the tissue sample is further dissected based on its anatomical, its microscopical architecture and/or based on an anti-tumorigenic or pro-tumorigenic protein or gene expression profile, including epigenetic differences and/or differences in microRNA.

45. The method according to claim 38, wherein the body liquid sample is cerebrospinal fluid, blood or synovial, pleural effusion, bone marrow or material from the peritoneum.

46. The method according to claim 38, wherein the expansion of anti-tumor directed immune cells is performed by adding solely culture medium, or by adding no culture medium or a limited amount of culture medium depending on the concentration of the starting solution plus
adding additional amino acids plus acetate, wherein the amino acids are in the range of 0.001 mg/L to 1 mg/L and the acetate is in the range of 0.001 mg/L to 1 mg/L;
adjusting the pH with sodium bicarbonate to the range of 7.3 to 7.5 and the glucose concentration between 1.6 and 7 g/L by adding extra glucose to the medium; and
allowing increasing lactate in the range of 1 mmol/L to 60 mmol/L.

47. The method according to claim 46, wherein the amino acids are selected from the group consisting of essential amino acids.

48. The method according to claim 38, wherein the culture medium in the first expansion step comprises interleukins selected from IL-7 ranging from 10 IU/mL to 6000 IU/mL, IL-15 ranging from 5 IU/mL to 1000 IU/mL, IL-21 ranging from 0.001 IU/mL to 100 IU/mL, cardiolipin ranging from 10 to 10000 nM, and human serum from 0.1 up to 10%.

49. The method according to claim 38, wherein the culture medium in the second expansion step comprises crosslinking of the T-cell receptor using an anti-CD3 antibody selected from the anti-CD3 complex and ranging from 10 to 3000 ng/mL.

50. The method according to claim 38, wherein the cytokine-activated irradiated feeder cells are ranging from 0.1 to 5 million feeder cells/well, preferably 1 million cells/well.

51. The method according to claim 38, wherein the culture medium in the third expansion step comprises interleukins selected from IL-2 ranging from 300 IU/mL to 6000 IU/mL, IL-7 ranging from 10 IU/mL to 6000 IU/mL and IL-15 ranging from 10 IU/mL to 1000 IU/mL and cardiolipin ranging from 10 to 1000 nM.

52. The method according to claim 38, wherein the culture medium in the second expansion step comprises crosslinking of the T-cell receptor using an anti-CD3 antibody selected from the anti-CD3 complex and ranging from 10 to 3000 ng/mL.

53. The method according to claim 38, wherein the cytokine-activated irradiated feeder cells are ranging from 0.1 to 5 million feeder cells/well, preferably 1 million cells/well.

54. The method according to claim 53, wherein the feeder cells are added every 7-14 days, preferably 7-10 days, in a ratio of feeder cells to the immune cells is in the range from 1:1 up to 400:1, preferably in a range of 10:1, along with a crosslinking anti-CD3 directed antibody in the range of 10 to 3000 ng/mL, preferably at 30 ng/mL.

55. A method to increase the frequency of γδ T-cells characterized by the fact that it occurs while expanding a population of immune cells.

56. The method according to claim 55, wherein the culture medium comprises cardiolipin ranging from 10 to 1000 nM in the presence or absence of interleukins.

57. A potency assay for tumor recognition characterized by comprising the steps of:
(i) challenging the clinically relevant immune cells as set for in SEQ ID NO 517 to SEQ ID NO 611 and for SEQ ID NO 796 to SEQ ID NO 832 with at least one target antigen of human, bacterial, viral, helminth, protozoan or bacteriophage origin or their related mimicry analogues, provided that the analogue presents 50-100% of similarity in amino acids or the analogue presents different amino acids, but with similarity based on size, structure, or charge and
(ii) detecting a change in a cytokine or immune effector molecule production selected from cytokines or chemokines, preferably in particular IFN-γ production, cell proliferation, cytotoxicity, immune signaling and/or intracellular phosphorylation of signaling molecules associated with an anti-tumor immune response.
Wherein the target antigen is a private or commonly shared tumor-associated antigen specifically presented by tumor cells or non-tumor cells supporting tumor-cells or driving tumorigenesis, the private or commonly shared tumor-associated antigen being wild-type or mutated comprising a length of 7 to 25 amino acids, and
wherein the change in the cytokine or immune effector molecules production is indicative of an anti-tumor response or pro-tumor response.

58. The potency assay according to claim 57, wherein the immune cells are selected from the group consisting of tumor-infiltrating lymphocytes and/or T-cells from healthy tissue and/or peripheral blood mononuclear cell, preferably selected from the group consisting of double-positive T-cells, double-negative T-cells, CD4⁺CD8⁻ or CD4⁻CD8⁺ T-cells, γδ T-cells, MAIT cells, MR1 reactive T-cells, T-cells producing Th1 cytokines, T-cells expressing cytotoxic molecules or producing IL-9, αβ T-cells, including mixed populations of tissue resident immune-cells comprising at least one of the listed immune cell subsets.

59. The potency assay according to claim 58, wherein the anti-tumor directed immune cells are tumor-infiltrating lymphocytes or T-cells isolated from PBMCs or skin, antibody-sorted or a recombinant classical or non-classical MHC molecule loaded with the appropriate target antigen guiding antigen-specific selection, comprising, but not limited to, the CDR3 region as set for in any of SEQ ID NO 1 to SEQ ID NO 410.

60. The potency assay according to claim 57, wherein the private or commonly shared tumor-associated antigen is selected from wild-type or mutant, not excluding fusion proteins or frameshift mutations, a 15-mer peptide or binding to or binding to MHC class II, or between 8-11 amino acids, preferably 9 amino acids, preferably binding to MHC class I target antigens or antigens presented by CD1 molecules or MR1 molecules.

61. The potency assay according to claim 57, wherein the private or commonly shared tumor-associated antigen is mutated by point mutations or frameshift mutations, the point mutation residing preferably in the middle of the antigen.

62. The potency assay according to claim 60, wherein the private tumor-associated antigen comprises a sequence as set for in any of SEQ ID NO 556 to SEQ ID NO 611.

63. The potency assay according to claim 60, wherein the commonly shared tumor-associated antigen comprises a sequence as set for in any of SEQ ID NO 517 to SEQ ID NO 555 and SEQ ID NO 796 to SEQ ID NO 832.

64. The potency assay according to claim 57, wherein the private tumor-associated targets are obtained from
providing a body sample from a patient, in particular a tumor sample;
performing whole tumor exome sequencing as compared to a non-tumor sample from the same patient;
performing RNA or DNA sequencing of the tumor sample;
preparing a construct with the mutation in the middle of the peptide sequence flanked by 7 amino acids to the left and to the right of the mutant amino acid residue;
constructing a synthetic peptide containing at least 12 to 17 amino acids of the identified frameshift mutation;
tailoring peptides to best fit according to the patient's individual MHC class I and MHC class II genetic background;
blocking classical MHC molecules, selected from MHC class I and MHC class II proteins, or non-classical MHC molecules, selected from MR1 or CD1α-d, preferably CD1d using appropriate blocking antibodies or using siRNA
analyzing the tumor sample to identify a specific T-cell receptor reaction with a wild-type or mutant target from a tumor-associated antigen provided from a cellular protein or a mitochondrial target;
identifying a mutated target antigen or a series of mutated target antigens in the tumor sample, provided that the mutated target antigen(s) do not elicit pro-tumorigenic or immune-suppressive functions; and
producing synthetic peptides with similar amino acid composition and/or a similar 3D structure with the mutated target antigen in lieu of the tumor cells or other target antigen presenting cells
wherein the specific T-cell receptor is T-cell receptor αβ, T-cell receptor γδ or T-cells receptors expressed by MAIT-cells or MR1 reactive T-cells.

65. The potency assay according to claim 54, wherein the specific T-cell receptor is a T-cell receptor as set for in any of SEQ ID NO 1 to SEQ ID NO 410.

66. The potency assay according to claim 54, wherein the reaction between the specific T-cell receptor and the target from the tumor-associated antigen give rise to immune effector functions in responding T-cells that are anti-tumor directed based on cytotoxicity, proliferation or the quality and quantity of the cytokine production, categorized by protein or RNA signatures into Th1/Th2, TH17 or TH9 responses.

67. The potency assay according to claim 54, wherein the wild-type or mutant target from a tumor-associated antigen is as set for in any of SEQ ID NO 517 to SEQ ID NO 555 and SEQ ID NO 796 to SEQ ID NO 832.

68. Biological vaccine preparation to provide anti-tumor response or antiviral response characterized by comprising target antigens that lead to the expansion of a certain set of T-cells or B-cells, wherein target antigens that show cross-reactivity to human self-proteins and antigenic structures that induce factors that are pro-tumorigenic and/or induce autoimmune responses are removed.

69. Biological vaccine preparation according to claim 68 wherein the target antigens are private or commonly shared tumor-associated targets specifically presented by tumor cells or non-tumor cells supporting tumor-cells or driving tumorigenesis by non-transformed cells that support malignant transformation, or support transformed cells.

70. Biological vaccine preparation according to claim 68 wherein the private or commonly shared tumor-associated target is selected from wild-type or mutant, not excluding fusion proteins or frameshift mutations, 15-mer peptides or binding to MHC class II, or between 8-11 amino acids, preferably 9 amino acids, preferably binding to MHC class I target antigens, antigens presented by CD1 molecules or MR1 molecules.

71. Biological vaccine preparation according to claim 68 wherein the private or commonly shared tumor-associated target is selected from wild-type or dysfunctional or damaged mitochondrial-associated molecules that act as tumor-associated targets in humans.

72. Biological vaccine preparation according to claim 71, wherein the private tumor-associated target is as set for in any of SEQ ID NO 556 to SEQ ID NO 611 or an amino acid sequence presenting up to 70% or more in amino acid exchanges, provided that the individual amino acids comprise a similar chemically 3D structure or an amino acid sequence presenting different amino acids, but with similarity based on size, structure or charge, or an amino acid sequence which is part of a chimeric antigen receptor construct.

73. Biological vaccine preparation according to claim 68, wherein the commonly shared tumor-associated target is as set for in any of SEQ ID NO 517 to SEQ ID NO 555 and SEQ ID NO 796 to SEQ ID NO 832 or an amino acid sequence presenting up to 70% or more in amino acid exchanges, provided that the individual amino acids comprise a similar chemically 3D structure or an amino acid sequence presenting different amino acids, but with similarity based on size, structure or charge, or an amino acid sequence which serves as the target antigen for an antibody or protein that binds specifically to it which can be used to construct a chimeric antigen receptor construct.

74. Epitope target characterized by the fact that the epitope is preferably the PVTSLSSVSTGDTTP from MUC4 or parts of the said epitope, preferably amino acids 3-5, 3-6, 3-7, 3-8 or amino acids of a similar size and charge, resulting in a similar 3D structure binding to the epitope from MUC4.


SEQUENCE LISTING (832 sequences)

SEQ ID NO: 1 - protein, 95 aa, Homo sapiens
PPPPPPPPPPPPPSDFITRTLGYLRYQCVTGVQTCALPILSLRIADTQTGDSAIYFCAEIRMDSSYKLIFGSGTRLLVRPDIQKPDPAVYQLRDZ

SEQ ID NO: 2 - DNA, 283 bp, Homo sapiens
  source 1..283: mol_type = genomic DNA
ccccccccccccccccccccccccccccccccccccccctctgattttattactcggactctgggctatctgcgataccagtgtgtgactggagttcagacgtgtgctcttccgatcttgtctctgcgcattgcagacacccagactggggactcagctatctacttctgtgcagagatccggatggatagcagctataaattgatcttcgggagtgggaccagactgctggtcaggcctgatatccagaagcctgaccctgccgtgtaccagctgagagact

SEQ ID NO: 3 - protein, 95 aa, Homo sapiens
PPPPPPPPPPPPPPPCFFSSRSRHTRYHCVTGVQTCALPICTLHITATQTTDVGTYFCAARGNAGNMLTFGGGTRLMVKPHIQKPDPAVYQLRDZ

SEQ ID NO: 4 - DNA, 283 bp, Homo sapiens
  source 1..283: mol_type = genomic DNA
ccccccccccccccccccccccccccccccccccccccccccccctgttttttttcaagcagaagccgccatacgcgctaccactgtgtgactggagttcagacgtgtgctcttccgatctgtaccctgcacatcacagccacccagactacagatgtaggaacctacttctgtgctgcgagggggaatgcaggcaacatgctcacctttggagggggaacaaggttaatggtcaaaccccatatccagaagcctgaccctgccgtgtaccagctgagagact

SEQ ID NO: 5 - protein, 95 aa, Homo sapiens
PPPPPPPPPPPPPPPRTTFFITHTTQYEIPLCDWSSDVCSSDLILHRATLRDAAVYYCIRLSSNTGKLIFGQGTTLQVKPDIQKPDPAVYQLRDZ

SEQ ID NO: 6 - DNA, 283 bp, Homo sapiens
  source 1..283: mol_type = genomic DNA
ccccccccccccccccccccccccccccccccccccccccccccacgcactactttttttatcacgcataccacgcaatacgagataccactgtgtgactggagttcagacgtgtgctcttccgatctaatcctgcaccgtgctaccttgagagatgctgctgtgtactactgcatccggctctctagcaacacaggcaaactaatctttgggcaagggacaactttacaagtaaaaccagatatccagaagcctgaccctgccgtgtaccagctgagagact

SEQ ID NO: 7 - protein, 95 aa, Homo sapiens
PPPPPPPPPPPPPPPPHFFFKKKTAYEIPLCDWSSDVCSSDLIVISASQLGDSAMYFCAMRDPGRRALTFGSGTRLQVQPNIQKPDPAVYQLRDZ

SEQ ID NO: 8 - DNA, 283 bp, Homo sapiens
  source 1..283: mol_type = genomic DNA
cccccccccccccccccccccccccccccccccccccccccccccccccatttttttttcaagaagaagacggcatacgagataccactgtgtgactggagttcagacgtgtgctcttccgatctaattgtcatctccgcttcacaactgggggactcagcaatgtacttctgtgcaatgagagacccgggcaggagagcacttacttttgggagtggaacaagactccaagtgcaaccaaatatccagaagcctgaccctgccgtgtaccagctgagagact

SEQ ID NO: 9 - protein, 95 aa, Homo sapiens
PPPPPPPPPPPPPPPPFFFSSRRRNTRYHCVTGVQTCALPILSLHITETQPEDSAVYFCAASIGDYKLSFGAGTTVTVRANIQKPDPAVYQLRDZ

SEQ ID NO: 10 - DNA, 283 bp, Homo sapiens
  source 1..283: mol_type = genomic DNA
cccccccccccccccccccccccccccccccccccccccccccccccattttttttttcaagcagaagacggaatacgagataccactgtgtgactggagttcagacgtgtgctcttccgatcttgtccctgcacatcacagagacccaacctgaagactcggctgtctacttctgtgcagcaagtataggcgactacaagctcagctttggagccggaaccacagtaactgtaagagcaaatatccagaagcctgaccctgccgtgtaccagctgagagact

SEQ ID NO: 11 - protein, 94 aa, Homo sapiens
PPPPPPEFVCSQTPATAIPLCDWSSDVCSSDLTLTVTSAHPEDSSFYICSAAALAERAYEQYFGPGTRLTVTEDLKNVFPPEVAVFEPSEAEIZ

SEQ ID NO: 12 - DNA, 283 bp, Homo sapiens
  source 1..283: mol_type = genomic DNA
ccccccccccccccccccccgaatttgtttgctcccagacgccggcaaccgcgataccactgtgtgactggagttcagacgtgtgctcttccgatctcactctgacagtgaccagtgcccatcctgaagacagcagcttctacatctgcagtgcagctgctctagcggaacgagcctacgagcagtacttcgggccgggcaccaggctcacggtcacagaggacctgaaaaacgtgttcccacccgaggtcgctgtgtttgagccatcagaagcagagatctc

SEQ ID NO: 13 - protein, 94 aa, Homo sapiens
PPPPPPPPPPPFFFPTRTRHTRYHCVTGVQTCALPISVTSAQKNPTAFYLCASSIRSSYEQYFGPGTRLTVTEDLKNVFPPEVAVFEPSEAEIZ

SEQ ID NO: 14 - DNA, 283 bp, Homo sapiens
  source 1..283: mol_type = genomic DNA
ccccccccccccccccccccccccccccccccccatttttttttccaaccagaacacggcatacgagataccactgtgtgactggagttcagacgtgtgctcttccgatctctgtgacatcggcccaaaagaacccgacagctttctatctctgtgccagtagtatccggagctcctacgagcagtacttcgggccgggcaccaggctcacggtcacagaggacctgaaaaacgtgttcccacccgaggtcgctgtgtttgagccatcagaagcagagatctc

SEQ ID NO: 15 - protein, 94 aa, Homo sapiens
PPPPPPPPIFFFTQTPEYEIPVCDWSADVCSSDLTLTVTSAHPEDSSFYICSAPRGETYEQYFGPGTRLTVTEDLKNVFPPEVAVFEPSEAEIZ

SEQ ID NO: 16 - DNA, 283 bp, Homo sapiens
  source 1..283: mol_type = genomic DNA
ccccccccccccccccccccccccccattttttttttcacccagacgccggaatacgagataccagtgtgtgactggagtgcagacgtgtgctcttccgatctcactctgacagtgaccagtgcccatcctgaagacagcagcttctacatctgcagtgcaccgaggggggaaacctacgagcagtacttcgggccgggcaccaggctcacggtcacagaggacctgaaaaacgtgttcccacccgaggtcgctgtgtttgagccatcagaagcagagatctc

SEQ ID NO: 17 - protein, 94 aa, Homo sapiens
  SITE 24: note = misc_feature - Xaa can be any naturally occurring amino acid
PPPPPPPPVFFFQAEAGIRDTTVXREFRRVLFRSLDIRSPGLGDAAMYLCATSRDRLYTEAFFGQGTRLTVVEDLNKVFPPEVAVFEPSEAEIZ

SEQ ID NO: 18 - DNA, 283 bp, Homo sapiens
  source 1..283: mol_type = genomic DNA
ccccccccccccccccccccccccccgtgtttttttttcaagcagaagccggcatccgcgataccactgtgtgacgggagttcagacgtgtgctcttccgatctcttgacatccgctcaccaggcctgggggacgcagccatgtacctgtgtgccaccagccgggacaggctttacactgaagctttctttggacaaggcaccagactcacagttgtagaggacctgaacaaggtgttcccacccgaggtcgctgtgtttgagccatcagaagcagagatctc

SEQ ID NO: 19 - protein, 94 aa, Homo sapiens
PLFFFTQKTASEIPLCDWSSDVCSSDLTLTVTSAHPEDSSFYICSAKIELGTGMGSNSYEQYFGPGTRLTVTEDLKNVFPPEVAVFEPSEAEIZ

SEQ ID NO: 20 - DNA, 283 bp, Homo sapiens
  source 1..283: mol_type = genomic DNA
ccccccttttttttttcacgcagaagacggcatccgagataccactgtgtgactggagttcagacgtgtgctcttccgatctaactctgacagtgaccagtgcccatcctgaagacagcagcttctacatctgcagtgctaaaatagagctagggacagggatgggaagtaactcctacgagcagtacttcgggccgggcaccaggctcacggtcacagaggacctgaaaaacgtgttcccacccgaggtcgctgtgtttgagccatcagaagcagagatctc

SEQ ID NO: 21 - protein, 95 aa, Homo sapiens
KYPNQGLQLLLKYTTGATLVKGINGFEAEFKKSETSFHLTKPSAHMSDAAEYFCAVSDNAGGTSYGKLTFGQGTILTVHPNIQNPDPAVYQLRDZ

SEQ ID NO: 22 - DNA, 283 bp, Homo sapiens
  source 1..283: mol_type = genomic DNA
aaataccccaaccaaggactccagcttctcctgaagtacacaacaggggccaccctggttaaaggcatcaacggttttgaggctgaatttaagaagagtgaaacctccttccacctgacgaaaccctcagcccatatgagcgacgcggctgagtacttctgtgctgtgagtgataatgctggtggtactagctatggaaagctgacatttggacaagggaccatcttgactgtccatccaaatatccagaaccctgaccctgccgtgtaccagctgagagact

SEQ ID NO: 23 - protein, 95 aa, Homo sapiens
GFNGLFWYQQHAGEAPTFLSYNVLDGLEEKGRFSSFLSRSKGYSYLLLKELQMKDSASYLCAVRVVDMRFGAGTRLTVKPNIQNPDPAVYQLRDZ

SEQ ID NO: 24 - DNA, 283 bp, Homo sapiens
  source 1..283: mol_type = genomic DNA
gggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagtcgtagacatgcgctttggagcagggaccagactgacagtaaaaccaaatatccagaaccctgaccctgccgtgtaccagctgagagact

SEQ ID NO: 25 - protein, 95 aa, Homo sapiens
SLQWYRQEPGEGPVLLVTVVTGGEVKKLKRLTFQFGDARKDSSLHITAAQPGDTGLYLCAGADYGQNFVFGPGTRLSVLPYIQKPDPAVYQLRDZ

SEQ ID NO: 26 - DNA, 283 bp, Homo sapiens
  source 1..283: mol_type = genomic DNA
agcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagcggactatggtcagaattttgtctttggtcccggaaccagattgtccgtgctgccctatatccagaagcctgaccctgccgtgtaccagctgagagact

SEQ ID NO: 27 - protein, 95 aa, Homo sapiens
FIWYKQESGKGPQFIIDIRSNMDKRQGQRVTVLLNKTVKHLSLQIAATQPGDSAVYFCAEKPPYGQNFVFGPGTRLSVLPYIQNPDPAVYQLRDZ

SEQ ID NO: 28 - DNA, 283 bp, Homo sapiens
  source 1..283: mol_type = genomic DNA
ttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaaacccccctatggtcagaattttgtctttggtcccggaaccagattgtccgtgctgccctatatccagaaccctgaccctgccgtgtaccagctgagagact

SEQ ID NO: 29 - protein, 95 aa, Homo sapiens
LFWYVQYPGEGLQLLLKATKADDKGSNKGFEATYRKETTSFHLEKGSVQVSDSAVYFCALTGNYGQNFVFGPGTRLSVLPYIQNPDPAVYQLRDZ

SEQ ID NO: 30 - DNA, 283 bp, Homo sapiens
  source 1..283: mol_type = genomic DNA
cttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctcacggggaactatggtcagaattttgtctttggtcccggaaccagattgtccgtgctgccctatatccagaaccctgaccctgccgtgtaccagctgagagact

SEQ ID NO: 31 - protein, 94 aa, Homo sapiens
GLRLIYYSQIVNDFQKGDIAEGYSVSREKKESFPLTVTSAQKNPTAFYLCASTLTGSYNEQFFGPGTRLTVLEDLKNVFPPEVAVFEPSEAEIZ

SEQ ID NO: 32 - DNA, 283 bp, Homo sapiens
  source 1..283: mol_type = genomic DNA
aagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtactctaacaggttcctacaatgagcagttcttcgggccagggacacggctcaccgtgctagaggacctgaaaaacgtgttcccacccgaggtcgctgtgtttgagccatcagaagcagagatctc

SEQ ID NO: 33 - protein, 94 aa, Homo sapiens
LRQIYYSMNVEVTDKGDVPEGYKVSRKEKRNFPLILESPSPNQTSLYFCASSLRTGTNYGYTFGSGTRLTVVEDLNKVFPPEVAVFEPSEAEIZ

SEQ ID NO: 34 - DNA, 283 bp, Homo sapiens
  source 1..283: mol_type = genomic DNA
gcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttaaggacaggcactaactatggctacaccttcggttcggggaccaggttaaccgttgtagaggacctgaacaaggtgttcccacccgaggtcgctgtgtttgagccatcagaagcagagatctc

SEQ ID NO: 35 - protein, 94 aa, Homo sapiens
LELLIYFNNNVPIDDSGMPEDRFSAKMPNASFSTLKIQPSEPRDSAVYFCASSPLSVNTEAFFGQGTRLTVVEDLNKVFPPEVAVFEPSEAEIZ

SEQ ID NO: 36 - DNA, 283 bp, Homo sapiens
  source 1..283: mol_type = genomic DNA
gactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtccactatcagtcaacactgaagctttctttggacaaggcaccagactcacagttgtagaggacctgaacaaggtgttcccacccgaggtcgctgtgtttgagccatcagaagcagagatctc

SEQ ID NO: 37 - protein, 94 aa, Homo sapiens
FLISFYEKMQSDKGSIPDRFSAQQFSDYHSELNMSSLELGDSALYFCASRPTGLTGEGTEAFFGQGTRLTVVEDLNKVFPPEVAVFEPSEAEIZ

SEQ ID NO: 38 - DNA, 283 bp, Homo sapiens
  source 1..283: mol_type = genomic DNA
agttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcaggccgaccgggctaacaggcgagggcactgaagctttctttggacaaggcaccagactcacagttgtagaggacctgaacaaggtgttcccacccgaggtcgctgtgtttgagccatcagaagcagagatctc

SEQ ID NO: 39 - protein, 94 aa, Homo sapiens
MATSNEGSKATYEQGVEKDKFLINHASLTLSTLTVTSAHPEDSSFYICSAWGAGRGPQYEQYFGPGTRLTVTEDLKNVFPPEVAVFEPSEAEIZ

SEQ ID NO: 40 - DNA, 283 bp, Homo sapiens
  source 1..283: mol_type = genomic DNA
tgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgcttggggggcgggcagggggccccaatacgagcagtacttcgggccgggcaccaggctcacggtcacagaggacctgaaaaacgtgttcccacccgaggtcgctgtgtttgagccatcagaagcagagatctc

SEQ ID NO: 41 - protein, 94 aa, Homo sapiens
HPPPPVFFFLHYDGKRENTVVLEFRRVLFRSQHNVEKQDIATYYCALWDYKKLFGSGTTLVVTDKQLDADVSPKPTIFLPSIAETKLQKAGTYL

SEQ ID NO: 42 - DNA, 283 bp, Homo sapiens
  source 1..283: mol_type = genomic DNA
ccacccccccccacccgtgtttttttttctacattacgacggaaaacgagagaacactgtggtactggagttcagacgtgtgctcttccgatctcaacacaatgtagagaaacaggacatagctacctactactgtgccttgtgggattataagaaactctttggcagtggaacaacactggttgtcacagataaacaacttgatgcagatgtttcccccaagcccactatttttcttccttcaattgctgaaacaaagctccagaaggctggaacatacctt

SEQ ID NO: 43 - protein, 95 aa, Homo sapiens
  SITE 8: note = misc_feature - Xaa can be any naturally occurring amino acid
  SITE 24: note = misc_feature - Xaa can be any naturally occurring amino acid
  SITE 26: note = misc_feature - Xaa can be any naturally occurring amino acid
  SITE 65: note = misc_feature - Xaa can be any naturally occurring amino acid
  SITE 68: note = misc_feature - Xaa can be any naturally occurring amino acid
  SITE 84: note = misc_feature - Xaa can be any naturally occurring amino acid
GIRDTTVXLEFRRVLFRSCAKSNXKXFWIYYCATWDRRNYKKLFGSGTTLVVTGKYRKNTTFPRXTTXCRCFPQAHYFSSFDCXNKTPEGWNIPZ

SEQ ID NO: 44 - DNA, 283 bp, Homo sapiens
  source 1..283: mol_type = genomic DNA
ggcatacgagataccactgtgtgactggagttcagacgtgtgctcttccgatcttgtgcaaaatctaattgaaaatgattctggatctattactgtgccacctgggacaggcggaattataagaaactctttggcagtggaacaacacttgttgtcacaggtaagtatcggaagaatacaacatttccaagataaacaacttgatgcagatgtttcccccaagcccactatttttcttccttcgattgctgaaacaaaactccagaaggctggaacatacctt

SEQ ID NO: 45 - protein, 94 aa, Homo sapiens
PPIFFFKQKTAYEIPLCDWSSDVCSSDLKENLIERVSGVYYCATWDRAYYKKLFGSGTTLVVTDKQLDADVSPKPTIFLPSIAETKLQKAGTYL

SEQ ID NO: 46 - DNA, 283 bp, Homo sapiens
  source 1..283: mol_type = genomic DNA
cccccccattttttttttcaagcagaagacggcatacgagataccactgtgtgactggagttcagacgtgtgctcttctgatctaaaggaaaatctaattgaacgtgtctctggggtctattactgtgccacctgggatagagcctattataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 47 - protein, 94 aa, Homo sapiens
  SITE 9: note = misc_feature - Xaa can be any naturally occurring amino acid
  SITE 15: note = misc_feature - Xaa can be any naturally occurring amino acid
FFLKAHEAXRDTTVXLEFRRLLFRSKHNVEKQDIATYYCALWESQELGKKIKVFGPGTKLIITDKQLDADVSPKPTIFLPPIAETKLQKAGTYL

SEQ ID NO: 48 - DNA, 283 bp, Homo sapiens
  source 1..283: mol_type = genomic DNA
tttttttttaaaagcacacgaggcctaacgagataccactgtgtgactggagttcagacgtttgctcttccgatctaagcacaatgtagagaaacaggacatagctacctactactgtgccttgtgggaatctcaagagttgggcaaaaaaatcaaggtatttggtcccggaacaaagcttatcattacagataaacaacttgatgcagatgtttcccccaagcccactatttttcttcctccaattgctgaaacaaagctccagaaggctggaacatacctt

SEQ ID NO: 49 - protein, 94 aa, Homo sapiens
  SITE 21: note = misc_feature - Xaa can be any naturally occurring amino acid
PPPPDVYFFNHHPAIPDTTVXLEFRRVLFRSTHNVEKQDIATYYCALLYYKKLFGSGTTLVVTDKQLDADVSPKPTIFLPSIAETKLQKAGTYL

SEQ ID NO: 50 - DNA, 283 bp, Homo sapiens
  source 1..283: mol_type = genomic DNA
ccccccccccccagacgtctattttttcaaccaccaccccgccatacccgataccactgtgtgactggagttcagacgtgtgctcttccgatctactcacaatgtagagaaacaggacatagctacctactactgtgccttgctttattataagaaactctttggcagtggaacaacacttgttgtcacagataaacaacttgatgcagatgtttcccccaagcccactatttttcttccttcgattgctgaaacaaaactccagaaggctggaacatacctt

SEQ ID NO: 51 - protein, 94 aa, Homo sapiens
PTVFFFKPTTAYEIPLCDWSSDVCSSDLLQNLIENDSGVYYCATWDWDYYKKLFGSGTTLVVTDKQLDADVSPKPTIFLPSIAETKLQKAGTYL

SEQ ID NO: 52 - DNA, 283 bp, Homo sapiens
  source 1..283: mol_type = genomic DNA
ccccaccgttttttttttcaagccgacgacggcatacgagataccactgtgtgactggagttcagacgtgtgctcttccgatcttctgcaaaatctaattgaaaatgattctggggtctattactgtgccacctgggactgggattattataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 53 - protein, 94 aa, Homo sapiens
PFFFSRRRRHTRDHCVTGVQTCALPIYMRNLIENDSGVYYCATWDGPDYYKKLFGSGTTLVVTDKQLDADVSPKPTIFLPSIAETKLQKARTYL

SEQ ID NO: 54 - DNA, 283 bp, Homo sapiens
  source 1..283: mol_type = genomic DNA
cccattttttttttcacgcagaagacggcatacgagagaccactgtgtgactggagttcagacgtgtgctcttccgatctatatgcgaaatcttattgaaaatgactctggagtctattactgtgccacctgggatgggccggattattataagaaactctttggcagtggaacaacacttgttgtcacagataaacaacttgatgcagatgtttcccccaagcccactatttttcttccttcgattgctgaaacaaaactccagaaggctagaacatatctt

SEQ ID NO: 55 - protein, 94 aa, Homo sapiens
FFSSRRRHTTYHCVTGVQTCALPISVRNLIENDFGVYYCATWERGSDWIKTFAKGTRLIVTSPDKQLDADVSPKPTIFLPSIAETKLQKAGTYL

SEQ ID NO: 56 - DNA, 283 bp, Homo sapiens
  source 1..283: mol_type = genomic DNA
ttttttttcaagcagaagacggcatacgacataccactgtgtgactggagttcagacgtgtgctcttccgatctcggtgcgaaatcttattgaaaatgactttggggtctattactgtgccacctgggaaaggggtagtgattggatcaagacgtttgcaaaagggactaggctcatagtaacttcgcctgataaacaacttgatgcagatgtttcccccaagcccactatttttcttccttcgattgctgaaacaaaactccagaaggctggaacatatctt

SEQ ID NO: 57 - protein, 94 aa, Homo sapiens
PFFFSSRRRLTRYQCVTGVQTCSLPISLLNLIENDFGVYYCATWDALNYYKKLFGSGTTLVVTDKQLDADVSPKPTIFLPSIAETKLQKAGTYL

SEQ ID NO: 58 - DNA, 283 bp, Homo sapiens
  source 1..283: mol_type = genomic DNA
acctttttttttttcaagccgaagacggcttacgagataccaatgtgtgactggagttcagacgtgttctcttcctatctcactgctaaatctaattgaaaatgactttggggtctattactgtgccacctgggacgccttgaattattataagaaactctttggcagtggaacaacacttgttgtcacagataaacaacttgatgcagatgtttcccccaagcccactatttttcttccttcgattgctgaaacgaaactccagaaggctggaacatatctt

SEQ ID NO: 59 - protein, 94 aa, Homo sapiens
  SITE 7: note = misc_feature - Xaa can be any naturally occurring amino acid
  SITE 29: note = misc_feature - Xaa can be any naturally occurring amino acid
PTQIFFXPTHRHHRYQCVTGVQTCAPPIXMRNLIENDSGVYYCATWGTYYKKLFGSGTTLVVTDKQLDADVSPKPTIFLPSIAETKLQKAGTYL

SEQ ID NO: 60 - DNA, 283 bp, Homo sapiens
  source 1..283: mol_type = genomic DNA
acccacacaaatttttttttaacccacacaccgccatcacagataccaatgtgtgactggagttcagacgtgtgctcctccgatctaaatgcgaaatcttattgaaaatgactctggagtctattactgtgccacctggggaacttattataagaaactctttggcagtggaacaacacttgttgtcacagataaacaacttgatgcagatgtttcccccaagcccactatttttcttccttcgattgctgaaacaaaactccagaaggctggaacatacctt

SEQ ID NO: 61 - protein, 94 aa, Homo sapiens
PPPPPPPPPPPPPPLFFFKQKTAYEIPLCDWSSDVCSSDLLTISALQLEDSAKYFCALGELNLLGVPRPDKLIFGKGTRVTVEPRSQPHTKPSZ

SEQ ID NO: 62 - DNA, 283 bp, Homo sapiens
  source 1..283: mol_type = genomic DNA
cccccccccccccccccccccccccccccccccccccccccccacttttttttttcaagcagaagacggcatacgagataccactgtgtgactggagttcagacgtgtgctcttccgatctgttaaccatttcagccttacagctagaagattcagcaaagtacttttgtgctcttggggaactgaacctactgggggttcccagacccgataaactcatctttggaaaaggaacccgtgtgactgtggaaccaagaagtcagcctcataccaaaccatccgt

SEQ ID NO: 63 - protein, 94 aa, Homo sapiens
PPPPPPPTPPPPPTVFFFTQIRETDIPLCDWSSDVCSSDLLLKILAPSERDEGSYYCACDEVGDNGEYTDKLIFGKGTRVTVEPRSQPHTKPSZ

SEQ ID NO: 64 - DNA, 283 bp, Homo sapiens
  source 1..283: mol_type = genomic DNA
cccccccccccccccccccccccacccccccccccccccccactgtattttttttcacgcagatcagggaaaccgacatcccactgtgtgactggagttcagacgtgtgctcttccgatctgctacttaagatacttgcaccatcagagagagatgaagggtcttactactgtgcctgtgacgaagttggggataatggggagtacaccgataaactcatctttggaaaaggaacccgtgtgactgtggaaccaagaagtcagcctcataccaaaccatccgt

SEQ ID NO: 65 - protein, 94 aa, Homo sapiens
PPPPPPPPPPPPPHPPPCFFFMQKTSYELPLCDWSSDVCSSDLTLKILAPSERDEGSYYCACESLGDSADKLIFGKGTRVTVEPRSQPHTKPSZ

SEQ ID NO: 66 - DNA, 283 bp, Homo sapiens
  source 1..283: mol_type = genomic DNA
ccccccccccccccccccccccccccccccccccccccccccaccctccaccgtgtttttttttcatgcagaagacgtcatacgagttaccactgtgtgactggagttcagacgtgtgctcttccgatctaacacttaagatacttgcaccatcagagagagatgaagggtcttactactgtgcctgtgagtccctgggggattccgccgataaactcatctttggaaaaggaacccgtgtgactgtggaaccaagaagtcagcctcataccaaaccatccgt

SEQ ID NO: 67 - protein, 94 aa, Homo sapiens
PPPPPPPFFFFKQTTAYAIPLCDWSSDVCSFDLGHLVISPVRTEDSATYYCALPLTSYDFKLGDTGSDADKLIFGKGTRVTVEPRSQPHTKPSZ

SEQ ID NO: 68 - DNA, 283 bp, Homo sapiens
  source 1..283: mol_type = genomic DNA
ccccccccccccccccccccccctttttttttttcaagcagacgacggcatacgcgataccactgtgtgactggagttcagacgtgtgctctttcgatctgggtcacttggtgatctctccagtaaggactgaagacagtgccacttactactgtgcccttcccctcacttcctacgattttaaactgggggatacagggtcggacgccgataaactcatctttggaaaaggaacccgtgtgactgtggaaccaagaagtcagcctcataccaaaccatccgt

SEQ ID NO: 69 - protein, 94 aa, Homo sapiens
PPPPPPPPPPPPPLIFFFTHKTAYEIPLCDWSSDVCSSDLVLKILAPSERDEGSYYCACDLLGDMGVSTDKLIFGKGTRVTVEPRSQPHTKPSZ

SEQ ID NO: 70 - DNA, 283 bp, Homo sapiens
  source 1..283: mol_type = genomic DNA
cccccccccccccccccccccccccccccccccccccccccctgattttttttttcacgcataagacggcatacgagattccactgtgtgactggagttcagacgtgtgctcttccgatctggtacttaagatacttgcaccatcagagagagatgaagggtcttactactgtgcctgtgacctactgggggatatgggtgtttcgaccgataaactcatctttggaaaaggaacccgtgtgactgtggaaccaagaagtcagcctcataccaaaccatccgt

SEQ ID NO: 71 - protein, 94 aa, Homo sapiens
  SITE 25: note = misc_feature - Xaa can be any naturally occurring amino acid
PPPPPPHPPPPPHPKKFYKNTKTEXAIQLCDWTSDVCSTDLLTISALQLEDSAKYFCALGEPRITWFRTDKLIFGKGTRVTVEPRSQPHTKPSZ

SEQ ID NO: 72 - DNA, 283 bp, Homo sapiens
  source 1..283: mol_type = genomic DNA
cccccccccccccccccccccacccccccccccccccacaccccaaaaaattttataaaaacacaaagaccgaataagcaatacaattgtgtgactggacttcagacgtgtgctctaccgatctactaaccatttcagccttacagctagaagattcagcaaagtacttttgtgctcttggggaacctaggataacttggttccgcaccgataaactcatctttggaaaaggaacccgtgtgactgtggaaccaagaagtcagcctcataccaaaccatccgt

SEQ ID NO: 73 - protein, 94 aa, Homo sapiens
PPPPPPPPPPPPPPPPLFFFKQKTAYEIPLCDWSSDVCSSDLRTISALQLEDSAKYFCALGESSYGDASRPLIFGKGTYLEVQQRSQPHTKPSZ

SEQ ID NO: 74 - DNA, 283 bp, Homo sapiens
  source 1..283: mol_type = genomic DNA
cccccccccccccccccccccccccccccccccccccccccccccccccccttttttttttcaagcagaagacggcatacgagataccactgtgtgactggagttcagacgtgtgctcttccgatcttcgaaccatttcagccttacagctagaagattcagcaaagtacttttgtgctcttggggaaagttcctacggggatgcctcccgacccctgatctttggcaaaggaacctatctggaggtacaacaacgaagtcagcctcataccaaaccatccgt

SEQ ID NO: 75 - protein, 94 aa, Homo sapiens
  SITE 21: note = misc_feature - Xaa can be any naturally occurring amino acid
PPPPPIFFFHAEDGIRDTTVXLGSDVCSSDLVLKILAPSERDEGSYYCACDTVLLGDTRDFFETTEPYTDKLIFGKGTRVTVEPRSQPHTKPSZ

SEQ ID NO: 76 - DNA, 283 bp, Homo sapiens
  source 1..283: mol_type = genomic DNA
cccccccccccccccccatttttttttttcacgcagaagacggcatacgcgataccactgtgtgactgggttcagacgtgtgctcttccgatctcgtacttaagatacttgcaccatcagagagagatgaagggtcttactactgtgcctgtgacaccgtacttctgggggatacgcgtgattttttcgaaacgacagaaccgtacaccgataaactcatctttggaaaaggaacccgtgtgactgtggaaccaagaagtcagcctcataccaaaccatccgt

SEQ ID NO: 77 - protein, 94 aa, Homo sapiens
PPPPPPPPPPPPPPPHIFFFKQKPQYEIPMCDWSSDVCSSDLVTISALQLEDSAKYFCALGESSYGDASLPLIFGKGTYLEVQQRSQPHTKPSZ

SEQ ID NO: 78 - DNA, 283 bp, Homo sapiens
  source 1..283: mol_type = genomic DNA
ccccccccccccccccccccccccccccccccccccccccccccccccacattttttttttcaagcagaagccgcaatacgagataccaatgtgtgactggagttcagacgtgtgctcttccgatctggtaaccatttcagccttacagctagaagattcagcaaagtacttttgtgctcttggggaaagttcctacggggatgcctccctacccctgatctttggcaaaggaacctatctggaggtacaacaacgaagtcagcctcataccaaaccatccgt

SEQ ID NO: 79 - protein, 94 aa, Homo sapiens
PPPPPPPPPPPPPPPTHFISQRPRPSHIPLCDWSSDVCSSDLVLKILAPSERDEGSYYCACDTLGLTDTDKLIFGKGTRVTVEPRSQPHTKPSZ

SEQ ID NO: 80 - DNA, 283 bp, Homo sapiens
  source 1..283: mol_type = genomic DNA
cccccccccccccccccccccccccccccccccccccccccccccccacgcattttatttcccagcgaccccgcccatcccacataccactgtgtgactggagttcagacgtgtgctcttccgatctagtacttaagatacttgcaccatcagagagagatgaagggtcttactactgtgcctgtgacactctgggactaaccgacaccgataaactcatctttggaaaaggaacccgtgtgactgtggaaccaagaagtcagcctcataccaaaccatccgt

SEQ ID NO: 81 - protein, 94 aa, Homo sapiens
SYNSKVVLESGVSPGKYYTYASTRNNLRLILRNLIENDFGVYYCATWDGPAKLFGSGTTLVVTDKQLDADVSPKPTIFLPSIAETKLQKAGTYL

SEQ ID NO: 82 - DNA, 283 bp, Homo sapiens
  source 1..283: mol_type = genomic DNA
atcctacaactccaaggttgtgttggaatcaggagtcagtccagggaagtattatacttacgcaagcacaaggaacaacttgagattgatactgcgaaatctaattgaaaatgactttggggtctattactgtgccacctgggacgggccggcgaaactctttggcagtggaacaacacttgttgtcacagataaacaacttgatgcagatgtttcccccaagcccactatttttcttccttcaattgctgaaacaaaactccagaaggctggaacatacctt

SEQ ID NO: 83 - protein, 94 aa, Homo sapiens
YDGTVRKESGIPAGKFEVDRIPETSTSTLTIHNVEKQDIATYYCALWEDYKKLFGSGTTLVVTDKQLDADVSPKPTIFLPSIAETKLQKAGTYL

SEQ ID NO: 84 - DNA, 283 bp, Homo sapiens
  source 1..283: mol_type = genomic DNA
atatgacggcactgtcagaaaggaatccggcattccggcaggcaaatttgaggtggataggatacctgaaacgtctacatccactctcaccattcacaatgtagagaaacaggacatagctacctactactgtgccttgtgggaggactataagaaactctttggcagtggaacaacacttgttgtcacagataaacaacttgatgcagatgtttcccccaagcccactatttttcttccttcaattgctgaaacaaaactccagaaggctggaacatatctt

SEQ ID NO: 85 - protein, 94 aa, Homo sapiens
SSVVLESGISPGKYDTYGSTRKNLRMILRNLIENDSGVYYCATWDGQGYYKKLFGSGTTLVVTDKQLDADVSPKPTIFLPSIAETKLQKAGTYL

SEQ ID NO: 86 - DNA, 283 bp, Homo sapiens
  source 1..283: mol_type = genomic DNA
ctccagcgttgtcttggaatcaggaatcagcccagggaagtatgatacttacggaagcacaaggaagaacttgagaatgatactgcgaaatcttattgaaaatgactctggagtctattactgtgccacctgggatgggcaaggttattataagaaactctttggcagtggaacaacacttgttgtcacagataaacaacttgatgcagatgtttcccccaagcccactatttttcttccttcaattgctgaaacaaaactccagaaggctggaacatatctt

SEQ ID NO: 87 - protein, 94 aa, Homo sapiens
KESGIPSGKFEVDRIPETSTSTLTIHNVEKQDIATYYCALWEVQGELGKKIKVFGPGTKLIITDKQLDADASPKPTIFLPSIAETKLQKAGTYL

SEQ ID NO: 88 - DNA, 283 bp, Homo sapiens
  source 1..283: mol_type = genomic DNA
aaaggaatccggcattccgtcaggcaaatttgaggtggataggatacctgaaacgtctacatccactctcaccattcacaatgtagagaaacaggacatagctacctactactgtgccttgtgggaggtgcaaggagagttgggcaaaaaaatcaaggtatttggtcccggaacaaagcttatcattacagataaacaacttgatgcagatgcttcccccaagcccactatttttcttccttcaattgctgaaacaaagctccagaaggctggaacatacctt

SEQ ID NO: 89 - protein, 94 aa, Homo sapiens
KVVLESGVSPGKYYTYASTRNNLRLILRNLIENDSGVYYCATFRSSDWIKTFAKGTKLIVTSPDKQLDADVSPKPTIFLPSIAETKLQKAGTYL

SEQ ID NO: 90 - DNA, 283 bp, Homo sapiens
  source 1..283: mol_type = genomic DNA
caaggttgtgttggaatcaggagtcagtccagggaagtattatacttacgcaagcacaaggaacaacttgagattgatactgcgaaatctaattgaaaatgactctggggtctattactgtgccacctttcgaagtagtgattggatcaagacgtttgcaaaagggactaagctcatagtaacttcgcctgataaacaacttgatgcagatgtttcccccaagcccactatttttcttccttcaattgctgaaacaaaactccagaaggctggaacatacctt

SEQ ID NO: 91 - protein, 94 aa, Homo sapiens
TSSVVGESGISPGKYDTYGRTRKNLRMILRNLIENDSGVYYCATWEVYYYKKLFGSGTTLVVTDKQFDADVSPKPTIFLPSIAETKLQKAGTYL

SEQ ID NO: 92 - DNA, 283 bp, Homo sapiens
  source 1..283: mol_type = genomic DNA
cacctccagcgttgtgggggaatcaggaatcagcccaggcaagtatgatacttacggaagaacaaggaagaacttgagaatgatactgcgaaatcttattgaaaatgactctggagtctattactgtgccacctgggaggtttattattataagaaactctttggcagtggaacaacacttgttgtcacagataaacaatttgatgcagatgtttcccccaagcccactatttttcttccttcaattgctgaaacaaaactccagaaggctggaacatatctt

SEQ ID NO: 93 - protein, 94 aa, Homo sapiens
VVLESGVSPGKYYTYESTRNNLRLILRNLIENDSGVYYCATWDGRKDWIKTFAKGTRLIVTSPDKQLDADVSPKPTIFLPSIAETKLQKAGTYL

SEQ ID NO: 94 - DNA, 283 bp, Homo sapiens
  source 1..283: mol_type = genomic DNA
ggttgtgttggaatcaggagtcagtccagggaagtattatacttacgaaagcacaaggaacaacttgagattgatactgcgaaatctaattgaaaatgactctggggtctattactgtgccacctgggacggtcgaaaagattggatcaagacgtttgcaaaagggactaggctcatagtaacttcgcctgataaacaacttgatgcagatgtttcccccaagcccactatttttcttccttcgattgctgaaacaaaactccagaaggctggaacatacctt

SEQ ID NO: 95 - protein, 94 aa, Homo sapiens
DSYNSRVVLESGISREKYHTYASTGKSLKFILENLIERDSGVYYCATWDREKLFGSGTTLVVTGKQLDADVSPKPTIFLPSIAETKLQKAGTYL

SEQ ID NO: 96 - DNA, 283 bp, Homo sapiens
  source 1..283: mol_type = genomic DNA
tgactcctacaactccagggttgtgttggaatcaggaatcagtcgagaaaagtatcatacttatgcaagcacagggaagagccttaaatttatactggaaaatctaattgaacgtgactctggggtctattactgtgccacctgggatagggagaaactctttggcagtggaacaacacttgttgtcacaggtaaacaacttgatgcagatgtttcccccaagcccactatttttcttccttcgattgctgaaacaaaactccagaaggctggaacatatctt

SEQ ID NO: 97 - protein, 94 aa, Homo sapiens
YVGTSRKEAGIPSGKFEVDRIPETSTSTVTIQNVEKQDIANYYCALWEDFKKLFGSGTTLVVTDKQLDADVSPKPTIFLPSIAETKLQKAGTYL

SEQ ID NO: 98 - DNA, 283 bp, Homo sapiens
  source 1..283: mol_type = genomic DNA
atatgtcggaactagcagaaaggaagccgggattccgtcagggaaatttgaggtggataggatacctgaaacgtctacatccactgtcaccattcagaatgtggagaaacaggacatagctaactactactgtgccttgtgggaggactttaagaaactctttggcagtggaacaacacttgttgtcacagataaacaacttgatgcagatgtttcccccaagcccactatttttcttccttcaattgctgaaacaaaactccagaaggctggaacatatctt

SEQ ID NO: 99 - protein, 94 aa, Homo sapiens
  SITE 34: note = misc_feature - Xaa can be any naturally occurring amino acid
TSSYVVDSVKSTGIHDTDGFTSKNFRMILRNIIXNDSGVFYCASLEVYYYKKLFGSGTTLVVTDKQFDADVSPKPTIFLPSIAETKLQKAGTYL

SEQ ID NO: 100 - DNA, 283 bp, Homo sapiens
  source 1..283: mol_type = genomic DNA
aacctccagctatgtggtggattcagtaaagagcactggcattcatgatactgacggattcacaagtaagaactttagaatgatactgcgaaatattatttaaaatgactctggagtcttttactgtgcctccttggaggtttattattataagaaactctttggcagtggaacaacacttgttgtcacagataaacaatttgatgcagatgtttcccccaagcccactatttttcttccttcaattgctgaaacaaaactccagaaggctggaacatatctt

SEQ ID NO: 101 - protein, 94 aa, Homo sapiens
  SITE 15: note = misc_feature - Xaa can be any naturally occurring amino acid
RKTQGNTMTFIYREXDIYGPGFKDNFQGDIDIAKNLAVLKILAPSERDEGSYYCACDTVLPTTGGYGDTDKLIFGKGTRVTVEPRSQPHTKPSZ

SEQ ID NO: 102 - DNA, 283 bp, Homo sapiens
  source 1..283: mol_type = genomic DNA
gcaggaagacccaaggtaacacaatgactttcatataccgagaataggacatctatggccctggtttcaaagacaatttccaaggtgacattgatattgcaaagaacctggctgtacttaagatacttgcaccatcagagagagatgaagggtcttactactgtgcctgtgacacggtccttcctacgactgggggatacggggacaccgataaactcatctttggaaaaggaacccgtgtgactgtggaaccaagaagtcagcctcataccaaaccatccgt

SEQ ID NO: 103 - protein, 94 aa, Homo sapiens
WYKQLPSKEMIFLIRQGSDEQNAKSGRYSVNFKKAAKSVALTISALQLEDSAKYFCALGGWYWGITTRTDKLIFGKGTRVTVEPRSQPHTKPSZ

SEQ ID NO: 104 - DNA, 283 bp, Homo sapiens
  source 1..283: mol_type = genomic DNA
tttggtacaagcaacttcccagcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggtggctggtactgggggataacgacgaggaccgataaactcatctttggaaaaggaacccgtgtgactgtggaaccaagaagtcagcctcataccaaaccatccgt

SEQ ID NO: 105 - protein, 94 aa, Homo sapiens
WYKQLPSKEMIFLIRQGSDEQNAKSGRYSVNFKKAAKSVALTISALQLEDSAKYFCALGGWYWGMTTRTDKLIFGKGTRVTVEPRSQPHTKPSZ

SEQ ID NO: 106 - DNA, 283 bp, Homo sapiens
  source 1..283: mol_type = genomic DNA
tttggtacaagcaacttcccagcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggtggctggtactgggggatgacgacgaggaccgataaactcatctttggaaaagggacccgtgtgactgtggaaccaagaagtcagcctcataccaaaccatccgt

SEQ ID NO: 107 - protein, 94 aa, Homo sapiens
  SITE 2: note = misc_feature - Xaa can be any naturally occurring amino acid
  SITE 51: note = misc_feature - Xaa can be any naturally occurring amino acid
FXYQFTIIEMFFIIRLGSDEQNSIIGSYSFNFKKAAKSVELTISALQLEDXAKFFCALGVWYCVITTWTDKLIFGKGTRVTVEPRSQPHTKPSZ

SEQ ID NO: 108 - DNA, 283 bp, Homo sapiens
  source 1..283: mol_type = genomic DNA
ttttttaatatcaatttacaatcattgagatgtttttcattattcgcctgggttctgatgaacagaattcaataattggtagctattctttcaacttcaagaaagcagcgaaatccgtcgagttaaccatttcagccttacagctagaagattaagcaaagttcttttgtgctcttggtgtctggtactgtgtgataacgacgtggaccgataaactcatctttggaaaaggaacacgtgtgactgtggaaccaagaagtcagcctcataccaaaccatccgt

SEQ ID NO: 109 - protein, 94 aa, Homo sapiens
  SITE 2: note = misc_feature - Xaa can be any naturally occurring amino acid
  SITE 28: note = misc_feature - Xaa can be any naturally occurring amino acid
  SITE 49: note = misc_feature - Xaa can be any naturally occurring amino acid
SXNANPIIVMFFLIRKVSDVNYSNSVRXSFNFMQEAKSVAINISALQLXDSAKYFCALGGWYLGITTRTDKLIFGKGTRVTVEPRSQPHTKPSZ

SEQ ID NO: 110 - DNA, 283 bp, Homo sapiens
  source 1..283: mol_type = genomic DNA
ttagttaaaatgctaatcccatcatagttatgttttttcttattcggaaggtttctgatgtaaattattcaaacagtgttcggtagtctttcaacttcatgcaagaagctaaatccgtcgccataaacatttcagccttacagctataagattcagcaaagtacttttgtgctcttggtggctggtacttgggtataacgacgaggaccgataaactcatctttggaaaaggaacccgtgtgactgtggaaccaagaagtcagcctcataccaaaccatccgt

SEQ ID NO: 111 - protein, 95 aa, Homo sapiens
  SITE 28: note = misc_feature - Xaa can be any naturally occurring amino acid
PPPPPPPPPPPPPPPRYFFSSRSRNTRXGVVTGVQTCALPISLSLPRVSLSDTAVYYCLVVGGGFQKLVFGTGTRLLVSPNIQNPDPAVYQLRDZ

SEQ ID NO: 112 - DNA, 283 bp, Homo sapiens
  source 1..283: mol_type = genomic DNA
ccccccccccccccccccccccccccccccccccccccccccccccgttattttttttcaagcagatcccggaatacgagatgaggggttgtgactggagttcagacgtgtgctcttccgatctctctgagcctgccccgggtttccctgagcgacactgctgtgtactactgcctcgtggtggggggaggctttcagaaacttgtatttggaactggcacccgacttctggtcagtccaaatatccagaaccctgaccctgccgtgtaccagctgagagact

SEQ ID NO: 113 - protein, 95 aa, Homo sapiens
  SITE 29: note = misc_feature - Xaa can be any naturally occurring amino acid
PPPPPPPPPPPPPPPPPFFFSSTRRQPRXGVVTGVQTCALPICSLHITATQTTDVGTYFCNSGGYQKVTFGIGTKLQVIPNIQNPDPAVYQLRDZ

SEQ ID NO: 114 - DNA, 283 bp, Homo sapiens
  source 1..283: mol_type = genomic DNA
ccccccccccccccccccccccccccccccccccccccccccccccccccattttttttttcaagcacacgccggcaaccgagatgaggggttgtgactggagttcagacgtgtgctcttccgatctgctccctgcacatcacagccacccagactacagatgtaggaacctacttctgtaattctgggggttaccagaaagttacctttggaattggaacaaagctccaagtcatcccaaatatccagaaccctgaccctgccgtgtaccagctgagagact

SEQ ID NO: 115 - protein, 95 aa, Homo sapiens
  SITE 26: note = misc_feature - Xaa can be any naturally occurring amino acid
PPPPPPPPPPPPPPIFFTRTRRHTRXGVVTGVQTCALPIFSLLIRDSQPSDSATYLCAVGLQGGGNKLTFGTGTQLKVELNIQNPDPAVYQLRDZ

SEQ ID NO: 116 - DNA, 283 bp, Homo sapiens
  source 1..283: mol_type = genomic DNA
ccccccccccccccccccccccccccccccccccccccccccattttttttacacgcacaagacgccatacgcgatgaggggttgtgactggagttcagacgtgtgctcttccgatcttttctctgctcatcagagactcccagcccagtgattcagccacctacctctgtgccgtggggcttcagggaggaggaaacaaactcacctttgggacaggcactcagctaaaagtggaactcaatatccagaaccctgaccctgccgtgtaccagctgagagact

SEQ ID NO: 117 - protein, 95 aa, Homo sapiens
  SITE 30: note = misc_feature - Xaa can be any naturally occurring amino acid
PPPPPPPPPPPPPPCFFFKAEEGIRDEGLXLEFRRVLFRSGRSTLYIAASQPGDSATYLCAPGDFNKFYFGSGTKLNVKPNIQNPDPAVYQLRDZ

SEQ ID NO: 118 - DNA, 283 bp, Homo sapiens
  source 1..283: mol_type = genomic DNA
cccccccccccccccccccccccccccccccccccccccccctgttttttttttaaagcagaagagggaatacgagatgaggggttgtgactggagttcagacgtgtgctcttccgatctggacgtagtactttatacattgcagcttctcagcctggtgactcagccacctacctctgtgctccaggtgacttcaacaaattttactttggatctgggaccaaactcaatgtaaaaccaaatatccagaaccctgaccctgccgtgtaccagctgagagact

SEQ ID NO: 119 - protein, 95 aa, Homo sapiens
PPPPPPPPPPPTTLFFYHSRRQKAMRGCDWSSDVCSSDLFSLHIMDSQPGDSATYFCAASMISGTYKYIFGTGTRLKVLANIQNPDPAVYQLRDZ

SEQ ID NO: 120 - DNA, 283 bp, Homo sapiens
  source 1..283: mol_type = genomic DNA
cccccccccccccccccccccccccccccccccaccaccctttttttttaccacagcaggaggcaaaaggccatgaggggttgtgactggagttcagacgtgtgctcttccgatctgttctcattgcatatcatggattcccagcctggagactcagccacctacttctgtgcagcaagcatgatctcaggaacctacaaatacatctttggaacaggcaccaggctgaaggttttagcaaatatccagaaccctgaccctgccgtgtaccagctgagagact

SEQ ID NO: 121 - protein, 94 aa, Homo sapiens
PPPPPPPPFFYPHTTANALRGGDWSSDVCSSDLLILESPSPNQTSLYFCASKDRENIGTEAFFGQGTRLTVVEDLNKVFPPEVAVFEPSEAEIZ

SEQ ID NO: 122 - DNA, 283 bp, Homo sapiens
  source 1..283: mol_type = genomic DNA
cccccccccccccccccccccccccttttttttacccccacacgacggcaaacgcgctgaggggtggggactggagttcagacgtgtgctcttccgatctcctgatcctggagtcgcccagccccaaccagacctctctgtacttctgtgccagcaaagacagggagaatattggcactgaagctttctttggacaaggcaccagactcacagttgtagaggacctgaacaaggtgttcccacccgaggtcgctgtgtttgagccatcagaagcagagatctc

SEQ ID NO: 123 - protein, 94 aa, Homo sapiens
  SITE 33: note = misc_feature - Xaa can be any naturally occurring amino acid
PPPPPPPFFYERRRRNTRGGVVTGVQTCALPIXLNVNALLLGDSALYLCASSLAKGAKTEAFFGQGTRLTVVEDLNKVFPPEVAVFEPSEAEIZ

SEQ ID NO: 124 - DNA, 283 bp, Homo sapiens
  source 1..283: mol_type = genomic DNA
ccccccccccccccccccccccttttttttatgaaagaagacgccggaatacgagaggaggggttgtgactggagttcagacgtgtgctcttccgatctagctgaatgtgaacgccttgttgctgggggactcggccctctatctctgtgccagcagcttggccaagggggcgaagactgaagctttctttggacaaggcaccagactcacagttgtagaggacctgaacaaggtgttcccacccgaggtcgctgtgtttgagccatcagaagcagagatctc

SEQ ID NO: 125 - protein, 94 aa, Homo sapiens
  SITE 22: note = misc_feature - Xaa can be any naturally occurring amino acid
PPPPPPPPPPFFLSRTPRQPPXGVVTGVQTCALPISLTIQRTQQEDSAVYLCASSQGTDVQYFGAGTRLSVLEDLKNVFPPEVAVFEPSEAEIZ

SEQ ID NO: 126 - DNA, 283 bp, Homo sapiens
  source 1..283: mol_type = genomic DNA
ccccccccccccccccccccccccccccccccttttttttatcacgcacaccccgccaaccgccctgaggggttgtgactggagttcagacgtgtgctcttccgatctctctgacgatccagcgcacacagcaggaggactcggccgtgtatctctgtgccagcagccaagggacagacgttcagtacttcggcgccgggacccggctctcagtgctggaggacctgaaaaacgtgttcccacccgaggtcgctgtgtttgagccatcagaagcagagatctc

SEQ ID NO: 127 - protein, 94 aa, Homo sapiens
PPPPPPPFFFATPTRHPRGGVVTGVQTCALPTYLEIQRTEQGDSAMYLCASTEARGANNEQFFGPGTRLTVLEDLKNVFPPEVAVFEPSEAEIZ

SEQ ID NO: 128 - DNA, 283 bp, Homo sapiens
  source 1..283: mol_type = genomic DNA
ccccccccccccccccccccccatttttttttgcaaccccaacacggcatccgagaggaggggttgtgactggagttcagacgtgtgctcttccgacctacttggagatccagcgcacagagcagggggactcggccatgtatctctgtgccagcaccgaagccaggggagctaacaatgagcagttcttcgggccagggacacggctcaccgtgctagaggacctgaaaaacgtgttcccacccgaggtcgctgtgtttgagccatcagaagcagagatctc

SEQ ID NO: 129 - protein, 94 aa, Homo sapiens
PPPPPPRFFFKPTTAYAMRGCDWSSDVCSSDLLKIRSTKLEDSAMYFCASSEQAGGHYNEQFFGPGTRLTVLEDLKNVFPPEVAVFEPSEAEIZ

SEQ ID NO: 130 - DNA, 283 bp, Homo sapiens
  source 1..283: mol_type = genomic DNA
cccccccccccccccccccccgtttttttttcaagccgacgacggcatacgcgatgaggggttgtgactggagttcagacgtgtgctcttccgatcttctgaagatccggtccacaaagctggaggactcagccatgtacttctgtgccagcagtgaacaagcgggagggcattacaatgagcagttcttcgggccagggacacggctcaccgtgctagaggacctgaaaaacgtgttcccacccgaggtcgctgtgtttgagccatcagaagcagagatctc

SEQ ID NO: 131 - protein, 95 aa, Homo sapiens
NLQWFRQDPGKGLTSLLLIQSSQREQTSGRLNASLDKSSGRSTLYIAASQPGDSATYLCAVSSNDYKLSFGAGTTVTVRANIQNPDPAVYQLRDZ

SEQ ID NO: 132 - DNA, 283 bp, Homo sapiens
  source 1..283: mol_type = genomic DNA
aacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtctcctctaacgactacaagctcagctttggagccggaaccacagtaactgtaagagcaaatatccagaaccctgaccctgccgtgtaccagctgagagact

SEQ ID NO: 133 - protein, 95 aa, Homo sapiens
DYFPWYQQFPGKGPALLIAIRPDVSEKKEGRFTISFNKSAKQFSLHIMDSQPGDSATYFCAAISYNDMRFGAGTRLTVKPNIQNPDPAVYQLRDZ

SEQ ID NO: 134 - DNA, 283 bp, Homo sapiens
  source 1..283: mol_type = genomic DNA
gactactttccatggtaccaacagttccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagcgatttcctacaatgacatgcgctttggagcagggaccagactgacagtaaaaccaaatatccagaaccctgaccctgccgtgtaccagctgagagact

SEQ ID NO: 135 - protein, 95 aa, Homo sapiens
GSQSFFWYRQYSGKSPELIMFIYSNGDKEDGRFTAQLNKASQYVSLLIRDSQPSDSATYLCAGNTDKLIFGTGTRLQVFPNIQNPDPAVYQLRDZ

SEQ ID NO: 136 - DNA, 283 bp, Homo sapiens
  source 1..283: mol_type = genomic DNA
ggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccggtaacaccgacaagctcatctttgggactgggaccagattacaagtctttccaaatatccagaaccctgaccctgccgtgtaccagctgagagact

SEQ ID NO: 137 - protein, 95 aa, Homo sapiens
SNAYNFFWYLHFPGCAPRLLVKGSKPSQQGRYNMTYERFSSSLLILQVREADAAVYYCAASTGGGNKLTFGTGTQLKVELNIQNPDPAVYQLRDZ

SEQ ID NO: 138 - DNA, 283 bp, Homo sapiens
  source 1..283: mol_type = genomic DNA
tccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgcctccacgggaggaggaaacaaactcacctttgggacaggcactcagctaaaagtggaactcaatatccagaaccctgaccctgccgtgtaccagctgagagact

SEQ ID NO: 139 - protein, 95 aa, Homo sapiens
PWYKQELGKGPQLIIDIRSNVGEKKDQRIAVTLNKTAKHFSLHITETQPEDSAVYFCAASIAGGYQKVTFGIGTKLQVIPNIQNPDPAVYQLRDZ

SEQ ID NO: 140 - DNA, 283 bp, Homo sapiens
  source 1..283: mol_type = genomic DNA
ccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtatagctgggggttaccagaaagttacctttggaattggaacaaagctccaagtcatcccaaatatccagaaccctgaccctgccgtgtaccagctgagagact

SEQ ID NO: 141 - protein, 95 aa, Homo sapiens
DYITWYQQFPSQGPRFIIQGYKTKVTNEVASLFIPADRKSSTLSLPRVSLSDTAVYYCLVVGGGFQKLVFGTGTRLLVSPNIQNPDPAVYQLRDZ

SEQ ID NO: 142 - DNA, 280 bp, Homo sapiens
  source 1..280: mol_type = genomic DNA
gattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtggtggggggaggctttcagaaacttgtatttggaactggcacccgacttctggtcagtccaaatatccagaaccctgaccctgccgtgtaccagctgagag

SEQ ID NO: 143 - protein, 94 aa, Homo sapiens
  SITE 87: note = misc_feature - Xaa can be any naturally occurring amino acid
GMGLRLIHYSVGAGITDQGEVPNGYNVSRSTTEDFPLRLLSAAPSQTSVYFCASRHRDSYEQYFGPGTRLTVTGPEKRVPTRGRCVXAIRSRDL

SEQ ID NO: 144 - DNA, 283 bp, Homo sapiens
  source 1..283: mol_type = genomic DNA
aggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagacaccgggacagctacgagcagtacttcgggccgggcaccaggctcacggtcacaggacctgaaaaacgtgttcccacccgaggtcgctgtgtttgagccatcagaagcagagatctc

SEQ ID NO: 145 - protein, 94 aa, Homo sapiens
PQFIFQYYEEEERQRGNFPDRFSGHQFPNYSSELNVNALLLGDSALYLCASSLAKGAKTEAFFGQGTRLTVVEDLNKVFPPEVAVFEPSEAEIZ

SEQ ID NO: 146 - DNA, 283 bp, Homo sapiens
  source 1..283: mol_type = genomic DNA
ggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttggccaagggggcgaagactgaagctttctttggacaaggcaccagactcacagttgtagaggacctgaacaaggtgttcccacccgaggtcgctgtgtttgagccatcagaagcagagatctc

SEQ ID NO: 147 - protein, 94 aa, Homo sapiens
MATSNEGSKATYEQGVEKDKFLINHASLTLSTLTVTSAHPEDSSFYICSAKSLRTGGRTEAFFGQGTRLTVVEDLNKVFPPEVAVFEPSEAEIZ

SEQ ID NO: 148 - DNA, 283 bp, Homo sapiens
  source 1..283: mol_type = genomic DNA
tgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaagagtcttaggacggggggcagaactgaagctttctttggacaaggcaccagactcacagttgtagaggacctgaacaaggtgttcccacccgaggtcgctgtgtttgagccatcagaagcagagatctc

SEQ ID NO: 149 - protein, 94 aa, Homo sapiens
GLGLRQIYYSMNVEVTDKGDVPEGYKVSRKEKRNFPLILESPSPNQTSLYFCASRTGQNIQYFGAGTRLSVLEDLKNVFPPEVAVFEPSEAEIZ

SEQ ID NO: 150 - DNA, 283 bp, Homo sapiens
  source 1..283: mol_type = genomic DNA
cagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagaacggggcaaaacattcagtacttcggcgccgggacccggctctcagtgctggaggacctgaaaaacgtgttcccacccgaggtcgctgtgtttgagccatcagaagcagagatctc

SEQ ID NO: 151 - protein, 94 aa, Homo sapiens
FLVSFYNNEISEKSEIFDDQFSVERPDGSNFTLKIRSTKLEDSAMYFCASSEQAGGHYNEQFFGPGTRLTVLEDLKNVFPPEVAVFEPSEAEIZ

SEQ ID NO: 152 - DNA, 283 bp, Homo sapiens
  source 1..283: mol_type = genomic DNA
agtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaacaagcgggagggcattacaatgagcagttcttcgggccagggacacggctcaccgtgctagaggacctgaaaaacgtgttcccacccgaggtcgctgtgtttgagccatcagaagcagagatctc

SEQ ID NO: 153 - protein, 95 aa, Homo sapiens
LFWYQQHAGEAPTFLSYNVLDGLEEKGRFSSFLSRSKGYSYLLLKELQMKDSASYLCAVRDGGSNYQLIWGAGTKLIIKPDIQNPDPAVYQLRDZ

SEQ ID NO: 154 - DNA, 283 bp, Homo sapiens
  source 1..283: mol_type = genomic DNA
ctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagatgggggtagcaactatcagttaatctggggcgctgggaccaagctaattataaagccagatatccagaaccctgaccctgccgtgtaccagctgagagact

SEQ ID NO: 155 - protein, 95 aa, Homo sapiens
GLFWYQQHAGEAPTFLSYNVLDGLEEKGRFSSFLSRSKGYSYLLLKELQMKDSASYLCAVRAPGGFKTIFGAGTRLFVKANIQNPDPAVYQLRDZ

SEQ ID NO: 156 - DNA, 283 bp, Homo sapiens
  source 1..283: mol_type = genomic DNA
gggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagggcccctggaggcttcaaaactatctttggagcaggaacaagactatttgttaaagcaaatatccagaaccctgaccctgccgtgtaccagctgagagact

SEQ ID NO: 157 - protein, 95 aa, Homo sapiens
WYKQPPSGELVFLIRRNSFDEQNEISGRYSWNFQKSTSSFNFTITASQVVDSAVYFCALSLYSSASKIIFGSGTRLSIRPNIQNPDPAVYQLRDZ

SEQ ID NO: 158 - DNA, 283 bp, Homo sapiens
  source 1..283: mol_type = genomic DNA
tggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtctttacagcagtgcttccaagataatctttggatcagggaccagactcagcatccggccaaatatccagaaccctgaccctgccgtgtaccagctgagagact

SEQ ID NO: 159 - protein, 95 aa, Homo sapiens
GLFWYQQHAGEAPTFLSYNVLDGLEEKGRFSSFLSRSKGYSYLLLKELQMKDSASYLCAVRGSSASKIIFGSGTRLSIRPNIQNPDPAVYQLRDZ

SEQ ID NO: 160 - DNA, 283 bp, Homo sapiens
  source 1..283: mol_type = genomic DNA
gggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtccgagggagcagtgcttccaagataatctttggatcagggaccagactcagcatccggccaaatatccagaaccctgaccctgccgtgtaccagctgagagact

SEQ ID NO: 161 - protein, 95 aa, Homo sapiens
GLFWYQQHAGEAPTFLSYNVLDGLEEKGRFSSFLSRSKGYSYLLLKELQMKDSASYLCAVRGQAGTALIFGKGTTLSVSSNIQNPDPAVYQLRDZ

SEQ ID NO: 162 - DNA, 283 bp, Homo sapiens
  source 1..283: mol_type = genomic DNA
gggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagggcaggcaggaactgctctgatctttgggaagggaaccaccttatcagtgagttccaatatccagaaccctgaccctgccgtgtaccagctgagagact

SEQ ID NO: 163 - protein, 94 aa, Homo sapiens
PGLGLRQIYYSMNVEVTDKGDVPEGYKVSRKEKRNFPLILESPSPNQTSLYFCASSLGGEQYFGPGTRLTVTEDLKNVFPPEVAVFEPSEAEIZ

SEQ ID NO: 164 - DNA, 283 bp, Homo sapiens
  source 1..283: mol_type = genomic DNA
acccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttgggcggcgagcagtacttcgggccgggcaccaggctcacggtcacagaggacctgaaaaacgtgttcccacccgaggtcgctgtgtttgagccatcagaagcagagatctc

SEQ ID NO: 165 - protein, 94 aa, Homo sapiens
GHGLRLIHYSYGVKDTDKGEVSDGYSVSRSKTEDFLLTLESATSSQTSVYFCAIGAGDETQYFGPGTRLLVLEDLKNVFPPEVAVFEPSEAEIZ

SEQ ID NO: 166 - DNA, 283 bp, Homo sapiens
  source 1..283: mol_type = genomic DNA
cggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcggggcgggagacgagacccagtacttcgggccaggcacgcggctcctggtgctcgaggacctgaaaaacgtgttcccacccgaggtcgctgtgtttgagccatcagaagcagagatctc

SEQ ID NO: 167 - protein, 94 aa, Homo sapiens
QFLISFYEKMQSDKGSIPDRFSAQQFSDYHSELNMSSLELGDSALYFCASSPGSSGFGNEQFFGPGTRLTVLEDLKNVFPPEVAVFEPSEAEIZ

SEQ ID NO: 168 - DNA, 283 bp, Homo sapiens
  source 1..283: mol_type = genomic DNA
cccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcccagggtctagcgggttcggtaatgagcagttcttcgggccagggacacggctcaccgtgctagaggacctgaaaaacgtgttcccacccgaggtcgctgtgtttgagccatcagaagcagagatctc

SEQ ID NO: 169 - protein, 94 aa, Homo sapiens
PGLGLRLIYYSFDVKDINKGEISDGYSVSRQAQAKFSLSLESAIPNQTALYFCATTLGHEQFFGPGTRLTVLEDLKNVFPPEVAVFEPSEAEIZ

SEQ ID NO: 170 - DNA, 283 bp, Homo sapiens
  source 1..283: mol_type = genomic DNA
acccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccacgctaggccatgagcagttcttcgggccagggacacggctcaccgtgctagaggacctgaaaaacgtgttcccacccgaggtcgctgtgtttgagccatcagaagcagagatctc

SEQ ID NO: 171 - protein, 94 aa, Homo sapiens
LMATSNEGSKATYEQGVEKDKFLINHASLTLSTLTVTSAHPEDSSFYICSARHGQGGSQPQHFGDGTRLSILEDLNKVFPPEVAVFEPSEAEIZ

SEQ ID NO: 172 - DNA, 283 bp, Homo sapiens
  source 1..283: mol_type = genomic DNA
tgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagacatgggcagggcgggagtcagccccagcattttggtgatgggactcgactctccatcctagaggacctgaacaaggtgttcccacccgaggtcgctgtgtttgagccatcagaagcagagatctc

SEQ ID NO: 173 - protein, 95 aa, Homo sapiens
WYKQELGKGPQLIIDIRSNVGEKKDQRIAVTLNKTAKHFSLHITETQPEDSAVYFCAASMRRGGSYIPTFGRGTSLIVHPYIQNPDPAVYQLRDZ

SEQ ID NO: 174 - DNA, 283 bp, Homo sapiens
  source 1..283: mol_type = genomic DNA
tggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtatgagacgaggaggaagctacatacctacatttggaagaggaaccagccttattgttcatccgtatatccagaaccctgaccctgccgtgtaccagctgagagact

SEQ ID NO: 175 - protein, 95 aa, Homo sapiens
PWYKQELGKRPQLIIDIRSNVGEKKDQRIAVTLNKTAKHFSLHITETQPEDSAVYFCAATNGGSQGNLIFGKGTKLSVKPNIQNPDPAVYQLRDZ

SEQ ID NO: 176 - DNA, 283 bp, Homo sapiens
  source 1..283: mol_type = genomic DNA
ccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaaccaatggaggaagccaaggaaatctcatctttggaaaaggcactaaactctctgttaaaccaaatatccagaaccctgaccctgccgtgtaccagctgagagact

SEQ ID NO: 177 - protein, 95 aa, Homo sapiens
NLQWFHQNPWGQLINLFYIPSGTKQNGRLSATTVATERYSLLYISSSQTTDSGVYFCAVGRYSSASKIIFGSGTRLSIRPNIQNPDPAVYQLRDZ

SEQ ID NO: 178 - DNA, 283 bp, Homo sapiens
  source 1..283: mol_type = genomic DNA
aatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtgggccggtacagcagtgcttccaagataatctttggatcagggaccagactcagcatccggccaaatatccagaaccctgaccctgccgtgtaccagctgagagact

SEQ ID NO: 179 - protein, 95 aa, Homo sapiens
QSFFWYRQYSGKSPELIMFIYSNGDKEDGRFTAQLNKASQYVSLLIRDSQPSDSATYLCAVNQGAQKLVFGQGTRLTINPNIQNPDPAVYQLRDZ

SEQ ID NO: 180 - DNA, 283 bp, Homo sapiens
  source 1..283: mol_type = genomic DNA
cagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaaccagggagcccagaagctggtatttggccaaggaaccaggctgactatcaacccaaatatccagaaccctgaccctgccgtgtaccagctgagagact

SEQ ID NO: 181 - protein, 95 aa, Homo sapiens
KYFMWYRQYSRKGPELLMYTYSSGNKEDGRFTAQVDKSSKYISLFIRDSQPSDSATYLCAVFTGGFKTIFGAGTRLFVKANIQNPDPAVYQLRDZ

SEQ ID NO: 182 - DNA, 283 bp, Homo sapiens
  source 1..283: mol_type = genomic DNA
aaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcagtgtttactggaggcttcaaaactatctttggagcaggaacaagactatttgttaaagcaaatatccagaaccctgaccctgccgtgtaccagctgagagact

SEQ ID NO: 183 - protein, 95 aa, Homo sapiens
WYRQIHSQGPQYIIHGLKNNETNEMASLIITEDRKSSTLILPHATLRDTAVYYCIVRVFDSGGSNYKLTFGKGTLLTVNPNIQNPDPAVYQLRDZ

SEQ ID NO: 184 - DNA, 283 bp, Homo sapiens
  source 1..283: mol_type = genomic DNA
tggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagagtcttcgatagtggaggtagcaactataaactgacatttggaaaaggaactctcttaaccgtgaatccaaatatccagaaccctgaccctgccgtgtaccagctgagagact

SEQ ID NO: 185 - protein, 95 aa, Homo sapiens
WYVQHPNKGLQLLLKYTSAATLVKGINGFEAEFKKSETSFHLTKPSAHMSDAAEYFCVVSDNKGAQKLVFGQGTRLTINPNIQNPDPAVYQLRDZ

SEQ ID NO: 186 - DNA, 283 bp, Homo sapiens
  source 1..283: mol_type = genomic DNA
tggtatgtgcaacaccccaacaaaggactccagcttctcctgaagtacacatcagcggccaccctggttaaaggtatcaacggttttgaggctgaatttaagaagagtgaaacctccttccacctgacgaaaccctcagcccatatgagcgacgcggctgagtacttctgtgttgtgagtgataataagggagcccagaagctggtatttggccaaggaaccaggctgactatcaacccaaatatccagaaccctgaccctgccgtgtaccagctgagagact

SEQ ID NO: 187 - protein, 95 aa, Homo sapiens
LQWFRQDPGKGLTSLLLIQSSQREQTSGRLNASLDKSSGRSTLYIAASQPGDSATYLCAVKGVRDDKIIFGKGTRLHILPNIQNPDPAVYQLRDZ

SEQ ID NO: 188 - DNA, 283 bp, Homo sapiens
  source 1..283: mol_type = genomic DNA
ctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaagggtgtaagagatgacaagatcatctttggaaaagggacacgacttcatattctccccaatatccagaaccctgaccctgccgtgtaccagctgagagact

SEQ ID NO: 189 - protein, 95 aa, Homo sapiens
EYVYWYRQIHSQGPQYIIHGLKNNETNEMASLIITEDRKSSTLILPHATLRDTAVYYCIVRANTGNQFYFGTGTSLTVIPNIQNPDPAVYQLRDZ

SEQ ID NO: 190 - DNA, 283 bp, Homo sapiens
  source 1..283: mol_type = genomic DNA
gagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagagccaacaccggtaaccagttctattttgggacagggacaagtttgacggtcattccaaatatccagaaccctgaccctgccgtgtaccagctgagagact

SEQ ID NO: 191 - protein, 95 aa, Homo sapiens
FPWYKQELGKGPQLIIDIRSNVGEKKDQRIAVTLNKTAKHFSLHITETQPEDSAVYFCAASSFGNEKLTFGTGTRLTIIPNIQNPDPAVYQLRDZ

SEQ ID NO: 192 - DNA, 283 bp, Homo sapiens
  source 1..283: mol_type = genomic DNA
ttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagttcgtttggaaatgagaaattaacctttgggactggaacaagactcaccatcatacccaatatccagaaccctgaccctgccgtgtaccagctgagagact

SEQ ID NO: 193 - protein, 94 aa, Homo sapiens
PEFLTYFQNEAQLEKSRLLSDRFSAERPKGSFSTLEIQRTEQGDSAMYLCASSFLVSEETQYFGPGTRLLVLEDLKNVFPPEVAVFEPSEAEIZ

SEQ ID NO: 194 - DNA, 283 bp, Homo sapiens
  source 1..283: mol_type = genomic DNA
gcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttcctcgtatcggaagagacccagtacttcgggccaggcacgcggctcctggtgctcgaggacctgaaaaacgtgttcccacccgaggtcgctgtgtttgagccatcagaagcagagatctc

SEQ ID NO: 195 - protein, 94 aa, Homo sapiens
GLQFLIQYYNGEERAKGNILERFSAQQFPDLHSELNLSSLELGDSALYFCASSPTGSGREQYFGPGTRLTVTEDLKNVFPPEVAVFEPSEAEIZ

SEQ ID NO: 196 - DNA, 283 bp, Homo sapiens
  source 1..283: mol_type = genomic DNA
agggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcccgacagggtcgggtcgcgagcagtacttcgggccgggcaccaggctcacggtcacagaggacctgaaaaacgtgttcccacccgaggtcgctgtgtttgagccatcagaagcagagatctc

SEQ ID NO: 197 - protein, 94 aa, Homo sapiens
EFLIYFQGTGAADDSGLPNDRFFAVRPEGSVSTLKIQRTERGDSAVYLCASSSRLAGYNEQFFGPGTRLTVLEDLKNVFPPEVAVFEPSEAEIZ

SEQ ID NO: 198 - DNA, 283 bp, Homo sapiens
  source 1..283: mol_type = genomic DNA
cagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagctcccgactagcgggatacaatgagcagttcttcgggccagggacacggctcaccgtgctagaggacctgaaaaacgtgttcccacccgaggtcgctgtgtttgagccatcagaagcagagatctc

SEQ ID NO: 199 - protein, 94 aa, Homo sapiens
GQGPKLLIQFQNNGVVDDSQLPKDRFSAERLKGVDSTLKIQPAKLEDSAVYLCASPEGGTQYFGPGTRLTVLEDLKNVFPPEVAVFEPSEAEIZ

SEQ ID NO: 200 - DNA, 283 bp, Homo sapiens
  source 1..283: mol_type = genomic DNA
tgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcccggagggtggtacgcagtattttggcccaggcacccggctgacagtgctcgaggacctgaaaaacgtgttcccacccgaggtcgctgtgtttgagccatcagaagcagagatctc

SEQ ID NO: 201 - protein, 94 aa, Homo sapiens
LIHYSVGAGITDQGEVPNGYNVSRSTTEDFPLRLLSAAPSQTSVYFCASSRKGRTFIPDTQYFGPGTRLTVLEDLKNVFPPEVAVFEPSEAEIZ

SEQ ID NO: 202 - DNA, 283 bp, Homo sapiens
  source 1..283: mol_type = genomic DNA
ggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagccggaagggacggacctttattccagatacgcagtattttggcccaggcacccggctgacagtgctcgaggacctgaaaaacgtgttcccacccgaggtcgctgtgtttgagccatcagaagcagagatctc

SEQ ID NO: 203 - protein, 94 aa, Homo sapiens
GLRLIYFSYDVKMKEKGDIPEGYSVSREKKERFSLILESASTNQTSMYLCASSPTGRGFVIYFGAGTRLSVLEDLKNVFPPEVAVFEPSEAEIZ

SEQ ID NO: 204 - DNA, 283 bp, Homo sapiens
  source 1..283: mol_type = genomic DNA
tggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtccgacagggcgaggctttgttatatacttcggcgccgggacccggctctcagtgctggaggacctgaaaaacgtgttcccacccgaggtcgctgtgtttgagccatcagaagcagagatctc

SEQ ID NO: 205 - protein, 94 aa, Homo sapiens
GLRQIYYSMNVEVTDKGDVPEGYKVSRKEKRNFPLILESPSPNQTSLYFCASSLGGHPVEQFFGPGTRLTVLEDLKNVFPPEVAVFEPSEAEIZ

SEQ ID NO: 206 - DNA, 283 bp, Homo sapiens
  source 1..283: mol_type = genomic DNA
tgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagcttagggggacacccggttgagcagttcttcgggccagggacacggctcaccgtgctagaggacctgaaaaacgtgttcccacccgaggtcgctgtgtttgagccatcagaagcagagatctc

SEQ ID NO: 207 - protein, 94 aa, Homo sapiens
LGLRQIYYSMNVEVTDKGDVPEGYKVSRKEKRNFPLILESPSPNQTSLYFCASSFSLGTEQFFGPGTRLTVLEDLKNVFPPEVAVFEPSEAEIZ

SEQ ID NO: 208 - DNA, 283 bp, Homo sapiens
  source 1..283: mol_type = genomic DNA
ggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttttccctcggaaccgagcagttcttcgggccagggacacggctcaccgtgctagaggacctgaaaaacgtgttcccacccgaggtcgctgtgtttgagccatcagaagcagagatctc

SEQ ID NO: 209 - protein, 94 aa, Homo sapiens
MATSNEGSKATYEQGVEKDKFLINHASLTLSTLTVTSAHPEDSSFYICSARVLAGSSYNEQFFGPGTRLTVLEDLKNVFPPEVAVFEPSEAEIZ

SEQ ID NO: 210 - DNA, 283 bp, Homo sapiens
  source 1..283: mol_type = genomic DNA
tgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagggtattagcggggagctcctacaatgagcagttcttcgggccagggacacggctcaccgtgctagaggacctgaaaaacgtgttcccacccgaggtcgctgtgtttgagccatcagaagcagagatctc

SEQ ID NO: 211 - protein, 94 aa, Homo sapiens
LMATSNEGSKATYEQGVEKDKFLINHASLTLSTLTVTSAHPEDSSFYICSARPTDILTYEQYFGPGTRLTVTEDLKNVFPPEVAVFEPSEAEIZ

SEQ ID NO: 212 - DNA, 283 bp, Homo sapiens
  source 1..283: mol_type = genomic DNA
tgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagacccacggacatactaacctacgagcagtacttcgggccgggcaccaggctcacggtcacagaggacctgaaaaacgtgttcccacccgaggtcgctgtgtttgagccatcagaagcagagatctc

SEQ ID NO: 213 - protein, 94 aa, Homo sapiens
VRKESGIPSGKFEVDRIPETSTSTLTIHNVEKQDIATYYCALWEAYIRLYKKLFGSGTTLVVTDKQLDADVSPKPTIFLPSIAETKLQKAGTYL

SEQ ID NO: 214 - DNA, 283 bp, Homo sapiens
  source 1..283: mol_type = genomic DNA
tgtcagaaaggaatccggcattccgtcaggcaaatttgaggtggataggatacctgaaacgtctacatccactctcaccattcacaatgtagagaaacaggacatagctacctactactgtgccttgtgggaggcctacatccgcctttataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 215 - protein, 94 aa, Homo sapiens
YDGTVRKESGIPSGKFEVDRIPETSTSTLTIHNVEKQDIATYYCALWGGYKKLFGSGTTLVVTDKQLDADVSPKPTIFLPSIAETKLQKAGTYL

SEQ ID NO: 216 - DNA, 283 bp, Homo sapiens
  source 1..283: mol_type = genomic DNA
atatgacggcactgtcagaaaggaatccggcattccgtcaggcaaatttgaggtggataggatacctgaaacgtctacatccactctcaccattcacaatgtagagaaacaggacatagctacctactactgtgccttgtggggggggtataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 217 - protein, 94 aa, Homo sapiens
TARDVLESGLSPGKYYTHTPRRWSWILRLQNLIENDSGVYYCATWDRHYYKKLFGSGTTLVVTDKQLDADVSPKPTIFLPSIAETKLQKAGTYL

SEQ ID NO: 218 - DNA, 283 bp, Homo sapiens
  source 1..283: mol_type = genomic DNA
caccgcaagggatgtgttggaatcaggactcagtccaggaaagtattatactcatacacccaggaggtggagctggatattgagactgcaaaatctaattgaaaatgattctggggtctattactgtgccacctgggacaggcattattataagaaactctttggcagtggaacaacacttgttgtcacagataaacaacttgatgcagatgtttcccccaagcccactatttttcttccttcgattgctgaaacaaaactccagaaggctggaacatacctt

SEQ ID NO: 219 - protein, 94 aa, Homo sapiens
GTVRKESGIPSGKFEVDRIPETSTSTLTIHNVEKQDIATYYCALWEENYYKKLFGSGTTLVVTDKQLDADVSPKPTIFLPSIAETKLQKAGTYL

SEQ ID NO: 220 - DNA, 283 bp, Homo sapiens
  source 1..283: mol_type = genomic DNA
cggcactgtcagaaaggaatccggcattccgtcaggcaaatttgaggtggataggatacctgaaacgtctacatccactctcaccattcacaatgtagagaaacaggacatagctacctactactgtgccttgtgggaggagaattattataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 221 - protein, 94 aa, Homo sapiens
YDSYNSKVVLESGVSPGKYYTYASTRNNLRLILRNLIENDFGVYYCATWDKKLFGSGTTLVVTDKQLDADVSPKPTIFLPSIAETKLQKAGTYL

SEQ ID NO: 222 - DNA, 283 bp, Homo sapiens
  source 1..283: mol_type = genomic DNA
atatgactcctacaactccaaggttgtgttggaatcaggagtcagtccagggaagtattatacttacgcaagcacaaggaacaacttgagattgatactgcgaaatctaattgaaaatgactttggggtctattactgtgccacctgggataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 223 - protein, 94 aa, Homo sapiens
ESGLSPGKYYTHTPRRWSWILRLQNLIENDSGVYYCATWDRGEHSSDWIKTFAKGTRLIVTSPDKQLDADVSPKPTIFLPSIAETKLQKAGTYL

SEQ ID NO: 224 - DNA, 283 bp, Homo sapiens
  source 1..283: mol_type = genomic DNA
tgaatcaggactcagtccaggaaagtattatactcatacacccaggaggtggagctggatattgagactgcaaaatctaattgaaaatgattctggggtctattactgtgccacctgggacaggggcgagcatagtagtgattggatcaagacgtttgcaaaagggactaggctcatagtaacttcgcctgataaacaacttgatgcagatgtttcccccaagcccactatttttcttccttcgattgctgaaacaaaactccagaaggctggaacatacctt

SEQ ID NO: 225 - protein, 94 aa, Homo sapiens
SYDGTVRKESGIPSGKFEVDRIPETSTSTLTIHNVEKQDIATYYCALCDYKKLFGSGTTLVVTDKQLDADVSPKPTIFLPSIAETKLQKAGTYL

SEQ ID NO: 226 - DNA, 283 bp, Homo sapiens
  source 1..283: mol_type = genomic DNA
ttcatatgacggcactgtcagaaaggaatccggcattccgtcaggcaaatttgaggtggataggatacctgaaacgtctacatccactctcaccattcacaatgtagagaaacaggacatagctacctactactgtgccttgtgtgattataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 227 - protein, 94 aa, Homo sapiens
SSVVLESGISPGKYDTYGSTRKNLRMILRNLIENDSGVYYCATWDGPVYYKKLFGSGTTLVVTDKQLDADVSPKPTIFLPSIAETKLQKAETYL

SEQ ID NO: 228 - DNA, 283 bp, Homo sapiens
  source 1..283: mol_type = genomic DNA
atccagcgttgtgttggaatcaggaatcagcccagggaagtatgatacttacggaagcacaaggaagaacttgagaatgatactgcgaaatcttattgaaaatgactctggagtctattactgtgccacctgggatggccccgtttattataagaaactctttggcagtggaacaacacttgttgtcacagataaacaacttgatgcagatgtttcccccaagcccactatttttcttccttcgattgctgaaacaaaactccagaaggctgaaacatacctt

SEQ ID NO: 229 - protein, 94 aa, Homo sapiens
VVLESGITREKYHTYASTGKSLKFILENLIERDSGVYYCATWEGASDWIKTFAKGTRLIVTSPDKQLDADVSPKPTIFLPSIAETKLQKAGTYL

SEQ ID NO: 230 - DNA, 283 bp, Homo sapiens
  source 1..283: mol_type = genomic DNA
ggttgtgttggaatcaggaatcactcgagaaaagtatcatacttatgcaagcacagggaagagccttaaatttatactggaaaatctaattgaacgtgactctggggtctattactgtgccacctgggagggggctagtgattggatcaagacgtttgcaaaagggactaggctcatagtaacttcgcctgataaacaacttgatgcagatgtttcccccaagcccactatttttcttccttcgattgctgaaacaaaactccagaaggctggaacatacctt

SEQ ID NO: 231 - protein, 94 aa, Homo sapiens
YSYNSRGVLESGIRREKYHTYASTGKSLKFILENLIERDSGVYYCATSLYKKLFGSGTTLVVTDKQLDADVSPKPTIFLPSIAETKLQKAGTYL

SEQ ID NO: 232 - DNA, 283 bp, Homo sapiens
  source 1..283: mol_type = genomic DNA
ttactcctacaactccaggggtgtgttggaatcaggaatcaggcgagaaaagtatcatacttatgcaagcacagggaagagccttaaatttatactggaaaatctaattgaacgtgactctggggtctattactgtgccacctcgctttataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 233 - protein, 94 aa, Homo sapiens
YKQPPSRQMILVIRQEAYKQQNATENRFSVNFQKAAKSFSLKISDSQLGDAAMYFCAYRTALGLGDTLTDKLIFGKGTRVTVEPRSQPHTKPSZ

SEQ ID NO: 234 - DNA, 283 bp, Homo sapiens
  source 1..283: mol_type = genomic DNA
ggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggacggccttgggcttgggggataccctcaccgataaactcatctttggaaaaggaacccgtgtgactgtggaaccaagaagtcagcctcataccaaaccatccgt

SEQ ID NO: 235 - protein, 94 aa, Homo sapiens
QLPSKEMIFLIRQGSDEQNAKSGRYSVNFKKAAKSVALTISALQLEDSAKYFCALGEFVSWFLALGDILFKLIFGKGTRVTVEPRSQPHTKPSZ

SEQ ID NO: 236 - DNA, 283 bp, Homo sapiens
  source 1..283: mol_type = genomic DNA
agcaacttcccagcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggaattcgtgtcttggttcttggcgctgggggatattctttttaaactcatctttggaaaaggaacccgtgtgactgtggaaccaagaagtcagcctcataccaaaccatccgt

SEQ ID NO: 237 - protein, 94 aa, Homo sapiens
TVLFLYRLRPDYSFQFVFYGDNSRSEGADFTQGRFSVKHILTQKAFHLVISPVRTEDSATYYCAYTEYTDKLIFGKGTRVTVEPRSQPHTKPSZ

SEQ ID NO: 238 - DNA, 283 bp, Homo sapiens
  source 1..283: mol_type = genomic DNA
atacagttttattcttgtaccggttaaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctataccgaatacaccgataaactcatctttggaaaaggaacccgtgtgactgtggaaccaagaagtcagcctcataccaaaccatccgt

SEQ ID NO: 239 - protein, 94 aa, Homo sapiens
KQLPIKEMIFLIRQGSDEQNAKSGRYSVNFKKAAKSVALTISALQLEDSAKYFCALGVTVPAFLRLGDTDKLIFGKGTRVTVEPRSQPHTKPSZ

SEQ ID NO: 240 - DNA, 283 bp, Homo sapiens
  source 1..283: mol_type = genomic DNA
acaagcaacttcccatcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggttaccgtcccagccttcctacggttgggggacaccgataaactcatctttggaaaaggaacccgtgtgactgtggaaccaagaagtcagcctcataccaaaccatccgt

SEQ ID NO: 241 - protein, 94 aa, Homo sapiens
YKQPPSRQMFLVIRQEAYKQQNATENRFSVNFQKAAKSFSLKISDSQLGDAAMYFCAYRTALGLGDTLTDKLIFGKGTRVTVEPRSQPHTKPSZ

SEQ ID NO: 242 - DNA, 283 bp, Homo sapiens
  source 1..283: mol_type = genomic DNA
ggtacaagcagcctcccagcaggcagatgtttctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggacggccttgggcttgggggacaccctcaccgataaactcatctttggaaaaggaacccgtgtgactgtggaaccaagaagtcagccccataccaaaccatccgt

SEQ ID NO: 243 - protein, 94 aa, Homo sapiens
QLPSNEMIFPIRQGSDEQNAKSGRYSVNFKKAAKSVALTISALQLEDSAKYFCALGESVSWFLALGDILFKLIFGKGTRVTVEPRSQPHTKPSZ

SEQ ID NO: 244 - DNA, 283 bp, Homo sapiens
  source 1..283: mol_type = genomic DNA
agcaacttcccagcaacgagatgattttccctattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggaatccgtgtcttggttcttggcgctgggggatattctttttaaactcatctttggaaaaggaacccgtgtgactgtggaaccaagaagtcagcctcataccaaaccatccgt

SEQ ID NO: 245 - protein, 94 aa, Homo sapiens
QLPCKEMSFLIRQGSDEQNAKSGRYSVNFKKAAQSVALTISALQLEDSAKYFCALGEFGSWFLALGDILFKLIFGKGTRVTVEPRSQPHTKPSZ

SEQ ID NO: 246 - DNA, 283 bp, Homo sapiens
  source 1..283: mol_type = genomic DNA
agcaacttccctgcaaagagatgagtttccttattcgccagggttctgatgaacagaatgcaaaaagtgggcgctattctgtcaacttcaagaaagcagcgcaatccgtcgccttaaccatttcagccttacagctagaagattcagcaaagtacttttgtgctcttggggaattcgggtcttggttcttggcgctgggggatattctttttaaactcatctttggaaaaggaacccgtgtgactgtggaaccaagaagtcagcctcataccaaaccatccgt

SEQ ID NO: 247 - protein, 94 aa, Homo sapiens
KLLCKEINLLLRHGSGEQNAKSGRYSVNFKKAAKSVALTISALQLEDSAKYFCALGEFVAWFLALGDILFKLIFGKGTRVTVEPRSQPHTKPSZ

SEQ ID NO: 248 - DNA, 283 bp, Homo sapiens
  source 1..283: mol_type = genomic DNA
tgaaacttctctgcaaagagattaatttgcttcttcgccacggttctggtgaacagaatgcaaaaagtggtcgctattctgtcaacttcaagaaagcagcgaaatccgtcgccttaaccatttcagccttacagctagaagattcagcaaagtacttttgtgctcttggggaattcgtggcttggttcttggcgctgggggatattctttttaaactcatctttggaaaaggaacccgtgtgactgtggaaccaagaagtcagcctcataccaaaccatccgt

SEQ ID NO: 249 - protein, 94 aa, Homo sapiens
YKQPTSRQMILVIRQEAYKQQNATENRFSVNFQKAAKSFSLKISDSQLGDAAMYFCAYRTALGLWDTLTDKLIFGKGTRVTVEPRSQPHTKPSZ

SEQ ID NO: 250 - DNA, 283 bp, Homo sapiens
  source 1..283: mol_type = genomic DNA
ggtacaagcagcctaccagcaggcagatgattctcgttatacgccaagaagcttataagcaacagaatgcaacagagaatcgtttctctgtgaacttccagaaagcagccaaatccttcagtctcaagatctcagactcacagctgggggatgccgcgatgtatttctgtgcttataggacggccttgggcttgtgggataccctcaccgataaactcatctttggaaaaggaacccgtgtgactgtggaaccaagaagtcagcctcataccaaaccatccgt

SEQ ID NO: 251 - protein, 94 aa, Homo sapiens
QLPSKEMFFHIRKCSDEENANSGRYSVNFKKAAKSVALTISALQLEDSAKYFCALGEFLSWFLALGDILFKLIFGKGTRVTVEPRSQPHTKPSZ

SEQ ID NO: 252 - DNA, 283 bp, Homo sapiens
  source 1..283: mol_type = genomic DNA
agcaacttcccagcaaagagatgtttttccatattcgcaagtgttctgatgaagagaatgcaaatagtggtcgctattctgtcaacttcaagaaagcagcgaaatccgtcgccttaaccatttcagccttacagctagaagattcagcaaagtacttttgtgctcttggggaattcttgtcttggttcttggcgctgggggatattctttttaaactcatctttggaaaaggaacccgtgtgactgtggaaccaagaagtcagcctcataccaaaccatccgt

SEQ ID NO: 253 - protein, 95 aa, Homo sapiens
  SITE 23: note = misc_feature - Xaa can be any naturally occurring amino acid
PPPPPPPPPPPPPPPPPSFFFNXKTATEIGWGDWSSDVCSSIFSLLITASRAADTASYFCATGNDYKLSFGAGTTVTVRANIQNPDPAVYQLRDZ

SEQ ID NO: 254 - DNA, 283 bp, Homo sapiens
  source 1..283: mol_type = genomic DNA
cccccccccccccccccccccccccccccccccccccccccccccccccccagtttttttttcaactagaagacggcaacggagataggttggggtgactggagttcagacgtgtgctcttcgatcttttccttgttgatcacggcttcccgggcagcagacactgcttcttacttctgtgctacgggtaacgactacaagctcagctttggagccggaaccacagtaactgtaagagcaaatatccagaaccctgaccctgccgtgtaccagctgagagact

SEQ ID NO: 255 - protein, 95 aa, Homo sapiens
  SITE 30: note = misc_feature - Xaa can be any naturally occurring amino acid
PPPPPPPPPPPPPPPFFFQAEDGIRDRLGXLEFRRVLFRSSLKISDSQLGDAAMYFCAYDEPGANSKLTFGKGITLSVRPDIQNPDPAVYQLRDZ

SEQ ID NO: 256 - DNA, 283 bp, Homo sapiens
  source 1..283: mol_type = genomic DNA
ccccccccccccccccccccccccccccccccccccccccccccttttttttttcaagcagaagacggcatacgagataggttggggtgactggagttcagacgtgtgctcttccgatctagtctcaagatctcagactcacagctgggggatgccgcgatgtatttctgtgcttatgacgagcctggagccaatagtaagctgacatttggaaaaggaataactctgagtgttagaccagatatccagaaccctgaccctgccgtgtaccagctgagagact

SEQ ID NO: 257 - protein, 95 aa, Homo sapiens
  SITE 26: note = misc_feature - Xaa can be any naturally occurring amino acid
PPPPPPPPPPPPPPFFFSSRRRHTRXVGVTGVQTCALPISILNITATQTGDSAVYLCAVERGGSQGNLIFGKGTKLSVKPNIQNPDPAVYQLRDZ

SEQ ID NO: 258 - DNA, 283 bp, Homo sapiens
  source 1..283: mol_type = genomic DNA
ccccccccccccccccccccccccccccccccccccccccctttttttttttcaagcagaagacggcatacgagataggttggggtgactggagttcagacgtgtgctcttccgatctccatcctgaacatcacagccacccagaccggagactcggccgtctacctctgtgctgtggagcgcggaggaagccaaggaaatctcatctttggaaaaggcactaaactctctgttaaaccaaatatccagaaccctgaccctgccgtgtaccagctgagagact

SEQ ID NO: 259 - protein, 95 aa, Homo sapiens
  SITE 32: note = misc_feature - Xaa can be any naturally occurring amino acid
PPPPPPPPPPPPPPPPPFFFQAEDGIRDRLGXLEFRRVLFRSGLLLKELQMKDSASYLCAVRDGGYKLSFGAGTTVTVRANIQNPDPAVYQLRDZ

SEQ ID NO: 260 - DNA, 283 bp, Homo sapiens
  source 1..283: mol_type = genomic DNA
ccccccccccccccccccccccccccccccccccccccccccccccccccctttttttttcaagcagaagacggcatacgagataggttggggtgactggagttcagacgtgtgctcttccgatctggcctccttttgaaggagctccagatgaaagactctgcctcttacctctgtgctgtgagagatggaggctacaagctcagctttggagccggaaccacagtaactgtaagagcaaatatccagaaccctgaccctgccgtgtaccagctgagagact

SEQ ID NO: 261 - protein, 95 aa, Homo sapiens
  SITE 26: note = misc_feature - Xaa can be any naturally occurring amino acid
  SITE 40: note = misc_feature - Xaa can be any naturally occurring amino acid
PPPPPPPPPPPPPPFFFSSRRRHTRXVGVTGVQTCALPIXSLLITASRAADTASYFCATGDAGNNRKLIWGLGTSLAVNPNIQNPDPAVYQLRDZ

SEQ ID NO: 262 - DNA, 283 bp, Homo sapiens
  source 1..283: mol_type = genomic DNA
ccccccccccccccccccccccccccccccccccccccccccttttttttttcaagcagaagacggcatacgagataggttggggtgactggagttcagacgtgtgctcttccgatctgatccttgttgatcacggcttcccgggcagcagacactgcttcttacttctgtgctacgggggatgctggcaacaaccgtaagctgatttggggattgggaacaagcctggcagtaaatccgaatatccagaaccctgaccctgccgtgtaccagctgagagact

SEQ ID NO: 263 - protein, 94 aa, Homo sapiens
  SITE 23: note = misc_feature - Xaa can be any naturally occurring amino acid
PPPPPPPPLFFQAEDGIRDRLGXLEFRRVLFRSLILESASTNQTSMYLCASSPVDPSRSGYTFGSGTRLTVVEDLNKVFPPEVAVFEPSEAEIZ

SEQ ID NO: 264 - DNA, 283 bp, Homo sapiens
  source 1..283: mol_type = genomic DNA
ccccccccccccccccccccccccctcttttttttcaagcagaagacggcatacgagataggttggggtgactggagttcagacgtgtgctcttccgatctttgattctggagtccgccagcaccaaccagacatctatgtacctctgtgccagcagccctgtggatccctcaagaagcggctacaccttcggttcggggaccaggttaaccgttgtagaggacctgaacaaggtgttcccacccgaggtcgctgtgtttgagccatcagaagcagagatctc

SEQ ID NO: 265 - protein, 94 aa, Homo sapiens
  SITE 21: note = misc_feature - Xaa can be any naturally occurring amino acid
PPPPPPLFFHAADAIRDRWGXLEFRRVLFRSVILESASTNQTSMYLCASSLSTSGSSYNEQFFGPGTRLTVLEDLKNVFPPEVAVFEPSEAEIZ

SEQ ID NO: 266 - DNA, 283 bp, Homo sapiens
  source 1..283: mol_type = genomic DNA
ccccccccccccccccccctctcttttttcacgccgcagacgccatacgcgataggtgggggtgactggagttcagacgtgtgctcttccgatctgtgattctggagtccgccagcaccaaccagacatctatgtacctctgtgccagcagcctctcgactagcgggtcgtcctacaatgagcagttcttcgggccagggacacggctcaccgtgctagaggacctgaaaaacgtgttcccacccgaggtcgctgtgtttgagccatcagaagcagagatctc

SEQ ID NO: 267 - protein, 94 aa, Homo sapiens
  SITE 23: note = misc_feature - Xaa can be any naturally occurring amino acid
PPPPPPPDFFFKAHDGIRDRLGXLEFRRVLFRSLILESASTNQTSMYLCASRPTSGIEVEQFFGPGTRLTVLEDLKNVFPPEVAVFEPSEAEIZ

SEQ ID NO: 268 - DNA, 283 bp, Homo sapiens
  source 1..283: mol_type = genomic DNA
cccccccccccccccccccccccgattttttttttaaagcacacgacggcatacgcgataggttggggtgactggagttcagacgtgtgctcttccgatctctgattctggagtccgccagcaccaaccagacatctatgtacctctgtgccagcagaccgactagcgggattgaagttgagcagttcttcgggccagggacacggctcaccgtgctagaggacctgaaaaacgtgttcccacccgaggtcgctgtgtttgagccatcagaagcagagatctc

SEQ ID NO: 269 - protein, 94 aa, Homo sapiens
  SITE 21: note = misc_feature - Xaa can be any naturally occurring amino acid
PPPPPHFFFQADDGIRDRLGXLEFRRVLFRSLILESASTNQTSMYLCASSRPGLAGGKETQYFGPGTRLLVLEDLKNVFPPEVAVFEPSEAEIZ

SEQ ID NO: 270 - DNA, 283 bp, Homo sapiens
  source 1..283: mol_type = genomic DNA
ccccccccccccccccacattttttttttcaagcagacgacggcatccgagataggttggggtgactggagttcagacgtgtgctcttccgatctctgattctggagtccgccagcaccaaccagacatctatgtacctctgtgccagcagtcgtcccggactagcgggagggaaggagacccagtacttcgggccaggcacgcggctcctggtgctcgaggacctgaaaaacgtgttcccacccgaggtcgctgtgtttgagccatcagaagcagagatctc

SEQ ID NO: 271 - protein, 94 aa, Homo sapiens
  SITE 19: note = misc_feature - Xaa can be any naturally occurring amino acid
  SITE 25: note = misc_feature - Xaa can be any naturally occurring amino acid
PPPPPPPPPPPPLFFFSSXRRHTRXVGVTGVQTCALPISVKIQRTEQGDSAVYLCASSLVQDFGPGTRLLVLEDLKNVFPPEVAVFEPSEAEIZ

SEQ ID NO: 272 - DNA, 283 bp, Homo sapiens
  source 1..283: mol_type = genomic DNA
ccccccccccccccccccccccccccccccccccccccctattttttttttcaagctgaagacggcatacgagataggttggggtgactggagttcagacgtgtgctcttccgatctcggtgaagatccagcgcacagagcagggggactcagctgtgtatctctgtgccagcagcttagttcaagacttcgggccaggcacgcggctcctggtgctcgaggacctgaaaaacgtgttcccacccgaggtcgctgtgtttgagccatcagaagcagagatctc

SEQ ID NO: 273 - protein, 95 aa, Homo sapiens
FPWYQQFPGKGPALLIAIRPDVSEKKEGRFTISFNKSAKQFSLHIMDSQPGDSATYFCAAKGSSYGKLTFGQGTILTVHPNIQNPDPAVYQLRDZ

SEQ ID NO: 274 - DNA, 283 bp, Homo sapiens
  source 1..283: mol_type = genomic DNA
tttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagcaaaagggagcagctatggaaagctgacatttggacaagggaccatcttgactgtccatccaaatatccagaaccctgaccctgccgtgtaccagctgagagact

SEQ ID NO: 275 - protein, 95 aa, Homo sapiens
FPWYQQFPGKGPALLIAIRPDVSEKKEGRFTISFNKSAKQFSLHIMDSQPGDSATYFCAAYGFGNEKLTFGTGTRLTIIPNIQNPDPAVYQLRDZ

SEQ ID NO: 276 - DNA, 283 bp, Homo sapiens
  source 1..283: mol_type = genomic DNA
tttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagcatatggctttggaaatgagaaattaacctttgggactggaacaagactcaccatcatacccaatatccagaaccctgaccctgccgtgtaccagctgagagact

SEQ ID NO: 277 - protein, 95 aa, Homo sapiens
WYKQDTGRGPVSLTIMTFSENTKSNGRYTATLDADTKQSSLHITASQLSDSASYICVVSPIGGSNYKLTFGKGTLLTVNPNIQNPDPAVYQLRDZ

SEQ ID NO: 278 - DNA, 283 bp, Homo sapiens
  source 1..283: mol_type = genomic DNA
tggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgagcccgattggaggtagcaactataaactgacatttggaaaaggaactctcttaaccgtgaatccaaatatccagaaccctgaccctgccgtgtaccagctgagagact

SEQ ID NO: 279 - protein, 95 aa, Homo sapiens
YKQESGKGPQFIIDIRSNMDKRQGQRVTVLLNKTVKHLSLQIAATQPGDSAVYFCAENNLADSGYSTLTFGKGTMLLVSPDIQNPDPAVYQLRDZ

SEQ ID NO: 280 - DNA, 283 bp, Homo sapiens
  source 1..283: mol_type = genomic DNA
tacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaataatctggcggattcaggatacagcaccctcacctttgggaaggggactatgcttctagtctctccagatatccagaaccctgaccctgccgtgtaccagctgagagact

SEQ ID NO: 281 - protein, 95 aa, Homo sapiens
WYKQPPSGELVFLIRRNSFDEQNEISGRYSWNFQKSTSSFNFTITASQVVDSAVYFCALMLAGGYQKVTFGIGTKLQVIPNIQNPDPAVYQLRDZ

SEQ ID NO: 282 - DNA, 283 bp, Homo sapiens
  source 1..283: mol_type = genomic DNA
tggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgatgctggccgggggttaccagaaagttacctttggaattggaacaaagctccaagtcatcccaaatatccagaaccctgaccctgccgtgtaccagctgagagact

SEQ ID NO: 283 - protein, 94 aa, Homo sapiens
SLTLIATANQGSEATYESGFVIDKFPISRPNLTFSTLTVSNMSPEDSSIYLCSVAGSESEQFFGPGTRLTVLEDLKNVFPPEVAVFEPSEAEIZ

SEQ ID NO: 284 - DNA, 283 bp, Homo sapiens
  source 1..283: mol_type = genomic DNA
agagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgcggggagcgagagcgagcagttcttcgggccagggacacggctcaccgtgctagaggacctgaaaaacgtgttcccacccgaggtcgctgtgtttgagccatcagaagcagagatctc

SEQ ID NO: 285 - protein, 94 aa, Homo sapiens
LMATSNEGSKATYEQGVEKDKFLINHASLTLSTLTVTSAHPEDSSFYICSARTDIGKGYEQYFGPGTRLTVTEDLKNVFPPEVAVFEPSEAEIZ

SEQ ID NO: 286 - DNA, 283 bp, Homo sapiens
  source 1..283: mol_type = genomic DNA
tgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaacagatattggaaagggatacgagcagtacttcgggccgggcaccaggctcacggtcacagaggacctgaaaaacgtgttcccacccgaggtcgctgtgtttgagccatcagaagcagagatctc

SEQ ID NO: 287 - protein, 94 aa, Homo sapiens
MVYLQKENIIDESGMPKERFSAEFPKEGPSILRIQQVVRGDSAAYFCASSPTLLEVPYNEQFFGPGTRLTVLEDLKNVFPPEVAVFEPSEAEIZ

SEQ ID NO: 288 - DNA, 283 bp, Homo sapiens
  source 1..283: mol_type = genomic DNA
tcatggtttatctccagaaagaaaatatcatagatgagtcaggaatgccaaaggaacgattttctgctgaatttcccaaagagggccccagcatcctgaggatccagcaggtagtgcgaggagattcggcagcttacttctgtgccagctcacccaccttactagaggtcccctacaatgagcagttcttcgggccagggacacggctcaccgtgctagaggacctgaaaaacgtgttcccacccgaggtcgctgtgtttgagccatcagaagcagagatctc

SEQ ID NO: 289 - protein, 94 aa, Homo sapiens
RLIYYSQIVNDFQKGDIAEGYSVSREKKESFPLTVTSAQKNPTAFYLCASSRLDRDLQETQYFGPGTRLLVLEDLKNVFPPEVAVFEPSEAEIZ

SEQ ID NO: 290 - DNA, 283 bp, Homo sapiens
  source 1..283: mol_type = genomic DNA
tgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtcgactggacagggacctacaagagacccagtacttcgggccaggcacgcggctcctggtgctcgaggacctgaaaaacgtgttcccacccgaggtcgctgtgtttgagccatcagaagcagagatctc

SEQ ID NO: 291 - protein, 94 aa, Homo sapiens
EFLIYFQGNRAPDKSGLPSDRFSAERTGGSVSTLTIQRTQQEDSAVYLCASSLEQGLNQPQHFGDGTRLSILEDLNKVFPPEVAVFEPSEAEIZ

SEQ ID NO: 292 - DNA, 283 bp, Homo sapiens
  source 1..283: mol_type = genomic DNA
tggagtttttaatttacttccaaggcaacagggcaccagacaaatcagggctgcccagtgatcgcttctctgcagagaggactgggggatccgtctccactctgacgatccagcgcacacagcaggaggactcggccgtgtatctctgtgccagcagcttagaacagggcctcaatcagccccagcattttggtgatgggactcgactctccatcctagaggacctgaacaaggtgttcccacccgaggtcgctgtgtttgagccatcagaagcagagatctc

SEQ ID NO: 293 - protein, 95 aa, Homo sapiens; identical to SEQ ID NO: 273

SEQ ID NO: 294 - DNA, 283 bp, Homo sapiens; identical to SEQ ID NO: 274
  source 1..283: mol_type = genomic DNA

SEQ ID NO: 295 - protein, 95 aa, Homo sapiens
GLFWYQQHAGEAPTFLSYNVLDGLEEKGRFSSFLSRSKGYSYLLLKELQMKDSASYLCAEGSSNTGKLIFGQGTTLQVKPDIQNPDPAVYQLRDZ

SEQ ID NO: 296 - DNA, 283 bp, Homo sapiens
  source 1..283: mol_type = genomic DNA
gggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgagggctctagcaacacaggcaaactaatctttgggcaagggacaactttacaagtaaaaccagatatccagaaccctgaccctgccgtgtaccagctgagagact

SEQ ID NO: 297 - protein, 12 aa, Homo sapiens
CAVKGNTGKLIF

SEQ ID NO: 298 - DNA, 30 bp, Homo sapiens
  source 1..30: mol_type = genomic DNA
gctgtgaagggaaacacaggcaaactaatc

SEQ ID NO: 299 - protein, 95 aa, Homo sapiens
NGLFWYQQHAGEAPTFLSYNVLDGLEEKGRFSSFLSRSKGYSYLLLKELQMKDSASYLCAVKGNTGKLIFGQGTTLQVKPDIQNPDPAVYQLRDZ

SEQ ID NO: 300 - DNA, 283 bp, Homo sapiens
  source 1..283: mol_type = genomic DNA
aacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgaagggaaacacaggcaaactaatctttgggcaagggacaactttacaagtaaaaccagatatccagaaccctgaccctgccgtgtaccagctgagagact

SEQ ID NO: 301 - protein, 95 aa, Homo sapiens
VFWYQQHAGEAPTFLSYNVLDGLEEKGRFSSFLSRSKGYSYLLLKELQMKDSASYLCAGLTTDSWGKLQFGAGTQVVVTPDIQNPDPAVYQLRDZ

SEQ ID NO: 302 - DNA, 283 bp, Homo sapiens
  source 1..283: mol_type = genomic DNA
gtgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgggttgacaactgacagctgggggaaattgcagtttggagcagggacccaggttgtggtcaccccagatatccagaaccctgaccctgccgtgtaccagctgagagact

SEQ ID NO: 303 - protein, 95 aa, Homo sapiens; identical to SEQ ID NO: 277

SEQ ID NO: 304 - DNA, 283 bp, Homo sapiens; identical to SEQ ID NO: 278
  source 1..283: mol_type = genomic DNA

SEQ ID NO: 305 - protein, 94 aa, Homo sapiens; identical to SEQ ID NO: 283

SEQ ID NO: 306 - DNA, 283 bp, Homo sapiens; identical to SEQ ID NO: 284
  source 1..283: mol_type = genomic DNA

SEQ ID NO: 307 - protein, 94 aa, Homo sapiens; identical to SEQ ID NO: 289

SEQ ID NO: 308 - DNA, 283 bp, Homo sapiens; identical to SEQ ID NO: 290
  source 1..283: mol_type = genomic DNA

SEQ ID NO: 309 - protein, 94 aa, Homo sapiens; identical to SEQ ID NO: 285

SEQ ID NO: 310 - DNA, 283 bp, Homo sapiens
  source 1..283: mol_type = genomic DNA
tgctgatggcaacttccaatgagggctccaaggccacatacgagcaaggcgttgagaaggacaagtttctcatcaaccatgcaagcctgaccttgtccactctgacagtgaccagtgcccatcctgaagacagcagcttctacatctgcagtgctagaacagatattggaaagggatacgagcagtacttcgggccgggcaccaggctcacggtcacagaggacctgaaaaacgtgttcccacccgaggtcgctgtgtttgagccatcagaagcagagatctc

SEQ ID NO: 311 - protein, 94 aa, Homo sapiens; identical to SEQ ID NO: 287

SEQ ID NO: 312 - DNA, 283 bp, Homo sapiens
  source 1..283: mol_type = genomic DNA
tcatggtttatctccagaaagaaaatatcatagatgagtcaggaatgccaaaggaacgattttctgctgaatttcccaaagagggccccagcatcctgaggatccagcaggtagtgcgaggagattcggcagcttacttctgtgccagctcacccaccttactagaggtcccctacaatgagcagttcttcggaccagggacacggctcaccgtgctagaggacctgaaaaacgtgttcccacccgaggtcgctgtgtttgagccatcagaagcagagatctc

SEQ ID NO: 313 - protein, 94 aa, Homo sapiens
QGPQFIFQYYREEENGRGNFPPRFSGLQFPNYSSELNVNALELDDSALYLCASSSGISYEQYFGPGTRLTVTEDLKNVFPPEVAVFEPSEAEIZ

SEQ ID NO: 314 - DNA, 283 bp, Homo sapiens
  source 1..283: mol_type = genomic DNA
gtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagctcggggatctcctacgagcagtacttcgggccgggcaccaggctcacggtcacagaggacctgaaaaacgtgttcccacccgaggtcgctgtgtttgagccatcagaagcagagatctc

SEQ ID NO: 315 - protein, 95 aa, Homo sapiens
  SITE 28: note = misc_feature - Xaa can be any naturally occurring amino acid
PPPPPPPPPPPPDRFFHAVDGIRDWIWXLEFRRVLFRSSLKISDSQLGDAAMYFCAYRSDYGGATNKLIFGTGTLLAVQPNIQNPDPAVYQLRDZ

SEQ ID NO: 316 - DNA, 283 bp, Homo sapiens
  source 1..283: mol_type = genomic DNA
ccccccccccccccccccccccccccccccccacccgatagattttttcacgcagtagacggcatacgagattggatctggtgactggagttcagacgtgtgctcttccgatctagtctcaagatctcagactcacagctgggggatgccgcgatgtatttctgtgcttataggagcgactatggtggtgctacaaacaagctcatctttggaactggcactctgcttgctgtccagccaaatatccagaaccctgaccctgccgtgtaccagctgagagact

SEQ ID NO: 317 - protein, 95 aa, Homo sapiens
PPPPPPPPPPPPPPPTFFFSSRRRNTRLDLVTGVQTCALPICFLHITAPKPEDSATYLCAVRSNDYKLSFGAGTTVTVRANIQNPDPAVYQLRDZ

SEQ ID NO: 318 - DNA, 283 bp, Homo sapiens
  source 1..283: mol_type = genomic DNA
cccccccccccccccccccccccccccccccccccccccccccccaccttttttttttcaagcagaagacggaatacgagattggatctggtgactggagttcagacgtgtgctcttccgatctgctttctgcacatcacagcccctaaacctgaagactcagccacttatctctgtgctgtgaggtctaacgactacaagctcagctttggagccggaaccacagtaactgtaagagcaaatatccagaaccctgaccctgccgtgtaccagctgagagact

SEQ ID NO: 319 - protein, 95 aa, Homo sapiens
PPPPPPPPPPPSFFFKQKTAYAIGSGDWSSDVCSSDLFTITASQVVDSAVYFCALRPRVFSSGSARQLTFGSGTQLTVLPDIQNPDPAVYQLRDZ

SEQ ID NO: 320 - DNA, 283 bp, Homo sapiens
  source 1..283: mol_type = genomic DNA
ccccccccccccccccccccccccccccccccctctttttttttcaagcagaagacggcatacgcgattggatctggtgactggagttcagacgtgtgctcttccgatctgttcaccatcacagcctcacaagtcgtggactcagcagtatacttctgtgctctgagacccagagttttttcttctggttctgcaaggcaactgacctttggatctgggacacaattgactgttttacctgatatccagaaccctgaccctgccgtgtaccagctgagagact

SEQ ID NO: 321 - protein, 95 aa, Homo sapiens
  SITE 39: note = misc_feature - Xaa can be any naturally occurring amino acid
PPPPPPPPPPPPPIFFSSRRRHTRLDLVTGVQTCALPIXLKKPSALVSDSALYFCAVRGLFGGATNKLIFGTGTLLAVQPNIQNPDPAVYQLRDZ

SEQ ID NO: 322 - DNA, 283 bp, Homo sapiens
  source 1..283: mol_type = genomic DNA
cccccccccccccccccccccccccccccccccccccccatttttttttcaagcagaagacggcatacgagattggatctggtgactggagttcagacgtgtgctcttccgatctgactgaagaaaccatctgcccttgtgagcgactccgctttgtacttctgtgctgtgagaggtctttttgggggtgctacaaacaagctcatctttggaactggcactctgcttgctgtccagccaaatatccagaaccctgaccctgccgtgtaccagctgagagact

SEQ ID NO: 323 - protein, 95 aa, Homo sapiens
  SITE 34: note = misc_feature - Xaa can be any naturally occurring amino acid
PPPPPPPPPPPPPPHFFFLEQMTSLDIGSGDWSXDLCSSDLILPHATLRDTAVYYCIVRAANTNAGKSTFGDGTTLTVKPNIQNPDPAVYQLRDZ

SEQ ID NO: 324 - DNA, 283 bp, Homo sapiens
  source 1..283: mol_type = genomic DNA
cccccccccccccccccccccccccccccccccccccccccccactttttttttttagagcagatgacgtcattggatattggttctggtgattggagttgagacttgtgctcttctgatctcatcctgccccacgctacgctgagagacactgctgtgtactattgcatcgtcagagcagcgaacaccaatgcaggcaaatcaacctttggggatgggactacgctcactgtgaagccaaatatccagaaccctgaccctgccgtgtaccagctgagagact

SEQ ID NO: 325 - protein, 94 aa, Homo sapiens
PPPPPPPPLFFFKQKTAYEIGSGDWSSDVCSSDLTLTVTSAHPEDSSFYICSASEDPDYGYTFGSGTRLTVVEDLNKVFPPEVAVFEPSEAEIZ

SEQ ID NO: 326 - DNA, 283 bp, Homo sapiens
  source 1..283: mol_type = genomic DNA
cccccccccccccccccccccccccccttttttttttcaagcagaagacggcatacgagattggatctggtgactggagttcagacgtgtgctcttccgatctcactctgacagtgaccagtgcccatcctgaagacagcagcttctacatctgcagtgctagtgaggacccggactatggctacaccttcggttcggggaccaggttaaccgttgtagaggacctgaacaaggtgttcccacccgaggtcgctgtgtttgagccatcagaagcagagatctc

SEQ ID NO: 327 - protein, 94 aa, Homo sapiens
PPPPPPTLFFYTQKTPYELGSGDWSADVCSSDLTLTVTSAHPEDSSFYICSARELAGAGEQYFGPGTRLTVTEDLKNVFPPEVAVFEPSEAEIZ

SEQ ID NO: 328 - DNA, 283 bp, Homo sapiens
  source 1..283: mol_type = genomic DNA
ccccccccccccccccccccaccctttttttttacacgcagaagacgccctacgagcttggatctggtgactggagtgcagacgtgtgctcttccgatctcactctgacagtgaccagtgcccatcctgaagacagcagcttctacatctgcagtgcccgggagctagcgggagcgggcgagcagtacttcgggccgggcaccaggctcacggtcacagaggacctgaaaaacgtgttcccacccgaggtcgctgtgtttgagccatcagaagcagagatctc

SEQ ID NO: 329 - protein, 94 aa, Homo sapiens
PPPRRDFFFKEKTASSIGSGDWSSDVCSSDLFLHLHTLQPEDSALYLCASSQEYETGGYGYTFGSGTRLTVVEDLNKVFPPEVAVFEPSEAEIZ

SEQ ID NO: 330 - DNA, 283 bp, Homo sapiens
  source 1..283: mol_type = genomic DNA
ccccacccccccgacgagatttttttttcaaggagaagacggcatcctcgattggatctggtgactggagttcagacgtgtgctcttccgatctattccttcacctacacaccctgcagccagaagactcggccctgtatctctgcgccagcagccaagaatatgaaacagggggctatggctacaccttcggttcggggaccaggttaaccgttgtagaggacctgaacaaggtgttcccacccgaggtcgctgtgtttgagccatcagaagcagagatctc

SEQ ID NO: 331 - protein, 94 aa, Homo sapiens
  SITE 11: note = misc_feature - Xaa can be any naturally occurring amino acid
PPPAPRILFYXHHSEYSFGYGEWSSEVCSYVLLKIRSTKLEDSAMYFCASSGQGLARAYEQYFGPGTRLTVTEDLKNVFPPEVAVFEPSEAEIZ

SEQ ID NO: 332 - DNA, 283 bp, Homo sapiens
  source 1..283: mol_type = genomic DNA
cccccccccccgccccccgcatcttgttttactagcaccactcggaatactcttttggatatggtgagtggagttcagaggtgtgctcttacgttcttctgaagatccggtccacaaagctggaggactcagccatgtacttctgtgccagcagtggacaaggactagcgagagcatacgagcagtacttcgggccgggcaccaggctcacggtcacagaggacctgaaaaacgtgttcccacccgaggtcgctgtgtttgagccatcagaagcagagatctc

SEQ ID NO: 333 - protein, 94 aa, Homo sapiens
PPPPPPTVFFFNDTPANELGSGAWSSAVCSSDLLILESPSPNQTSLYFCASSQGQGRYNEQFFGPGTRLTVLEDLKNVFPPEVAVFEPSEAEIZ

SEQ ID NO: 334 - DNA, 283 bp, Homo sapiens
  source 1..283: mol_type = genomic DNA
ccccccccccccccccccccacagttttttttttcaacgacacgccggcaaacgagcttggctctggggcatggagttcagccgtgtgctcttccgatctgctgatcctggagtcgcccagccccaaccagacctctctgtacttctgtgccagctcccaaggacaggggcgatacaatgagcagttcttcgggccagggacacggctcaccgtgctagaggacctgaaaaacgtgttcccacccgaggtcgctgtgtttgagccatcagaagcagagatctc

SEQ ID NO: 335 - protein, 95 aa, Homo sapiens
WYKQPPSRQMILVIRQEAYKQQNATENRFSVNFQKAAKSFSLKISDSQLGDAAMYFCAYRRKAAGNKLTFGGGTRVLVKPNIQNPDPAVYQLRDZ

SEQ ID NO: 336 - DNA, 283 bp, Homo sapiens
  source 1..283: mol_type = genomic DNA
tggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagaaaagctgcaggcaacaagctaacttttggaggaggaaccagggtgctagttaaaccaaatatccagaaccctgaccctgccgtgtaccagctgagagact

SEQ ID NO: 337 - protein, 95 aa, Homo sapiens
IHWYRQLPSQGPEYVIHGLTSNVNNRMASLAIAEDRKSSTLILHRATLRDAAVYYCISGAGGTSYGKLTFGQGTILTVHPNIQNPDPAVYQLRDZ

SEQ ID NO: 338 - DNA, 283 bp, Homo sapiens
  source 1..283: mol_type = genomic DNA
atacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatctctggggctggtggtactagctatggaaagctgacatttggacaagggaccatcttgactgtccatccaaatatccagaaccctgaccctgccgtgtaccagctgagagact

SEQ ID NO: 339 - protein, 95 aa, Homo sapiens
YPNRGLQFLLKYITGDNLVKGSYGFEAEFNKSQTSFHLKKPSALVSDSALYFCAVRDMRLAGGGADGLTFGKGTHLIIQPYIQNPDPAVYQLRDZ

SEQ ID NO: 340 - DNA, 283 bp, Homo sapiens
  source 1..283: mol_type = genomic DNA
taccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacatgagactggcaggaggaggtgctgacggactcacctttggcaaagggactcatctaatcatccagccctatatccagaaccctgaccctgccgtgtaccagctgagagact

SEQ ID NO: 341 - protein, 95 aa, Homo sapiens
NLQWFRQDPGKGLTSLLLIQSSQREQTSGRLNASLDKSSGRSTLYIAASQPGDSATYLCAALYNFNKFYFGSGTKLNVKPNIQNPDPAVYQLRDZ

SEQ ID NO: 342 - DNA, 283 bp, Homo sapiens
  source 1..283: mol_type = genomic DNA
aacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgccttatacaacttcaacaaattttactttggatctgggaccaaactcaatgtaaaaccaaatatccagaaccctgaccctgccgtgtaccagctgagagact

SEQ ID NO: 343 - protein, 95 aa, Homo sapiens
WYVQYPNRGLQFLLKYITGDNLVKGSYGFEAEFNKSQTSFHLKKPSALVSDSALYFCAVSQNTNAGKSTFGDGTTLTVKPNIQNPDPAVYQLRDZ

SEQ ID NO: 344 - DNA, 283 bp, Homo sapiens
  source 1..283: mol_type = genomic DNA
tggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagccaaaacaccaatgcaggcaaatcaacctttggggatgggactacgctcactgtgaagccaaatatccagaaccctgaccctgccgtgtaccagctgagagact

SEQ ID NO: 345 - protein, 94 aa, Homo sapiens
KVEFLVSFYNNEISEKSEIFDDQFSVERPDGSNFTLKIRSTKLEDSAMYFCASSDRMDYEQYFGPGTRLTVTEDLKNVFPPEVAVFEPSEAEIZ

SEQ ID NO: 346 - DNA, 283 bp, Homo sapiens
  source 1..283: mol_type = genomic DNA
agaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgataggatggactacgagcagtacttcgggccgggcaccaggctcacggtcacagaggacctgaaaaacgtgttcccacccgaggtcgctgtgtttgagccatcagaagcagagatctc

SEQ ID NO: 347 - protein, 94 aa, Homo sapiens
LQFLFEYFSETQRNKGNFPGRFSGRQFSNSRSEMNVSTLELGDSALYLCASSLSGGWADTQYFGPGTRLTVLEDLKNVFPPEVAVFEPSEAEIZ

SEQ ID NO: 348 - DNA, 283 bp, Homo sapiens
  source 1..283: mol_type = genomic DNA
gccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttgtcagggggttgggcagatacgcagtattttggcccaggcacccggctgacagtgctcgaggacctgaaaaacgtgttcccacccgaggtcgctgtgtttgagccatcagaagcagagatctc

SEQ ID NO: 349 - protein, 94 aa, Homo sapiens
MGLKLIYYSVGAGITDKGEVPNGYNVSRSTTEDFPLRLELAAPSQTSVYFCASSREGYNEQFFGPGTRLTVLEDLKNVFPPEVAVFEPSEAEIZ

SEQ ID NO: 350 - DNA, 283 bp, Homo sapiens
  source 1..283: mol_type = genomic DNA
gcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagtagggaagggtacaatgagcagttcttcgggccagggacacggctcaccgtgctagaggacctgaaaaacgtgttcccacccgaggtcgctgtgtttgagccatcagaagcagagatctc

SEQ ID NO: 351 - protein, 94 aa, Homo sapiens
HGLRLIHYSYGVKDTDKGEVSDGYSVSRSKTEDFLLTLESATSSQTSVYFCAITAREGETQYFGPGTRLLVLEDLKNVFPPEVAVFEPSEAEIZ

SEQ ID NO: 352 - DNA, 283 bp, Homo sapiens
  source 1..283: mol_type = genomic DNA
ggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcacggcgagggagggagagacccagtacttcgggccaggcacgcggctcctggtgctcgaggacctgaaaaacgtgttcccacccgaggtcgctgtgtttgagccatcagaagcagagatctc

SEQ ID NO: 353 - protein, 94 aa, Homo sapiens; identical to SEQ ID NO: 347

SEQ ID NO: 354 - DNA, 283 bp, Homo sapiens
  source 1..283: mol_type = genomic DNA
gccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttgtcaggggggtgggcagatacgcagtattttggcccaggcacccggctgacagtgctcgaggacctgaaaaacgtgttcccacccgaggtcgctgtgtttgagccatcagaagcagagatctc

SEQ ID NO: 355 - protein, 94 aa, Homo sapiens
PTPVFIFKPTPAYEIGSGDCSSDVCSSDLPENLIERDSGVYYCATWDNLYKKLFGSGTTLVVTDKQLDADVSPKPTIFLPSIAETKLQKAGTYL

SEQ ID NO: 356 - DNA, 283 bp, Homo sapiens
  source 1..283: mol_type = genomic DNA
acccacccccgtttttattttcaagccgactccggcatacgagattggatctggtgactgtagttcagacgtgtgctcttccgatctgccggaaaatctaattgaacgtgactctggggtctattactgtgccacctgggataatttgtataagaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 357 - protein, 94 aa, Homo sapiens
PLFFFKQKTAYAIGSGDWSSDVCSSDLLRNLIENDSGVYYCATWDRRGYYKKLFGSGTTLVVTDKQLDADVSPKPTIFLPSIAETKLQKAGTYL

SEQ ID NO: 358 - DNA, 283 bp, Homo sapiens
  source 1..283: mol_type = genomic DNA
accccttttttttttcaagcagaagacggcatacgcgattggatctggtgactggagttcagacgtgtgctcttccgatctcctacgaaatctaattgaaaatgattctggggtctattactgtgccacctgggacaggcggggttattataagaaactctttggcagtggaacaacactcgttgtcacagataaacaacttgatgcagatgtttcccccaagcccactatttttcttccttcaattgctgaaacaaaactccagaaggctggaacatacctt

SEQ ID NO: 359 - protein, 94 aa, Homo sapiens
  SITE 8: note = misc_feature - Xaa can be any naturally occurring amino acid
  SITE 17: note = misc_feature - Xaa can be any naturally occurring amino acid
  SITE 33: note = misc_feature - Xaa can be any naturally occurring amino acid
  SITE 35: note = misc_feature - Xaa can be any naturally occurring amino acid
PTFFFQAXDGIRDWIWXLEFRRVLFRSWAKSNXKXFWIYYCATWDTPNYYKKLFGSGTTLVVTDKQLDADVSPKPTIFLPSIAETKLQKAGTYL

SEQ ID NO: 360 - DNA, 283 bp, Homo sapiens
  source 1..283: mol_type = genomic DNA
ccccacttttttttttcaagcataagacggcatacgagattggatctggtgactggagttcagacgtgtgctcttccgatcttgggcaaaatctaattgaaaatgattctggatctattactgtgccacctgggacaccccgaattattataagaaactctttggcagtggaacaacacttgttgtcacagataaacaacttgatgcagatgtttcccccaagcccactatttttcttccttcaattgctgaaacaaaactccagaaggctggaacatatctt

SEQ ID NO: 361 - protein, 94 aa, Homo sapiens
  SITE 30: note = misc_feature - Xaa can be any naturally occurring amino acid
PPPLFFFKQKTAYEIGSGDWSSDVCSSDLXQNLIENDSGVYYCATWDYYYKKLFGSGTTLVVTDKQLDADASPKPTIFLPSIAETKLQKAGTYL

SEQ ID NO: 362 - DNA, 283 bp, Homo sapiens
  source 1..283: mol_type = genomic DNA
cccccccccccttttttttttcaagcagaagacggcatacgagattggatctggtgactggagttcagacgtgtgctcttccgatctgtagcaaaatctaattgaaaatgattctggggtctattactgtgccacctgggactattattataagaaactctttggcagtggaacaacacttgttgtcacagataaacaacttgatgcagatgcttcccccaagcccactatttttcttccttcgattgctgaaacaaaactccagaaggctggaacatatctt

SEQ ID NO: 363 - protein, 94 aa, Homo sapiens
PPPHIIFFKQKTAYEIGSGDWSSDVCSSDPLENLIERDSGVYYCATWDKLGKLFGSGTTLVVTDKQLDADVSPKPTIFLPSIAETKLQKAGTYL

SEQ ID NO: 364 - DNA, 283 bp, Homo sapiens
  source 1..283: mol_type = genomic DNA
cccccccccccacattatttttttcaagcagaagacggcatacgagattggatctggtgactggagttcagacgtgtgctcttccgatccactggaaaatctaattgaacgtgactctggggtctattactgtgccacctgggataaattagggaaactctttggcagtggaacaacacttgttgtcacagataaacaacttgatgcagatgtttcccccaagcccactatttttcttccttcgattgctgaaacaaaactccagaaggctggaacatatctt

SEQ ID NO: 365 - protein, 94 aa, Homo sapiens
KTFFFKQKSAIEIGCGDLSSDVVSSVLERNLIENDSGVYYCATWGMQDYYKKLFGSGTTLVVTDKQLDADVSPKPTIFLPSIAETKLQKAGTYL

SEQ ID NO: 366 - DNA, 283 bp, Homo sapiens
  source 1..283: mol_type = genomic DNA
gaagactttttttttcaagcagaagtccgcaatcgagattggatgtggtgacttgagttcagacgtggtctcttccgttctagaaagaaatctaattgaaaatgattctggagtctattactgtgccacctggggtatgcaggattattataagaaactctttggcagtggaacaacacttgttgtcacagataaacaacttgatgcagatgtttcccccaagcccactatttttcttccttcaattgctgaaacaaaactccagaaggctggaacatacctt

SEQ ID NO: 367 - protein, 94 aa, Homo sapiens
  SITE 7: note = misc_feature - Xaa can be any naturally occurring amino acid
PPTDIFXTRTPANASGSGDWSSDVCSSDLLRNLIENDSGVYYCATWGNYYKKLFGSGTTLVVTDKQLDADVSPKPTIFLPSIAETKLQKAGTYL

SEQ ID NO: 368 - DNA, 283 bp, Homo sapiens
  source 1..283: mol_type = genomic DNA
ccccccgactgatattttttaaacgcggacgccggcaaacgcgagtggatctggtgactggagttcagacgtgtgctcttccgatctcctacgaaatctaattgaaaatgattctggggtctattactgtgccacctgggggaattattataagaaactctttggcagtggaacaacacttgttgtcacagataaacaacttgatgcagatgtttcccccaagcccactatttttcttccttcaattgctgaaacaaagctccagaaggctggaacatacctt

SEQ ID NO: 369 - protein, 94 aa, Homo sapiens
  SITE 27: note = misc_feature - Xaa can be any naturally occurring amino acid
LYFYKHTTPYDIGSGDSSSDVCSSDLXRNLIENDSGVYYCATWEKAGVDYKKLFGSGTTLVVTDKQLDADVSPKPTIFLPSIAETKLQKAGTYL

SEQ ID NO: 370 - DNA, 283 bp, Homo sapiens
  source 1..283: mol_type = genomic DNA
actttatttttacaagcacacgacgccatacgacattggatctggtgactcgagttcagacgtgtgctcttccgatctataacgaaatctaattgaaaatgattctggggtctattactgtgccacctgggagaaggcgggagttgattataagaaactctttggcagtggaacaacacttgttgtcacagataaacaacttgatgcagatgtttcccccaagcccactatttttcttccttcaattgctgaaacaaagctccagaaggctggaacatatctt

SEQ ID NO: 371 - protein, 94 aa, Homo sapiens
HPIFLFKQKTAYEIGSGAWSSDVCSSGLLRNLIENDSGVYYCATWDFGYYKKLFGSGTTLVVIDKQLDADVSPKPTIFLPSIAETKLQKAGTYL

SEQ ID NO: 372 - DNA, 283 bp, Homo sapiens
  source 1..283: mol_type = genomic DNA
ccaccccatttttttattcaagcagaagacggcatacgagattggatctggtgcctggagttcagacgtgtgctcttccggtctattacgaaatctaattgaaaatgattctggggtctattactgtgccacctgggacttcggttattataagaaactctttggcagtggaacaacactcgttgtcatagataaacaacttgatgcagatgtttcccccaagcccactatttttcttccttcaattgctgaaacaaaactccagaaggctggaacatacctt

SEQ ID NO: 373 - protein, 94 aa, Homo sapiens
HYCFFRSDRRSYPIVCGSCCSDCVCSYYMEYLIERDSGVYYCATWEPGNFEKLFGSGTTLVVTDKQLDADVSPKPTIFLPSIAETKLQKAGTYL

SEQ ID NO: 374 - DNA, 283 bp, Homo sapiens
  source 1..283: mol_type = genomic DNA
acactattgtttttttagaagcgatcgtcgctcataccctattgtatgtggttcctgttgttcagactgtgtctgttcctattacatggaatatctaattgaacgtgactctggggtctattactgtgccacctgggagcccggcaattttgagaaactctttggcagtggaacaacacttgttgtcacagataaacaacttgatgcagatgtttcccccaagcccactatttttcttccttcaattgctgaaacaaaactccagaaggctggaacatacctt

SEQ ID NO: 375 - protein, 93 aa, Homo sapiens
PPPPPPPPPPPPLFFFKQKTAYEIGSGDWSSDVCSSDLIHLVISPVRTEDSATYYCAFSGSLVLGDRSTDKLIFGKGTRVTVEPRSQPHTKPS

SEQ ID NO: 376 - DNA, 283 bp, Homo sapiens
  source 1..283: mol_type = genomic DNA
cccccccccccccccccccccccccccccccccccccccttttttttttcaagcagaagacggcatacgagattggatctggtgactggagttcagacgtgtgctcttccgatctcattcacttggtgatctctccagtaaggactgaagacagtgccacttactactgtgccttttcaggctctttagtactgggggataggagcaccgataaactcatctttggaaaaggaacccgtgtgactgtggaaccaagaagtcagcctcataccaaaccatccgt

SEQ ID NO: 377 - protein, 94 aa, Homo sapiens
PPPPPPPPPPHFFLKQKTAYEIGSGDWSSDVCSSDLLTISALQLEDSAKYFCALGDLAFLLPGGWHRWTDKLIFGKGTRVTVEPRSQPHTKPSZ

SEQ ID NO: 378 - DNA, 283 bp, Homo sapiens
  source 1..283: mol_type = genomic DNA
cccccccccccccccccccccccccccccccccatttttttttaaagcagaagacggcatacgagattggatctggtgactggagttcagacgtgtgctcttccgatcttttaaccatttcagccttacagctagaagattcagcaaagtacttttgtgctcttggggacctcgcattcctactccctgggggatggcatcggtggaccgataaactcatctttggaaaaggaacccgtgtgactgtggaaccaagaagtcagcctcataccaaaccatccgt

SEQ ID NO: 379 - protein, 94 aa, Homo sapiens
  SITE 41: note = misc_feature - Xaa can be any naturally occurring amino acid
PPPPPPPPPPPPPPHFFFKQKTAYEIGSGDWSSDVCSSDLXLKILAPSERDEGSYYCACDTVAGLGVDTDKLIFGKGTRVTVEPRSQPHTKPSZ

SEQ ID NO: 380 - DNA, 283 bp, Homo sapiens
  source 1..283: mol_type = genomic DNA
cccccccccccccccccccccccccccccccccccccccccccccatttttttttcaagcagaagacggcatacgagattggatctggtgactggagttcagacgtgtgctcttccgatctgtgacttaagatacttgcaccatcagagagagatgaagggtcttactactgtgcctgtgacaccgtagccggactgggggtcgacaccgataaactcatctttggaaaaggaacccgtgtgactgtggaaccaagaagtcagcctcataccaaaccatccgt

SEQ ID NO: 381 - protein, 94 aa, Homo sapiens
PPPPPPPPPPPPPIFFFKQKTAYEIGSGDWSSDVCSSDLRLKILAPSERDEGSYYCACDTVDRLGDTSTDKLIFGKGTRVTVEPRSQPHTKPSZ

SEQ ID NO: 382 - DNA, 283 bp, Homo sapiens
  source 1..283: mol_type = genomic DNA
cccccccccccccccccccccccccccccccccccccccccattttttttttcaagcagaagacggcatacgagattggatctggtgactggagttcagacgtgtgctcttccgatctcagacttaagatacttgcaccatcagagagagatgaagggtcttactactgtgcctgtgacacggtcgaccgactgggggatacgtccaccgataaactcatctttggaaaaggaacccgtgtgactgtggaaccaagaagtcagcctcataccaaaccatccgt

SEQ ID NO: 383 - protein, 94 aa, Homo sapiens
PPPPPPPPPPPLYFFKQKTAYEIGSGDWSSDVCSSDLLLKILAPSERDEGSYYCACDTVLPTTGGYGDTDKLIFGKGTRVTVEPRSQPHTKPSZ

SEQ ID NO: 384 - DNA, 283 bp, Homo sapiens
  source 1..283: mol_type = genomic DNA
cccccccccccccccccccccccccccccccccccctttattttttcaagcagaagacggcatacgagattggatctggtgactggagttcagacgtgtgctcttccgatctgctacttaagatacttgcaccatcagagagagatgaagggtcttactactgtgcctgtgacacggtccttcctacgactgggggatacggggacaccgataaactcatctttggaaaaggaacccgtgtgactgtggaaccaagaagtcagcctcataccaaaccatccgt

SEQ ID NO: 385 - protein, 94 aa, Homo sapiens
  SITE 44: note = misc_feature - Xaa can be any naturally occurring amino acid
PPPPPPPPPPPPPPPPHDFFLKHKTAYEIGSGDWSSDVCSSDLXLKILAPSERDEGSYYCACDTLLGDTDKLIFGKGTRVTVEPRSQPHTKPSZ

SEQ ID NO: 386 - DNA, 283 bp, Homo sapiens
  source 1..283: mol_type = genomic DNA
cccccccccccccccccccccccccccccccccccccccccccccccccccacgatttttttttaaagcataagacggcatacgagattggatctggtgactggagttcagacgtgtgctcttccgatctttgacttaagatacttgcaccatcagagagagatgaagggtcttactactgtgcctgtgacaccctactgggggataccgataaactcatctttggaaaaggaacccgtgtgactgtggaaccaagaagtcagcctcataccaaaccatccgt

SEQ ID NO: 387 - protein, 94 aa, Homo sapiens
  SITE 33: note = misc_feature - Xaa can be any naturally occurring amino acid
PPPPPPPPPPPPPPPPPLFFFQAEDGIRYWIWXLEFRRVLFRSKLKISDSQLGDAAMYFCAYRSLFSHTDKLIFGKGTRVTVEPRSQPHTKPSZ

SEQ ID NO: 388 - DNA, 283 bp, Homo sapiens
  source 1..283: mol_type = genomic DNA
cccccccccccccccccccccccccccccccccccccccccccccccccccccctgtttttttttcaagcagaagacggcatacgatattggatctggtgactggagttcagacgtgtgctcttccgatctaagctcaagatctcagactcacagctgggggatgccgcgatgtatttctgtgcttataggagccttttcagccacaccgataaactcatctttggaaaaggaacccgtgtgactgtggaaccaagaagtcagcctcataccaaaccatccgt

SEQ ID NO: 389 - protein, 94 aa, Homo sapiens
  SITE 16: note = misc_feature - Xaa can be any naturally occurring amino acid
  SITE 32: note = misc_feature - Xaa can be any naturally occurring amino acid
PPHPPPPPPHHFFFKXNTAYDIGSGDWSSDVXSSDLVTISALQLEDSAKYFCALGELVAYEIYWGTSAADKLIFGKGTRVTVEPRSQPHTKPSZ

SEQ ID NO: 390 - DNA, 283 bp, Homo sapiens
  source 1..283: mol_type = genomic DNA
cccccccccaccccccccccccccccccccaccatttttttttcaagtagaatacggcatacgatattggatctggtgactggagttcagacgtgtgatcttccgatctggtaaccatttcagccttacagctagaagattcagcaaagtacttttgtgctcttggggaactagtcgcctacgaaatttactgggggacatccgcggccgataaactcatctttggaaaaggaacccgtgtgactgtggaaccaagaagtcagcctcataccaaaccatccgt

SEQ ID NO: 391 - protein, 94 aa, Homo sapiens
  SITE 18: note = misc_feature - Xaa can be any naturally occurring amino acid
PPPPPPPPTPPPHPSCFXQLKTANAIGSGDWSSVVCSSDLLLKILAPSERDEGSYYCACDILSGGSPYTDKLIFGKGTRVTVEPRSQPHTKPSZ

SEQ ID NO: 392 - DNA, 283 bp, Homo sapiens
  source 1..283: mol_type = genomic DNA
ccccccccccccccccccccccccccaccccccccccccaccccagttgtttttaacagctgaagacggcaaacgctattggatctggtgactggagttcagtcgtgtgctcttccgatctgttacttaagatacttgcaccatcagagagagatgaagggtcttactactgtgcctgtgacatattgtctgggggatccccgtacaccgataaactcatctttggaaaaggaacccgtgtgactgtggaaccaagaagtcagcctcataccaaaccatccgt

SEQ ID NO: 393 - protein, 94 aa, Homo sapiens
PPPPPRPPPTPPPPPPTPCFFTLRSRNTRLDLVTGVQTCALPILSLDIVPSQPGDSAVYFCAATHSGDTDKLIFGKGTRVTVEPRSQPHTKPSZ

SEQ ID NO: 394 - DNA, 283 bp, Homo sapiens
  source 1..283: mol_type = genomic DNA
ccccccccccccccccccgccccccccccaccccccccccccccccccccaccccctgtttttttacactcagaagcaggaatactagattggatctggtgacgggagttcagacgtgtgctcttccgatcttatctctcgacattgtgccctcccagcctggagactctgcagtgtacttctgtgcagcgactcattcgggggacaccgataaactcatctttggaaaaggaacccgtgtgactgtggaaccaagaagtcagcctcataccaaaccatccgt

SEQ ID NO: 395 - protein, 109 aa, Homo sapiens
DCTYDTSDPSYGLFWYKQPSSGEMIFLIYQGSYDQQNATEGRYSLNFQKARKSANLVISASQLGDSAMYFCAMREVSNTGKLIFGQGTTLQVKPDIQKPDPAVYQLRDZ

SEQ ID NO: 396 - DNA, 326 bp, Homo sapiens
  source 1..326: mol_type = genomic DNA
ggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtacttctgtgcaatgagagaggttagcaacacaggcaaactaatctttgggcaagggacaactttacaagtaaaaccagatatccagaagcctgaccctgccgtgtaccagctgagagact

SEQ ID NO: 397 - protein, 104 aa, Homo sapiens
KCTYSDSASNYFPWYKQELGKRPQLIIDIRSNVGEKKDQRIAVTLNKTAKHFSLHITETQPEDSAVYFCAASIGGQFYFGTGTSLTVIPNIQNPDPAVYQLRDZ

SEQ ID NO: 398 - DNA, 311 bp, Homo sapiens
  source 1..311: mol_type = genomic DNA
caagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtataggcggccagttctattttgggacagggacaagtttgacggtcattccaaatatccagaaccctgaccctgccgtgtaccagctgagagact

SEQ ID NO: 399 - protein, 121 aa, Homo sapiens
LTIQCQVDSQVTMMFWYRQQPGQSLTLIATANQGSEATYESGFVIDKFPISRPNLTFSTLTVSNMSPEDSSIYLCSVAWGQSTNTGELFFGEGSRLTVLEDLKNVFPPEVAVFEPSEAEIZ

SEQ ID NO: 400 - DNA, 363 bp, Homo sapiens
  source 1..363: mol_type = genomic DNA
cctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgcctggggacagagtacgaacaccggggagctgttttttggagaaggctctaggctgaccgtactggaggacctgaaaaacgtgttcccacccgaggtcgctgtgtttgagccatcagaagcagagatctc

SEQ ID NO: 401 - protein, 108 aa, Homo sapiens
CTYDTSESDYYLFWYKQPPSRQMILVIRQEAYKQQNATENRFSVNFQKAAKSFSLKISDSQLGDAAMYFCAYRGKAAGNKLTFGGGTRVLVKPNIQNPDPAVYQLRDZ

SEQ ID NO: 402 - DNA, 324 bp, Homo sapiens
  source 1..324: mol_type = genomic DNA
gctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggggaaaagctgcaggcaacaagctaacttttggaggaggaaccagggtgctagttaaaccaaatatccagaaccctgaccctgccgtgtaccagctgagagact

SEQ ID NO: 403 - protein, 116 aa, Homo sapiens
LQCAQDMNHEYMSWYRQDPGMGLRLIHYSVGAGITDQGEVPNGYNVSRSTTEDFPLRLLSAAPSQTSVYFCASSYEGTGGYEQYFGPGTRLTVTEDLKNVFPPEVAVFEPSEAEIZ

SEQ ID NO: 404 - DNA, 348 bp, Homo sapiens
  source 1..348: mol_type = genomic DNA
actgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgaagggacagggggctacgagcagtacttcgggccgggcaccaggctcacggtcacagaggacctgaaaaacgtgttcccacccgaggtcgctgtgtttgagccatcagaagcagagatctc

SEQ ID NO: 405 - protein, 106 aa, Homo sapiens
CNSSSVFSSLQWYRQEPGEGPVLLVTVVTGGEVKKLKRLTFQFGDARKDSSLHITAAQTGDTGLYLCAGVGGGTSYGKLTFGQGTILTVHPNIQNPDPAVYQLRDZ

SEQ ID NO: 406 - DNA, 318 bp, Homo sapiens
  source 1..318: mol_type = genomic DNA
actgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagactggtgatacaggcctctacctctgtgcaggagtaggtggtggtactagctatggaaagctgacatttggacaagggaccatcttgactgtccatccaaatatccagaaccctgaccctgccgtgtaccagctgagagact

SEQ ID NO: 407 - protein, 112 aa, Homo sapiens
CSPRSGDLSVYWYQQSLDQGLQFLIQYYNGEERAKGNILERFSAQQFPDLHSELNLSSLELGDSALYFCASSVGARDTQYFGPGTRLTVLEDLKNVFPPEVAVFEPSEAEIZ

SEQ ID NO: 408 - DNA, 337 bp, Homo sapiens
  source 1..337: mol_type = genomic DNA
gatgctcccctaggtctggagacctctctgtgtactggtaccaacagagcctggaccagggcctccaattcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtgggggccagagatacgcagtattttggcccaggcacccggctgacagtgctcgaggacctgaaaaacgtgttcccacccgaggtcgctgtgtttgagccatcagaagcagagatctc

SEQ ID NO: 409 - protein, 114 aa, Homo sapiens
LLCAQDMNHEYMYWYRQDPGMGLRLIHYSVGEGTTAKGEVPDGYNVSRLKKQNFLLGLESAAPSQTSVYFCASSWDRGIDYTFGSGTRLTVVEDLNKVFPPEVAVFEPSEAEIZ

SEQ ID NO: 410 - DNA, 342 bp, Homo sapiens
  source 1..342: mol_type = genomic DNA
actgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagttgggacaggggaattgactacaccttcggttcggggaccaggttaaccgttgtagaggacctgaacaaggtgttcccacccgaggtcgctgtgtttgagccatcagaagcagagatctc

SEQ ID NO: 411 - protein, 101 aa, Homo sapiens
CTYQTSGFNGLFWYQQHAGEAPTFLSYNVLDGLEEKGRFSSFLSRSKGYSYLLLKELQMKDSASYLCAVGVVDMRFGAGTRLTVKPNIQNPDPAVYQLRDZ

SEQ ID NO: 412 - DNA, 303 bp, Homo sapiens
  source 1..303: mol_type = genomic DNA
actgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgggagtcgtagacatgcgctttggagcagggaccagactgacagtaaaaccaaatatccagaaccctgaccctgccgtgtaccagctgagagact

SEQ ID NO: 413 - protein, 106 aa, Homo sapiens
CSYKTSINNLQWYRQNSGRGLVHLILIRSNEREKHSGRLRVTLDTSKKSSSLLITASRAADTASYFCATALASGGSYIPTFGRGTSLIVHPYIQNPDPAVYQLRDZ

SEQ ID NO: 414 - DNA, 318 bp, Homo sapiens
  source 1..318: mol_type = genomic DNA
actgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggcactggcgtcaggaggaagctacatacctacatttggaagaggaaccagccttattgttcatccgtatatccagaaccctgaccctgccgtgtaccagctgagagact

SEQ ID NO: 415 - protein, 104 aa, Homo sapiens
CSFTDSAIYNLQWFRQDPGKGLTSLLLIQSSQREQTSGRLNASLDKSSGRSTLYIAASQPGDSATYLCSPQGGSEKLVFGKGTKLTVNPYIQNPDPAVYQLRDZ

SEQ ID NO: 416 - DNA, 312 bp, Homo sapiens
  source 1..312: mol_type = genomic DNA
actgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtagtccccagggcggatctgaaaagctggtctttggaaagggaacgaaactgacagtaaacccatatatccagaaccctgaccctgccgtgtaccagctgagagact

SEQ ID NO: 417 - protein, 108 aa, Homo sapiens
TCSFPSSNFYALHWYRWETAKSPEALFVMTLNGDEKKKGRISATLNTKEGYSYLYIKGSQPEDSATYLCAFNAGGTSYGKLTFGQGTILTVHPNIQKPDPAVYQLRDZ

SEQ ID NO: 418 - DNA, 323 bp, Homo sapiens
  source 1..323: mol_type = genomic DNA
cacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctttaatgctggtggtactagctatggaaagctgacatttggacaagggaccatcttgactgtccatccaaatatccagaagcctgaccctgccgtgtaccagctgagagact

SEQ ID NO: 419 - protein, 106 aa, Homo sapiens
KCTYSDSASNYFPWYKQELRKRPQLIIDIRSNVGEKKDQRIAVTLNKTAKHFSLHITETQPEDSAVYFCAALEGGGNKLTFGTGTQLKVELNIQKPDPAVYQLRDZ

SEQ ID NO: 420 - DNA, 317 bp, Homo sapiens
  source 1..317: mol_type = genomic DNA
caagtgtacttattcagacagtgcctcaaactacttcccttggtataagcaagaacttagaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcacttgagggaggaggaaacaaactcacctttgggacaggcactcagctaaaagtggaactcaatatccagaagcctgaccctgccgtgtaccagctgagagact

SEQ ID NO: 421 - protein, 113 aa, Homo sapiens
LMCHQTWSHSYMFWYRQDLGHGLRLIYYSAAADITDKGEVPDGYVVSRSKTENFPLTLESATRSQTSVYFCASSEWIYEQYFGPGTRLTVTEDLKNVFPPEVAVFEPSEAEIZ

SEQ ID NO: 422 - DNA, 340 bp, Homo sapiens
  source 1..340: mol_type = genomic DNA
ccttgatgtgtcaccagacttggagccatagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgaatggatctacgagcagtacttcgggccgggcaccaggctcacggtcacagaggacctgaaaaacgtgttcccacccgaggtcgctgtgtttgagccatcagaagcagagatctc

SEQ ID NO: 423 - protein, 116 aa, Homo sapiens
RCKPISGHNSLFWYRQTMMRGLELLIYFNNNVPIDDSGMPEDRFSAKMPNASFSTLKIQPSEPRDSAVYFCASSLTSGSLQTQYFGPGTRLLVLEDLKNVFPPEVAVFEPSEAEIZ

SEQ ID NO: 424 - DNA, 348 bp, Homo sapiens
  source 1..348: mol_type = genomic DNA
gagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttaactagcgggagtctacagacccagtacttcgggccaggcacgcggctcctggtgctcgaggacctgaaaaacgtgttcccacccgaggtcgctgtgtttgagccatcagaagcagagatctc

SEQ ID NO: 425 - protein, 119 aa, Homo sapiens
ECRSLDFQATTMFWYRQFPKQSLMLMATSNEGSKATYEQGVEKDKFLINHASLTLSTLTVTSAHPEDSSFYICSAEGAGAGGNYGYTFGSGTRLTVVEDLNKVFPPEVAVFEPSEAEIZ

SEQ ID NO: 426 - DNA, 357 bp, Homo sapiens
  source 1..357: mol_type = genomic DNA
cgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctgagggggccggggcagggggcaactatggctacaccttcggttcggggaccaggttaaccgttgtagaggacctgaacaaggtgttcccacccgaggtcgctgtgtttgagccatcagaagcagagatctc

SEQ ID NO: 427 - protein, 116 aa, Homo sapiens
VTLSCSPISGHRSVSWYQQTPGQGLQFLFEYFSETQRNKGNFPGRFSGRQFSNSRSEMNVSTLELGDSALYLCASSRDGNTEAFFGQGTRLTVVEDLNKVFPPEVAVFEPSEAEIZ

SEQ ID NO: 428 - DNA, 347 bp, Homo sapiens
  source 1..347: mol_type = genomic DNA
gtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccgggacggaaacactgaagctttctttggacaaggcaccagactcacagttgtagaggacctgaacaaggtgttcccacccgaggtcgctgtgtttgagccatcagaagcagagatctc

SEQ ID NO: 429 - protein, 118 aa, Homo sapiens
LTIQCQVDSQVTMMFWYRQQPGQSLTLIATANQGSEATYESGFVIDKFPISRPNLTFSTLTVSNMSPEDSSIYLCSVETLAGSEQFFGPGTRLTVLEDLKNVFPPEVAVFEPSEAEIZ

SEQ ID NO: 430 - DNA, 354 bp, Homo sapiens
  source 1..354: mol_type = genomic DNA
cctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgagacactagcgggaagcgagcagttcttcgggccagggacacggctcaccgtgctagaggacctgaaaaacgtgttcccacccgaggtcgctgtgtttgagccatcagaagcagagatctc

SEQ ID NO: 431 - protein, 115 aa, Homo sapiens
ECRSLDFQATTMFWYRQFPKQSLMLMATSNEGSKATYEQGVEKDKFLINHASLTLSTLTVTSAHPEDSSFYICSASGTHNEQFFGPGTRLTVLEDLKNVFPPEVAVFEPSEAEIZ

SEQ ID NO: 432 - DNA, 345 bp, Homo sapiens
  source 1..345: mol_type = genomic DNA
cgagtgccgttccctggactttcaggccacaactatgttttggtatcgtcagttcccgaaacagagtctcatgctgatggcaacttccaatgagggctccaaggccacatacgagcaaggcgtcgaaaaggacaagtttctcatcaaccatgcaagcctgaccttgtccactctgacagtgaccagtgcccatcctgaagacagcagcttctacatctgcagtgcttccgggacccacaatgagcagttcttcgggccagggacacggctcaccgtgctagaggacctgaaaaacgtgttcccacccgaggtcgctgtgtttgagccatcagaagcagagatctc

SEQ ID NO: 433 - protein, 115 aa, Homo sapiens
RCHQTENHRYMYWYRQDPGHGLRLIHYSYGVKDTDKGEVSDGYSVSRSKTEDFLLTLESATSSQTSVYFCAISEAQDNQETQYFGPGTRLLVLEDLKNVFPPEVAVFEPSEAEIZ

SEQ ID NO: 434 - DNA, 345 bp, Homo sapiens
  source 1..345: mol_type = genomic DNA
gagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgaggcgcaggacaaccaagagacccagtacttcgggccaggcacgcggctcctggtgctcgaggacctgaaaaacgtgttcccacccgaggtcgctgtgtttgagccatcagaagcagagatctc

SEQ ID NO: 435 - protein, 119 aa, Homo sapiens
ECRSLDFQATTMFWYRQFPKQSLMLMATSNEGSKATYEQGVEKDKFLINHASLTLSTLTVTSAHPEDSSFYICSARDASGQGNYGYTFGSGTRLTVVEDLNKVFPPEVAVFEPSEAEIZ

SEQ ID NO: 436 - DNA, 357 bp, Homo sapiens
  source 1..357: mol_type = genomic DNA
cgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagatgcttcgggacaggggaactatggctacaccttcggttcggggaccaggttaaccgttgtagaggacctgaacaaggtgttcccacccgaggtcgctgtgtttgagccatcagaagcagagatctc

SEQ ID NO: 437 - protein, 118 aa, Homo sapiens
ECRSLDFQATTMFWYRQFPKQSLMLMATSNEGSKATYEQGVEKDKFLINHASLTLSTLTVTSAHPEDSSFYICSASGAGIEYNEQFFGPGTRLTVLEDLKNVFPPEVAVFEPSEAEIZ

SEQ ID NO: 438 - DNA, 354 bp, Homo sapiens
  source 1..354: mol_type = genomic DNA
cgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagtggagcgggtatagagtacaatgagcagttcttcgggccagggacacggctcaccgtgctagaggacctgaaaaacgtgttcccacccgaggtcgctgtgtttgagccatcagaagcagagatctc

SEQ ID NO: 439 - protein, 116 aa, Homo sapiens
ECRSLDFQATTMFWYRQFPKQSLMLMATSNEGSKATYEQGVEKDKFLINHASLTLSTLTVTSAHPEDSSFYICSARDLNSQTQYFGPGTRLTVLEDLKNVFPPEVAVFEPSEAEIZ

SEQ ID NO: 440 - DNA, 348 bp, Homo sapiens
  source 1..348: mol_type = genomic DNA
cgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagatcttaactcccagacgcagtattttggcccaggcacccggctgacagtgctcgaggacctgaaaaacgtgttcccacccgaggtcgctgtgtttgagccatcagaagcagagatctc

SEQ ID NO: 441 - protein, 118 aa, Homo sapiens
RCKPISGHDYLFWYRQTMMRGLELLIYFNNNVPIDDSGMPEDRFSAKMPNASFSTLKIQPSEPRDSAVYFCASSLSGGGSYTGELFFGEGSRLTVLEDLKNVFPPEVAVFEPSEAEIZ

SEQ ID NO: 442 - DNA, 354 bp, Homo sapiens
  source 1..354: mol_type = genomic DNA
gagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagagactcagccgtgtacttctgtgccagcagtttaagcggtggtggcagctacaccggggagctgttttttggagaaggctctaggctgaccgtactggaggacctgaaaaacgtgttcccacccgaggtcgctgtgtttgagccatcagaagcagagatctc

SEQ ID NO: 443 - protein, 104 aa, Homo sapiens
CSYKTSINNLQWYRQNSGRGLVHLILIRSNEREKHSGRLRVTLDTSKKSSSLLITASRAADTASYFCATDEDSNYQLIWGAGTKLIIKPDIQNPDPAVYQLRDZ

SEQ ID NO: 444 - DNA, 312 bp, Homo sapiens
  source 1..312: mol_type = genomic DNA
cctgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgaggatagcaactatcagttaatctggggcgctgggaccaagctaattataaagccagatatccagaaccctgaccctgccgtgtaccagctgagagact

SEQ ID NO: 445 - protein, 104 aa, Homo sapiens; identical to SEQ ID NO: 415

SEQ ID NO: 446 - DNA, 312 bp, Homo sapiens; identical to SEQ ID NO: 416
  source 1..312: mol_type = genomic DNA

SEQ ID NO: 447 - protein, 106 aa, Homo sapiens
KCTYSDSASNYFPWYKQELGKRPQLIIDIRSNVGEKKDQRIAVTLNKTAKHFSLHITETQPEDSAVYFCAALEGGGNKLTFGTGTQLKVELNIQKPDPAVYQLRDZ

SEQ ID NO: 448 - DNA, 317 bp, Homo sapiens
  source 1..317: mol_type = genomic DNA
caagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcacttgagggaggaggaaacaaactcacctttgggacaggcactcagctaaaagtggaactcaatatccagaagcctgaccctgccgtgtaccagctgagagact

SEQ ID NO: 449 - protein, 108 aa, Homo sapiens; identical to SEQ ID NO: 417

SEQ ID NO: 450 - DNA, 323 bp, Homo sapiens; identical to SEQ ID NO: 418
  source 1..323: mol_type = genomic DNA

SEQ ID NO: 451 - protein, 114 aa, Homo sapiens
CSPRSGDLSVYWYQQSLDQGLQFLIQYYNGEERAKGNILERFSAQQFPDLHSELNLSSLELGDSALYFCASSVGGGSDNEQFFGPGTRLTVLEDLKNVFPPEVAVFEPSEAEIZ

SEQ ID NO: 452 - DNA, 343 bp, Homo sapiens
  source 1..343: mol_type = genomic DNA
gatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtagggggcgggagtgataatgagcagttcttcgggccagggacacggctcaccgtgctagaggacctgaaaaacgtgttcccacccgaggtcgctgtgtttgagccatcagaagcagagatctc

SEQ ID NO: 453 - protein, 116 aa, Homo sapiens
LQCAQDMNHEYMSWYRQDPGMGLRLIHYSVGAGITDQGEVPNGYNVSRSTTEDFPLRLLSAAPSQTSVYFCASSYGRVPTYEQYFGPGTRLTVTEDLKNVFPPEVAVFEPSEAEIZ

SEQ ID NO: 454 - DNA, 348 bp, Homo sapiens
  source 1..348: mol_type = genomic DNA
actgcagtgtgcccaggatatgaaccatgaatacatgtcctggtatcggcaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagctacgggcgggtcccaacctacgagcagtacttcgggccgggcaccaggctcacggtcacagaggacctgaaaaacgtgttcccacccgaggtcgctgtgtttgagccatcagaagcagagatctc

SEQ ID NO: 455 - protein, 118 aa, Homo sapiens; identical to SEQ ID NO: 437

SEQ ID NO: 456 - DNA, 354 bp, Homo sapiens; identical to SEQ ID NO: 438
  source 1..354: mol_type = genomic DNA

SEQ ID NO: 457 - protein, 118 aa, Homo sapiens; identical to SEQ ID NO: 441

SEQ ID NO: 458 - DNA, 354 bp, Homo sapiens
  source 1..354: mol_type = genomic DNA
gagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttaagcggtggtggcagctacaccggggagctgttttttggagaaggctctaggctgaccgtactggaggacctgaaaaacgtgttcccacccgaggtcgctgtgtttgagccatcagaagcagagatctc

SEQ ID NO: 459 - protein, 119 aa, Homo sapiens; identical to SEQ ID NO: 425

SEQ ID NO: 460 - DNA, 357 bp, Homo sapiens; identical to SEQ ID NO: 426
  source 1..357: mol_type = genomic DNA

SEQ ID NO: 461 - protein, 114 aa, Homo sapiens
CHQTENHRYMYWYRQDPGHGLRLIHYSYGVKDTDKGEVSDGYSVSRSKTEDFLLTLESATSSQTSVYFCAISEAQDNQETQYFGPGTRLLVLEDLKNVFPPEVAVFEPSEAEIZ

SEQ ID NO: 462 - DNA, 343 bp, Homo sapiens
  source 1..343: mol_type = genomic DNA
gatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgaggcgcaggacaaccaagagacccagtacttcgggccaggcacgcggctcctggtgctcgaggacctgaaaaacgtgttcccacccgaggtcgctgtgtttgagccatcagaagcagagatctc

SEQ ID NO: 463 - protein, 119 aa, Homo sapiens; identical to SEQ ID NO: 435

SEQ ID NO: 464 - DNA, 357 bp, Homo sapiens; identical to SEQ ID NO: 436
  source 1..357: mol_type = genomic DNA

SEQ ID NO: 465 - protein, 115 aa, Homo sapiens; identical to SEQ ID NO: 431

SEQ ID NO: 466 - DNA, 345 bp, Homo sapiens
  source 1..345: mol_type = genomic DNA
cgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgcttccgggacccacaatgagcagttcttcgggccagggacacggctcaccgtgctagaggacctgaaaaacgtgttcccacccgaggtcgctgtgtttgagccatcagaagcagagatctc

SEQ ID NO: 467 - protein, 112 aa, Homo sapiens
CSPISGHRSVSWYQRTPGQGLQFLFEYFSETQRNKGNFPGRFSGRQFSNSRSEMNVSTLELGDSALYLCASSRDGNTEAFFGQGTRLTVVEDLNKVFPPEVAVFEPSEAEIZ

SEQ ID NO: 468 - DNA, 337 bp, Homo sapiens
  source 1..337: mol_type = genomic DNA
gatgctcccctatctctgggcataggagtgtatcctggtaccaacggaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccgggacggaaacactgaagctttctttggacaaggcaccagactcacagttgtagaggacctgaacaaggtgttcccacccgaggtcgctgtgtttgagccatcagaagcagagatctc

SEQ ID NO: 469 - protein, 13 aa, Homo sapiens
CSARDLNSQTQYF

SEQ ID NO: 470 - DNA, 33 bp, Homo sapiens
  source 1..33: mol_type = genomic DNA
agtgctagagatcttaactcccagacgcagtat

SEQ ID NO: 471 - protein, 116 aa, Homo sapiens; identical to SEQ ID NO: 439

SEQ ID NO: 472 - DNA, 348 bp, Homo sapiens; identical to SEQ ID NO: 440
  source 1..348: mol_type = genomic DNA

SEQ ID NO: 473 - protein, 118 aa, Homo sapiens; identical to SEQ ID NO: 429

SEQ ID NO: 474 - DNA, 354 bp, Homo sapiens; identical to SEQ ID NO: 430
  source 1..354: mol_type = genomic DNA

SEQ ID NO: 475 - protein, 108 aa, Homo sapiens
YSSSYSPSLFWYVQHPNKGLQLLLKYTSAATLVKGINGFEAEFKKSETSFHLTKPSAHMSDAAEYFCVVSDNAGGTSYGKLTFGQGTILTVHPNIQKPNPAVYQLRDZ

SEQ ID NO: 476 - DNA, 322 bp, Homo sapiens
  source 1..322: mol_type = genomic DNA
tactcatcttcttattcaccgtctctcttctggtatgtgcaacaccccaacaaaggactccagcttctcctgaagtacacatcagcggccaccctggttaaaggtatcaacggttttgaggctgaatttaagaagagtgaaacctccttccacctgacgaaaccctcagcccatatgagcgacgcggctgagtacttctgtgttgtgagtgataatgctggtggtactagctatggaaagctgacatttggacaagggaccatcttgactgtccatccaaatatccagaagcctaaccctgccgtgtaccagctgagagact

SEQ ID NO: 477 - protein, 117 aa, Homo sapiens
LTIQCQVDSQVTMMFWYRQQPGQSLTLIATANQGSEATYESGFVIDKFPISRPNLTFSTLTVSNMSPEDSSIYLCSVAGSESEQFFGPGTRLTVLEDLKNVFPPEVAVFEPSEAEIZ

SEQ ID NO: 478 - DNA, 351 bp, Homo sapiens
  source 1..351: mol_type = genomic DNA
cctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgcggggagcgagagcgagcagttcttcgggccagggacacggctcaccgtgctagaggacctgaaaaacgtgttcccacccgaggtcgctgtgtttgagccatcagaagcagagatctc

SEQ ID NO: 479 - protein, 108 aa, Homo sapiens
CTYDTSESDYYLFWYKQPPSRQMILVIRQEAYKQQNATENRFSVNFQKAAKSFSLKISDSQLGDAAMYFCAYRRRAAGNKLTFGGGTRVLVKPNIQNPDPAVYQLRDZ

SEQ ID NO: 480 - DNA, 324 bp, Homo sapiens
  source 1..324: mol_type = genomic DNA
gctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagaagagctgcaggcaacaagctaacttttggaggaggaaccagggtgctagttaaaccaaatatccagaaccctgaccctgccgtgtaccagctgagagact

SEQ ID NO: 481 - protein, 115 aa, Homo sapiens
CSPRSGDLSVYWYQQSLDQGLQFLIQYYNGEERAKGNILERFSAQQFPDLHSELNLSSLELGDSALYFCASSVLERLPYNEQFFGPGTRLTVLEDLKNMFPPEVAVFEPSEAEIZ

SEQ ID NO: 482 - DNA, 346 bp, Homo sapiens
  source 1..346: mol_type = genomic DNA
gatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtattggagcggctaccttacaatgagcagttcttcgggccagggacacggctcaccgtgctagaggacctgaaaaacatgttcccacccgaggtcgctgtgtttgagccatcagaagcagagatctc

SEQ ID NO: 483 - protein, 115 aa, Homo sapiens
RCKPISGHNSLFWYRQTMMRGLELLIYFNNNVPIDDSGMPEDRFSAKMPNASFSTLKIQPSEPRDSAVYFCASSLDGVGGEQFFGPGTRLTVLEDLKNVFPPEVAVFEPSEAEIZ

SEQ ID NO: 484 - DNA, 345 bp, Homo sapiens
  source 1..345: mol_type = genomic DNA
gagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagcctagatggggtgggcggtgagcagttcttcgggccagggacacggctcaccgtgctagaggacctgaaaaacgtgttcccacccgaggtcgctgtgtttgagccatcagaagcagagatctc

SEQ ID NO: 485 - protein, 106 aa, Homo sapiens
CNYTNYSPAYLQWYQQDPGRGPVFLLLIRENEKEKRKERLKVTFDTTLKQSLFHITASQPADSATYLCALDHTGTASKLTFGTGTRLQVTLDIQNPDPAVYQLRDZ

SEQ ID NO: 486 - DNA, 318 bp, Homo sapiens
  source 1..318: mol_type = genomic DNA
cctgcaactatacaaactattccccagcatacttacagtggtaccaacaagatccaggaagaggccctgttttcttgctactcatacgtgaaaatgagaaagaaaaaaggaaagaaagactgaaggtcacctttgataccacccttaaacagagtttgtttcatatcacagcctcccagcctgcagactcagctacctacctctgtgctctagatcataccggcactgccagtaaactcacctttgggactggaacaagacttcaggtcacgctcgatatccagaaccctgaccctgccgtgtaccagctgagagact

SEQ ID NO: 487 - protein, 106 aa, Homo sapiens
CNHSTISGTDYIHWYRQLPSQGPEYVIHGLTSNVNNRMASLAIAEDRKSSTLILHRATLRDAAVYYCISGTGGTSYGKLTSGQGTILTVHPNIQNPDPAVYQLRDZ

SEQ ID NO: 488 - DNA, 318 bp, Homo sapiens
  source 1..318: mol_type = genomic DNA
cttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatctctgggactggtggtactagctatggaaagctgacatctggacaagggaccatcttgactgtccatccaaatatccagaaccctgaccctgccgtgtaccagctgagagact

SEQ ID NO: 489 - protein, 111 aa, Homo sapiens
CTYSVSGNPYLFWYVQYPNRGLQFLLKYITGDNLVKGSYGFEAEFNKSQTSFHLKKPSALVSDSALYFCAVRDMGLAGGGADGLTFGKGTHLIIQPYIQNPDPAVYQLRDZ

SEQ ID NO: 490 - DNA, 333 bp, Homo sapiens
  source 1..333: mol_type = genomic DNA
aatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacatgggactggcaggaggaggtgctgacggactcacctttggcaaagggactcatctaatcatccagccctatatccagaaccctgaccctgccgtataccagctgagagact

SEQ ID NO: 491 - protein, 119 aa, Homo sapiens
LTIQCQVDSQVTMMFWYRQQPGQSLTLIATANQGSEATYESGFVIDKFPISRPNLTFSTLTVSNMSPEDSSIYLCSVVRQGGQETQYFGPGTRLLVLEDLKNVFPPEVAVFEPSEAEIZ

SEQ ID NO: 492 - DNA, 357 bp, Homo sapiens
  source 1..357: mol_type = genomic DNA
cctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgttaggcaggggggtcaagagacccagtacttcgggccaggcacgcggctcctggtgctcgaggacctgaaaaacgtgttcccacccgaggtcgctgtgtttgagccatcagaagcagagatctc

SEQ ID NO: 493 - protein, 114 aa, Homo sapiens
LQCAQDMNHNYMYWYRQDPGMGLKLIYYSVGAGITDKGEVPNGYNVSRSTTEYFPLRLELAAPSQTSVYFCASSREGYNEQFFGPGTRLTVLEDLKNVFPPEVAVFEPSEAEIZ

SEQ ID NO: 494 - DNA, 342 bp, Homo sapiens
  source 1..342: mol_type = genomic DNA
actgcagtgtgcccaggatatgaaccataactacatgtactggtatcgacaagacccaggcatggggctgaagctgatttattattcagttggtgctggtatcactgataaaggagaagtcccgaatggctacaacgtctccagatcaaccacagagtatttcccgctcaggctggagttggctgctccctcccagacatctgtgtacttctgtgccagcagtagggaagggtacaatgagcagttcttcgggccagggacacggctcaccgtgctagaggacctgaaaaacgtgttcccacccgaggtcgctgtgtttgagccatcagaagcagagatctc

SEQ ID NO: 495 - protein, 115 aa, Homo sapiens
RCKPISGHNSLFWYRQTMMRGLELLIYFNNNVPIDDSGMPEDRFSAKMPNTSFSTLKIQPSEPRDSAVYFCASSLDGVGGEQFFGPGTRLTVLEDLKNVFPPEVAVFEPSEAEIZ

SEQ ID NO: 496 - DNA, 345 bp, Homo sapiens
  source 1..345: mol_type = genomic DNA
gagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatacatcattctccactctgaagatccagccctcagaacccagggactcagctgtgtacttctgtgccagcagcctagatggggtgggcggtgagcagttcttcgggccagggacacggctcaccgtgctagaggacctgaaaaacgtgttcccacccgaggtcgctgtgtttgagccatcagaagcagagatctc

SEQ ID NO: 497 - protein, 111 aa, Homo sapiens
CTYSVSGNPYLFWYVQYPNRGLQFLLKYITGDNLVKGSYGFEAEFNKSQTSFRLKKPSALVSDSALYFCAVRDMSLAGGGADGLTFGKGTHLIIQPYIQNPDPAVYQLRDZ

SEQ ID NO: 498 - DNA, 333 bp, Homo sapiens
  source 1..333: mol_type = genomic DNA
aatgcacctattcagtctctggaaacccttatcttttttggtatgttcaataccccaaccgaggcctccagttccttctgaaatacatcacaggggataacctggttaaaggcagctatggctttgaggctgaatttaacaagagccaaacctccttccgcctgaagaaaccatctgcccttgtgagcgactccgctttgtacttctgtgctgtgagagacatgagtctggcaggaggaggtgctgacggactcacctttggcaaagggactcatctaatcatccagccctatatccagaaccctgaccctgccgtgtaccagctgagagact

SEQ ID NO: 499 - protein, 106 aa, Homo sapiens
NCAYENTAFDYFPWYQQSPGKGPALLIAIRPDVSEKKEGRFTISFNKSAKQFSLHIMDSQPGDSATYFCAAYGFGNEKLTFGTGTRLTIIPNIQNPDPAVYQLRDZ

SEQ ID NO: 500 - DNA, 317 bp, Homo sapiens
  source 1..317: mol_type = genomic DNA
aaactgtgcttatgagaacactgcgtttgactactttccatggtaccaacaatcccctgggaagggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagcatatggctttggaaatgagaaattaacctttgggactggaacaagactcaccatcatacccaatatccagaaccctgaccctgccgtgtaccagctgagagact

SEQ ID NO: 501 - protein, 114 aa, Homo sapiens
VTLRCSPKSWHDTVSWYQQALGQGPQFIFQYYEEEERQRGNFPDRFSGHQFPNYSSELNVNALLLGDSALYLCASSRNYEQYFGPGTRLTVTEDLKNVFPPEVAVFEPSEAEIZ

SEQ ID NO: 502 - DNA, 341 bp, Homo sapiens
  source 1..341: mol_type = genomic DNA
gtgactctgagatgctctcctaagtcttggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagccgtaactacgagcagtacttcgggccgggcaccaggctcacggtcacagaggacctgaaaaacgtgttcccacccgaggtcgctgtgtttgagccatcagaagcagagatctc

SEQ ID NO: 503 - protein, 111 aa, Homo sapiens
PISGHTALYWYRQSLGQGPEFLIYFQGTGAADDSGLPNDRFFAVRPEGSVSTLKIQRTERGDSAVYLCASSFGGGNEQYFGPGTRLTVTEDLKNVFPPEVAVFEPSEAEIZ

SEQ ID NO: 504 - DNA, 332 bp, Homo sapiens
  source 1..332: mol_type = genomic DNA
ccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcgtggggactcagccgtgtatctctgtgccagcagctttgggggtggtaatgagcagtacttcgggccgggcaccaggctcacggtcacagaggacctgaaaaacgtgttcccacccgaggtcgctgtgtttgagccatcagaagcagagatctc

SEQ ID NO: 505 - protein, 106 aa, Homo sapiens
CNHSTISGNEYVYWYRQIHSQGPQYIIHGLKNNETNEMASLIITEDRKSSTLILPHATLRDTAVYYCIVRVWGAAGNKLTFGGGTRVLVKPNIQNPDPAVYQLRDZ

SEQ ID NO: 506 - DNA, 318 bp, Homo sapiens
  source 1..318: mol_type = genomic DNA
cttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagagtctggggagctgcaggcaacaagctaacttttggaggaggaaccagggtgctagttaaaccaaatatccagaaccctgaccctgccgtgtaccagctgagagact

SEQ ID NO: 507 - protein, 106 aa, Homo sapiens
NCAYENTAFDYFPWYQQFPGKGPALLIAIRPDVSERKEGRFTISFNKSAKQFSLHIMDSQPGDSATYFCAAYGFGNEKLTFGTGTRLTIIPNIQNPDPAVYQLRDZ

SEQ ID NO: 508 - DNA, 317 bp, Homo sapiens
  source 1..317: mol_type = genomic DNA
aaactgtgcttatgagaacactgcgtttgactactttccatggtaccaacaattccctgggaaaggccctgcattattgatagccatacgtccagatgtgagtgaaaggaaagaaggaagattcacaatctccttcaataaaagtgccaagcagttctcattgcatatcatggattcccagcctggagactcagccacctacttctgtgcagcatatggctttggaaatgagaaattaacctttgggactggaacaagactcaccatcatacccaatatccagaaccctgaccctgccgtgtaccagctgagagact

SEQ ID NO: 509 - protein, 116 aa, Homo sapiens
VTLSCSPISGHRSVSWYQQTPGQGLQFLFEYFSETQRNKGNFPGRFSGRQFSNSRSEMNVSTLGLGDSALYLCASSLAPEETQYFGPGTRLLVLEDLKNVFPPEVAVFEPSEAEIZ

SEQ ID NO: 510 - DNA, 347 bp, Homo sapiens
  source 1..347: mol_type = genomic DNA
gtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggggctgggggactcggccctttatctttgcgccagcagcttggccccggaagagacccagtacttcgggccaggcacgcggctcctggtgctcgaggacctgaaaaacgtgttcccacccgaggtcgctgtgtttgagccatcagaagcagagatctc

SEQ ID NO: 511 - protein, 111 aa, Homo sapiens; identical to SEQ ID NO: 503

SEQ ID NO: 512 - DNA, 332 bp, Homo sapiens
  source 1..332: mol_type = genomic DNA
ccaatttcaggtcatactgccctttactggtaccgacaaagcctggggcagggcccagagtttctaatttacttccaaggcacgggggcggcagatgactcagggctgcccaacgatcggttctttgcagtcaggcctgagggatccgtctctactctgaagatccagcgcacagagcggggggactcagccgtgtatctctgtgccagcagctttgggggtggtaatgagcagtacttcgggccgggcaccaggctcacggtcacagaggacctgaaaaacgtgttcccacccgaggtcgctgtgtttgagccatcagaagcagagatctc

SEQ ID NO: 513 - protein, 112 aa, Homo sapiens
NCTYSVSGNPYLFWYVQYPNRGLQFLLKYITGDNLVKGSYGFEAEFNKSQTSFHLKKPSALVSDSALYFCAVRDMRQAGGGADGLTFGRGTHLIIQPYIQNPDPAVYQLRDZ

SEQ ID NO: 514 - DNA, 335 bp, Homo sapiens
  source 1..335: mol_type = genomic DNA
gaactgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacatgagacaggcaggaggaggtgctgacggactcacctttggcagagggactcatctaatcatccagccctatatccagaaccctgaccctgccgtgtaccagctgagagact

SEQ ID NO: 515 - protein, 115 aa, Homo sapiens
ECRSLDFQATTMFWYRQFPKQSLMLMATSNEGSKATYEQGVEKDKFLINHASLTLSTLTVTSAHPEDSSFYICSAPGGNNEQFFGPGTRLTVLEDLKNVFPPEVAVFEPSEAEIZ

SEQ ID NO: 516 - DNA, 345 bp, Homo sapiens
  source 1..345: mol_type = genomic DNA
cgagtgccgttccctggactttcaggccacaactatgttttggtatcgtcagttcccgaaacagagtctcatgctgatggcaacttccaatgagggctccaaggccacatacgaacaaggcgtcgagaaggacaagtttctcatcaaccatgcgagcctgaccttgtccactctgacagtgaccagtgcccatcctgaagacagcagcttctacatctgcagtgctcccgggggaaacaatgagcagttcttcgggccagggacacggctcaccgtgctagaggacctgaaaaacgtgttcccacccgaggtcgctgtgtttgagccatcagaagcagagatctc

SEQ ID NO: 517 - protein, 17 aa, synthetic construct
  REGION 1..17: note = Homo sapiens KRAS EnsemblID ENST00000311936.8 positions 4-20
YKLVVVGAGGVGKSALT

SEQ ID NO: 518 - protein, 17 aa, synthetic construct
  REGION 1..17: note = Cancer mutation
YKLVVVGAVGVGKSALT

SEQ ID NO: 519 - protein, 17 aa, synthetic construct
  REGION 1..17: note = Cancer mutation
YKLVVVGADGVGKSALT

SEQ ID NO: 520 - protein, 17 aa, synthetic construct
  REGION 1..17: note = Cancer mutation
YKLVVVGARGVGKSALT

SEQ ID NO: 521 - protein, 17 aa, synthetic construct
  REGION 1..17: note = Cancer mutation
YKLVVVGASGVGKSALT

SEQ ID NO: 522 - protein, 17 aa, synthetic construct
  REGION 1..17: note = Cancer mutation
YKLVVVGACGVGKSALT

SEQ ID NO: 523 - protein, 17 aa, synthetic construct
  REGION 1..17: note = Cancer mutation
YKLVVVGAAGVGKSALT

SEQ ID NO: 524 - protein, 17 aa, synthetic construct
  REGION 1..17: note = Homo sapiens KRAS EnsemblID ENST00000311936.8 positions 5-21
KLVVVGAGGVGKSALTI

SEQ ID NO: 525 - protein, 17 aa, synthetic construct
  REGION 1..17: note = Cancer mutation
KLVVVGAGDVGKSALTI

SEQ ID NO: 526 - protein, 17 aa, synthetic construct
  REGION 1..17: note = Homo sapiens KRAS EnsemblID ENST00000311936.8 positions 53-69
LDILDTAGQEEYSAMRD

SEQ ID NO: 527 - protein, 17 aa, synthetic construct
  REGION 1..17: note = Cancer mutation
LDILDTAGKEEYSAMRD

SEQ ID NO: 528 - protein, 17 aa, synthetic construct
  REGION 1..17: note = Cancer mutation
LDILDTAGHEEYSAMRD

SEQ ID NO: 529 - protein, 17 aa, synthetic construct
  REGION 1..17: note = Cancer mutation
LDILDTAGREEYSAMRD

SEQ ID NO: 530 - protein, 17 aa, synthetic construct
  REGION 1..17: note = Homo sapiens KRAS EnsemblID ENST00000256078.10 positions 138-154
GIPFIETSAKTRQRVED

SEQ ID NO: 531 - protein, 17 aa, synthetic construct
  REGION 1..17: note = Cancer mutation
GIPFIETSTKTRQRVED

SEQ ID NO: 532 - protein, 15 aa, synthetic construct
  REGION 1..15: note = Cancer mutation
KLVVVGAVGVGKSAL

SEQ ID NO: 533 - protein, 15 aa, synthetic construct
  REGION 1..15: note = Cancer mutation
LVVVGAGCVGKSALT

SEQ ID NO: 534 - protein, 15 aa, synthetic construct
  REGION 1..15: note = Cancer mutation
LVVVGAGSVGKSALT

SEQ ID NO: 535 - protein, 15 aa, synthetic construct
  REGION 1..15: note = Cancer mutation
LVVVGAGRVGKSALT

SEQ ID NO: 536 - protein, 15 aa, synthetic construct
  REGION 1..15: note = Cancer mutation
LVVVGAGAVGKSALT

SEQ ID NO: 537 - protein, 15 aa, synthetic construct
  REGION 1..15: note = Cancer mutation
LVVVGAGVVGKSALT

SEQ ID NO: 538 - protein, 15 aa, synthetic construct
  REGION 1..15: note = Cancer mutation
DILDTAGLEEYSAMR

SEQ ID NO: 539 - protein, 15 aa, synthetic construct
  REGION 1..15: note = Cancer mutation
PMVLVGNNCDLPSRT

SEQ ID NO: 540 - protein, 15 aa, synthetic construct
  REGION 1..15: note = Cancer mutation
IPFIETSVKTRQRVE

SEQ ID NO: 541 - protein, 15 aa, synthetic construct
  REGION 1..15: note = Cancer mutation
LVVVGAVGVGKSALT

SEQ ID NO: 542 - protein, 15 aa, synthetic construct
  REGION 1..15: note = Homo sapiens MUC4 EnsemblID ENST00000463781.8 positions 3823-3837
TPLPVTDASSASTGH

SEQ ID NO: 543 - protein, 15 aa, synthetic construct
  REGION 1..15: note = Home Sapiens MUC4 EnsemblID ENST00000463781.8 positions 1055-1069
TPLPVTDTSSASTGH

SEQ ID NO: 544 - protein, 15 aa, synthetic construct
  REGION 1..15: note = Homo sapiens MUC4 EnsemblID ENST00000463781.8 positions 1170-1184
PVTSLSSVSTGDTTP

SEQ ID NO: 545 - protein, 15 aa, synthetic construct
  REGION 1..15: note = Homo sapiens MUC4 EnsemblID ENST00000463781.8 positions 1074-1088
PVTSLSSASTGDTTP

SEQ ID NO: 546 - protein, 15 aa, synthetic construct
  REGION 1..15: note = Homo sapiens MUC4 EnsemblID ENST00000463781.8 positions 4142-4156
TTPLPVTIPSSASSG

SEQ ID NO: 547 - protein, 15 aa, synthetic construct
  REGION 1..15: note = Homo sapiens MUC4 EnsemblID ENST00000463781.8 positions 1934-1948
TTPLPVTSPSSASSG

SEQ ID NO: 548 - protein, 15 aa, synthetic construct
  REGION 1..15: note = Homo sapiens MUC4 EnsemblID ENST00000463781.8 positions 4002-4016
PVTSTSSVSTGHATP

SEQ ID NO: 549 - protein, 15 aa, synthetic construct
  REGION 1..15: note = Homo sapiens MUC4 EnsemblID ENST00000463781.8 positions 1666-1680
PVTSTSSASTGHATP

SEQ ID NO: 550 - protein, 15 aa, synthetic construct
  REGION 1..15: note = Cancer mutation
PVTSPSSESTGHAIP

SEQ ID NO: 551 - protein, 15 aa, synthetic construct
  REGION 1..15: note = Homo sapiens MUC4 EnsemblID ENST00000463781.8 positions 2210-2224
PVTSPSSASTGHAIP

SEQ ID NO: 552 - protein, 15 aa, synthetic construct
  REGION 1..15: note = Cancer mutation
TPLPVTDNSSASTGD

SEQ ID NO: 553 - protein, 15 aa, synthetic construct
  REGION 1..15: note = Homo sapiens MUC4 EnsemblID ENST00000463781.8 positions 1615-1629
TPLPVTDTSSASTGD

SEQ ID NO: 554 - protein, 11 aa, synthetic construct
  REGION 1..11: note = Homo sapiens MUC4 EnsemblID ENST00000463781.8 positions 1059-1069
VTDTSSASTGH

SEQ ID NO: 555 - protein, 11 aa, synthetic construct
  REGION 1..11: note = Homo sapiens MUC4 EnsemblID ENST00000463781.8 positions 2307-2317
VTDASSASTGH

SEQ ID NO: 556 - protein, 15 aa, synthetic construct
  REGION 1..15: note = Cancer mutation
VSKRLKFSRDLSLDP

SEQ ID NO: 557 - protein, 15 aa, synthetic construct
  REGION 1..15: note = Cancer mutation
YSHTEKPDVFYQQVL

SEQ ID NO: 558 - protein, 15 aa, synthetic construct
  REGION 1..15: note = Cancer mutation
PVGTIIGVVTAQDLD

SEQ ID NO: 559 - protein, 15 aa, synthetic construct
  REGION 1..15: note = Cancer mutation
IGKGSCNSLVVTSSP

SEQ ID NO: 560 - protein, 15 aa, synthetic construct
  REGION 1..15: note = Cancer mutation
LDRYWAISNAIEYAR

SEQ ID NO: 561 - protein, 15 aa, synthetic construct
  REGION 1..15: note = Cancer mutation
PIAAAEKMAKAQEKH

SEQ ID NO: 562 - protein, 15 aa, synthetic construct
  REGION 1..15: note = Cancer mutation
TLTPLQPQPPISTVS

SEQ ID NO: 563 - protein, 15 aa, synthetic construct
  REGION 1..15: note = Cancer mutation
SMVKLILLWLTAIVL

SEQ ID NO: 564 - protein, 15 aa, Homo sapiens
VSKRLKFPRDLSLDP

SEQ ID NO: 565 - protein, 15 aa, Homo sapiens
YSHTEKPGVFYQQVL

SEQ ID NO: 566 - protein, 15 aa, Homo sapiens
PVGTIIGAVTAQDLD

SEQ ID NO: 567 - protein, 15 aa, Homo sapiens
IGKGSCNNLVVTSSP

SEQ ID NO: 568 - protein, 15 aa, Homo sapiens
LDRYWAITNAIEYAR

SEQ ID NO: 569 - protein, 15 aa, Homo sapiens
PIAAAEKLAKAQEKH

SEQ ID NO: 570 - protein, 15 aa, Homo sapiens
TLTPLQPLPPISTVS

SEQ ID NO: 571 - protein, 15 aa, Homo sapiens
SMVKLILVWLTAIVL

SEQ ID NO: 572 - protein, 15 aa, synthetic construct
  REGION 1..15: note = Cancer mutation
TTNETATSDDGDEVF

SEQ ID NO: 573 - protein, 15 aa, synthetic construct
  REGION 1..15: note = Cancer mutation
EPCRSLYSNLPASRQ

SEQ ID NO: 574 - protein, 15 aa, synthetic construct
  REGION 1..15: note = Cancer mutation
QRCFDIEMQYLNRRR

SEQ ID NO: 575 - protein, 15 aa, synthetic construct
  REGION 1..15: note = Cancer mutation
LHLEEMGMSEEMQAR

SEQ ID NO: 576 - protein, 15 aa, synthetic construct
  REGION 1..15: note = Cancer mutation
FSFVRGQLLFKGCDV

SEQ ID NO: 577 - protein, 15 aa, synthetic construct
  REGION 1..15: note = Cancer mutation
CQCAPCPKNGRPTLS

SEQ ID NO: 578 - protein, 15 aa, synthetic construct
  REGION 1..15: note = Cancer mutation
VQKAELTGLANTFRQ

SEQ ID NO: 579 - protein, 15 aa, synthetic construct
  REGION 1..15: note = Cancer mutation
LGAVISADSIAVGLN

SEQ ID NO: 580 - protein, 15 aa, synthetic construct
  REGION 1..15: note = Cancer mutation
GGAEEAAGHSFRAAE

SEQ ID NO: 581 - protein, 15 aa, synthetic construct
  REGION 1..15: note = Cancer mutation
KSRLYWLASKLHMLS

SEQ ID NO: 582 - protein, 15 aa, synthetic construct
  REGION 1..15: note = Cancer mutation
LLETCQNKLKPGSVL

SEQ ID NO: 583 - protein, 15 aa, synthetic construct
  REGION 1..15: note = Cancer mutation
AELFRKICIQMRALD

SEQ ID NO: 584 - protein, 15 aa, synthetic construct
  REGION 1..15: note = Cancer mutation
LQSQSWKVKAQGAIA

SEQ ID NO: 585 - protein, 15 aa, synthetic construct
  REGION 1..15: note = Cancer mutation
QSCDPHTIQRRTRHL

SEQ ID NO: 586 - protein, 15 aa, synthetic construct
  REGION 1..15: note = Cancer mutation
PVLTLVDKAIVKQIN

SEQ ID NO: 587 - protein, 15 aa, synthetic construct
  REGION 1..15: note = Cancer mutation
SYGAIGVTVGHEFTH

SEQ ID NO: 588 - protein, 15 aa, synthetic construct
  REGION 1..15: note = Cancer mutation
LMLKPILWPLHNSEI

SEQ ID NO: 589 - protein, 15 aa, synthetic construct
  REGION 1..15: note = Cancer mutation
DQLIKCPCRNQPVIV

SEQ ID NO: 590 - protein, 15 aa, synthetic construct
  REGION 1..15: note = Cancer mutation
LPQAIIIRVKKGGTR

SEQ ID NO: 591 - protein, 15 aa, synthetic construct
  REGION 1..15: note = Cancer mutation
TNNPRLSFIHPNAFF

SEQ ID NO: 592 - protein, 15 aa, Homo sapiens
TTNETATGDDGDEVF

SEQ ID NO: 593 - protein, 15 aa, Homo sapiens
EPCRSLYTNLPASRQ

SEQ ID NO: 594 - protein, 15 aa, Homo sapiens
QRCFDIEKQYLNRRR

SEQ ID NO: 595 - protein, 15 aa, Homo sapiens
LHLEEMGVSEEMQAR

SEQ ID NO: 596 - protein, 15 aa, Homo sapiens
FSFVRGQVLFKGCDV

SEQ ID NO: 597 - protein, 15 aa, Homo sapiens
CQCAPCPNNGRPTLS

SEQ ID NO: 598 - protein, 15 aa, Homo sapiens
VQKAELTRLANTFRQ

SEQ ID NO: 599 - protein, 15 aa, Homo sapiens
LGAVISAVSIAVGLN

SEQ ID NO: 600 - protein, 9 aa, synthetic construct
  REGION 1..9: note = Cancer mutation
KLVISPLEL

SEQ ID NO: 601 - protein, 10 aa, synthetic construct
  REGION 1..10: note = Cancer mutation
LMLKPILWPL

SEQ ID NO: 602 - protein, 9 aa, synthetic construct
  REGION 1..9: note = Cancer mutation
ALQSQSWKV

SEQ ID NO: 603 - protein, 10 aa, synthetic construct
  REGION 1..10: note = Cancer mutation
RLLLETCQNK

SEQ ID NO: 604 - protein, 9 aa, synthetic construct
  REGION 1..9: note = Cancer mutation
CPKNGRPTL

SEQ ID NO: 605 - protein, 10 aa, synthetic construct
  REGION 1..10: note = Cancer mutation
RVKKGGTRAL

SEQ ID NO: 606 - protein, 14 aa, synthetic construct
  REGION 1..14: note = Cancer mutation
IEMQYLNRRRQNEI

SEQ ID NO: 607 - protein, 11 aa, synthetic construct
  REGION 1..11: note = Cancer mutation
SDDGDEVFVEA

SEQ ID NO: 608 - protein, 15 aa, synthetic construct
  REGION 1..15: note = Cancer mutation
RSLSYGAIGVTVGHE

SEQ ID NO: 609 - protein, 14 aa, synthetic construct
  REGION 1..14: note = Cancer mutation
KLVISPLELRMEEL

SEQ ID NO: 610 - protein, 28 aa, synthetic construct
  REGION 1..28: note = Cancer mutation
LKDPSIFQVARQLRLIAAKPDQLIKCPC

SEQ ID NO: 611 - protein, 23 aa, synthetic construct
  REGION 1..23: note = Cancer mutation
FVSSMSASAVVLALAKIEIKLSD

SEQ ID NO: 612 - protein, 15 aa, SARS-CoV-2
MFVFLVLLPLVSSQC

SEQ ID NO: 613 - protein, 15 aa, SARS-CoV-2
VNLTTRTQLPPAYTN

SEQ ID NO: 614 - protein, 15 aa, SARS-CoV-2
SFTRGVYYPDKVFRS

SEQ ID NO: 615 - protein, 15 aa, SARS-CoV-2
SVLHSTQDLFLPFFS

SEQ ID NO: 616 - protein, 15 aa, SARS-CoV-2
NVTWFHAIHVSGTNG

SEQ ID NO: 617 - protein, 15 aa, SARS-CoV-2
TKRFDNPVLPFNDGV

SEQ ID NO: 618 - protein, 15 aa, SARS-CoV-2
YFASTEKSNIIRGWI

SEQ ID NO: 619 - protein, 15 aa, SARS-CoV-2
FGTTLDSKTQSLLIV

SEQ ID NO: 620 - protein, 15 aa, SARS-CoV-2
NNATNVVIKVCEFQF

SEQ ID NO: 621 - protein, 15 aa, SARS-CoV-2
CNDPFLGVYYHKNNK

SEQ ID NO: 622 - protein, 15 aa, SARS-CoV-2
SWMESEFRVYSSANN

SEQ ID NO: 623 - protein, 15 aa, SARS-CoV-2
CTFEYVSQPFLMDLE

SEQ ID NO: 624 - protein, 15 aa, SARS-CoV-2
GKQGNFKNLREFVFK

SEQ ID NO: 625 - protein, 15 aa, SARS-CoV-2
NIDGYFKIYSKHTPI

SEQ ID NO: 626 - protein, 15 aa, SARS-CoV-2
NLVRDLPQGFSALEP

SEQ ID NO: 627 - protein, 15 aa, SARS-CoV-2
LVDLPIGINITRFQT

SEQ ID NO: 628 - protein, 15 aa, SARS-CoV-2
LLALHRSYLTPGDSS

SEQ ID NO: 629 - protein, 15 aa, SARS-CoV-2
SGWTAGAAAYYVGYL

SEQ ID NO: 630 - protein, 15 aa, SARS-CoV-2
QPRTFLLKYNENGTI

SEQ ID NO: 631 - protein, 15 aa, SARS-CoV-2
NITNLCPFGEVFNAT

SEQ ID NO: 632 - protein, 15 aa, SARS-CoV-2
RFASVYAWNRKRISN

SEQ ID NO: 633 - protein, 15 aa, SARS-CoV-2
CVADYSVLYNSASFS

SEQ ID NO: 634 - protein, 15 aa, SARS-CoV-2
TFKCYGVSPTKLNDL

SEQ ID NO: 635 - protein, 15 aa, SARS-CoV-2
CFTNVYADSFVIRGD

SEQ ID NO: 636 - protein, 15 aa, SARS-CoV-2
EVRQIAPGQTGKIAD

SEQ ID NO: 637 - protein, 15 aa, SARS-CoV-2
YNYKLPDDFTGCVIA

SEQ ID NO: 638 - protein, 15 aa, SARS-CoV-2
WNSNNLDSKVGGNYN

SEQ ID NO: 639 - protein, 15 aa, SARS-CoV-2
YLYRLFRKSNLKPFE

SEQ ID NO: 640 - protein, 15 aa, SARS-CoV-2
RDISTEIYQAGSTPC

SEQ ID NO: 641 - protein, 15 aa, SARS-CoV-2
NGVEGFNCYFPLQSY

SEQ ID NO: 642 - protein, 15 aa, SARS-CoV-2
GFQPTNGVGYQPYRV

SEQ ID NO: 643 - protein, 15 aa, SARS-CoV-2
VVLSFELLHAPATVC

SEQ ID NO: 644 - protein, 15 aa, SARS-CoV-2
GPKKSTNLVKNKCVN

SEQ ID NO: 645 - protein, 15 aa, SARS-CoV-2
FNFNGLTGTGVLTES

SEQ ID NO: 646 - protein, 15 aa, SARS-CoV-2
NKKFLPFQQFGRDIA

SEQ ID NO: 647 - protein, 15 aa, SARS-CoV-2
DTTDAVRDPQTLEIL

SEQ ID NO: 648 - protein, 15 aa, SARS-CoV-2
DITPCSFGGVSVITP

SEQ ID NO: 649 - protein, 15 aa, SARS-CoV-2
GTNTSNQVAVLYQDV

SEQ ID NO: 650 - protein, 15 aa, SARS-CoV-2
NCTEVPVAIHADQLT

SEQ ID NO: 651 - protein, 15 aa, SARS-CoV-2
PTWRVYSTGSNVFQT

SEQ ID NO: 652 - protein, 15 aa, SARS-CoV-2
RAGCLIGAEHVNNSY

SEQ ID NO: 653 - protein, 15 aa, SARS-CoV-2
ECDIPIGAGICASYQ

SEQ ID NO: 654 - protein, 15 aa, SARS-CoV-2
TQTNSPRRARSVASQ

SEQ ID NO: 655 - protein, 15 aa, SARS-CoV-2
SIIAYTMSLGAENSV

SEQ ID NO: 656 - protein, 15 aa, SARS-CoV-2
AYSNNSIAIPTNFTI

SEQ ID NO: 657 - protein, 15 aa, SARS-CoV-2
SVTTEILPVSMTKTS

SEQ ID NO: 658 - protein, 15 aa, SARS-CoV-2
VDCTMYICGDSTECS

SEQ ID NO: 659 - protein, 15 aa, SARS-CoV-2
NLLLQYGSFCTQLNR

SEQ ID NO: 660 - protein, 15 aa, SARS-CoV-2
ALTGIAVEQDKNTQE

SEQ ID NO: 661 - protein, 15 aa, SARS-CoV-2
VFAQVKQIYKTPPIK

SEQ ID NO: 662 - protein, 15 aa, SARS-CoV-2
DFGGFNFSQILPDPS

SEQ ID NO: 663 - protein, 15 aa, SARS-CoV-2
KPSKRSFIEDLLFNK

SEQ ID NO: 664 - protein, 15 aa, SARS-CoV-2
VTLADAGFIKQYGDC

SEQ ID NO: 665 - protein, 15 aa, SARS-CoV-2
LGDIAARDLICAQKF

SEQ ID NO: 666 - protein, 15 aa, SARS-CoV-2
NGLTVLPPLLTDEMI

SEQ ID NO: 667 - protein, 15 aa, SARS-CoV-2
AQYTSALLAGTITSG

SEQ ID NO: 668 - protein, 15 aa, SARS-CoV-2
WTFGAGAALQIPFAM

SEQ ID NO: 669 - protein, 15 aa, SARS-CoV-2
WTFGAGAALQIPFAM

SEQ ID NO: 670 - protein, 15 aa, SARS-CoV-2
LYENQKLIANQFNSA

SEQ ID NO: 671 - protein, 15 aa, SARS-CoV-2
IGKIQDSLSSTASAL

SEQ ID NO: 672 - protein, 15 aa, SARS-CoV-2
GKLQDVVNQNAQALN

SEQ ID NO: 673 - protein, 15 aa, SARS-CoV-2
TLVKQLSSNFGAISS

SEQ ID NO: 674 - protein, 15 aa, SARS-CoV-2
VLNDILSRLDKVEAE

SEQ ID NO: 675 - protein, 15 aa, SARS-CoV-2
VQIDRLITGRLQSLQ

SEQ ID NO: 676 - protein, 15 aa, SARS-CoV-2
TYVTQQLIRAAEIRA

SEQ ID NO: 677 - protein, 15 aa, SARS-CoV-2
SANLAATKMSECVLG

SEQ ID NO: 678 - protein, 15 aa, SARS-CoV-2
QSKRVDFCGKGYHLM

SEQ ID NO: 679 - protein, 15 aa, SARS-CoV-2
SFPQSAPHGVVFLHV

SEQ ID NO: 680 - protein, 15 aa, SARS-CoV-2
TYVPAQEKNFTTAPA

SEQ ID NO: 681 - protein, 15 aa, SARS-CoV-2
ICHDGKAHFPREGVF

SEQ ID NO: 682 - protein, 15 aa, SARS-CoV-2
VSNGTHWFVTQRNFY

SEQ ID NO: 683 - protein, 15 aa, SARS-CoV-2
EPQIITTDNTFVSGN

SEQ ID NO: 684 - protein, 15 aa, SARS-CoV-2
CDVVIGIVNNTVYDP

SEQ ID NO: 685 - protein, 15 aa, SARS-CoV-2
LQPELDSFKEELDKY

SEQ ID NO: 686 - protein, 15 aa, SARS-CoV-2
FKNHTSPDVDLGDIS

SEQ ID NO: 687 - protein, 15 aa, SARS-CoV-2
GINASVVNIQKEIDR

SEQ ID NO: 688 - protein, 15 aa, SARS-CoV-2
LNEVAKNLNESLIDL

SEQ ID NO: 689 - protein, 15 aa, SARS-CoV-2
QELGKYEQYIKWPWY

SEQ ID NO: 690 - protein, 15 aa, SARS-CoV-2
IWLGFIAGLIAIVMV

SEQ ID NO: 691 - protein, 15 aa, SARS-CoV-2
TIMLCCMTSCCSCLK

SEQ ID NO: 692 - protein, 15 aa, SARS-CoV-2
GCCSCGSCCKFDEDD

SEQ ID NO: 693 - protein, 13 aa, SARS-CoV-2
SEPVLKGVKLHYT

SEQ ID NO: 694 - protein, 15 aa, SARS-CoV-2
MSDNGPQNQRNAPRI

SEQ ID NO: 695 - protein, 15 aa, SARS-CoV-2
TFGGPSDSTGSNQNG

SEQ ID NO: 696 - protein, 15 aa, SARS-CoV-2
ERSGARSKQRRPQGL

SEQ ID NO: 697 - protein, 15 aa, SARS-CoV-2
PNNTASWFTALTQHG

SEQ ID NO: 698 - protein, 15 aa, SARS-CoV-2
KEDLKFPRGQGVPIN

SEQ ID NO: 699 - protein, 15 aa, SARS-CoV-2
TNSSPDDQIGYYRRA

SEQ ID NO: 700 - protein, 15 aa, SARS-CoV-2
TRRIRGGDGKMKDLS

SEQ ID NO: 701 - protein, 15 aa, SARS-CoV-2
PRWYFYYLGTGPEAG

SEQ ID NO: 702 - protein, 15 aa, SARS-CoV-2
LPYGANKDGIIWVAT

SEQ ID NO: 703 - protein, 15 aa, SARS-CoV-2
EGALNTPKDHIGTRN

SEQ ID NO: 704 - protein, 15 aa, SARS-CoV-2
PANNAAIVLQLPQGT

SEQ ID NO: 705 - protein, 15 aa, SARS-CoV-2
TLPKGFYAEGSRGGS

SEQ ID NO: 706 - protein, 15 aa, SARS-CoV-2
QASSRSSSRSRNSSR

SEQ ID NO: 707 - protein, 15 aa, SARS-CoV-2
NSTPGSSRGTSPARM

SEQ ID NO: 708 - protein, 15 aa, SARS-CoV-2
AGNGGDAALALLLLD

SEQ ID NO: 709 - protein, 15 aa, SARS-CoV-2
RLNQLESKMSGKGQQ

SEQ ID NO: 710 - protein, 15 aa, SARS-CoV-2
QQGQTVTKKSAAEAS

SEQ ID NO: 711 - protein, 15 aa, SARS-CoV-2
KKPRQKRTATKAYNV

SEQ ID NO: 712 - protein, 15 aa, SARS-CoV-2
TQAFGRRGPEQTQGN

SEQ ID NO: 713 - protein, 15 aa, SARS-CoV-2
FGDQELIRQGTDYKH

SEQ ID NO: 714 - protein, 15 aa, SARS-CoV-2
WPQIAQFAPSASAFF

SEQ ID NO: 715 - protein, 15 aa, SARS-CoV-2
GMSRIGMEVTPSGTW

SEQ ID NO: 716 - protein, 15 aa, SARS-CoV-2
LTYTGAIKLDDKDPN

SEQ ID NO: 717 - protein, 15 aa, SARS-CoV-2
FKDQVILLNKHIDAY

SEQ ID NO: 718 - protein, 15 aa, SARS-CoV-2
KTFPPTEPKKDKKKK

SEQ ID NO: 719 - protein, 15 aa, SARS-CoV-2
ADETQALPQRQKKQQ

SEQ ID NO: 720 - protein, 15 aa, SARS-CoV-2
TVTLLPAADLDDFSK

SEQ ID NO: 721 - protein, 14 aa, SARS-CoV-2
QLQQSMSSADSTQA

SEQ ID NO: 722 - protein, 15 aa, SARS-CoV-2
MADSNGTITVEELKK

SEQ ID NO: 723 - protein, 15 aa, SARS-CoV-2
ITVEELKKLLEQWNL

SEQ ID NO: 724 - protein, 15 aa, SARS-CoV-2
KKLLEQWNLVIGFLF

SEQ ID NO: 725 - protein, 15 aa, SARS-CoV-2
EQWNLVIGFLFLTWI

SEQ ID NO: 726 - protein, 15 aa, SARS-CoV-2
LLESELVIGAVILRG

SEQ ID NO: 727 - protein, 15 aa, SARS-CoV-2
IGAVILRGHLRIAGH

SEQ ID NO: 728 - protein, 15 aa, SARS-CoV-2
HLRIAGHHLGRCDIK

SEQ ID NO: 729 - protein, 15 aa, SARS-CoV-2
MKFLVFLGIITTVAA

SEQ ID NO: 730 - protein, 15 aa, SARS-CoV-2
GIITTVAAFHQECSL

SEQ ID NO: 731 - protein, 15 aa, SARS-CoV-2
AFHQECSLQSCTQHQ

SEQ ID NO: 732 - protein, 15 aa, SARS-CoV-2
LQSCTQHQPYVVDDP

SEQ ID NO: 733 - protein, 15 aa, SARS-CoV-2
QPYVVDDPCPIHFYS

SEQ ID NO: 734 - protein, 15 aa, SARS-CoV-2
PCPIHFYSKWYIRVG

SEQ ID NO: 735 - protein, 15 aa, SARS-CoV-2
SKWYIRVGARKSAPL

SEQ ID NO: 736 - protein, 15 aa, SARS-CoV-2
GARKSAPLIELCVDE

SEQ ID NO: 737 - protein, 15 aa, SARS-CoV-2
LIELCVDEAGSKSPI

SEQ ID NO: 738 - protein, 15 aa, SARS-CoV-2
EAGSKSPIQYIDIGN

SEQ ID NO: 739 - protein, 15 aa, SARS-CoV-2
IQYIDIGNYTVSCLP

SEQ ID NO: 740 - protein, 15 aa, SARS-CoV-2
NYTVSCLPFTINCQE

SEQ ID NO: 741 - protein, 15 aa, SARS-CoV-2
PFTINCQEPKLGSLV

SEQ ID NO: 742 - protein, 15 aa, SARS-CoV-2
EPKLGSLVVRCSFYE

SEQ ID NO: 743 - protein, 15 aa, SARS-CoV-2
VVRCSFYEDFLEYHD

SEQ ID NO: 744 - protein, 15 aa, SARS-CoV-2
EDFLEYHDVRVVLDF

SEQ ID NO: 745 - protein, 9 aa, SARS-CoV-2
DVRVVLDFI

SEQ ID NO: 746 - protein, 15 aa, SARS-CoV-2
MGYINVFAFPFTIYS

SEQ ID NO: 747 - protein, 15 aa, SARS-CoV-2
AFPFTIYSLLLCRMN

SEQ ID NO: 748 - protein, 15 aa, SARS-CoV-2
SLLLCRMNSRNYIAQ

SEQ ID NO: 749 - protein, 15 aa, SARS-CoV-2
NSRNYIAQVDVVNFN

SEQ ID NO: 750 - protein, 10 aa, SARS-CoV-2
QVDVVNFNLT

SEQ ID NO: 751 - protein, 15 aa, SARS-CoV-2
GNYTVSCSPFTINCQ

SEQ ID NO: 752 - protein, 15 aa, SARS-CoV-2
GNYTVSCLPFTINCQ

SEQ ID NO: 753 - protein, 15 aa, SARS-CoV-2
VQIHTIDGSSGVVNP

SEQ ID NO: 754 - protein, 15 aa, SARS-CoV-2
VQIHTIDVSSGVVNP

SEQ ID NO: 755 - protein, 9 aa, SARS-CoV-2
RTQLPPAYT

SEQ ID NO: 756 - protein, 9 aa, Homo sapiens
RTQSPPVYT

SEQ ID NO: 757 - protein, 9 aa, SARS-CoV-2
TQLPPAYTN

SEQ ID NO: 758 - protein, 9 aa, Homo sapiens
TQTPPAYIN

SEQ ID NO: 759 - protein, 9 aa, SARS-CoV-2
LVRDLPQGF

SEQ ID NO: 760 - protein, 9 aa, Homo sapiens
LVQDLAQGF

SEQ ID NO: 761 - protein, 10 aa, SARS-CoV-2
LVRDLPQGFS

SEQ ID NO: 762 - protein, 10 aa, Homo sapiens
LVRDLVTGFS

SEQ ID NO: 763 - protein, 9 aa, SARS-CoV-2
KSFTVEKGI

SEQ ID NO: 764 - protein, 9 aa, Homo sapiens
KTFTVQKGI

SEQ ID NO: 765 - protein, 9 aa, SARS-CoV-2
RQIAPGQTG

SEQ ID NO: 766 - protein, 9 aa, Homo sapiens
REIAPGLTG

SEQ ID NO: 767 - protein, 9 aa, SARS-CoV-2
EVRQIAPGQ

SEQ ID NO: 768 - protein, 9 aa, Homo sapiens
ELRQGAPGQ

SEQ ID NO: 769 - protein, 9 aa, SARS-CoV-2
LDSKVGGNY

SEQ ID NO: 770 - protein, 9 aa, Homo sapiens
LKSKHGGNY

SEQ ID NO: 771 - protein, 9 aa, SARS-CoV-2
YRLFRKSNL

SEQ ID NO: 772 - protein, 9 aa, Homo sapiens
YMLFRKYNL

SEQ ID NO: 773 - protein, 9 aa, SARS-CoV-2
LYRLFRKSN

SEQ ID NO: 774 - protein, 9 aa, Homo sapiens
LWRLFRKKN

SEQ ID NO: 775 - protein, 9 aa, Homo sapiens
LYNLFTKSN

SEQ ID NO: 776 - protein, 9 aa, Homo sapiens
LYRDFRKEN

SEQ ID NO: 777 - protein, 9 aa, SARS-CoV-2
STEIYQAGS

SEQ ID NO: 778 - protein, 9 aa, Homo sapiens
STQIYQAVS

SEQ ID NO: 779 - protein, 9 aa, synthetic construct
  REGION 1..9: note = SARS-CoV-2 mimicry
LVDDLPQGF

SEQ ID NO: 780 - protein, 9 aa, synthetic construct
  REGION 1..9: note = SARS-CoV-2 mimicry
LPRDLPQGF

SEQ ID NO: 781 - protein, 9 aa, synthetic construct
  REGION 1..9: note = SARS-CoV-2 mimicry
LRRDLPQGF

SEQ ID NO: 782 - protein, 9 aa, synthetic construct
  REGION 1..9: note = SARS-CoV-2 mimicry
LVDGLPQGF

SEQ ID NO: 783 - protein, 9 aa, synthetic construct
  REGION 1..9: note = SARS-CoV-2 mimicry
LPRGLPQGF

SEQ ID NO: 784 - protein, 9 aa, synthetic construct
  REGION 1..9: note = SARS-CoV-2 mimicry
LRRGLPQGF

SEQ ID NO: 785 - protein, 9 aa, synthetic construct
  REGION 1..9: note = SARS-CoV-2 mimicry
LVQDLAQGY

SEQ ID NO: 786 - protein, 9 aa, synthetic construct
  REGION 1..9: note = SARS-CoV-2 mimicry
LRQDLAQGF

SEQ ID NO: 787 - protein, 10 aa, synthetic construct
  REGION 1..10: note = SARS-CoV-2 mimicry
LVRDLVTGFY

SEQ ID NO: 788 - protein, 9 aa, synthetic construct
  REGION 1..9: note = SARS-CoV-2 mimicry
LPRDLVTGF

SEQ ID NO: 789 - protein, 9 aa, synthetic construct
  REGION 1..9: note = SARS-CoV-2 mimicry
LRRDLVTGF

SEQ ID NO: 790 - protein, 9 aa, synthetic construct
  REGION 1..9: note = SARS-CoV-2 mimicry
LTSKVGGNY

SEQ ID NO: 791 - protein, 9 aa, synthetic construct
  REGION 1..9: note = SARS-CoV-2 mimicry
LPSKVGGNY

SEQ ID NO: 792 - protein, 9 aa, synthetic construct
  REGION 1..9: note = SARS-CoV-2 mimicry
LRSKVGGNY

SEQ ID NO: 793 - protein, 9 aa, synthetic construct
  REGION 1..9: note = SARS-CoV-2 mimicry
LTSKHGGNY

SEQ ID NO: 794 - protein, 9 aa, synthetic construct
  REGION 1..9: note = SARS-CoV-2 mimicry
LKSKHGGNY

SEQ ID NO: 795 - protein, 9 aa, synthetic construct
  REGION 1..9: note = SARS-CoV-2 mimicry
LRSKHGGNY

SEQ ID NO: 796 - protein, 9 aa, Mycobacterium tuberculosis
ELNNALQNL

SEQ ID NO: 797 - protein, 13 aa, Homo sapiens
LEEKKGNYVVTDH

SEQ ID NO: 798 - protein, 15 aa, Influenza virus
VEPGDKITFEATGNL

SEQ ID NO: 799 - protein, 15 aa, synthetic construct
  REGION 1..15: note = Cancer mutation
LAFQPPISNHPAPEY

SEQ ID NO: 800 - protein, 9 aa, Homo sapiens
AAGIGILTV

SEQ ID NO: 801 - protein, 10 aa, Homo sapiens
ELAGIGILTV

SEQ ID NO: 802 - protein, 15 aa, Homo sapiens
TLGEFLKLDRERAKN

SEQ ID NO: 803 - protein, 15 aa, Homo sapiens
ARGPERSLLEFYLAM

SEQ ID NO: 804 - protein, 15 aa, Homo sapiens
EFYLAMPFATPMEAE

SEQ ID NO: 805 - protein, 15 aa, Homo sapiens
SLLMWITQCFLPVFL

SEQ ID NO: 806 - protein, 15 aa, Homo sapiens
MALPTARPLLGSCGT

SEQ ID NO: 807 - protein, 15 aa, Homo sapiens
HRLSEPPEDLDALPL

SEQ ID NO: 808 - protein, 15 aa, Homo sapiens
LLPRGAPERQRLLPA

SEQ ID NO: 809 - protein, 15 aa, Homo sapiens
ALACWGVRGSLLSEA

SEQ ID NO: 810 - protein, 15 aa, Homo sapiens
FVAESAEVLLPRLVS

SEQ ID NO: 811 - protein, 15 aa, Homo sapiens
SVSTMDALRGLLPVL

SEQ ID NO: 812 - protein, 15 aa, Homo sapiens
LQGGIPNGYLVLDLS

SEQ ID NO: 813 - protein, 15 aa, Homo sapiens
MQEALSGTPCLLGPG

SEQ ID NO: 814 - protein, 15 aa, Homo sapiens
YEQLDVLKHKLDELY

SEQ ID NO: 815 - protein, 15 aa, synthetic construct
  REGION 1..15: note = Cancer mutation
WAVRPQDLDTCDPRQ

SEQ ID NO: 816 - protein, 15 aa, synthetic construct
  REGION 1..15: note = Cancer mutation
LDVLYPKARLAFQNM

SEQ ID NO: 817 - protein, 9 aa, synthetic construct
  REGION 1..9: note = Cancer mutation
MGKSTHTSM

SEQ ID NO: 818 - protein, 9 aa, synthetic construct
  REGION 1..9: note = Cancer mutation
VMKHLLSPL

SEQ ID NO: 819 - protein, 9 aa, Homo sapiens
TTSPSNTLV

SEQ ID NO: 820 - protein, 9 aa, Homo sapiens
EASSAVPTV

SEQ ID NO: 821 - protein, 9 aa, Homo sapiens
MGNSTHTSM

SEQ ID NO: 822 - protein, 9 aa, Homo sapiens
VMQHLLSPL

SEQ ID NO: 823 - protein, 9 aa, synthetic construct
  REGION 1..9: note = Cancer mutation
TTSPSTTLV

SEQ ID NO: 824 - protein, 9 aa, synthetic construct
  REGION 1..9: note = Cancer mutation
EASSTVPTV

SEQ ID NO: 825 - protein, 15 aa, synthetic construct
  REGION 1..15: note = Cancer mutation
SKSLSMGKSTHTSMT

SEQ ID NO: 826 - protein, 15 aa, synthetic construct
  REGION 1..15: note = Cancer mutation
NITDNVMKHLLSPLE

SEQ ID NO: 827 - protein, 15 aa, Homo sapiens
LTTTSPSNTLVSEET

SEQ ID NO: 828 - protein, 15 aa, Homo sapiens
HGAEASSAVPTVSPE

SEQ ID NO: 829 - protein, 15 aa, Homo sapiens
SKSLSMGNSTHTSMT

SEQ ID NO: 830 - protein, 15 aa, Homo sapiens
NITDNVMQHLLSPLE

SEQ ID NO: 831 - protein, 15 aa, Homo sapiens
LTTTSPSTTLVSEET

SEQ ID NO: 832 - protein, 15 aa, Homo sapiens
HGAEASSTVPTVSPE